(19) United States
(12) Patent Application Publication
(10) Pub. No.: US 2023/0303569 A1
(43) Pub. Date: Sep. 28, 2023

(54) HETEROCYCLIC COMPOUNDS AS THERAPEUTIC AGENTS

(71) Applicant: Arisan Therapeutics Inc., Carlsbad, CA (US)

(72) Inventors: Nadezda Sokolova, San Diego, CA (US); Kenneth McCormack, Oceanside, CA (US); Gregory Henkel, Carlsbad, CA (US); Vidyasagar Reddy Gantla, San Diego, CA (US); Eric Brown, Santee, CA (US)

(73) Assignee: Arisan Therapeutics Inc., Carlsbad, CA (US)

(21) Appl. No.: 18/316,004

(22) Filed: May 11, 2023

Related U.S. Application Data
(63) Continuation-in-part of application No. PCT/US2021/059268, filed on Nov. 12, 2021.

(51) Int. Cl. C07D 471/04

(52) U.S. Cl. CPC C07D 471/04

(57) ABSTRACT

The invention relates to compounds of structural formula 1

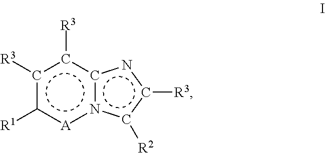

wherein A is independently N or C—R³, R¹ is selected from (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₂ to C₆) heterocycloalkyl, (C₆ to C₁₀) aryl, and (C₂ to C₉) heteroaryl, wherein each of the said (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₂ to C₉) heterocycloalkyl, (C₆ to C₁₀) aryl, and (C₂ to C₉) heteroaryl is optionally substituted with at least one R⁴ group, and wherein said (C₂ to C₉) heteroaryl is C-attached, and R² is selected from the group consisting of

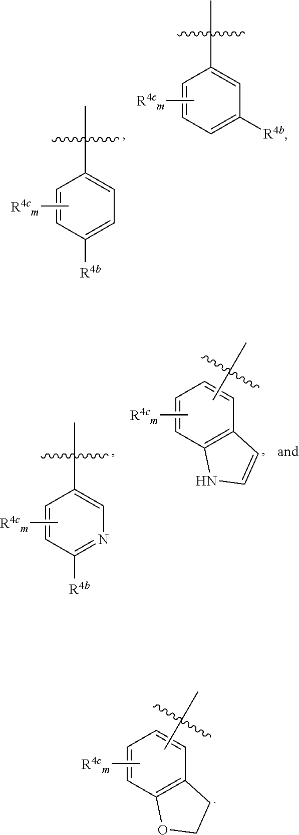

### CROSS REFERENCES TO RELATED APPLICATIONS

This patent application is a continuation-in-part of and claims the benefit of priority to United States Provisional Patent Application Ser. Nos. 63/114,980, filed Nov. 17, 2020 and 63/182,618, filed Apr. 30, 2021 and is a continuation-in-part of PCT application PCT/US2021/059268, filed Nov. 12, 2021, all applications are herein incorporated by reference in their entirety and for all purposes.

### STATEMENT AS TO RIGHTS TO INVENTIONS MADE UNDER FEDERALLY SPONSORED RESEARCH AND DEVELOPMENT

This invention was made with government support under R44 AI112097 awarded by U.S. National Institutes of Health. The government has certain rights in the invention.

### REFERENCE TO A “SEQUENCE LISTING, A TABLE, OR A COMPUTER PROGRAM LISTING APPENDIX SUBMITTED ON A COMPACT DISK

NOT APPLICABLE

### FIELD OF THE INVENTION

The present invention relates to methods of modulating voltage-gated sodium channels in humans, other mammals, or in cell culture, to methods of treating diseases or conditions associated with pathophysiological voltage-gated sodium channel activity or other pathophysiological disorders of ion homeostasis that may be influenced by voltage-gated sodium channel activity, including cardiovascular diseases, neurological disorders and other diseases or conditions associated with or influenced by voltage-gated sodium channels.

### BACKGROUND OF THE INVENTION

Voltage-gated sodium channels (VGSCs or Na_{V} channels) are trans-membrane proteins that play a fundamental role in controlling cellular excitability. VGSCs are responsible for the initiation and propagation of action potentials in excitable cells including myocytes of muscle and neurons of the central and peripheral nervous system. VGSCs play a key role in a range of diseases including cardiovascular and neurological disorders [George, A. L., Jr., Inherited disorders of voltage-gated sodium channels, J. Clin. Invest. (2005); 115(8): 1990-1999], and thus, targeting VGSCs can be used for the treatment of such conditions, including arrhythmia [Remme, C. A. and Bezzina, C. R., Sodium channel (dys)function and cardiac arrhythmias, Cardiovasc. Ther. (2010), 28(5): 287-294], epilepsy [Yogeeswari, P. et al., Ion Channels as Important Targets for Antiepileptic Drug Design, Curr. Drug Targets (2004), 5(7): 589-602; Mantegazza, M. et al., Voltage-gated sodium channels as therapeutic targets in epilepsy and other neurological disorders, Lancet Neurol. (2010), 9: 413-424; Brodie, M. J., Sodium Channel Blockers in the Treatment of Epilepsy, CNS Drugs (2017), 31(7): 527-534], pain, including acute, chronic, inflammatory, neuropathic, and other types of pain [Kyle, D. J. and Ilyin, V. I., Sodium Channel Blockers, J. Med. Chem. (2007), 50:2583-2588; Wood, J. N. et al., Voltage-gated sodium channels and pain pathways, J. Neurobiol. (2004), 61(1):55-71; Bhattacharya, A. Sodium channel blockers for the treatment of neuropathic pain, Neurotherapeutics (2009), 6(4): 663-678], myotonia and periodic paralysis [Cannon, S. C., Spectrum of sodium channel disturbances in the nondystrophic myotonias and periodic paralyses, Kidney Int. (2000), 57(3): 772-779], multiple sclerosis [Black, J. A. et al., Sensory neuron-specific sodium channel SNS is abnormally expressed in the brains of mice with experimental allergic encephalomyelitis and humans with multiple sclerosis, Proc. Natl. Acad. Sci. USA. (2000); 97(21): 11598-11602], irritable bowel syndrome [Beyder, A. et al., Loss-of-function of the Voltage-gated Sodium Channel NaV1.5 (Channelopathies) in Patients with Irritable Bowel Syndrome, Gastroenterology (2014), 146(7): 1659-1668], cognitive dysfunctions associated with Alzheimer's disease and schizophrenia [Jensen, H. S et al., Therapeutic potential of Nav1.1 activators, Trends Pharmacol. Sci. (2014), 35(3): 113-118], and psychiatric disorders, including autism, cerebellar atrophy, ataxia, and mental retardation [Chahine, M. et al., Voltage-gated sodium channel in neurological disorders, CNS Neurol. Disord. Drug Targets (2008), 7(2):144-158; Eijkelkamp, N., Neurological perspectives on voltage-gated sodium channels, Brain (2012), 135: 2585-2612].

VGSC are composed of a highly processed a subunit (230-260 kDa) associated with one or more auxiliary β subunits (30-40 kDa) [Catterall, W. A., From Ionic Currents to Molecular Mechanisms: The Structure and Function of Voltage-Gated Sodium Channels, Neuron (2000), 26: 13-25]. The pore-forming α subunit is essential for the sodium channel function—channel opening, ion selectivity and rapid inactivation, but the kinetics and voltage dependence of the channel gating are modified by the β subunits.

At the resting membrane potential, the majority of sodium channels are in their closed state. Membrane depolarization facilitates a conformational change of the a subunit, resulting in the opening of the Na⁺-selective channel pore. Opening of sodium channels mediate the inward current of Na⁺ that is responsible for the upstroke of the action potential (AP) in nerve and muscle cells. Within milliseconds of opening, VGSCs transition into an inactivated state, facilitating repolarization to the resting potential.

Sodium channel inactivation involves fast and slow kinetic components. The current associated with the slow inactivating component has been referred to as late or persistent Na+ current (I_{Na,late}). An enhanced I_{Na,late} was shown to play an important pathophysiological role in neurological and cardiac conditions [see, for example, Stafstrom, C. E., Persistent Sodium Current and Its Role in Epilepsy, Epilepsy Curr. (2007); 7(1): 15-22; Zaza, A., Pathophysiology and pharmacology of the cardiac “late sodium current”, Pharmacol. Ther. (2008), 119(3): 326-39].

The family of voltage-gated sodium channels comprises nine homologous Na_{V}1.x channel subtypes Nav1.1-Nav1.9 encoded by the genes SCN1A-SCN5A and SCN8A-SCN11A. The Na_{X} channel (also known as Na_{V}2 and NaG) encoded by the SCN7A gene has been identified and classified as a subfamily of VGSC, but it is activated by changes in the extracellular sodium concentration rather than membrane depolarization [Watanabe, E. et al., Nav2/NaG channel is involved in control salt-intake behavior in the CNS, J. Neurosci. (2000), 20(20): 7743-7751; Hiyama, T. Y. et al., Na_{X} channel involved in CNS sodium-level sensing, Nature Neuroscience (2002), 5: 511-512]. These subtypes show tissue specific expression profiles and functional differences [Goldin, A. L., Resurgence of sodium channel research, Annu. Rev. Physiol. (2001), 63: 871-894]. Nav1.1, Nav1.2, Nav1.3, and Nav1.6 are found in both the central and peripheral nervous systems. Nav1.7, Na_{V}1.8, and Na_{V}1.9 are primarily expressed in the peripheral nervous system. Na_{V}1.4 and Na_{V}1.5 isoform are abundant in skeletal and cardiac muscles, respectively. According to their sensitivity to the guanidine-based neurotoxin tetrodotoxin (TTX), a well-known sodium channel blocker, these voltage-gated sodium channels are classified as TTX-sensitive channels (Nav1.1, Nav1.2, Nav1.3, Nav1.4, Nav1.6, and Nav1.7) and TTX-resistant channels (Nav1.5, Nav1.8, and Nav1.9).

Mutations in SCN1A (Na_{V}1.1) and SCN2A (Na_{V}1.2) have been linked to epilepsy conditions, including generalized epilepsy with febrile seizures plus (GEFS+), Dravet's syndrome, and intractable childhood epilepsy with generalized tonic-clonic seizures [Escayg, A. et al., Mutations of SCN1A, encoding a neuronal sodium channel, in two families with GEFS+2, Nat. Genet. (2000), 24:343-345; Sugawara, T. et al., A missense mutation of the Na+ channel αII subunit gene Nav1.2 in a patient with febrile and afebrile seizures causes channel dysfunction, Proc. Natl. Acad. Sci. USA. (2001), 98:6384-6389; Mulley, J. C. et al., SCN1A mutations and epilepsy, Human Mutation (2005); 25:535-542; Fujiwara, T., Clinical spectrum of mutations in SCN1A gene: severe myoclonic epilepsy in infancy and related epilepsies, Epilepsy Res. (2006); 70: S223-S230]. Na_{V}1.1 mutations are also associated with familial hemiplegic migraine [Dichgans, M. et al., Mutation in the neuronal voltage-gated sodium channel SCN1A in familial hemiplegic migraine, Lancet (2005), 366: 371-7]. Na_{V}1.1 channel activators were proposed for the potential symptomatic treatment of cognitive dysfunctions associated with Alzheimer's disease and schizophrenia [Jensen, H. S et al., Therapeutic potential of Nav1.1 activators, Trends Pharmacol. Sci. (2014), 35(3): 113-118].

Nav1.3 is known to be highly expressed in embryonic brain [Cheah, C. S. et al., Correlations in timing of sodium channel expression, epilepsy, and sudden death in Dravet syndrome, Channels. (2013), 7(6):4 68-472]. Mutations in SCN3A (Na_{V}1.3) have been associated with some forms of epilepsy, including cryptogenic paediatric partial epilepsy [Holland, K. D. et al., Mutation of sodium channel SCN3A in a patient with cryptogenic pediatric partial epilepsy, Neurosci. Lett. (2008), 433: 65-70], focal epilepsy [Vanoye, C. G. et al., Neurobiology of Disease Novel SCN3A variants associated with focal epilepsy in children, Neurobiol. Dis. (2014), 62:313-322], and early infantile epileptic encephalopathy [Zaman, T., Mutations in SCN3A cause early infantile epileptic encephalopathy, Ann Neurol. (2018), 83(4): 703-717].

Mutations in SCN1A, SCN2A, and SCN3A have been also associated with autism disorders [Weiss, L. A. et al., Sodium channels SCN1A, SCN2A and SCN3A in familial autism, Mol. Psychiatry (2003); 8: 186-94; O'Roak, B. J. et al., Exome sequencing in sporadic autism spectrum disorders identifies severe de novo mutations, Nat. Genet. (2011); 43: 585-589; O'Roak, B. J. et al., Sporadic autism exomes reveal a highly interconnected protein network of de novo mutations, Nature (2012); 485: 246-250; Sanders, S. J. et al., De novo mutations revealed by whole-exome sequencing are strongly associated with autism, Nature (2012), 485: 237-241].

Gain-of-function variants in Nav1.4, a skeletal muscle voltage-gated sodium channel, encoded by the gene SCN4A, typically cause myotonia or periodic paralysis [Cannon, S. C., Channelopathies of skeletal muscle excitability, Compr. Physiol. (2015), 5: 761-790]. Loss-of-function variants in Nav1.4 have been reported in patients with congenital myasthenic syndrome [Tsujino, A. et al., Myasthenic syndrome caused by mutation of the SCN4A sodium channel, Proc. Natl. Acad. Sci. USA (2003); 100:7377-7382] and congenital myopathies [Zaharieva, I. T. et al., Loss-of-function mutations in SCN4A cause severe foetal hypokinesia or ‘classical’ congenital myopathy, Brain (2016), 139: 674-691].

Mutations in SCN5A, the human gene coding for Na_{V}1.5, have been associated with a variety of arrhythmia syndromes, including long QT syndrome (type 3), Brugada syndrome, sick sinus syndrome, cardiac conduction disease, atrial fibrillation, atrial standstill, and dilated cardiomyopathy [Wilde, A. A. M. et al., Phenotypical manifestations of mutations in the genes encoding subunits of the cardiac sodium channel, Circ. Res. (2011), 108(7): 884-897]. Blockers of Na_{V}1.5 have been used extensively in treating cardiac arrhythmias [Srivatsa, U. et al., Mechanisms of antiarrhythmic drug actions and their clinical relevance for controlling disorders of cardiac rhythm, Current Cardiology Reports (2002), 4: 401-410; Remme, C. A. and Bezzina, C. R., Sodium Channel (Dys)Function and Cardiac Arrhythmias, Cardiovascular Therapeutics (2010), 28:287-294; Roden, D. M., Pharmacology and Toxicology of Nav1.5-Class 1 anti-arrhythmic drugs, Card. Electrophysiol. Clin. (2014), 6(4): 695-704].

Mutations in SCN8A, the human gene coding for Na_{V}1.6, have been linked with epilepsy (including Dravet syndrome and infantile epileptic encephalopathy) and autism spectrum disorders (Estacion, M. et al., A novel de novo mutation of SCN8A (Naᵥ1.6) with enhanced channel activation in a child with epileptic encephalopathy, Neurobiology of Disease (2014), 69: 117-123; Ohba, C. et al., Early onset epileptic encephalopathy caused by de novo SCN8A mutations, Epilepsia (2014), 55: 994-1000; Butler, K. M. et al., De novo and inherited SCN8A epilepsy mutations detected by gene panel analysis, Epilepsy Res (2017), 129:17-25]. The Na_{V}1.6 isoform has been also associated with axonal loss following demyelination in both the experimental autoimmune encephalomyelitis [Craner, M. J., Abnormal sodium channel distribution in optic nerve axons in a model of inflammatory demyelination, Brain (2003), 126:1552-61] and multiple sclerosis [Craner, M. J. et al., Molecular changes in neurons in multiple sclerosis: altered axonal expression of Nav1.2 and Nav1.6 sodium channels and Na⁺/Ca2⁺ exchanger, Proc. Natl. Acad. Sci. (2004), 101: 8168-73].

Voltage-gated sodium channels Na_{V}1.7 (SCN9A), Na_{V}1.8 (SCN10A), and Na_{V}1.9 (SCN11A) are predominantly associated with peripheral neurons and have all been linked to pain disorders, including acute, inflammatory, and neuropathic pain [Akopian, A. N. et al., The tetrodotoxin-resistant sodium channel SNS has a specialized function in pain pathways, Nat. Neurosci. (1999), 2(6): 541-8; Nassar, M. A. et al., Nociceptor-specific gene deletion reveals a major role for Nav1.7 (PN1) in acute and inflammatory pain, Proc. Natl. Acad. Sci. USA (2004), 101(34): 12706-12711; Yang, Y. et al., Mutations in SCN9A, encoding a sodium channel alpha subunit, in patients with primary erythermalgia, J. Med. Genet. (2004), 41(3):171-174; Bierhaus, A. et al., Methylglyoxal modification of Nav1.8 facilitates nociceptive neuron firing and causes hyperalgesia in diabetic neuropathy, Nat. Med. (2012); 18(6): 926-933; Dib-Hajj, S. D. et al., Naᵥ1.9: a sodium channel linked to human pain, Nat. Rev. Neurosci. (2015), 16(9): 511-519].

Abnormal upregulation of mRNA and protein for Naᵥ1.8 within Purkinje cells has been found in the brains of mice with experimental autoimmune encephalomyelitis (EAE) and humans with multiple sclerosis (MS) [Black, J. A. et al., Sensory neuron-specific sodium channel SNS is abnormally expressed in the brains of mice with experimental allergic encephalomyelitis and humans with multiple sclerosis, Proc. Natl. Acad. Sci. USA (2000), 97: 11598-11602]. It has been also found that the SCN10A gene encoding Nav1.8 plays a role in some cardiac disorders [Chambers, J. C. et al., Genetic variation in SCN10A influences cardiac conduction, Nat. Genet. (2010), 42: 149-52].

Sodium channel blockers have been shown to be effective in the treatment of a variety of diseases, and have found use as local analgesics, such as bupivacaine and lidocaine, in the treatment of cardiac arrhythmias, such as propafenone, disopyramide, amiodarone, and flecainide [Razavi, M., Safe and Effective Pharmacologic Management of Arrhythmias, Tex. Heart Inst. J. (2005), 32(2): 209-211], and epilepsy, such as phenytoin, carbamazepine, lamotrigine, oxcarbazepine, rufinamide, lacosamide, and eslicarbazepine acetate [Brodie, M. J., Sodium Channel Blockers in the Treatment of Epilepsy, CNS Drugs (2017), 31(7): 527-534]. In addition, local anesthetics, such as lidocaine, anticonvulsants, such as carbamazepine, phenytoin, and lamotrigine, and tricyclic antidepressants that have proven to be effective in reducing pain have been suggested to act as sodium channel inhibitors [Soderpalm, B., Anticonvulsants: aspects of their mechanisms of action, Eur. J. Pain (2002); 6 Suppl A:3-9; Wang, G. K. et al., Block of persistent late Na⁺ currents by antidepressant sertraline and paroxetine, J. Membr. Biol. (2008), 222(2): 79-90; Yang, Y.-C. et al., Lidocaine, Carbamazepine, and Imipramine Have Partially Overlapping Binding Sites and Additive Inhibitory Effect on Neuronal Na⁺ Channels, Anesthesiology (2010), 113: 160-174].

VGSCs have been shown to be abnormally expressed in tumor cells in a variety of different types of cancer, including cancers of the breast, lung, prostate, pancreatic, colon, stomach, ovary, cervix, bladder, oral squamous cell carcinoma, endometrium, connective tissues, skin, astrocytoma, lymphoma, neuroblastoma, mesothelioma, myeloma, hepatocellular carcinoma, leukaemia, and osteosarcoma, where they regulate cancer cell proliferation, migration, invasion and metastasis, suggesting VGSCs may be attractive anti-metastatic targets [Brackenbury, W. J., Voltage-gated sodium channels and metastatic disease, Channels (Austin) (2012), 6 (5): 352-361; Roger, S. L. et al., Voltage-gated sodium channels and cancer: is excitability their primary role?, Front. Pharmacol. (2015), 6; Leslie, T. K. et al., Sodium homeostasis in the tumour microenvironment, Biochim. Biophys. Acta Rev. Cancer (2019), 1872:188304; Patel, F. and Brackenbury, W. J., Dual roles of voltage-gated sodium channels in development and cancer, Int. J. Dev. Biol. (2015), 59(0): 357-366; Djamgoz, M. B. A. et al., In Vivo Evidence for Voltage-Gated Sodium Channel Expression in Carcinomas and Potentiation of Metastasis, Cancers (2019), 11, 1675]. It has been shown that VGSC-inhibiting drugs, including riluzole, sodium valproate, valproic acid, carbamazepine, phenytoin, ranolazine, tetracaine, and lidocaine inhibit various aspects of the hallmarks of cancer, including proliferation, migration, and invasion [Martin, F. et al., Therapeutic value of voltage gated sodium channel inhibitors in breast, colorectal and prostate cancer: a systematic review, Front. Pharmacol. (2015), 6273; Nelson, M. et al., The sodium channel-blocking antiepileptic drug phenytoin inhibits breast tumour growth and metastasis, Mol. Cancer (2015), 14, 13; Capatina, A. et al., Targeting Ion Channels for Cancer Treatment: Current Progress and Future Challenges, Rev. Physiol. Biochem Pharmacol. (2020)]. The above papers are herein incorporated by reference in their entirety for all purposes.

In addition, Ranolazine, a voltage-gated sodium channel modulator, has been approved by the FDA as a drug for the treatment of chronic stable angina pectorus. Similar to a number of pharmaceutical compounds the heterocyclic compounds of the present invention are VGSC modulators that may be useful in treating a number of neurological and cardiac conditions, including the above-mentioned indications.

### BRIEF SUMMARY OF THE INVENTION

The present invention relates to methods of modulating voltage-gated sodium channels in humans, other mammals, or in cell culture, to methods of treating diseases or conditions associated with pathophysiological voltage-gated sodium channel activity or other pathophysiological disorders of ion homeostasis that may be influenced by voltage-gated sodium channel activity, including cardiovascular diseases, neurological disorders and other diseases or conditions associated with or influenced by voltage-gated sodium channels.

In one embodiment, the method comprises administering to humans, other mammals, cell culture, or biological sample a therapeutically effective amount of a compound represented by Structural Formula I

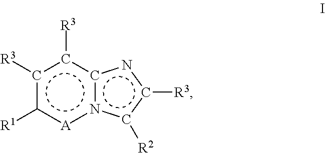

or a pharmaceutically acceptable salt, and a pharmaceutically acceptable carrier, diluent, or vehicle thereof, wherein:
A is independently N or C—R³;
R¹ is selected from halogen, cyano, (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₂ to C₉) heterocycloalkyl, (C₆ to C₁₀) aryl, and (C₂ to C₉) heteroaryl, wherein
each of the said (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₂ to C₉) heterocycloalkyl, (C₆ to C₁₀) aryl, and (C₂ to C₉) heteroaryl is optionally substituted with at least one R⁴ group, and wherein said (C₂ to C₉) heteroaryl is C-attached;
R² is selected from halogen, cyano, (C₂ to C₆) alkenyl, (C₂ to C₆) alkynyl, (C₃ to C₁₀) cycloalkyl, (C₅ to C₁₀) cycloalkenyl, (C₂ to C₉) heterocycloalkyl, (C₆ to C₁₀) aryl, and (C₂ to C₉) heteroaryl, wherein
each of the said (C₂ to C₆) alkenyl, (C₂ to C₆) alkynyl, (C₃ to C₁₀) cycloalkyl, (C₅ to C₁₀) cycloalkenyl, (C₂ to C₉) heterocycloalkyl, (C₆ to C₁₀) aryl, and (C₂ to C₉) is optionally substituted with at least one R⁴ group;
and wherein at least one of R¹ and R² is selected from (C₃ to C₁₀) cycloalkyl, (C₅ to C₁₀) cycloalkenyl, (C₂ to C₉) heterocycloalkyl, (C₆ to C₁₀) aryl, and (C₂ to C₉) heteroaryl;
each of the R³ is independently selected from hydrogen, deuterium, halogen, cyano, —CD₃, OH, CF₃, (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, (C₂ to C₉) heteroaryl, —C(O)R⁵, —C(O)NR^{6a}R^{6b}, —C(O)NR^{6a}S(O)ₘR⁵, —C(O)NR^{6a}S(O)ₘNR^{6a}R^{6b}, —NR^{6a}R^{6b}, —S(O)ₘR⁵, —S(O)ₘNR^{6a}R^{6b}, —NR^{6a}S(O)ₘR⁵, —(CH₂)ₙC(O)OR⁵, —(CH₂)ₙC(O)NR^{6a}R^{6b}, —OC(O)R⁵, —NR⁶C(O)R⁵, and —NR^{6c}C(O)NR^{6a}R^{6b}, wherein
each of the said (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, and (C₂ to C₉) heteroaryl is optionally substituted with at least one R⁴ group;
R⁴ is independently selected from hydrogen, deuterium, halogen, cyano, OH, CF₃, (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, (C₂ to C₉) heteroaryl, —C(O)R⁵, —C(O)NR^{6a}R^{6b}, —C(O)NR^{6a}S(O)ₘR⁵, —C(O)NR^{6a}S(O)ₘNR^{6a}R^{6b}, —NR^{6a}R_{6b}, —S(O)ₘR⁵, —S(O)ₘNR^{6a}R^{6b}, —NR^{6a}S(O)ₘR⁵, —(CH₂)ₙC(O)OR⁵, —(CH₂)ₙC(O)NR^{6a}R^{6b}, —OC(O)R⁵, —NR^{6a}C(O)R⁵, and —NR^{6c}C(O)NR^{6a}R^{6b}, wherein
each of the said (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, and (C₂ to C₉) heteroaryl is optionally substituted with at least one R⁷ group;
each of the R⁵ is independently selected from hydrogen, deuterium, (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl, wherein each of the said (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁷ group;
each of the R^{6a}, R^{6b}, and R^{6c} are independently selected from hydrogen, deuterium, (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl,
or R^{6a} and R^{6b} may be taken together with the nitrogen atom to which they are attached to form a 4 to 8 membered cycloheteroalkyl ring, wherein
said 4 to 8 membered cycloheteroalkyl ring has 1 to 3 ring heteroatoms selected from the group consisting of N, O, and S, and wherein
the said 4 to 8 membered cycloheteroalkyl ring is optionally substituted with at least one R⁷ group;
R⁷ is independently selected from hydrogen, deuterium, halogen, OH, CF₃, (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, (C₂ to C₉) heteroaryl, —C(O)R⁸, —C(O)NR^{9a}R^{9b}, —NR^{9a}R^{9b}, —S(O)ₘR⁸, —S(O)ₘNR^{9a}R^{9b}, —NR^{9a}S(O)ₘR⁸, —(CH₂)ₙC(O)OR⁸, —(CH₂)ₙC(O)N(R^{9a}R^{9b}), —OC(O)R⁸, —NR⁹C(O)R⁸, and —NR^{9a}C(O)N(R^{9a}R^{9b}), wherein
each of the said (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, and (C₂ to C₉) heteroaryl is optionally substituted with at least one R¹⁰ group;
each of the R⁸ is independently selected from hydrogen, deuterium, (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl, wherein
each of the said (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R¹⁰ group;
each of the R^{9a}, R^{9b}, and R^{9c} are independently selected from hydrogen, (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl,
or R^{9a} and R^{9b} may be taken together with the nitrogen atom to which they are attached to form a 4 to 8 membered cycloheteroalkyl ring, wherein
said 4 to 8 membered cycloheteroalkyl ring has 1 to 3 ring heteroatoms selected from the group consisting of N, O, and S, and wherein
the said 4 to 8 membered cycloheteroalkyl ring is optionally substituted with hydrogen, deuterium, halogen, cyano, —CD₃, OH, CF₃, (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, and (C₂ to C₉) heteroaryl;
R¹⁰ is independently selected from hydrogen, deuterium, halogen, cyano, OH, CF₃, (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, and (C₂ to C₉) heteroaryl, wherein
each of the said (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, and (C₂ to C₉) heteroaryl is optionally substituted with hydrogen, deuterium, halogen, cyano, —CD₃, OH, CF₃, (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, and (C₂ to C₉) heteroaryl;
m is 0, 1, 2, 3, or 4;
and n is 0, 1, 2, 3, 4, 5, or 6.

### DETAILED DESCRIPTION OF THE INVENTION

The present invention relates to methods of modulating voltage-gated sodium channels in humans, other mammals, or in cell culture, to methods of treating diseases or conditions associated with pathophysiological voltage-gated sodium channel activity or other pathophysiological disorders of ion homeostasis that may be influenced by voltage-gated sodium channel activity, including cardiovascular diseases, neurological disorders, irritable bowel syndrome, cancer, and other diseases or conditions associated with or influenced by voltage-gated sodium channels.

It was unexpectedly discovered that when some compounds of the invention that were being developed as Arena virus treatments, in particular for Lassa fever, that these compounds were very potent voltage-gated sodium channel modulators and have very interesting pharmacological properties. These compounds of the invention show sodium channel modulation of one or more of the Nav 1.1, 1.2, 1.3, 1.5. 1.6, 1.7 and 1.8 channels.

In one embodiment, the method comprises administering to humans, other mammals, cell culture, or biological sample a therapeutically effective amount of a compound represented by Structural Formula I

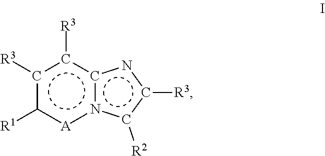

or a pharmaceutically acceptable salt, and a pharmaceutically acceptable carrier, diluent, or vehicle thereof, wherein:
A is independently N or C—R³;
R¹ is selected from halogen, cyano, (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₂ to C₉) heterocycloalkyl, (C₆ to C₁₀) aryl, and (C₂ to C₉) heteroaryl, wherein
each of the said (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₂ to C₉) heterocycloalkyl, (C₆ to C₁₀) aryl, and (C₂ to C₉) heteroaryl is optionally substituted with at least one R⁴ group, and wherein said (C₂ to C₉) heteroaryl is C-attached;
R² is selected from halogen, cyano, (C₂ to C₆) alkenyl, (C₂ to C₆) alkynyl, (C₃ to C₁₀) cycloalkyl, (C₅ to C₁₀) cycloalkenyl, (C₂ to C₉) heterocycloalkyl, (C₆ to C₁₀) aryl, and (C₂ to C₉) heteroaryl, wherein
each of the said (C₂ to C₆) alkenyl, (C₂ to C₆) alkynyl, (C₃ to C₁₀) cycloalkyl, (C₅ to C₁₀) cycloalkenyl, (C₂ to C₉) heterocycloalkyl, (C₆ to C₁₀) aryl, and (C₂ to C₉) heteroaryl is optionally substituted with at least one R⁴ group;
and wherein at least one of R¹ and R² is selected from (C₃ to C₁₀) cycloalkyl, (C₅ to C₁₀) cycloalkenyl, (C₂ to C₉) heterocycloalkyl, (C₆ to C₁₀) aryl, and (C₂ to C₉) heteroaryl;
each of the R³ is independently selected from hydrogen, deuterium, halogen, cyano, —CD₃, OH, CF₃, (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, (C₂ to C₉) heteroaryl, —C(O)R⁵, —C(O)NR^{6a}R^{6b}, —C(O)NR^{6a}S(O)ₘR⁵, —C(O)NR^{6a}S(O)ₘNR^{6a}R^{6b}, —NR^{6a}R^{6b}, —S(O)ₘR⁵, —S(O)ₘNR^{6a}R^{6b}, —NR^{6a}(O)ₘR⁵, —(CH₂)ₙC(O)OR⁵, —(CH₂)ₙC(O)NR^{6a}R^{6b}, —OC(O)R⁵, —NR^{6a}C(O)R⁵, and —NR^{6c}C(O)NR^{6a}R^{6b}, wherein
each of the said (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, and (C₂ to C₉) heteroaryl is optionally substituted with at least one R⁴ group;
R⁴ is independently selected from hydrogen, deuterium, halogen, cyano, OH, CF₃, (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, (C₂ to C₉) heteroaryl, —C(O)R⁵, —C(O)NR^{6a}R^{6b}, —C(O)NR^{6a}S(O)ₘR⁵, —C(O)NR^{6a}S(O)ₘNR^{6a}R^{6b}, —NR^{6a}R^{6b}, —S(O)ₘR⁵, —S(O)ₘNR^{6a}R^{6b}, —NR^{6a}S(O)ₘR⁵, —(CH₂)ₙC(O)OR⁵, —(CH₂)ₙC(O)NR^{6a}R^{6b}, —OC(O)R⁵, —NR^{6a}C(O)R⁵, and —NR^{6c}C(O)NR^{6a}R^{6b}, wherein
each of the said (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, and (C₂ to C₉) heteroaryl is optionally substituted with at least one R⁷ group;
each of the R⁵ is independently selected from hydrogen, deuterium, (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl, wherein
each of the said (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁷ group;
each of the R^{6a}, R^{6b}, and R^{6c} are independently selected from hydrogen, deuterium, (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl,
or R^{6a} and R^{6b} may be taken together with the nitrogen atom to which they are attached to form a 4 to 8 membered cycloheteroalkyl ring, wherein
said 4 to 8 membered cycloheteroalkyl ring has 1 to 3 ring heteroatoms selected from the group consisting of N, O, and S, and wherein
the said 4 to 8 membered cycloheteroalkyl ring is optionally substituted with at least one R⁷ group;
R⁷ is independently selected from hydrogen, deuterium, halogen, OH, CF₃, (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, (C₂ to C₉) heteroaryl, —C(O)R⁸, —C(O)NR^{9a}R^{9b}, —NR^{9a}R^{9b}, —S(O)ₘR⁸, —S(O)ₘNR^{9a}R^{9b}, —NR^{9a}S(O)ₘR⁸, —(CH₂)ₙC(O)OR⁸, —(CH₂)ₙC(O)N(R^{9a}R^{9b}), —OC(O)R⁸, —NR⁹C(O)R⁸, and —NR^{9a}C(O)N(R^{9a}R^{9b}), wherein
each of the said (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, and (C₂ to C₉) heteroaryl is optionally substituted with at least one R¹⁰ group;
each of the R⁸ is independently selected from hydrogen, deuterium, (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl, wherein
each of the said (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R¹⁰ group;
each of the R^{9a}, R^{9b}, and R^{9c} are independently selected from hydrogen, (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl,
or R^{9a} and R^{9b} may be taken together with the nitrogen atom to which they are attached to form a 4 to 8 membered cycloheteroalkyl ring, wherein
said 4 to 8 membered cycloheteroalkyl ring has 1 to 3 ring heteroatoms selected from the group consisting of N, O, and S, and wherein
the said 4 to 8 membered cycloheteroalkyl ring is optionally substituted with hydrogen, deuterium, halogen, cyano, —CD₃, OH, CF₃, (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, and (C₂ to C₉) heteroaryl;
R¹⁰ is independently selected from hydrogen, deuterium, halogen, cyano, OH, CF₃, (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(CG to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, and (C₂ to C₆) heteroaryl, wherein
each of the said (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₆) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, and (C₂ to C₆) heteroaryl is optionally substituted with hydrogen, deuterium, halogen, cyano, —CD₃, OH, CF₃, (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₆) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, and (C₂ to C₉) heteroaryl;
m is 0, 1, 2, 3, or 4;
and n is 0, 1, 2, 3, 4, 5, or 6.

In another embodiment, the method comprises administering to humans, other mammals, cell culture, or biological sample an effective amount of a compound represented by Structural Formula I, or a pharmaceutically acceptable salt, and a pharmaceutically acceptable carrier, diluent, or vehicle thereof, wherein:
A is independently N or C—R³;
R¹ and R² are independently selected from the group consisting of

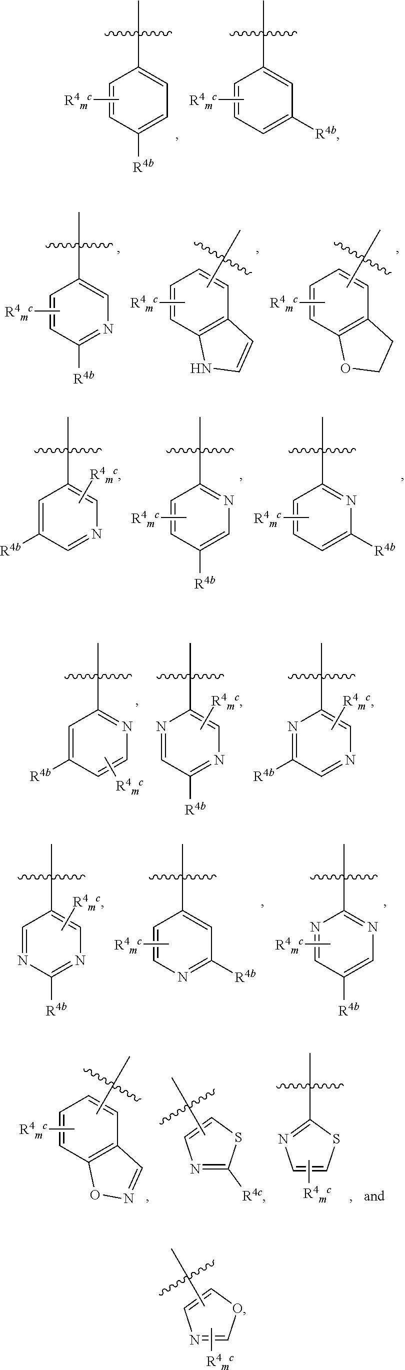

wherein
R^{4b} is selected from (C₁ to C₆) alkyl, (C₂ to C₆) alkenyl, (C₂ to C₆) alkynyl, —O—(C₁ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, and —C(O)R⁵, wherein
each of the said (C₁ to C₆) alkyl, (C₂ to C₆) alkenyl, (C₂ to C₆) alkynyl, —O—(C₁ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁷ group as defined above;
R^{4c} is independently selected from hydrogen, deuterium, halogen, cyano, OH, CF₃, (C₁ to C₆) alkyl, (C₂ to C₆) alkenyl, (C₂ to C₆) alkynyl, —O—(C₁ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, —C(O)R⁵, —NR^{6a}R^{6b}, —S(O)ₘR⁵, —S(O)ₘNR^{6a}R^{6b}, —NR^{6a}S(O)ₘR⁵, —OC(O)R⁵, —NR^{6a}C(O)R⁵, and —NR^{6c}C(O)NR^{6a}R^{6b},
wherein each of the said (C₁ to C₆) alkyl, (C₂ to C₆) alkenyl, (C₂ to C₆) alkynyl, —O—(C₁ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁷ group as defined above;
R³ is selected from hydrogen, deuterium, halogen, cyano, —CD₃, OH, CF₃, (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, (C₂ to C₉) heteroaryl, —C(O)R⁵, —C(O)NR^{6a}R^{6b}, —C(O)NR^{6a}S(O)ₘR⁵, —C(O)NR^{6a}S(O)ₘNR^{6a}R^{6b}, —NR^{6a}R^{6b}, —S(O)ₘR⁵, —S(O)ₘNR^{6a}R^{6b}, —NR^{6a}S(O)ₘR⁵, —(CH₂)ₙC(O)OR⁵, —(CH₂)ₙC(O)NR^{6a}R^{6b}, —OC(O)R⁵, —NR^{6a}C(O)R⁵, and —NR^{6c}C(O)NR^{6a}R^{6b}, wherein
each of the said (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, and (C₂ to C₉) heteroaryl is optionally substituted with at least one R⁴ group as defined above.

In another embodiment, the method comprises administering to humans, other mammals, cell culture, or biological sample an effective amount of a compound represented by Structural Formula I, or a pharmaceutically acceptable salt, and a pharmaceutically acceptable carrier, diluent, or vehicle thereof, wherein:
A is independently N or C—R³;
R¹ and R² are independently selected from the group consisting of

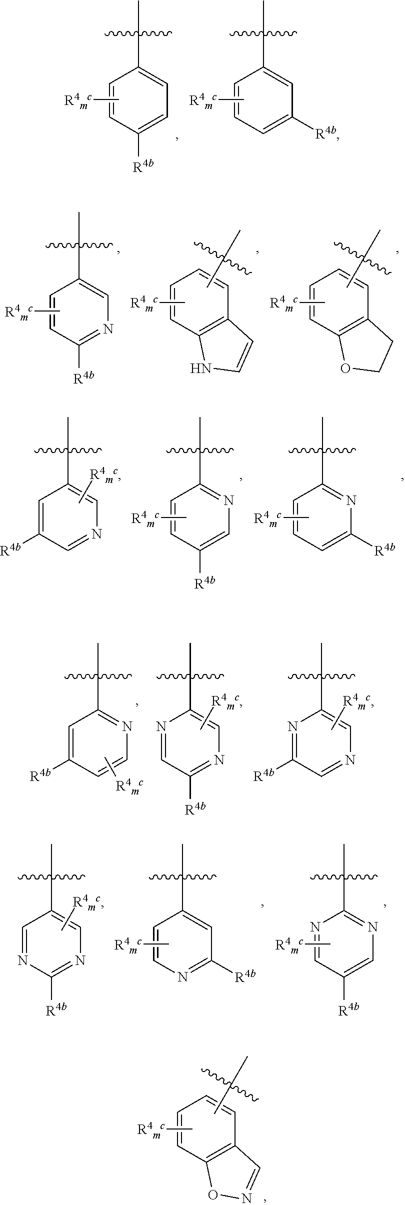

wherein
R^{b} is selected from (C₁ to C₆) alkyl, (C₂ to C₆) alkenyl, (C₂ to C₆) alkynyl, —O—(C₁ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, and —C(O)R⁵, wherein
each of the said (C₁ to C₆) alkyl, (C₂ to C₆) alkenyl, (C₂ to C₆) alkynyl, —O—(C₁ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁷ group as defined above;
R^{4c} is independently selected from hydrogen, deuterium, halogen, cyano, OH, CF₃, (C₁ to C₆) alkyl, (C₂ to C₆) alkenyl, (C₂ to C₆) alkynyl, —O—(C₁ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, —C(O)R⁵, —NR^{6a}R^{6b}, —S(O)ₘR⁵, —S(O)ₘNR^{6a}R^{6b}, —NR^{6a}S(O)ₘR⁵, —OC(O)R⁵, —NR^{6a}C(O)R⁵, and —NR^{6c}C(O)NR^{6a}R^{6b},
wherein each of the said (C₁ to C₆) alkyl, (C₂ to C₆) alkenyl, (C₂ to C₆) alkynyl, —O—(C₁ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁷ group as defined above;
R³ is selected from hydrogen, deuterium, halogen, cyano, —CD₃, OH, CF₃, (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, (C₂ to C₉) heteroaryl, —C(O)R⁵, —C(O)NR^{6a}R^{6b}, —C(O)NR^{6a}S(O)ₘR⁵, —C(O)NR^{6a}S(O)ₘNR^{6a}R^{6b}, —NR^{6a}R^{6b}, —S(O)ₘR⁵, —S(O)ₘNR^{6a}R^{6b}, —NR^{6a}S(O)ₘR⁵, —(CH₂)ₙC(O)OR⁵, —(CH₂)ₙC(O)NR^{6a}R^{6b}, —OC(O)R⁵, —NR^{6a}C(O)R⁵, and —NR^{6c}C(O)NR^{6a}R^{6b}, wherein
each of the said (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, and (C₂ to C₉) heteroaryl is optionally substituted with at least one R⁴ group as defined above.

In another embodiment, the method comprises administering to humans, other mammals, cell culture, or biological sample an effective amount of a compound represented by Structural Formula I, wherein A, R¹, and R³ are defined as above, or a pharmaceutically acceptable salt, and a pharmaceutically acceptable carrier, diluent, or vehicle thereof, wherein:
R² is selected from the group consisting of

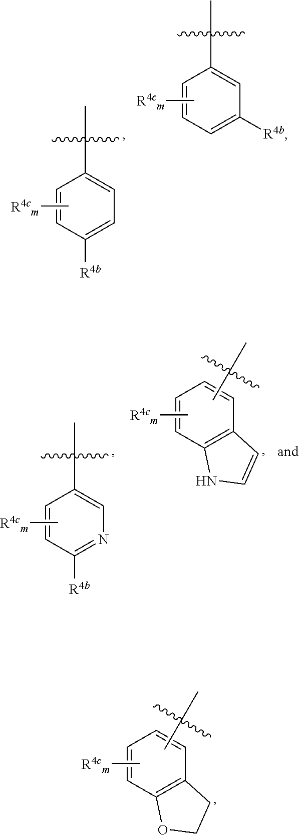

wherein
R^{4b} is selected from (C₁ to C₆) alkyl, (C₂ to C₆) alkenyl, (C₂ to C₆) alkynyl, —O—(C₁ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, and —C(O)R⁵, wherein
each of the said (C₁ to C₆) alkyl, (C₂ to C₆) alkenyl, (C₂ to C₆) alkynyl, —O—(C₁ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁷ group as defined above;
R^{4c} is independently selected from hydrogen, deuterium, halogen, cyano, OH, CF₃, (C₁ to C₆) alkyl, (C₂ to C₆) alkenyl, (C₂ to C₆) alkynyl, —O—(C₁ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, —C(O)R⁵, —NR^{6a}R^{6b}, —S(O)ₘR⁵, —S(O)ₘNR^{6a}R^{6b}, —NR^{6a}S(O)ₘR⁵, —OC(O)R⁵, —NR^{6a}C(O)R⁵, and —NR^{6c}C(O)NR^{6a}R^{6b},
   wherein each of the said (C₁ to C₆) alkyl, (C₂ to C₆) alkenyl, (C₂ to C₆) alkynyl, —O—(C₁ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁷ group as defined above.

In another embodiment, the method comprises administering to humans, other mammals, cell culture, or biological sample an effective amount of a compound represented by Structural Formula I, wherein A, R¹, R² and R³ are defined as above, or a pharmaceutically acceptable salt, and a pharmaceutically acceptable carrier, diluent, or vehicle thereof, wherein
R^{4b} is selected from the group consisting of halogen, methoxy, isopropoxy, cyclopropoxy, tert-butoxy, cyclopropylmethoxy, (2-hydroxypropan)-2-yl, (1-hydroxycyclopropan)-1-yl, (1-hydroxycyclobutan)-1-yl, difluoromethoxy, trifluoromethoxy, trifluoromethyl, trifluoroethoxy, methoxyethoxy, phenoxy, benzyloxy, difluoromethyl, cyclopropyl, acetyl, n-propyloxy, n-butyloxy, isobutyloxy, sec-butyloxy, cyclobutyloxy, cyclopentyloxy, (1-hydroxycyclopentan)-1-yl, hydroxyethoxy, ethoxyethoxy, isopropoxyethoxy, isopropyl, n-propyl, n-butyl, isobutyl, sec-butyl, cyclobutyl, cyclopentyl, tert-butyl, propanoyl, isobutanoyl, sec-butanoyl, tert-butanoyl, cyclopropanoyl, cyclobutanoyl, and cyclopentanoyl.

In another embodiment, the method comprises administering to humans, other mammals, cell culture, or biological sample an effective amount of a compound represented by Structural Formula I, wherein A, R¹, R² and R³ are defined as above, or a pharmaceutically acceptable salt, and a pharmaceutically acceptable carrier, diluent, or vehicle thereof, wherein:
R^{4b} is isopropoxy or tert-butoxy, these compounds surprisingly providing improved metabolic stability in multi-species microsomal assays as shown in the Plewe, M. et al. PCT patent application, publication number PCT/US2017/041218, 7 Jul. 2017 and Brown, E. et al. PCT patent application, publication number PCT/US2019/064223, 3 Dec. 2019, and both references are herein incorporated by reference in its entirety for all purposes.

In another embodiment, the method comprises administering to humans, other mammals, cell culture, or biological sample an effective amount of a compound represented by Structural Formula I, wherein A, R¹, R² and R³ are defined as above, or a pharmaceutically acceptable salt, and a pharmaceutically acceptable carrier, diluent, or vehicle thereof, wherein
R^{4b} is isopropoxy, tert-butoxy, phenoxy, isopropyl, or tert-butyl.

In another embodiment, the method comprises administering to humans, other mammals, cell culture, or biological sample an effective amount of a compound represented by Structural Formula I, wherein A, R² and R³ are defined as above, or a pharmaceutically acceptable salt, and a pharmaceutically acceptable carrier, diluent, or vehicle thereof, wherein R¹ is

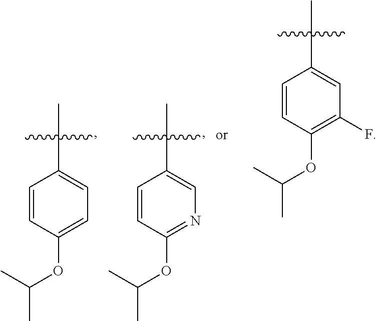 [SMILES: *c1ccc(OC(C)C)cc1 ; *c1ccc(OC(C)C)nc1 ; *c1ccc(OC(C)C)c(F)c1]

In another embodiment, the method comprises administering to humans, other mammals, cell culture, or biological sample an effective amount of a compound represented by Structural Formula B

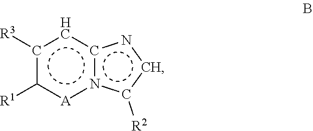

wherein A, R¹, and R² are defined as above, and R³ is hydrogen, deuterium, CH₃ or CD₃, or a pharmaceutically acceptable salt, and a pharmaceutically acceptable carrier, diluent, or vehicle thereof.

In another embodiment, the method comprises administering to humans, other mammals, cell culture, or biological sample an effective amount of a compound represented by Structural Formula I, wherein A, R², and R³ are defined as above, or a pharmaceutically acceptable salt, and a pharmaceutically acceptable carrier, diluent, or vehicle thereof, wherein:
R¹ is selected from the group consisting of

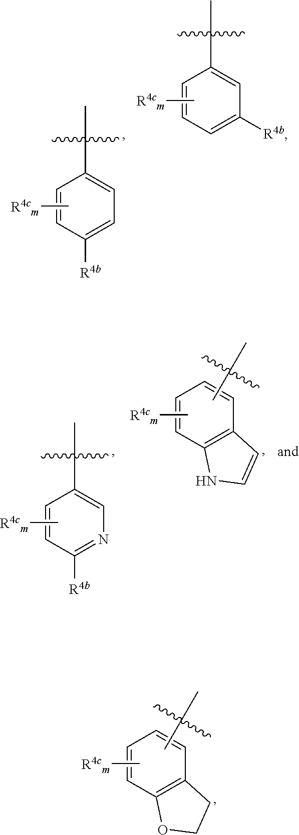

wherein
R⁴ is selected from (C₁ to C₆) alkyl, (C₂ to C₆) alkenyl, (C₂ to C₆) alkynyl, —O—(C₁ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, and —C(O)R⁵, wherein
each of the said (C₁ to C₆) alkyl, (C₂ to C₆) alkenyl, (C₂ to C₆) alkynyl, —O—(C₁ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁷ group as defined above;
R^{4c} is independently selected from hydrogen, deuterium, halogen, cyano, OH, CF₃, (C₁ to C₆) alkyl, (C₂ to C₆) alkenyl, (C₂ to C₆) alkynyl, —O—(C₁ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, —C(O)R⁵, —NR^{6a}R^{6b}, —S(O)ₘR⁵, —S(O)ₘNR^{6a}R^{6b}, —NR^{6a}S(O)ₘR⁵, —OC(O)R⁵, —NR^{6a}C(O)R⁵, and —NR^{6c}C(O)NR^{6a}R^{6b},
wherein each of the said (C₁ to C₆) alkyl, (C₂ to C₆) alkenyl, (C₂ to C₆) alkynyl, —O—(C₁ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁷ group as defined above.

In another embodiment, the method comprises administering to humans, other mammals, cell culture, or biological sample an effective amount of a compound represented by Structural Formula I, wherein A and R³ are defined as above, or a pharmaceutically acceptable salt, and a pharmaceutically acceptable carrier, diluent, or vehicle thereof, wherein:
R¹ and R² are independently selected from the group consisting of

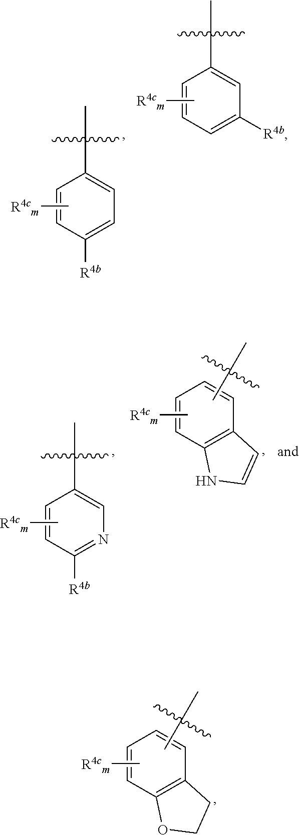

wherein
R^{4b} is selected from (C₁ to C₆) alkyl, (C₂ to C₆) alkenyl, (C₂ to C₆) alkynyl, —O—(C₁ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, and —C(O)R⁵, wherein
each of the said (C₁ to C₆) alkyl, (C₂ to C₆) alkenyl, (C₂ to C₆) alkynyl, —O—(C₁ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁷ group as defined above;
R^{4c} is independently selected from hydrogen, deuterium, halogen, cyano, OH, CF₃, (C₁ to C₆) alkyl, (C₂ to C₆) alkenyl, (C₂ to C₆) alkynyl, —O—(C₁ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, —C(O)R⁵, —NR^{6a}R^{6b}, —S(O)ₘR⁵, —S(O)ₘNR^{6a}R^{6b}, —NR^{6a}S(O)ₘR⁵, —OC(O)R⁵, —NR^{6a}C(O)R⁵, and —NR^{6c}C(O)NR^{6a}R^{6b},
   wherein each of the said (C₁ to C₆) alkyl, (C₂ to C₆) alkenyl, (C₂ to C₆) alkynyl, —O—(C₁ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁷ group as defined above.

In another embodiment, the method comprises administering to humans, other mammals, cell culture, or biological sample an effective amount of a compound represented by Structural Formula I, wherein A, R¹, R², and R³ are defined as above or a pharmaceutically acceptable salt, and a pharmaceutically acceptable carrier, diluent, or vehicle thereof, wherein:
R^{4b} is selected from the group consisting of halogen, methoxy, isopropoxy, cyclopropoxy, tert-butoxy, cyclopropylmethoxy, (2-hydroxypropan)-2-yl, (1-hydroxycyclopropan)-1-yl, (1-hydroxycyclobutan)-1-yl, difluoromethoxy, trifluoromethoxy, trifluoromethyl, trifluoroethoxy, methoxyethoxy, phenoxy, benzyloxy, difluoromethyl, cyclopropyl, acetyl, n-propyloxy, n-butyloxy, isobutyloxy, sec-butyloxy, cyclobutyloxy, cyclopentyloxy, (1-hydroxycyclopentan)-1-yl, hydroxyethoxy, ethoxyethoxy, isopropoxyethoxy, isopropyl, n-propyl, n-butyl, isobutyl, sec-butyl, cyclobutyl, cyclopentyl, tert-butyl, propanoyl, isobutanoyl, sec-butanoyl, tert-butanoyl, cyclopropanoyl, cyclobutanoyl, and cyclopentanoyl.

In another embodiment, the method comprises administering to humans, other mammals, cell culture, or biological sample an effective amount of a compound represented by Structural Formula I, wherein A, R¹, and R² are defined as above, or a pharmaceutically acceptable salt, and a pharmaceutically acceptable carrier, diluent, or vehicle thereof, wherein:
R³ is hydrogen, deuterium, methyl, or CD₃.

In another embodiment, the method comprises administering to humans, other mammals, cell culture, or biological sample an effective amount of a compound represented by Structural Formula I, A, R¹, and R² are defined as above, or a pharmaceutically acceptable salt, and a pharmaceutically acceptable carrier, diluent, or vehicle thereof, wherein:
R^{4b} is isopropoxy, tert-butoxy, phenoxy, isopropyl, or tert-butyl.

In another embodiment, the method comprises administering to humans, other mammals, cell culture, or biological sample an effective amount of a compound represented by Structural Formula I, A, R¹, and R² are defined as above, or a pharmaceutically acceptable salt, and a pharmaceutically acceptable carrier, diluent, or vehicle thereof, wherein:
R³ is hydrogen, deuterium, methyl, or CD₃;
R^{4b} is isopropoxy, tert-butoxy, phenoxy, isopropyl, or tert-butyl.

In another embodiment, the method comprises administering to humans, other mammals, cell culture, or biological sample an effective amount of a compound represented by Structural Formula I, wherein A is C—R³ as defined above, or a pharmaceutically acceptable salt, and a pharmaceutically acceptable carrier, diluent, or vehicle thereof, wherein:
R¹ and R² are independently selected from the group consisting of

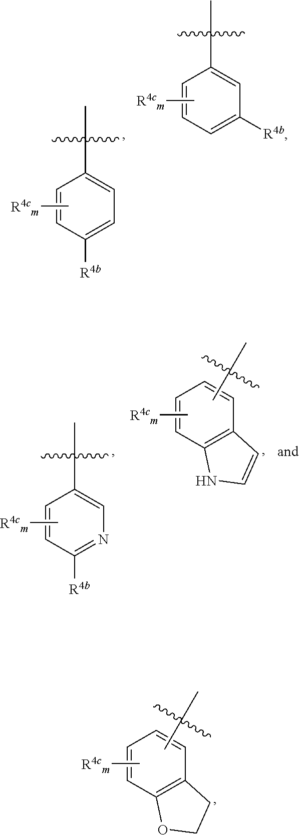

wherein
R^{4b} is selected from (C₁ to C₆) alkyl, (C₂ to C₆) alkenyl, (C₂ to C₆) alkynyl, —O—(C₁ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, and —C(O)R⁵, wherein
each of the said (C₁ to C₆) alkyl, (C₂ to C₆) alkenyl, (C₂ to C₆) alkynyl, —O—(C₁ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁷ group as defined above;
R^{4c} is independently selected from hydrogen, deuterium, halogen, cyano, OH, CF₃, (C₁ to C₆) alkyl, (C₂ to C₆) alkenyl, (C₂ to C₆) alkynyl, —O—(C₁ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, —C(O)R⁵, —NR^{6a}R^{6b}, —S(O)ₘR⁵, —S(O)ₘNR^{6a}R^{6b}, —NR^{6a}S(O)ₘR⁵, —OC(O)R⁵, —NR⁶C(O)R⁵, and —NR^{6c}C(O)NR^{6a}R^{6b},
   wherein each of the said (C₁ to C₆) alkyl, (C₂ to C₆) alkenyl, (C₂ to C₆) alkynyl, —O—(C₁ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁷ group as defined above.

In another embodiment, the method comprises administering to humans, other mammals, cell culture, or biological sample an effective amount of a compound represented by Structural Formula I, wherein A is C—R³, R¹, R², and R³ are defined as above, or a pharmaceutically acceptable salt, and a pharmaceutically acceptable carrier, diluent, or vehicle thereof, wherein:
R^{4b} is selected from the group consisting of halogen, methoxy, isopropoxy, cyclopropoxy, tert-butoxy, cyclopropylmethoxy, (2-hydroxypropan)-2-yl, (1-hydroxycyclopropan)-1-yl, (1-hydroxycyclobutan)-1-yl, difluoromethoxy, trifluoromethoxy, trifluoromethyl, trifluoroethoxy, methoxyethoxy, phenoxy, benzyloxy, difluoromethyl, cyclopropyl, acetyl, n-propyloxy, n-butyloxy, isobutyloxy, sec-butyloxy, cyclobutyloxy, cyclopentyloxy, (1-hydroxycyclopentan)-1-yl, hydroxyethoxy, ethoxyethoxy, isopropoxyethoxy, isopropyl, n-propyl, n-butyl, isobutyl, sec-butyl, cyclobutyl, cyclopentyl, tert-butyl, propanoyl, isobutanoyl, sec-butanoyl, tert-butanoyl, cyclopropanoyl, cyclobutanoyl, and cyclopentanoyl.

In another embodiment, the method comprises administering to humans, other mammals, cell culture, or biological sample an effective amount of a compound represented by Structural Formula I, wherein A is C—R³, R¹ and R² are defined as above, or a pharmaceutically acceptable salt, and a pharmaceutically acceptable carrier, diluent, or vehicle thereof, wherein:
R³ is hydrogen, deuterium, methyl, or CD₃.

In another embodiment, the method comprises administering to humans, other mammals, cell culture, or biological sample an effective amount of a compound represented by Structural Formula I, wherein A is C—R³, R¹, R², and R³ are defined as above, or a pharmaceutically acceptable salt, and a pharmaceutically acceptable carrier, diluent, or vehicle thereof, wherein:
R^{4b} is isopropoxy, tert-butoxy, phenoxy, isopropyl, or tert-butyl.

In another embodiment, the method comprises administering to humans, other mammals, cell culture, or biological sample an effective amount of a compound represented by Structural Formula I, wherein A is N, R³ is defined as above, or a pharmaceutically acceptable salt, and a pharmaceutically acceptable carrier, diluent, or vehicle thereof, wherein:
R¹ and R² are independently selected from the group consisting of

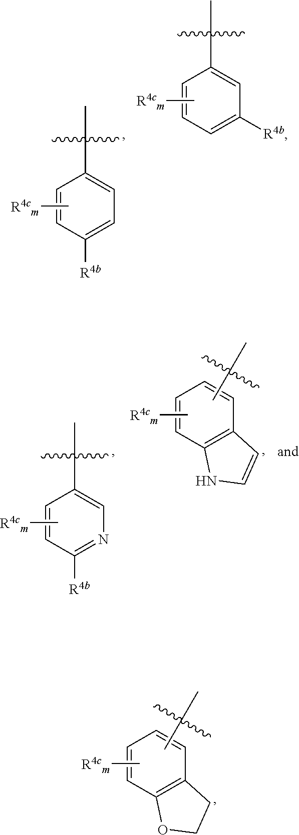

wherein
R^{4b} is selected from (C₁ to C₆) alkyl, (C₂ to C₆) alkenyl, (C₂ to C₆) alkynyl, —O—(C₁ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, and —C(O)R⁵, wherein
each of the said (C₁ to C₆) alkyl, (C₂ to C₆) alkenyl, (C₂ to C₆) alkynyl, —O—(C₁ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁷ group as defined above;
R^{4c} is independently selected from hydrogen, deuterium, halogen, cyano, OH, CF₃, (C₁ to C₆) alkyl, (C₂ to C₆) alkenyl, (C₂ to C₆) alkynyl, —O—(C₁ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, —C(O)R⁵, —NR^{6a}R^{6b}, —S(O)ₘR⁵, —S(O)ₘNR^{6a}R^{6b}, —NR^{6a}S(O)ₘR⁵, —OC(O)R⁵, —NR^{6a}C(O)R⁵, and —NR^{6c}C(O)NR^{6a}R^{6b},
   wherein each of the said (C₁ to C₆) alkyl, (C₂ to C₆) alkenyl, (C₂ to C₆) alkynyl, —O—(C₁ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁷ group as defined above.

In another embodiment, the method comprises administering to humans, other mammals, cell culture, or biological sample an effective amount of a compound represented by Structural Formula I, wherein A is N, R¹, R², and R³ are defined as above, or a pharmaceutically acceptable salt, and a pharmaceutically acceptable carrier, diluent, or vehicle thereof, wherein:
R^{4b} is selected from the group consisting of halogen, methoxy, isopropoxy, cyclopropoxy, tert-butoxy, cyclopropylmethoxy, (2-hydroxypropan)-2-yl, (1-hydroxycyclopropan)-1-yl, (1-hydroxycyclobutan)-1-yl, difluoromethoxy, trifluoromethoxy, trifluoromethyl, trifluoroethoxy, methoxyethoxy, phenoxy, benzyloxy, difluoromethyl, cyclopropyl, acetyl, n-propyloxy, n-butyloxy, isobutyloxy, sec-butyloxy, cyclobutyloxy, cyclopentyloxy, (1-hydroxycyclopentan)-1-yl, hydroxyethoxy, ethoxyethoxy, isopropoxyethoxy, isopropyl, n-propyl, n-butyl, isobutyl, sec-butyl, cyclobutyl, cyclopentyl, tert-butyl, propanoyl, isobutanoyl, sec-butanoyl, tert-butanoyl, cyclopropanoyl, cyclobutanoyl, and cyclopentanoyl.

In another embodiment, the method comprises administering to humans, other mammals, cell culture, or biological sample an effective amount of a compound represented by Structural Formula I, wherein A is N, R¹ and R² are defined as above, or a pharmaceutically acceptable salt, and a pharmaceutically acceptable carrier, diluent, or vehicle thereof, wherein:
R³ is hydrogen, deuterium, methyl, or CD₃.

In another embodiment, the method comprises administering to humans, other mammals, cell culture, or biological sample an effective amount of a compound represented by Structural Formula I, wherein A is N, R¹, R², and R³ are defined as above, or a pharmaceutically acceptable salt, and a pharmaceutically acceptable carrier, diluent, or vehicle thereof, wherein:
R^{4b} is isopropoxy, tert-butoxy, phenoxy, isopropyl, or tert-butyl.

In another embodiment, the method comprises administering to humans, other mammals, cell culture, or biological sample an effective amount of a compound represented by Structural Formula I, wherein R³ is defined as above, or a pharmaceutically acceptable salt, and a pharmaceutically acceptable carrier, diluent, or vehicle thereof, wherein:
A is C—R³ or N;
R¹ is selected from
halogen, cyano, (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₂ to C₉) heterocycloalkyl, (C₆ to C₁₀) aryl, and (C₂ to C₉) heteroaryl, wherein each of the said (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₂ to C₉) heterocycloalkyl, (C₆ to C₁₀) aryl, and (C₂ to C₉) heteroaryl is optionally substituted with at least one R⁴ group as defined above;
R² is 4-alkoxyphenyl, 4-alkylphenyl, or 4-aryloxyphenyl.

In another embodiment, the method comprises administering to humans, other mammals, cell culture, or biological sample an effective amount of a compound represented by Structural Formula I, wherein R³ is defined as above, or a pharmaceutically acceptable salt, and a pharmaceutically acceptable carrier, diluent, or vehicle thereof, wherein:
A is C—R³ or N;
R¹ is selected from halogen, cyano, (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₂ to C₉) heterocycloalkyl, (C₆ to C₁₀) aryl, and (C₂ to C₉) heteroaryl, wherein
each of the said (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₂ to C₉) heterocycloalkyl, (C₆ to C₁₀) aryl, and (C₂ to C₉) heteroaryl is optionally substituted with at least one R⁴ group as defined above;
R² is 4-isopropyloxyphenyl, 4-tert-butoxyphenyl, 4-phenoxyphenyl, 4-isopropylphenyl, or 4-tert-butylphenyl.

In another embodiment, the method comprises of administering to humans, other mammals, cell culture, or biological sample a pharmaceutically effective amount of a pharmaceutical composition comprising a compound selected from the group of compounds described as Examples C58 to C61, C66 to C91, and D23 with a pharmaceutically acceptable carrier, dilutant, or vehicle.

In another embodiment, the method comprises of administering to humans, other mammals, cell culture, or biological sample a pharmaceutically effective amount of a pharmaceutical composition comprising a compound selected from the group consisting of:

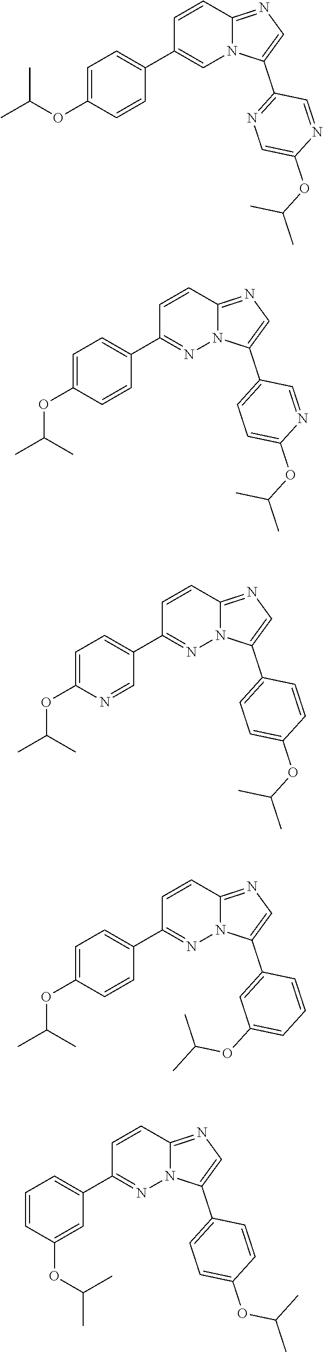 [SMILES: CC(C)Oc1ccc(-c2ccc3ncc(-c4cnc(OC(C)C)cn4)n3c2)cc1 ; CC(C)Oc1ccc(-c2ccc3ncc(-c4ccc(OC(C)C)nc4)n3n2)cc1 ; CC(C)Oc1ccc(-c2cnc3ccc(-c4ccc(OC(C)C)nc4)nn23)cc1 ; CC(C)Oc1ccc(-c2ccc3ncc(-c4cccc(OC(C)C)c4)n3n2)cc1 ; CC(C)Oc1ccc(-c2cnc3ccc(-c4cccc(OC(C)C)c4)nn23)cc1]

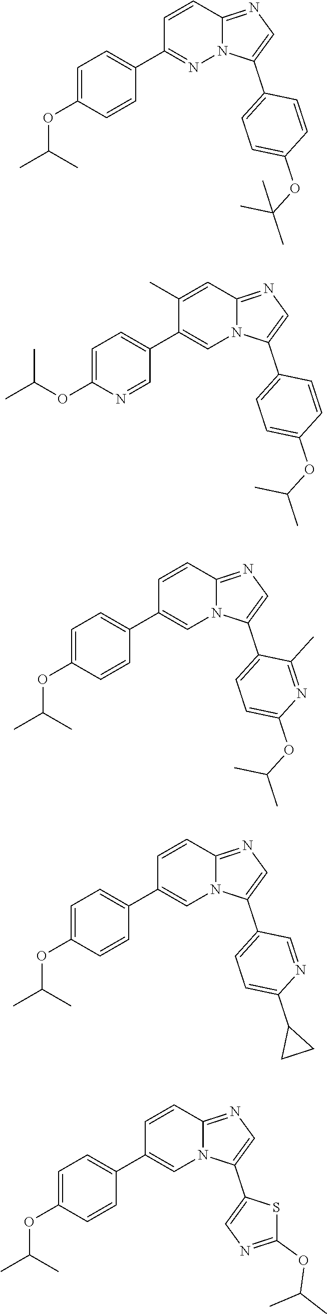

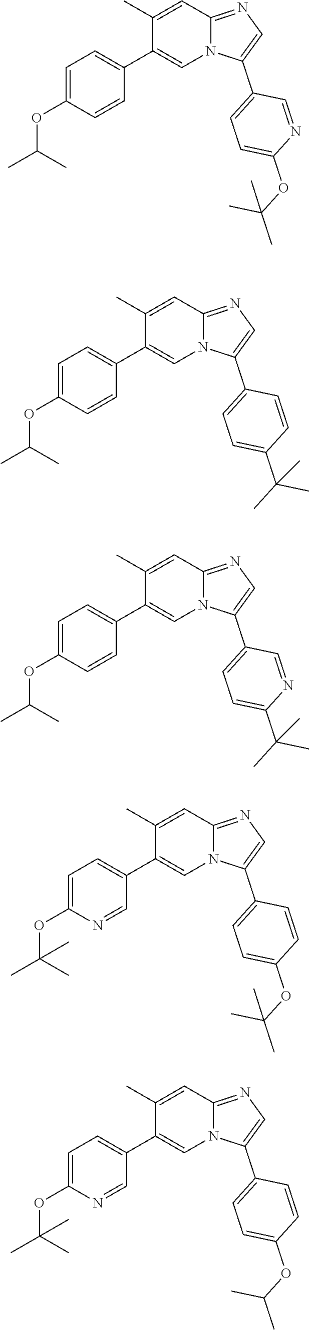

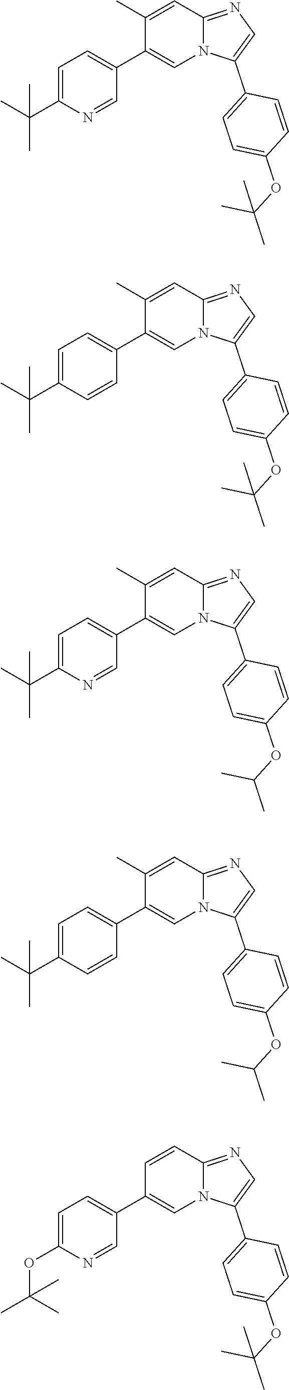 [SMILES: Cc1cc2ncc(-c3ccc(OC(C)(C)C)cc3)n2cc1-c1ccc(C(C)(C)C)nc1 ; Cc1cc2ncc(-c3ccc(OC(C)(C)C)cc3)n2cc1-c1ccc(C(C)(C)C)cc1 ; Cc1cc2ncc(-c3ccc(OC(C)C)cc3)n2cc1-c1ccc(C(C)(C)C)nc1 ; Cc1cc2ncc(-c3ccc(OC(C)C)cc3)n2cc1-c1ccc(C(C)(C)C)cc1 ; CC(C)(C)Oc1ccc(-c2cnc3ccc(-c4ccc(OC(C)(C)C)nc4)cn23)cc1]

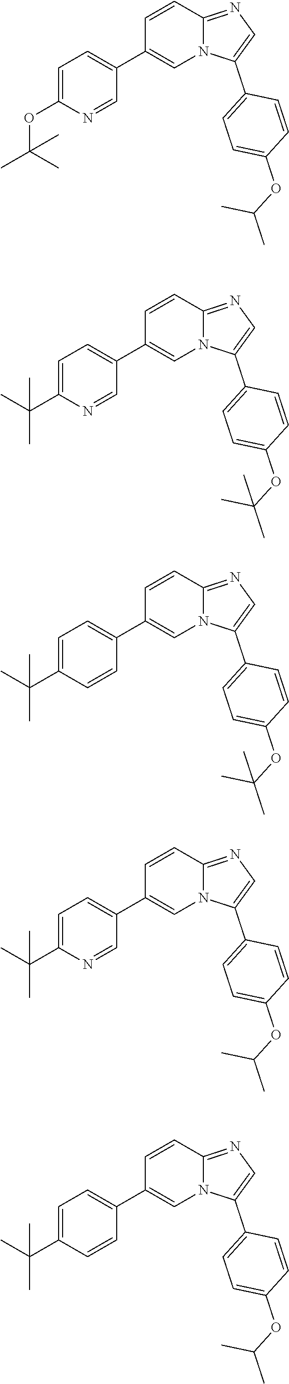 [SMILES: CC(C)Oc1ccc(-c2cnc3ccc(-c4ccc(OC(C)(C)C)nc4)cn23)cc1 ; CC(C)(C)Oc1ccc(-c2cnc3ccc(-c4ccc(C(C)(C)C)nc4)cn23)cc1 ; CC(C)(C)Oc1ccc(-c2cnc3ccc(-c4ccc(C(C)(C)C)cc4)cn23)cc1 ; CC(C)Oc1ccc(-c2cnc3ccc(-c4ccc(C(C)(C)C)nc4)cn23)cc1 ; CC(C)Oc1ccc(-c2cnc3ccc(-c4ccc(C(C)(C)C)cc4)cn23)cc1]

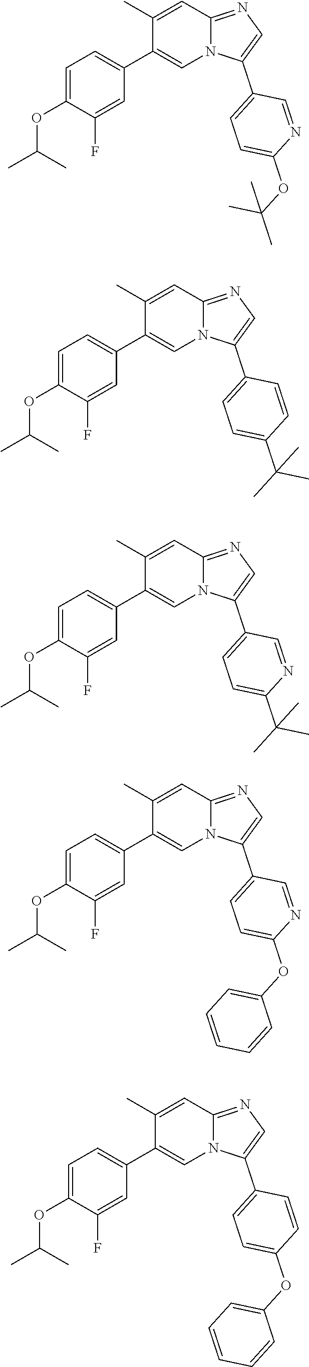

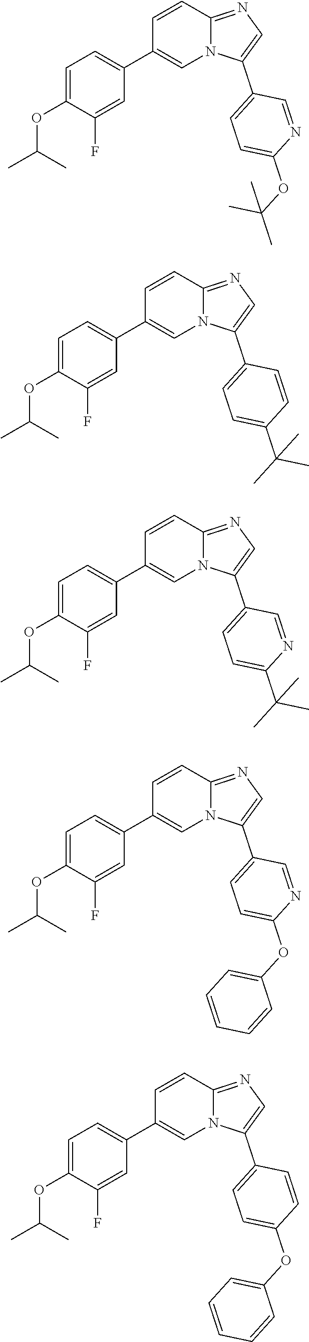

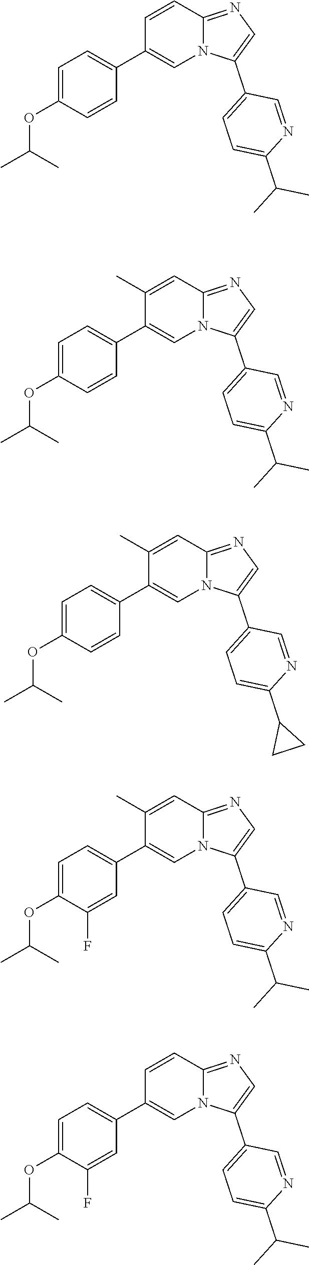

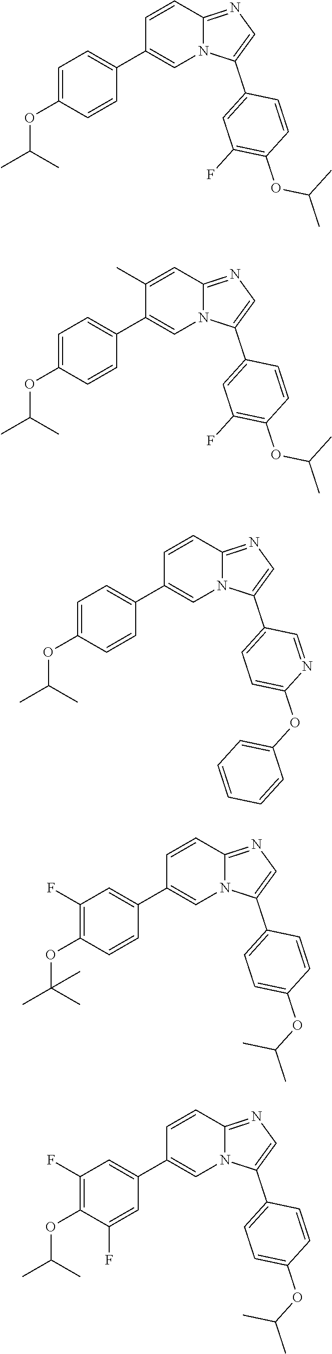

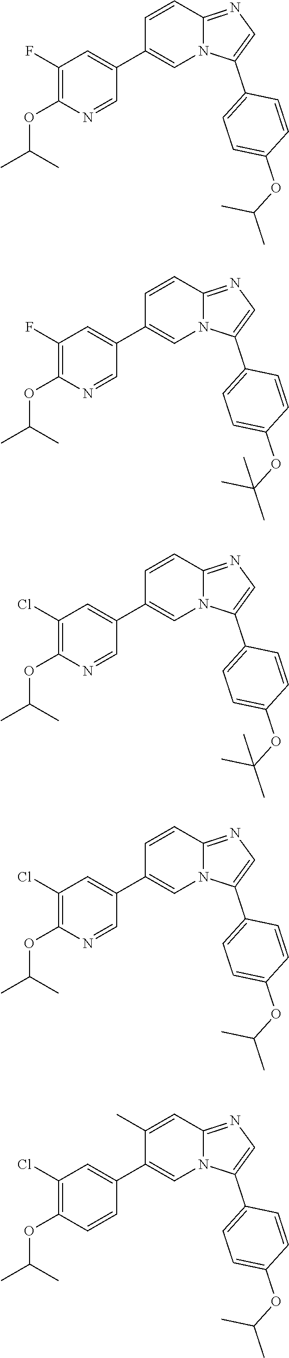

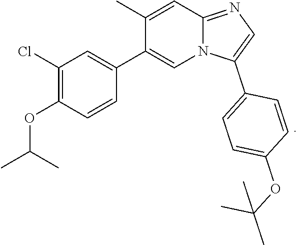 [SMILES: Cc1cc2ncc(-c3ccc(OC(C)(C)C)cc3)n2cc1-c1ccc(OC(C)C)c(Cl)c1]

In another embodiment, the invention relates to compounds of Structural Formulae Ia and Ib

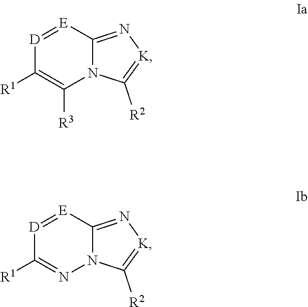

or a pharmaceutically acceptable salt, and a pharmaceutically acceptable carrier, diluent, or

vehicle thereof, wherein:
D and E are independently selected from C—R³;
K is selected from CH and CD;
R¹ is selected from (C₆ to C₁₀) aryl and (C₂ to C₉) heteroaryl, wherein each of the said (C₆ to C₁₀) aryl and (C₂ to C₉) heteroaryl is substituted with at least one R^{4a} group, and wherein said (C₂ to C₉) heteroaryl is C-attached;
with the proviso that R¹ is not selected from the group consisting of substituted 3-carbamoyl-2-phenyl-1-benzofuran-5-yl, substituted 1,3,4-oxadiazolyl, substituted 1,3,4-triazolyl, substituted 1,3,4-thiadiazolyl, substituted oxazoyl, substituted thiazoyl, substituted 1H-pyrazol-4-yl, substituted 1H-pyrazol-5-yl, optionally substituted 1-phenyl-1H-imidazol-5-yl, 4-{[(2-aminoethyl)amino]methyl}phenyl, (2-amino-1,3-benzoxazol)-5-yl; (2-amino-1,3-benzoxazol)-4-yl, 2-chloropyridyl-3-yl, 2-methylpyridinyl-4-yl, 2-fluoropyridyl-4-yl, 6-aminopyridyl-3-yl, 6-methoxypyridyl-3-yl, pyridyl-4-yl-N-oxide, 3,4-difluorphenyl, substituted 1H-pyrrol-3-yl, 6-methylpyridyl-3-yl, 2-methoxypyridyl-3-yl, 6-cyanopyridyl-3-yl, pyridyl-4-yl, 4-(methylsulfonyl)phenyl, thien-3-yl, and fur-3-yl;
R² is selected from the group consisting of

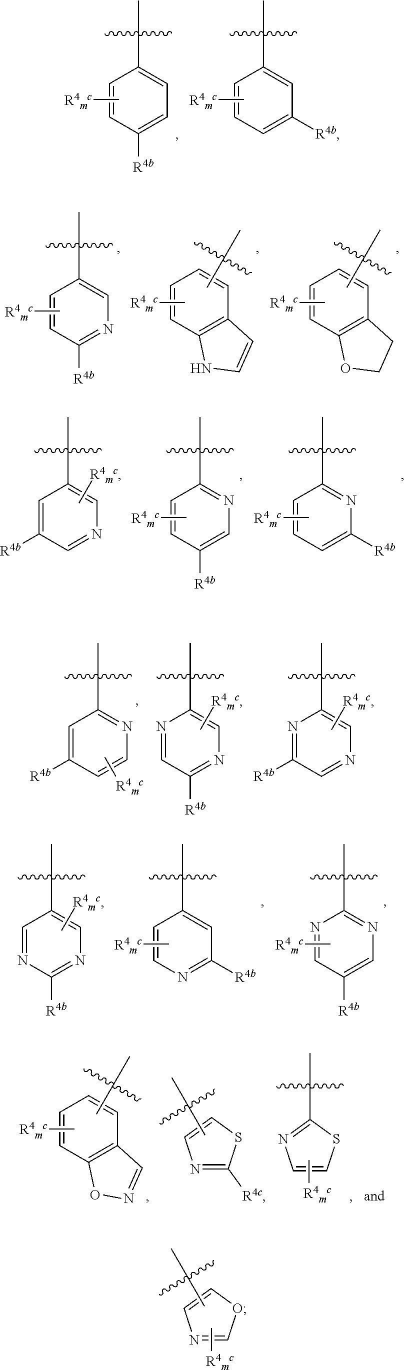

each of the R³ is independently selected from H, deuterium, halogen, cyano, CF₃, (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₆) cycloheteroalkyl, —O—(C₃ to C₁₀) cycloalkyl, —O—(C₅ to C₁₀) cycloalkenyl, —O—(C₂ to C₆) cycloheteroalkyl, C(O)R⁵, —S(O)ₘR⁵, —S(O)ₘNR^{6a}R^{6b}, —NR^{6a}S(O)ₘR⁵, —(CH₂)ₙC(O)OR⁵, —OC(O)R⁵, and —NR^{6c}—C(O)NR^{5a}R^{5b}, wherein
each of the said (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, and —O—(C₂ to C₉) cycloheteroalkyl is optionally substituted with at least one R⁷ group;
R^{4a} is independently selected from halogen, cyano, OH, CF₃, (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, (C₂ to C₉) heteroaryl, —C(O)R⁵, —NR^{6a}R^{6b}, —S(O)ₘR⁵, —S(O)ₘNR^{6a}R^{6b}, —N^{6a}S(O)ₘR⁵, —OC(O)R⁵, and —NR^{6c}C(O)NR^{6a}R^{6b}, wherein
each of the said (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, and (C₂ to C₉) heteroaryl is optionally substituted with at least one R⁷ group;
R^{b} is selected from the group consisting of
(C₂ to C₆) alkyl, (C₂ to C₆) alkenyl, (C₂ to C₆) alkynyl, —O—(C₂ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₅ to C₁₀) cycloalkenyl, —O—(C₂ to C₉) cycloheteroalkyl, (C₆ to C₁₀) aryl, (C₂ to C₉) heteroaryl, isopropoxy, cyclopropoxy, tert-butoxy, cyclopropylmethoxy, (2-hydroxypropan)-2-yl, (1-hydroxycyclopropan)-1-yl, (1-hydroxycyclobutan)-1-yl, trifluoromethoxy, trifluoromethyl, difluoromethoxy, trifluoroethoxy, methoxyethoxy, phenoxy, benzyloxy, difluoromethyl, cyclopropyl, acetyl, n-propyloxy, n-butyloxy, isobutyloxy, sec-butyloxy, cyclobutyloxy, cyclopentyloxy, (1-hydroxycyclopentan)-1-yl, hydroxyethoxy, ethoxyethoxy, isopropoxyethoxy, isopropyl, n-propyl, n-butyl, sec-butyl, tert-butyl, cyclobutyl, cyclopentyl, propanoyl, isobutanoyl, sec-butanoyl, tert-butanoyl, cyclopropanoyl, cyclobutanoyl, and cyclopentanoyl;
wherein each of the said (C₂ to C₆) alkyl, (C₂ to C₆) alkenyl, (C₂ to C₆) alkynyl, —O—(C₂ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₅ to C₁₀) cycloalkenyl, —O—(C₂ to C₉) cycloheteroalkyl, (C₆ to C₁₀) aryl, and (C₂ to C₉) heteroaryl is optionally substituted with at least one R⁷ group;
R^{4c} is independently selected from hydrogen, deuterium, halogen, cyano, OH, CF₃, (C₁ to C₆) alkyl, (C₂ to C₆) alkenyl, (C₂ to C₆) alkynyl, —O—(C₂ to C₆) alkyl, —O—(C₂ to C₆) alkenyl, —O—(C₂ to C₆) alkynyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₅ to C₁₀) cycloalkenyl, —O—(C₂ to C₉) cycloheteroalkyl, (C₆ to C₁₀) aryl, (C₂ to C₉) heteroaryl, —C(O)R⁵, —NR^{6a}R^{6b}, —S(O)ₘR⁵, —S(O)ₘNR^{6a}R^{6b}, —NR^{6a}S(O)ₘR⁵, —OC(O)R⁵, —NR^{6a}C(O)R⁵, and —NR^{6c}C(O)NR^{6a}R^{6b}, wherein
each of the said (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₅ to C₁₀) cycloalkenyl, —O—(C₂ to C₉) cycloheteroalkyl, (C₆ to C₁₀) aryl, and (C₂ to C₉) heteroaryl is optionally substituted with at least one R⁷ group;
each of the R⁵ is independently selected from hydrogen, deuterium, (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₆ to C₁₀) aryl, and (C₂ to C₉) heteroaryl, wherein
each of the said (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁷ group;
each of the R^{6a}, R^{6b}, and R^{6c} are independently selected from hydrogen, deuterium, (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl,
or R^{6a} and R^{6b} may be taken together with the nitrogen atom to which they are attached to form a 4 to 8 membered cycloheteroalkyl ring, wherein
said 4 to 8 membered cycloheteroalkyl ring has 1 to 3 ring heteroatoms selected from the group consisting of N, O, and S, and wherein
the said 4 to 8 membered cycloheteroalkyl ring is optionally substituted with at least one R⁷ group;
R⁷ is independently selected from hydrogen, deuterium, halogen, OH, CF₃, (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, (C₂ to C₉) heteroaryl, —C(O)R⁸, —C(O)NR^{9a}R^{9b}, —NR^{9a}R^{9b}, —S(O)ₘR⁸, —S(O)ₘNR^{9a}R^{9b}, —NR⁹S(O)ₘR⁸, —(CH₂)ₙC(O)OR⁸, —(CH₂)ₙC(O)N(R^{9a}R^{9b}), —OC(O)R⁸, —NR^{9a}C(O)R⁸, and —NR^{9a}C(O)N(R^{9a}R^{9b}), wherein
each of the said (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, and (C₂ to C₉) heteroaryl is optionally substituted with at least one R¹⁰ group;
each of the R⁸ is independently selected from hydrogen, deuterium, (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl, wherein
each of the said (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R¹⁰ group;
each of the R^{9a}, R^{9b}, and R^{9c} are independently selected from hydrogen, deuterium, (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl,
or R^{9a} and R^{9b} may be taken together with the nitrogen atom to which they are attached to form a 4 to 8 membered cycloheteroalkyl ring, wherein
said 4 to 8 membered cycloheteroalkyl ring has 1 to 3 ring heteroatoms selected from the group consisting of N, O, and S, and wherein
the said 4 to 8 membered cycloheteroalkyl ring is optionally substituted;
R¹⁰ is independently selected from hydrogen, deuterium, halogen, cyano, OH, CF₃, (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, and (C₂ to C₉) heteroaryl, wherein
each of the said (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, and (C₂ to C₉) heteroaryl is optionally substituted with hydrogen, deuterium, halogen, cyano, —CD₃, OH, CF₃, (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, and (C₂ to C₉) heteroaryl;
m is 0, 1, or 2;
and n is 1, 2, 3, 4, 5, or 6;
with the proviso that
the following compounds are excluded:

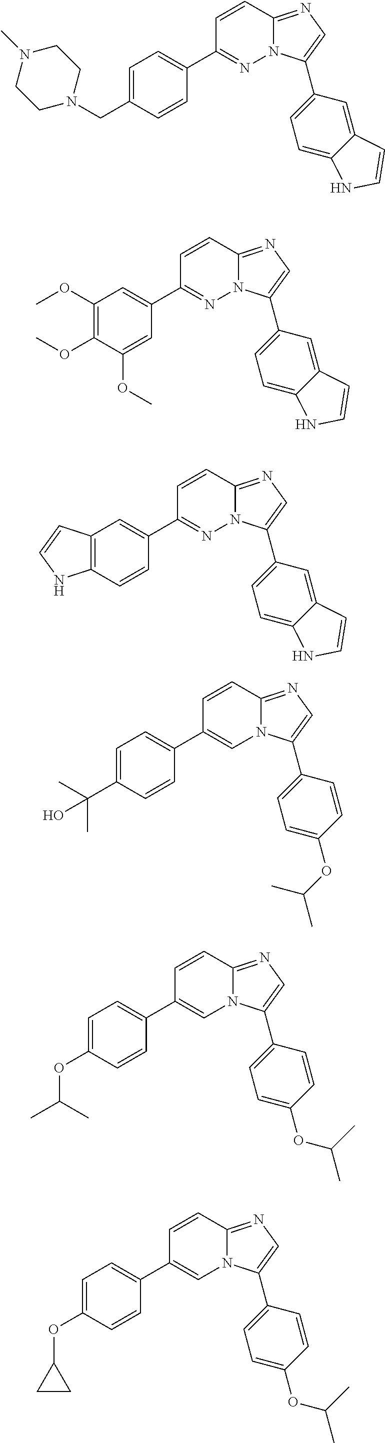

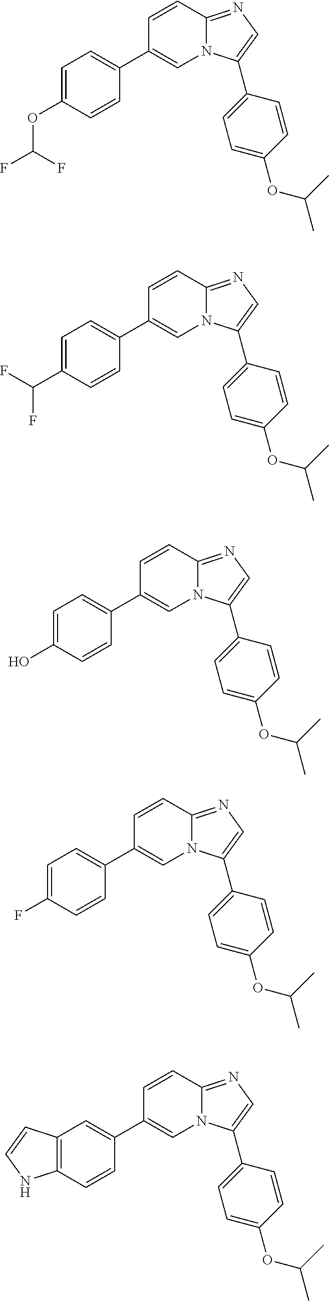 [SMILES: CC(C)Oc1ccc(-c2cnc3ccc(-c4ccc(OC(F)F)cc4)cn23)cc1 ; CC(C)Oc1ccc(-c2cnc3ccc(-c4ccc(C(F)F)cc4)cn23)cc1 ; CC(C)Oc1ccc(-c2cnc3ccc(-c4ccc(O)cc4)cn23)cc1 ; CC(C)Oc1ccc(-c2cnc3ccc(-c4ccc(F)cc4)cn23)cc1 ; CC(C)Oc1ccc(-c2cnc3ccc(-c4ccc5[nH]ccc5c4)cn23)cc1]

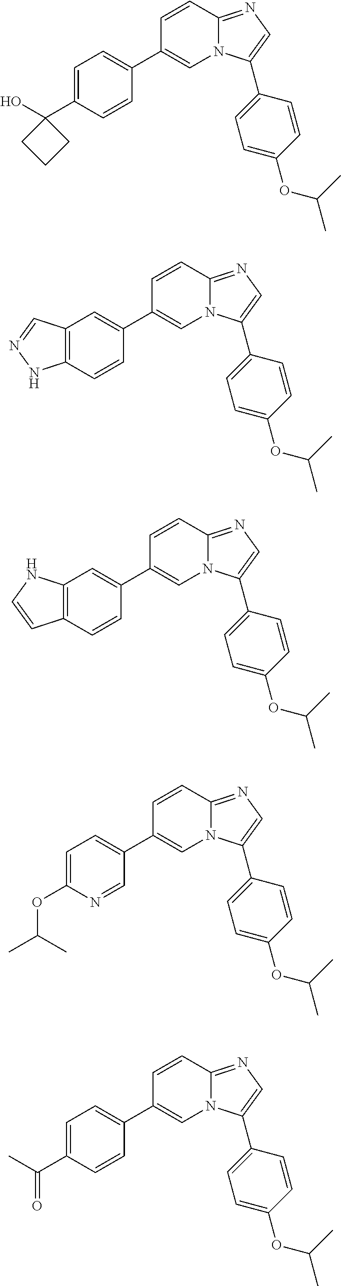

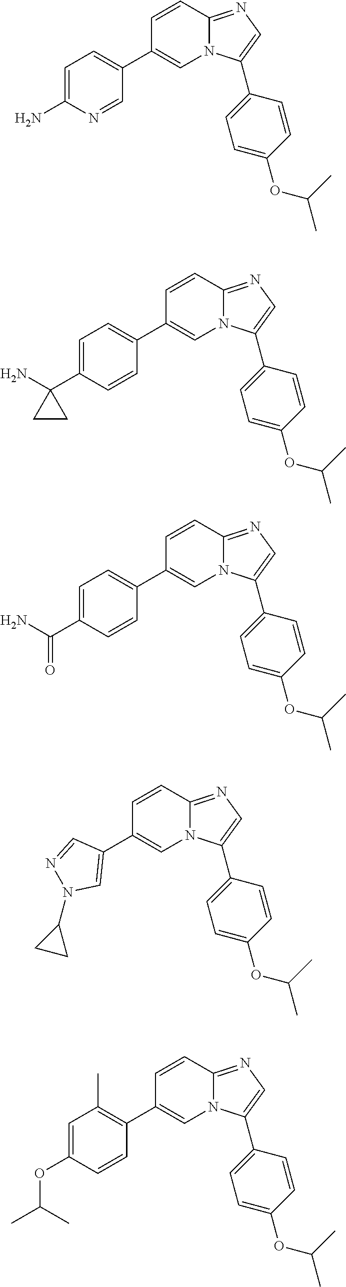

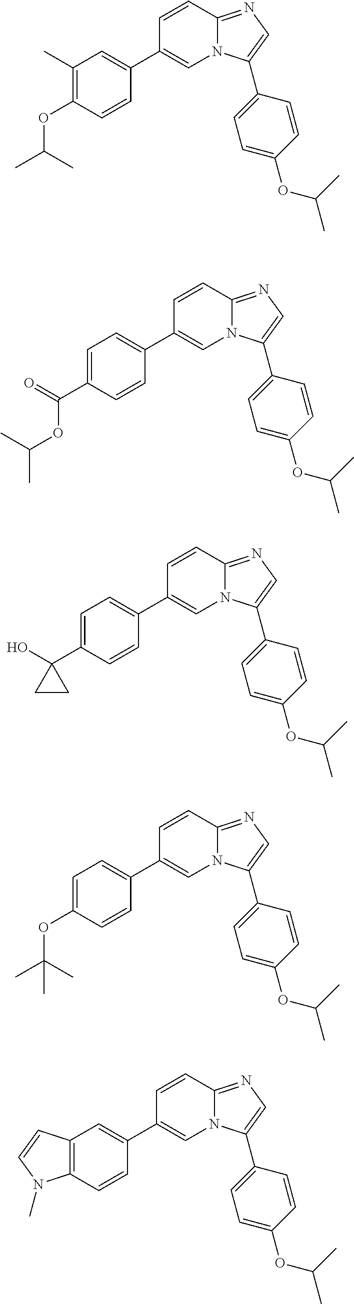

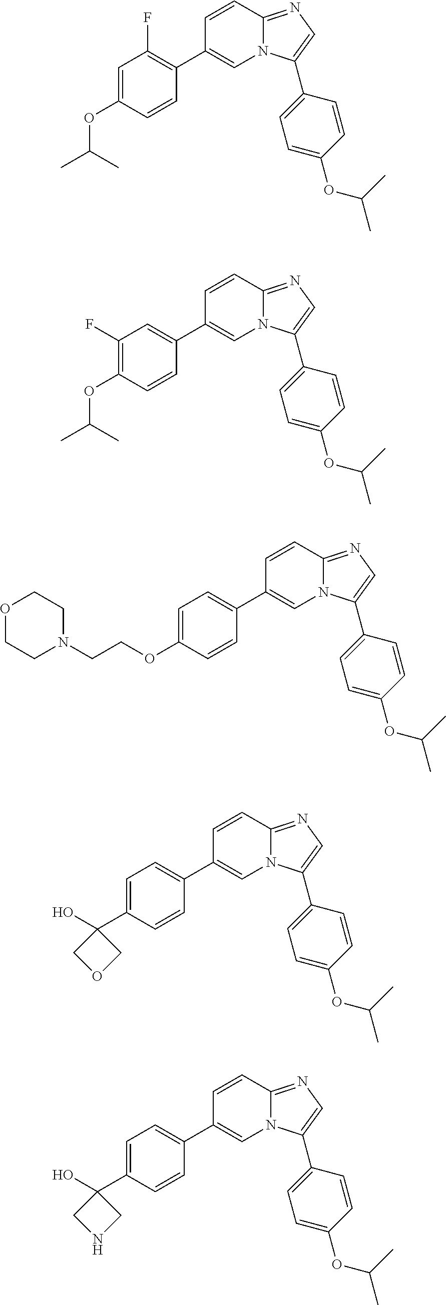

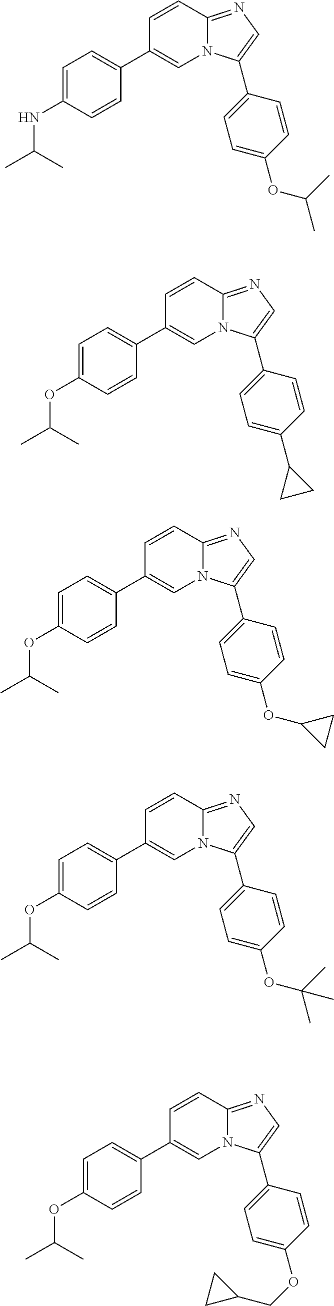

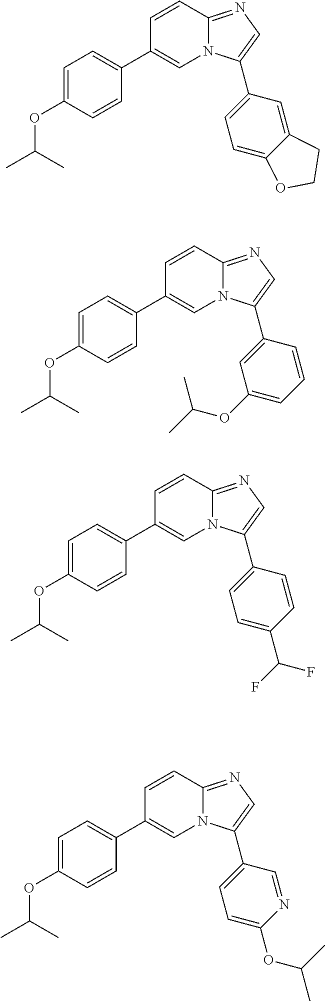

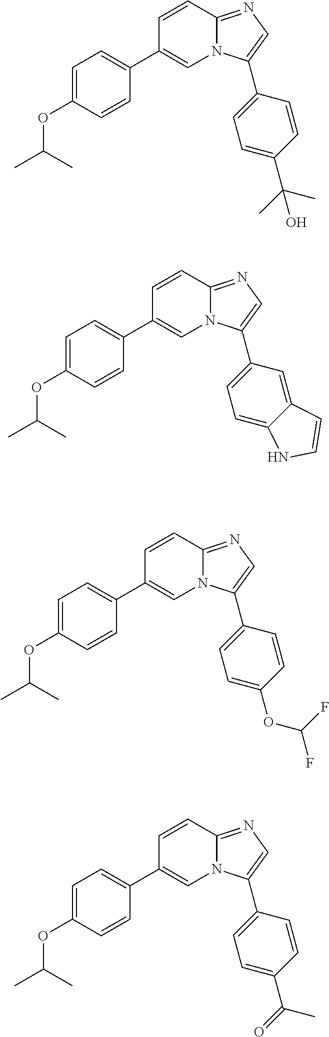

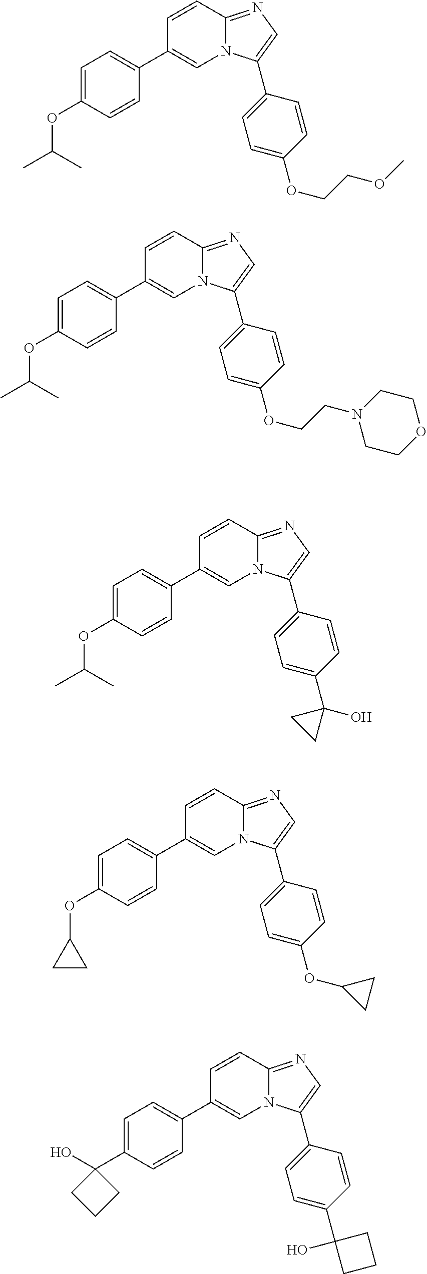

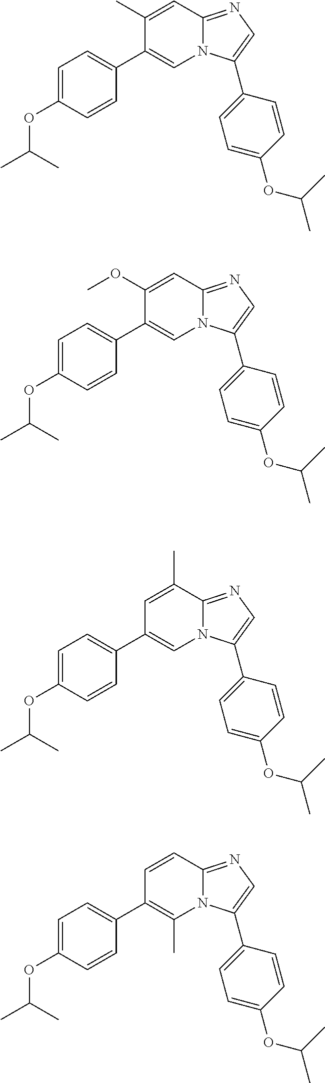

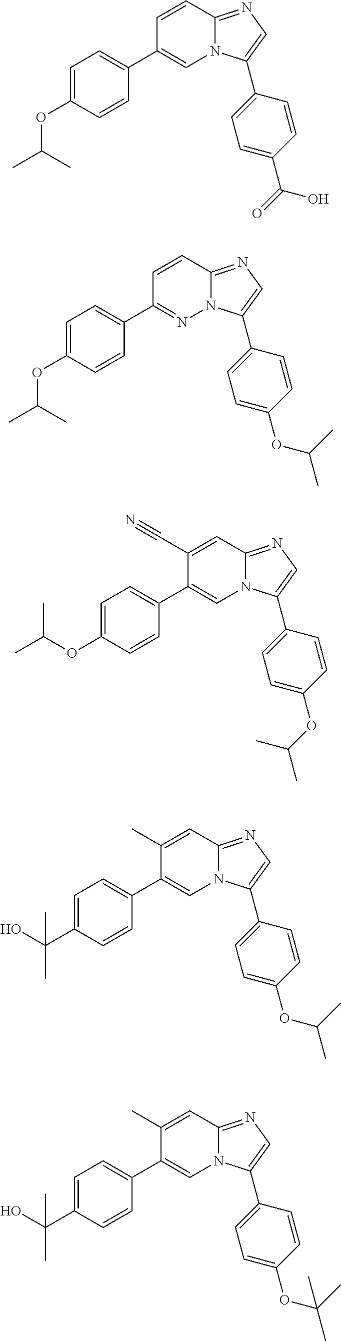

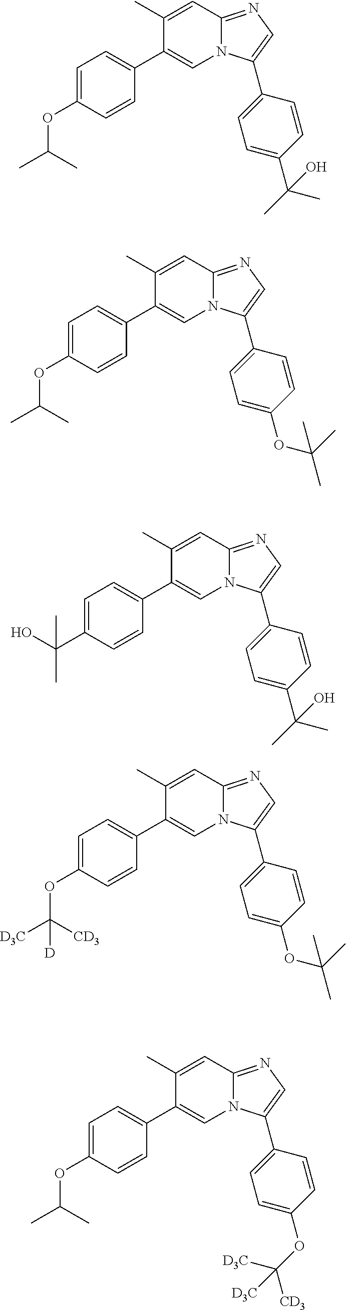

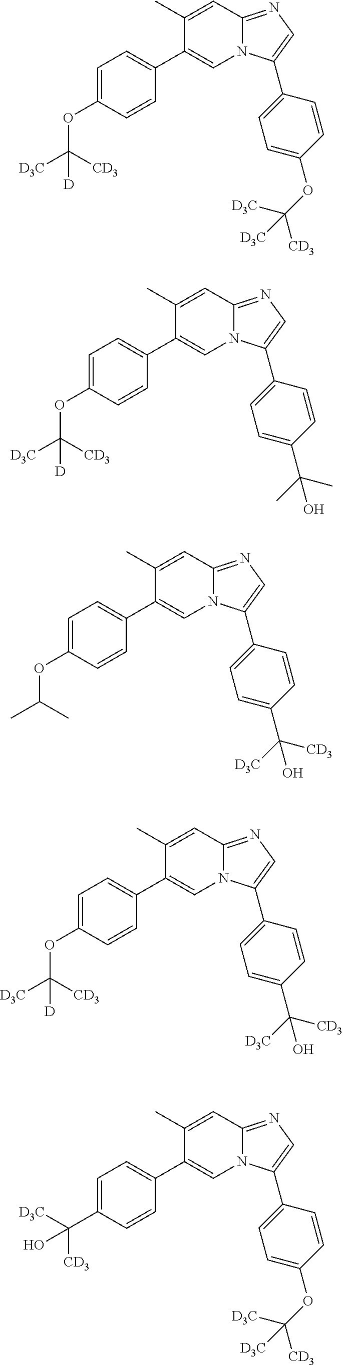

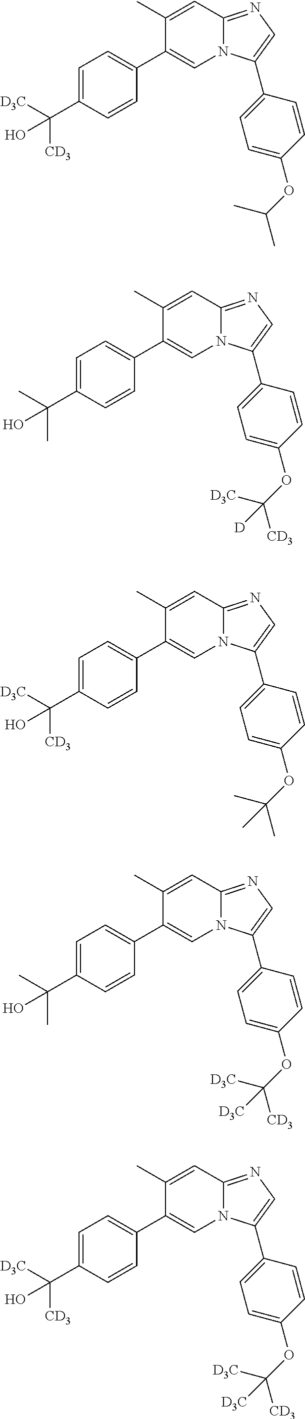

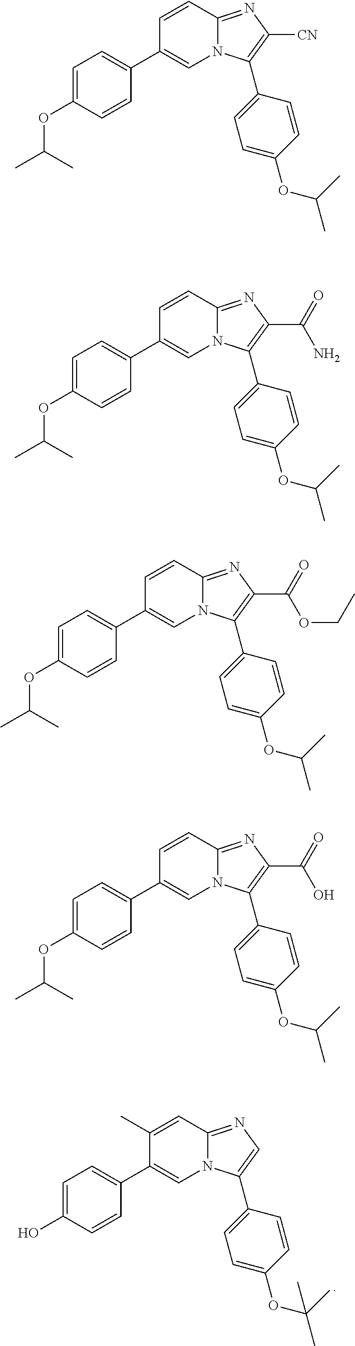

In another embodiment, the invention relates to compounds of Structural Formulae Ia and Ib, or a pharmaceutically acceptable salt, and a pharmaceutically acceptable carrier, diluent, or vehicle thereof, wherein R¹ and R³ are defined as above and wherein
R² is selected from the group consisting of

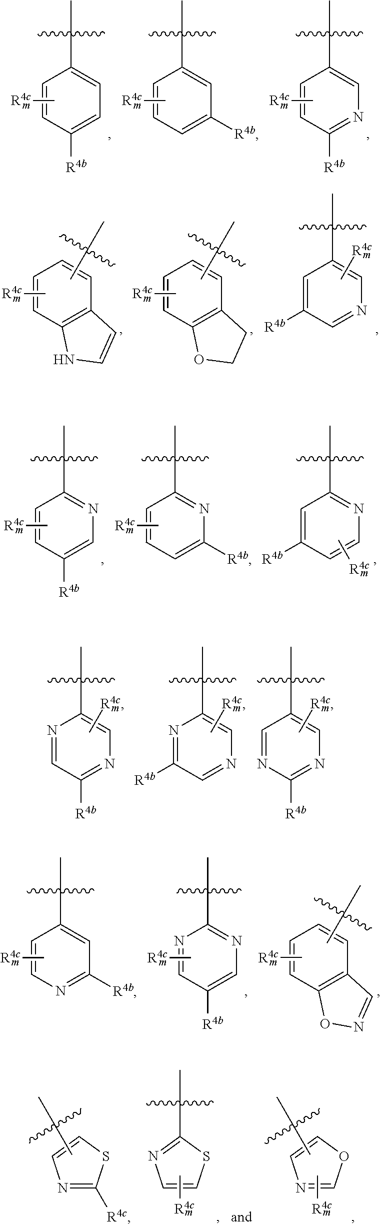

wherein
R^{4b} is selected from the group consisting of
(C₃ to C₆) alkyl, (C₂ to C₆) alkenyl, (C₂ to C₆) alkynyl, —O—(C₃ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₅ to C₁₀) cycloalkenyl, —O—(C₂ to C₆) cycloheteroalkyl, (C₆ to C₁₀) aryl, (C₂ to C₉) heteroaryl, isopropoxy, cyclopropoxy, tert-butoxy, cyclopropylmethoxy, (2-hydroxypropan)-2-yl, (1-hydroxycyclopropan)-1-yl, (1-hydroxycyclobutan)-1-yl, trifluoromethoxy, trifluoromethyl, difluoromethoxy, trifluoroethoxy, methoxyethoxy, phenoxy, benzyloxy, difluoromethyl, cyclopropyl, acetyl, n-propyloxy, n-butyloxy, isobutyloxy, sec-butyloxy, cyclobutyloxy, cyclopentyloxy, (1-hydroxycyclopentan)-1-yl, hydroxyethoxy, ethoxyethoxy, isopropoxyethoxy, isopropyl, n-propyl, n-butyl, sec-butyl, tert-butyl, cyclobutyl, cyclopentyl, propanoyl, isobutanoyl, sec-butanoyl, tert-butanoyl, cyclopropanoyl, cyclobutanoyl, and cyclopentanoyl;
wherein each of the said (C₃ to C₆) alkyl, (C₂ to C₆) alkenyl, (C₂ to C₆) alkynyl, —O—(C₃ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₅ to C₁₀) cycloalkenyl, —O—(C₂ to C₉) cycloheteroalkyl, (C₆ to C₁₀) aryl, and (C₂ to C₉) heteroaryl is optionally substituted with at least one R⁷ group as defined above;
R^{4c} is independently selected from hydrogen, deuterium, halogen, cyano, OH, CF₃, (C₁ to C₆) alkyl, (C₂ to C₆) alkenyl, (C₂ to C₆) alkynyl, —O—(C₂ to C₆) alkyl, —O—(C₂ to C₆) alkenyl, —O—(C₂ to C₆) alkynyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₅ to C₁₀) cycloalkenyl, —O—(C₂ to C₉) cycloheteroalkyl, (C₆ to C₁₀) aryl, (C₂ to C₉) heteroaryl, —C(O)R⁵, —NR^{6a}R^{6b}, —S(O)ₘR⁵, —S(O)ₘNR^{6a}R^{6b}, —NR^{6a}S(O)ₘR⁵, —OC(O)R⁵, —NR^{6a}C(O)R⁵, and —NR^{6c}C(O)NR^{6a}R^{6b}, wherein
each of the said (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₅ to C₁₀) cycloalkenyl, —O—(C₂ to C₉) cycloheteroalkyl, (C₆ to C₁₀) aryl, and (C₂ to C₉) heteroaryl is optionally substituted with at least one R⁷ group as defined above.

In another embodiment, the invention relates to compounds of Structural Formulae Ia and Ib, or a pharmaceutically acceptable salt, and a pharmaceutically acceptable carrier, diluent, or vehicle thereof, wherein R¹ and R³ are defined as above and wherein
R² is selected from the group consisting of

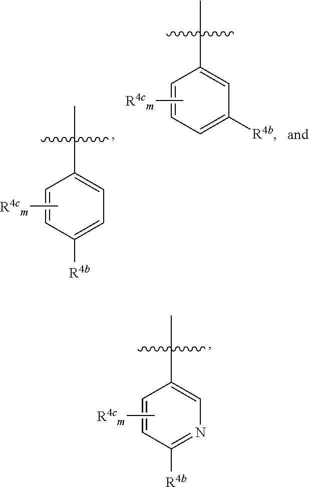

wherein
R^{4b} is selected from the group consisting of
(C₂ to C₆) alkyl, (C₂ to C₆) alkenyl, (C₂ to C₆) alkynyl, —O—(C₂ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₅ to C₁₀) cycloalkenyl, —O—(C₂ to C₉) cycloheteroalkyl, (C₆ to C₁₀) aryl, (C₂ to C₉) heteroaryl, isopropoxy, cyclopropoxy, tert-butoxy, cyclopropylmethoxy, (2-hydroxypropan)-2-yl, (1-hydroxycyclopropan)-1-yl, (1-hydroxycyclobutan)-1-yl, trifluoromethoxy, trifluoromethyl, difluoromethoxy, trifluoroethoxy, methoxyethoxy, phenoxy, benzyloxy, difluoromethyl, cyclopropyl, acetyl, n-propyloxy, n-butyloxy, isobutyloxy, sec-butyloxy, cyclobutyloxy, cyclopentyloxy, (1-hydroxycyclopentan)-1-yl, hydroxyethoxy, ethoxyethoxy, isopropoxyethoxy, isopropyl, n-propyl, n-butyl, sec-butyl, tert-butyl, cyclobutyl, cyclopentyl, propanoyl, isobutanoyl, sec-butanoyl, tert-butanoyl, cyclopropanoyl, cyclobutanoyl, and cyclopentanoyl;
wherein each of the said (C₂ to C₆) alkyl, (C₂ to C₆) alkenyl, (C₂ to C₆) alkynyl, —O—(C₂ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₅ to C₁₀) cycloalkenyl, —O—(C₂ to C₉) cycloheteroalkyl, (C₆ to C₁₀) aryl, and (C₂ to C₉) heteroaryl is optionally substituted with at least one R⁷ group as defined above;
R^{4c} is independently selected from hydrogen, deuterium, halogen, cyano, OH, CF₃, (C₁ to C₆) alkyl, (C₂ to C₆) alkenyl, (C₂ to C₆) alkynyl, —O—(C₂ to C₆) alkyl, —O—(C₂ to C₆) alkenyl, —O—(C₂ to C₆) alkynyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₅ to C₁₀) cycloalkenyl, —O—(C₂ to C₉) cycloheteroalkyl, (C₆ to C₁₀) aryl, (C₂ to C₉) heteroaryl, —C(O)R⁵, —NR^{6a}R^{6b}, —S(O)ₘR⁵, —S(O)ₘNR^{6a}R^{6b}, —NR^{6a}S(O)ₘR⁵, —OC(O)R⁵, —NR^{6a}C(O)R⁵, and —NR^{6c}C(O)NR^{6a}R^{6b}, wherein
   each of the said (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₅ to C₁₀) cycloalkenyl, —O—(C₂ to C₉) cycloheteroalkyl, (C₆ to C₁₀) aryl, and (C₂ to C₉) heteroaryl is optionally substituted with at least one R⁷ group as defined above.

In another embodiment, the invention relates to compounds of Structural Formulae Ia and Ib, or a pharmaceutically acceptable salt, and a pharmaceutically acceptable carrier, diluent, or vehicle thereof, wherein R³ is defined as above and wherein
R¹ and R² are independently selected from the group consisting of

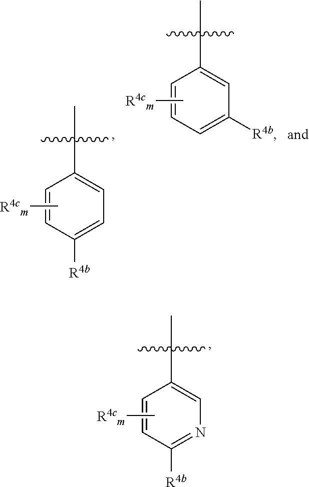

R^{4b} is selected from the group consisting of
(C₂ to C₆) alkyl, (C₂ to C₆) alkenyl, (C₂ to C₆) alkynyl, —O—(C₂ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₅ to C₁₀) cycloalkenyl, —O—(C₂ to C₉) cycloheteroalkyl, (C₆ to C₁₀) aryl, (C₂ to C₉) heteroaryl, isopropoxy, cyclopropoxy, tert-butoxy, cyclopropylmethoxy, (2-hydroxypropan)-2-yl, (1-hydroxycyclopropan)-1-yl, (1-hydroxycyclobutan)-1-yl, trifluoromethoxy, trifluoromethyl, difluoromethoxy, trifluoroethoxy, methoxyethoxy, phenoxy, benzyloxy, difluoromethyl, cyclopropyl, acetyl, n-propyloxy, n-butyloxy, isobutyloxy, sec-butyloxy, cyclobutyloxy, cyclopentyloxy, (1-hydroxycyclopentan)-1-yl, hydroxyethoxy, ethoxyethoxy, isopropoxyethoxy, isopropyl, n-propyl, n-butyl, sec-butyl, tert-butyl, cyclobutyl, cyclopentyl, propanoyl, isobutanoyl, sec-butanoyl, tert-butanoyl, cyclopropanoyl, cyclobutanoyl, and cyclopentanoyl;
wherein each of the said (C₂ to C₆) alkyl, (C₂ to C₆) alkenyl, (C₂ to C₆) alkynyl, —O—(C₂ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₅ to C₁₀) cycloalkenyl, —O—(C₂ to C₉) cycloheteroalkyl, (C₆ to C₁₀) aryl, and (C₂ to C₉) heteroaryl is optionally substituted with at least one R⁷ group as defined above;
R^{4c} is independently selected from hydrogen, deuterium, halogen, cyano, OH, CF₃, (C₁ to C₆) alkyl, (C₂ to C₆) alkenyl, (C₂ to C₆) alkynyl, —O—(C₂ to C₆) alkyl, —O—(C₂ to C₆) alkenyl, —O—(C₂ to C₆) alkynyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₅ to C₁₀) cycloalkenyl, —O—(C₂ to C₉) cycloheteroalkyl, (C₆ to C₁₀) aryl, (C₂ to C₉) heteroaryl, —C(O)R⁵, —NR^{6a}R^{6b}, —S(O)ₘR⁵, —S(O)ₘNR^{6a}R^{6b}, —NR^{6a}S(O)ₘR⁵, —OC(O)R⁵, —NR^{6a}C(O)R⁵, and —NR^{6c}C(O)NR^{6a}R^{6b}, wherein
   each of the said (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₅ to C₁₀) cycloalkenyl, —O—(C₂ to C₉) cycloheteroalkyl, (C₆ to C₁₀) aryl, and (C₂ to C₉) heteroaryl is optionally substituted with at least one R⁷ group as defined above.

In another embodiment, the invention relates to compounds of Structural Formulae Ia and Ib, or a pharmaceutically acceptable salt, and a pharmaceutically acceptable carrier, diluent, or vehicle thereof, wherein R¹ and R³ are defined as above and wherein
R² is selected from the group consisting of

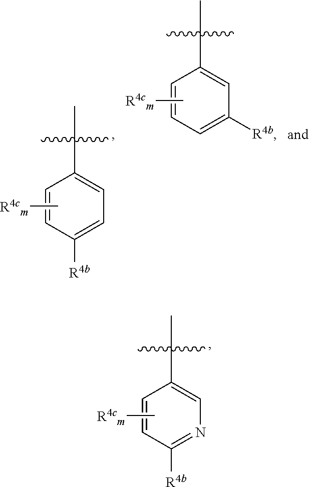

wherein
R^{b} is selected from the group consisting of
(C₃ to C₆) alkyl, (C₂ to C₆) alkenyl, (C₂ to C₆) alkynyl, —O—(C₃ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₅ to C₁₀) cycloalkenyl, —O—(C₂ to C₉) cycloheteroalkyl, (C₆ to C₁₀) aryl, (C₂ to C₉) heteroaryl, isopropoxy, cyclopropoxy, tert-butoxy, cyclopropylmethoxy, (2-hydroxypropan)-2-yl, (1-hydroxycyclopropan)-1-yl, (1-hydroxycyclobutan)-1-yl, trifluoromethoxy, trifluoromethyl, difluoromethoxy, trifluoroethoxy, methoxyethoxy, phenoxy, benzyloxy, difluoromethyl, cyclopropyl, acetyl, n-propyloxy, n-butyloxy, isobutyloxy, sec-butyloxy, cyclobutyloxy, cyclopentyloxy, (1-hydroxycyclopentan)-1-yl, hydroxyethoxy, ethoxyethoxy, isopropoxyethoxy, isopropyl, n-propyl, n-butyl, sec-butyl, tert-butyl, cyclobutyl, cyclopentyl, propanoyl, isobutanoyl, sec-butanoyl, tert-butanoyl, cyclopropanoyl, cyclobutanoyl, and cyclopentanoyl;
wherein each of the said (C₃ to C₆) alkyl, (C₂ to C₆) alkenyl, (C₂ to C₆) alkynyl, —O—(C₃ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₅ to C₁₀) cycloalkenyl, —O—(C₂ to C₉) cycloheteroalkyl, (C₆ to C₁₀) aryl, and (C₂ to C₉) heteroaryl is optionally substituted with at least one R⁷ group as defined above;
R^{4c} is independently selected from hydrogen, deuterium, halogen, cyano, OH, CF₃, (C₁ to C₆) alkyl, (C₂ to C₆) alkenyl, (C₂ to C₆) alkynyl, —O—(C₂ to C₆) alkyl, —O—(C₂ to C₆) alkenyl, —O—(C₂ to C₆) alkynyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₅ to C₁₀) cycloalkenyl, —O—(C₂ to C₉) cycloheteroalkyl, (C₆ to C₁₀) aryl, (C₂ to C₉) heteroaryl, —C(O)R⁵, —NR^{6a}R^{6b}, —S(O)ₘR⁵, —S(O)ₘNR^{6a}R^{6b}, —NR^{6a}S(O)ₘR⁵, —OC(O)R⁵, —NR^{6a}C(O)R⁵, and —NR^{6c}C(O)NR^{6a}R^{6b}, wherein
   each of the said (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₅ to C₁₀) cycloalkenyl, —O—(C₂ to C₉) cycloheteroalkyl, (C₆ to C₁₀) aryl, and (C₂ to C₉) heteroaryl is optionally substituted with at least one R⁷ group as defined above.

In another embodiment, the method comprises administering to humans, other mammals, cell culture, or biological sample an effective amount of a compound represented by Structural Formulae Ia and Ib, wherein R¹, R², and R³ are defined as above or a pharmaceutically acceptable salt, and a pharmaceutically acceptable carrier, diluent, or vehicle thereof, wherein:
R^{4b} is isopropoxy, tert-butoxy, phenoxy, isopropyl, or tert-butyl.

In another embodiment, the method comprises administering to humans, other mammals, cell culture, or biological sample an effective amount of a compound represented by Structural Formulae Ia and Ib, wherein R¹ and R², are defined as above, or a pharmaceutically acceptable salt, and a pharmaceutically acceptable carrier, diluent, or vehicle thereof, wherein:
R³ is hydrogen, deuterium, methyl, or CD₃.

In another embodiment, the method comprises administering to humans, other mammals, cell culture, or biological sample an effective amount of a compound represented by Structural Formulae Ia and Ib, wherein R² and R³ are defined as above, or a pharmaceutically acceptable salt, and a pharmaceutically acceptable carrier, diluent, or vehicle thereof, wherein:
R¹ is

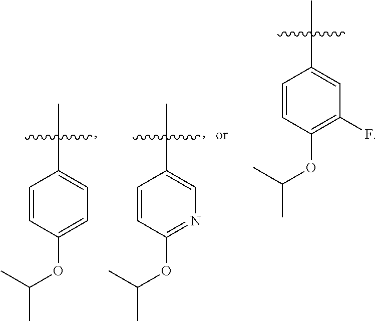 [SMILES: *c1ccc(OC(C)C)cc1 ; *c1ccc(OC(C)C)nc1 ; *c1ccc(OC(C)C)c(F)c1]

In another embodiment, the invention relates to compounds of Structural Formula Ia, or a pharmaceutically acceptable salt, and a pharmaceutically acceptable carrier, diluent, or vehicle thereof, wherein R¹ and R³ are defined as above and wherein
R² is selected from the group consisting of

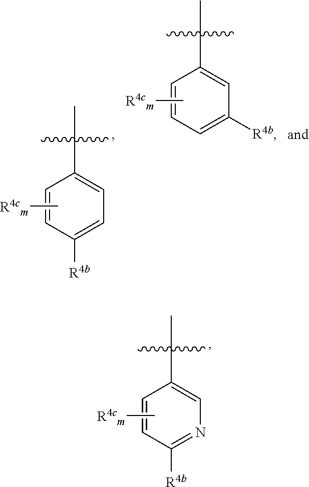

wherein
R^{4b} is selected from the group consisting of
(C₂ to C₆) alkyl, (C₂ to C₆) alkenyl, (C₂ to C₆) alkynyl, —O—(C₂ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₅ to C₁₀) cycloalkenyl, —O—(C₂ to C₉) cycloheteroalkyl, (C₆ to C₁₀) aryl, (C₂ to C₉) heteroaryl, isopropoxy, cyclopropoxy, tert-butoxy, cyclopropylmethoxy, (2-hydroxypropan)-2-yl, (1-hydroxycyclopropan)-1-yl, (1-hydroxycyclobutan)-1-yl, trifluoromethoxy, trifluoromethyl, difluoromethoxy, trifluoroethoxy, methoxyethoxy, phenoxy, benzyloxy, difluoromethyl, cyclopropyl, acetyl, n-propyloxy, n-butyloxy, isobutyloxy, sec-butyloxy, cyclobutyloxy, cyclopentyloxy, (1-hydroxycyclopentan)-1-yl, hydroxyethoxy, ethoxyethoxy, isopropoxyethoxy, isopropyl, n-propyl, n-butyl, sec-butyl, tert-butyl, cyclobutyl, cyclopentyl, propanoyl, isobutanoyl, sec-butanoyl, tert-butanoyl, cyclopropanoyl, cyclobutanoyl, and cyclopentanoyl;
wherein each of the said (C₂ to C₆) alkyl, (C₂ to C₆) alkenyl, (C₂ to C₆) alkynyl, —O—(C₂ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₅ to C₁₀) cycloalkenyl, —O—(C₂ to C₉) cycloheteroalkyl, (C₆ to C₁₀) aryl, and (C₂ to C₉) heteroaryl is optionally substituted with at least one R⁷ group as defined above;
R^{4c} is independently selected from hydrogen, deuterium, halogen, cyano, OH, CF₃, (C₁ to C₆) alkyl, (C₂ to C₆) alkenyl, (C₂ to C₆) alkynyl, —O—(C₂ to C₆) alkyl, —O—(C₂ to C₆) alkenyl, —O—(C₂ to C₆) alkynyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₅ to C₁₀) cycloalkenyl, —O—(C₂ to C₉) cycloheteroalkyl, (C₆ to C₁₀) aryl, (C₂ to C₉) heteroaryl, —C(O)R⁵, —NR^{6a}R^{6b}, —S(O)ₘR⁵, —S(O)ₘNR^{6a}R^{6b}, —NR^{6a}S(O)ₘR⁵, —OC(O)R⁵, —NR^{6a}C(O)R⁵, and —NR^{6c}C(O)NR^{6a}R^{6b}, wherein
   each of the said (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₅ to C₁₀) cycloalkenyl, —O—(C₂ to C₉) cycloheteroalkyl, (C₆ to C₁₀) aryl, and (C₂ to C₆) heteroaryl is optionally substituted with at least one R⁷ group as defined above.

In another embodiment, the method comprises administering to humans, other mammals, cell culture, or biological sample an effective amount of a compound represented by Structural Formula Ia, wherein R¹, R², and R³ are defined as above or a pharmaceutically acceptable salt, and a pharmaceutically acceptable carrier, diluent, or vehicle thereof, wherein:
R^{4b} is isopropoxy, tert-butoxy, phenoxy, isopropyl, or tert-butyl.

In another embodiment, the method comprises administering to humans, other mammals, cell culture, or biological sample an effective amount of a compound represented by Structural Formula Ia, wherein R¹ and R², are defined as above, or a pharmaceutically acceptable salt, and a pharmaceutically acceptable carrier, diluent, or vehicle thereof, wherein:
R³ is hydrogen, deuterium, methyl, or CD₃.

In another embodiment, the method comprises administering to humans, other mammals, cell culture, or biological sample an effective amount of a compound represented by Structural Formula Ia, wherein R¹, R², and R³ are defined as above or a pharmaceutically acceptable salt, and a pharmaceutically acceptable carrier, diluent, or vehicle thereof, wherein:
R^{4c} is hydrogen, deuterium, fluoro, chloro, methyl, or CD₃.

In another embodiment, the method comprises administering to humans, other mammals, cell culture, or biological sample an effective amount of a compound represented by Structural Formula Ia, wherein R² and R³ are defined as above, or a pharmaceutically acceptable salt, and a pharmaceutically acceptable carrier, diluent, or vehicle thereof, wherein:
R¹ is

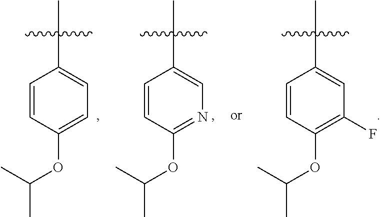 [SMILES: *c1ccc(OC(C)C)cc1 ; *c1ccc(OC(C)C)nc1 ; *c1ccc(OC(C)C)c(F)c1]

In another embodiment, the invention relates to compounds of Structural Formula Ib, or a pharmaceutically acceptable salt, and a pharmaceutically acceptable carrier, diluent, or vehicle thereof, wherein R¹ and R³ are defined as above and wherein
R² is selected from the group consisting of

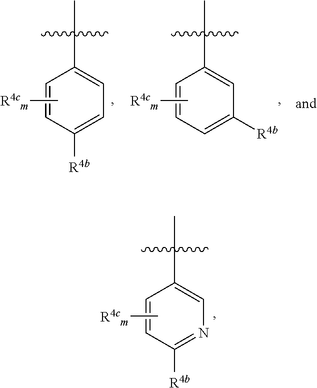

wherein
R^{4b} is selected from the group consisting of
(C₂ to C₆) alkyl, (C₂ to C₆) alkenyl, (C₂ to C₆) alkynyl, —O—(C₂ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₅ to C₁₀) cycloalkenyl, —O—(C₂ to C₉) cycloheteroalkyl, (C₆ to C₁₀) aryl, (C₂ to C₉) heteroaryl, isopropoxy, cyclopropoxy, tert-butoxy, cyclopropylmethoxy, (2-hydroxypropan)-2-yl, (1-hydroxycyclopropan)-1-yl, (1-hydroxycyclobutan)-1-yl, trifluoromethoxy, trifluoromethyl, difluoromethoxy, trifluoroethoxy, methoxyethoxy, phenoxy, benzyloxy, difluoromethyl, cyclopropyl, acetyl, n-propyloxy, n-butyloxy, isobutyloxy, sec-butyloxy, cyclobutyloxy, cyclopentyloxy, (1-hydroxycyclopentan)-1-yl, hydroxyethoxy, ethoxyethoxy, isopropoxyethoxy, isopropyl, n-propyl, n-butyl, sec-butyl, tert-butyl, cyclobutyl, cyclopentyl, propanoyl, isobutanoyl, sec-butanoyl, tert-butanoyl, cyclopropanoyl, cyclobutanoyl, and cyclopentanoyl;
wherein each of the said (C₂ to C₆) alkyl, (C₂ to C₆) alkenyl, (C₂ to C₆) alkynyl, —O—(C₂ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₅ to C₁₀) cycloalkenyl, —O—(C₂ to C₉) cycloheteroalkyl, (C₆ to C₁₀) aryl, and (C₂ to C₉) heteroaryl is optionally substituted with at least one R⁷ group as defined above;
R^{4c} is independently selected from hydrogen, deuterium, halogen, cyano, OH, CF₃, (C₁ to C₆) alkyl, (C₂ to C₆) alkenyl, (C₂ to C₆) alkynyl, —O—(C₂ to C₆) alkyl, —O—(C₂ to C₆) alkenyl, —O—(C₂ to C₆) alkynyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₅ to C₁₀) cycloalkenyl, —O—(C₂ to C₉) cycloheteroalkyl, (C₆ to C₁₀) aryl, (C₂ to C₉) heteroaryl, —C(O)R⁵, —NR^{6a}R^{6b}, —S(O)ₘR⁵, —S(O)ₘNR^{6a}R^{6b}, —NR^{6a}S(O)ₘR⁵, —OC(O)R⁵, —NR^{6a}C(O)R⁵, and —NR^{6c}C(O)NR^{6a}R^{6b}, wherein
   each of the said (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₅ to C₁₀) cycloalkenyl, —O—(C₂ to C₉) cycloheteroalkyl, (C₆ to C₁₀) aryl, and (C₂ to C₉) heteroaryl is optionally substituted with at least one R⁷ group as defined above.

In another embodiment, the invention relates to compounds of Structural Formula Ib, or a pharmaceutically acceptable salt, and a pharmaceutically acceptable carrier, diluent, or vehicle thereof, wherein R¹, R², and R³ are defined as above and wherein:
R^{4b} is isopropoxy, tert-butoxy, phenoxy, isopropyl, or tert-butyl.

In another embodiment, the invention relates to compounds of Structural Formula Ib, or a pharmaceutically acceptable salt, and a pharmaceutically acceptable carrier, diluent, or vehicle thereof, wherein R¹ and R² are defined as above and wherein:
R³ is hydrogen, deuterium, methyl, or CD₃.

In another embodiment, the invention relates to compounds of Structural Formula Ib, or a pharmaceutically acceptable salt, and a pharmaceutically acceptable carrier, diluent, or vehicle thereof, wherein R¹, R², and R³ are defined as above and wherein:
R^{4c} is hydrogen, deuterium, fluoro, chloro, methyl, or CD₃.

In another embodiment, the invention relates to compounds of Structural Formula Ib, or a pharmaceutically acceptable salt, and a pharmaceutically acceptable carrier, diluent, or vehicle thereof, wherein R² and R³ are defined as above and wherein:
R¹ is

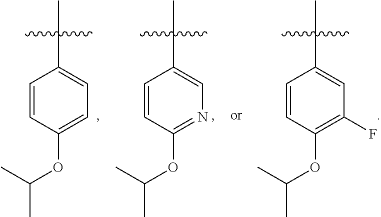 [SMILES: *c1ccc(OC(C)C)cc1 ; *c1ccc(OC(C)C)nc1 ; *c1ccc(OC(C)C)c(F)c1]

In another embodiment, the invention comprises compounds and the use of the compounds in modulating at least one voltage-gated sodium channel.

In another embodiment, the invention relates to compounds, or a pharmaceutically acceptable salt, and a pharmaceutically acceptable carrier, diluent, or vehicle thereof, selected from the group consisting of the compounds described as Examples C58 to C61, C66 to C91, and D23.

In another embodiment, the invention relates to compounds, or a pharmaceutically acceptable salt, and a pharmaceutically acceptable carrier, diluent, or vehicle thereof, selected from the group consisting of:

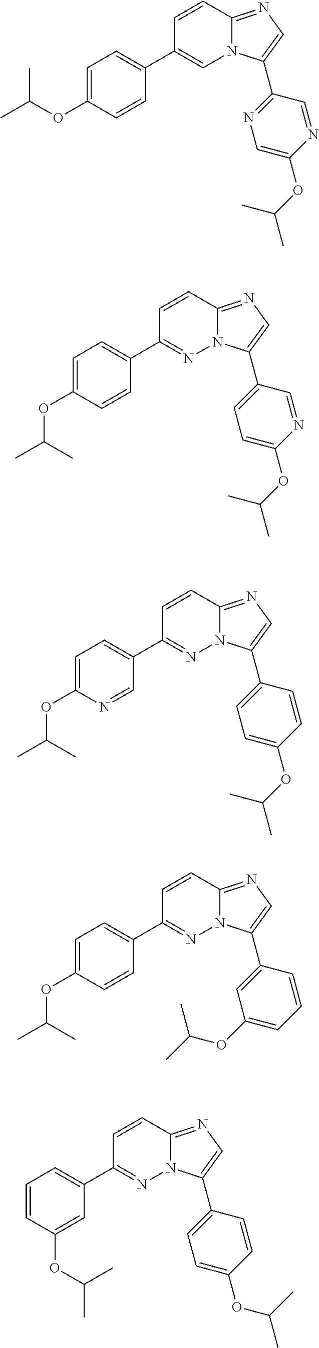 [SMILES: CC(C)Oc1ccc(-c2ccc3ncc(-c4cnc(OC(C)C)cn4)n3c2)cc1 ; CC(C)Oc1ccc(-c2ccc3ncc(-c4ccc(OC(C)C)nc4)n3n2)cc1 ; CC(C)Oc1ccc(-c2cnc3ccc(-c4ccc(OC(C)C)nc4)nn23)cc1 ; CC(C)Oc1ccc(-c2ccc3ncc(-c4cccc(OC(C)C)c4)n3n2)cc1 ; CC(C)Oc1ccc(-c2cnc3ccc(-c4cccc(OC(C)C)c4)nn23)cc1]

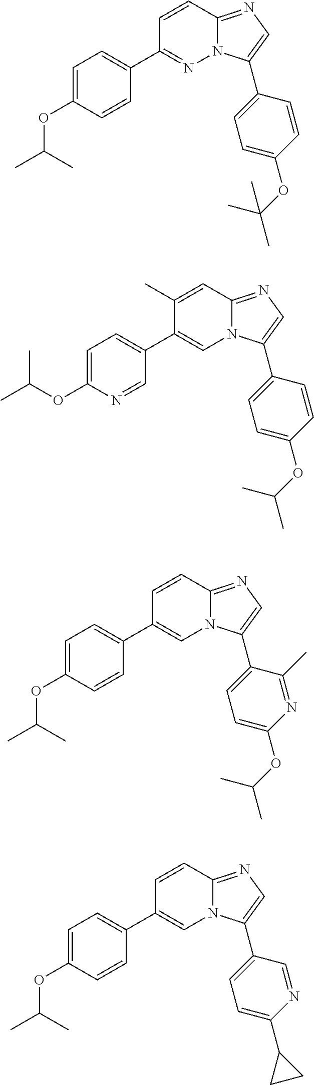

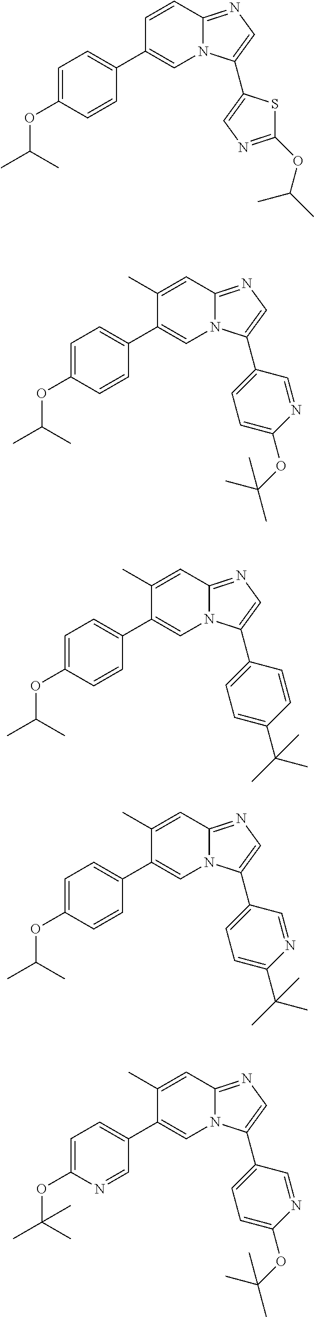

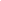

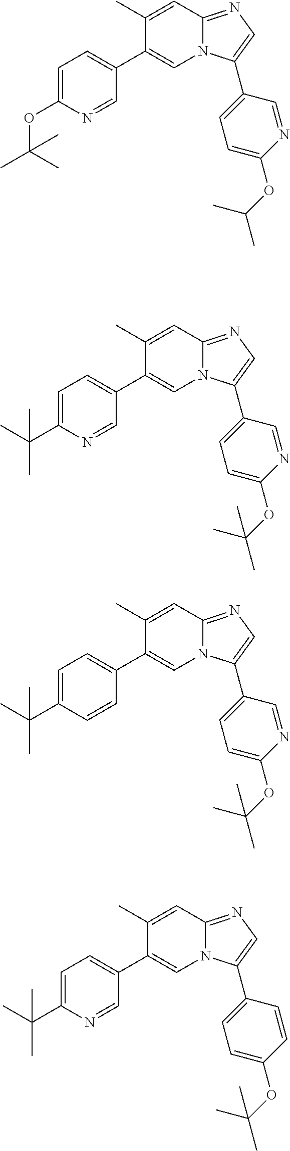

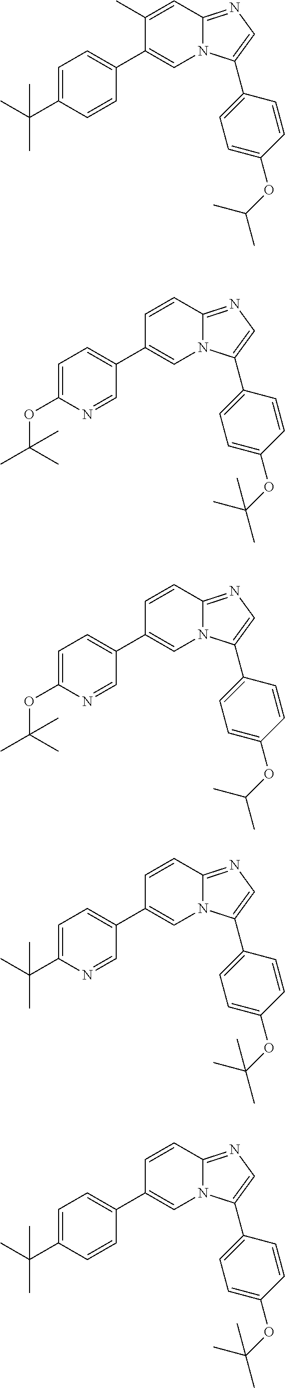

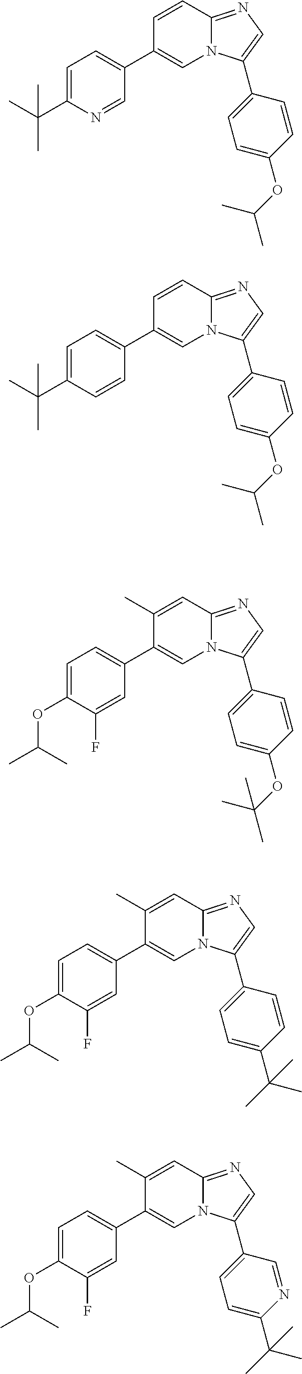

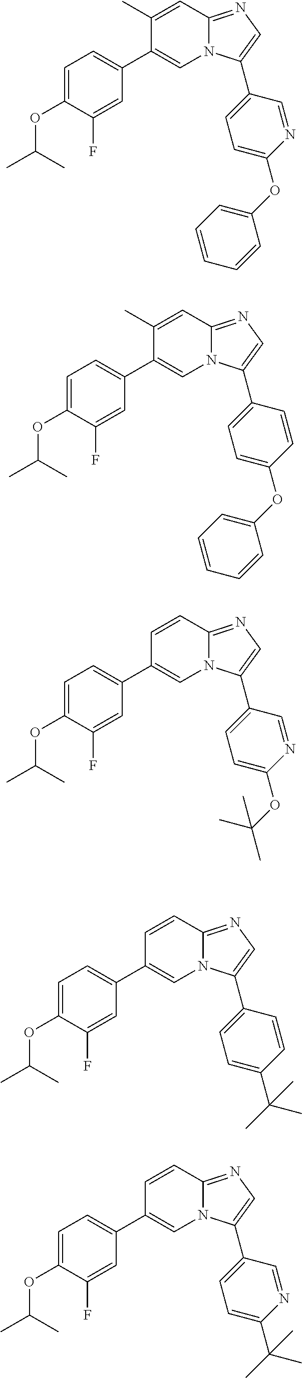

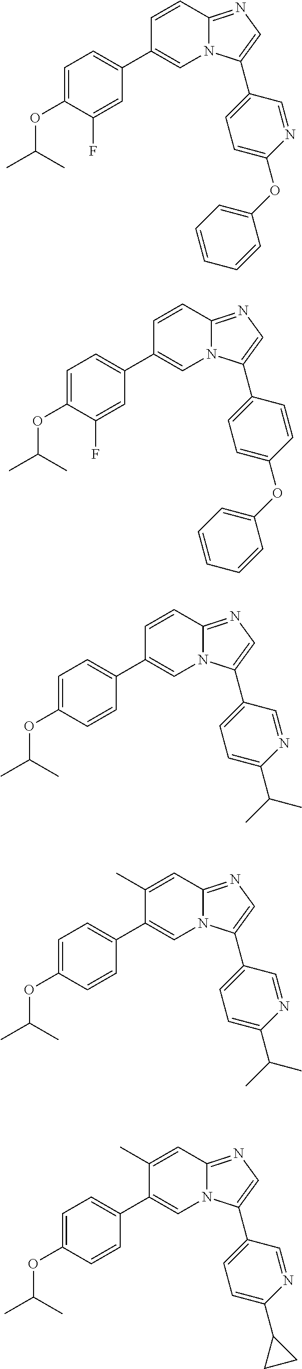

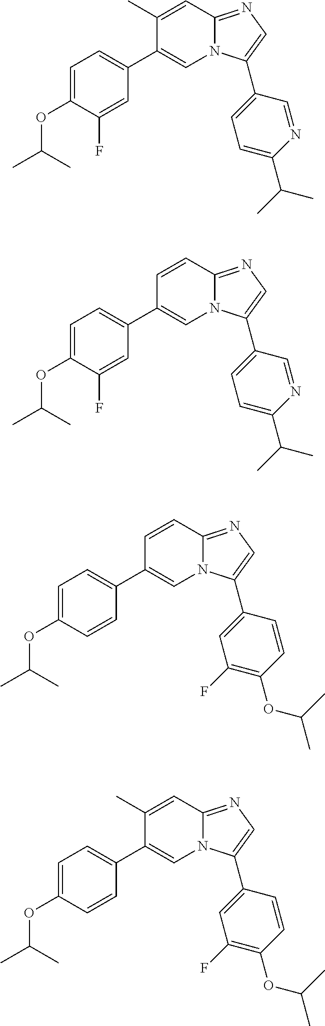

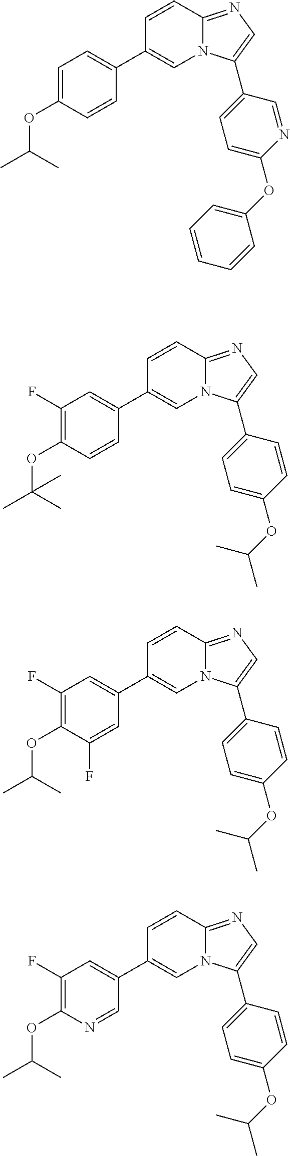 [SMILES: CC(C)Oc1ccc(-c2ccc3ncc(-c4ccc(Oc5ccccc5)nc4)n3c2)cc1 ; CC(C)Oc1ccc(-c2cnc3ccc(-c4ccc(OC(C)(C)C)c(F)c4)cn23)cc1 ; CC(C)Oc1ccc(-c2cnc3ccc(-c4cc(F)c(OC(C)C)c(F)c4)cn23)cc1 ; CC(C)Oc1ccc(-c2cnc3ccc(-c4cnc(OC(C)C)c(F)c4)cn23)cc1]

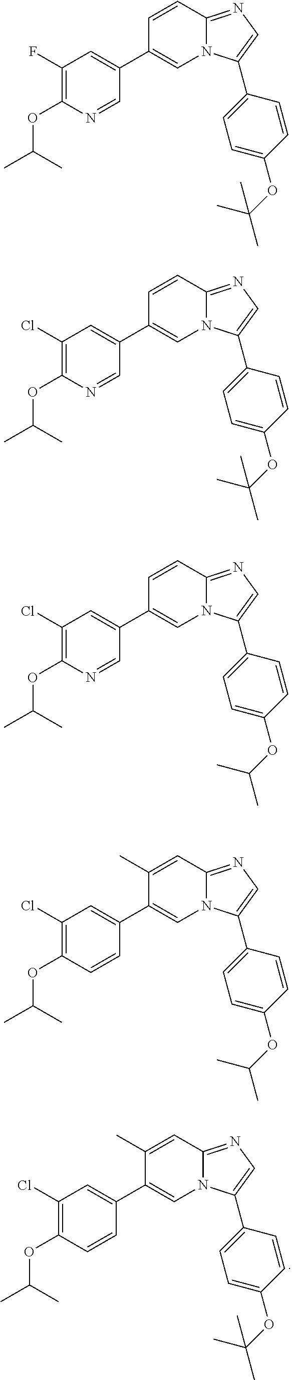

One can treat a disease or condition in a mammal or human that is amenable to treatment by a voltage-gated sodium channel modulator by administering a pharmaceutically effective amount of a pharmaceutical composition comprising a compound selected of Structural Formula I or a compound as shown above with a pharmaceutically acceptable carrier, dilutant, or vehicle.

In another embodiment, compounds of Structural Formula I modulate one or more voltage-gated sodium channels.

In another embodiment, compounds of Structural Formula I may be specific to one or a plurality of voltage-gated sodium channels, including Na_{V}1.1, 1.2, 1.3, 1.4, 1.5, 1.6, 1.7, 1.8, or 1.9.

In another embodiment, the compounds of the invention and their therapeutically acceptable salts, esters, tautomeric forms are potentially of use as medicaments for the treatment of diseases and conditions associated with or influenced by abnormal expression, function or activity of voltage-gated sodium channels.

In another embodiment, the compounds of the invention and their therapeutically acceptable salts, esters, tautomeric forms are potentially of use as medicaments for the treatment of diseases and conditions associated with dysfunction of cell excitability that may be influenced by voltage-gated sodium channel activity.

In another embodiment, the compounds of the invention and their therapeutically acceptable salts, esters, tautomeric forms are potentially of use as medicaments for the treatment of diseases and conditions associated with dysfunction of ion homeostasis that may be influenced by voltage-gated sodium channel activity.

In another embodiment, the compounds of the invention and their therapeutically acceptable salts, esters, tautomeric forms are potentially of use as medicaments for the treatment of various types of cancer, which include, but are not limited to, cancers of the breast, lung, prostate, pancreatic, colon, stomach, ovary, cervix, bladder, oral squamous cell, endometrium, connective tissue, skin, astrocytoma, lymphoma, neuroblastoma, mesothelioma, myeloma, hepatocellular carcinoma, leukaemia, and osteosarcoma.

In another embodiment, the compounds of the invention and their therapeutically acceptable salts, esters, tautomeric forms are potentially of use as medicaments for the treatment of diseases and conditions associated with or influenced by abnormal late or persistent sodium current enhancement.

In another embodiment, the compounds of the invention and their therapeutically acceptable salts, esters, tautomeric forms are potentially of use as medicaments for the treatment of cardiovascular diseases and neurological disorders.

In another embodiment, the compounds of the invention and their therapeutically acceptable salts, esters, tautomeric forms are potentially of use as medicaments for the treatment of cardiovascular diseases, which include, but are not limited to, ventricular tachycardia, ventricular fibrillation, ventricular arrhythmia, atrial arrhythmia, stable angina, unstable angina, Prinzmetal's angina, ischemia, recurrent ischemia, cerebrovascular ischemia, stroke, renal ischemia, ischemia and reperfusion injury, heart failure, congestive heart failure, systolic heart failure, diastolic heart failure, acute heart failure, myocardial infarction, reperfusion injury, intermittent claudication, peripheral artery disease, acute coronary syndrome, hypertrophic cardiomyopathy, and inherited arrhythmia syndromes (including Brugada syndrome and long QT syndrome).

In another embodiment, the compounds of the invention and their therapeutically acceptable salts, esters, tautomeric forms are potentially of use as medicaments for the treatment of neurological disorders, which include, but are not limited to, epilepsy or an epilepsy syndrome (including benign neonatal-infantile familial seizures, simple febrile seizures, infantile spasms, generalized epilepsy with febrile seizures plus (GEFS+), epileptic encephalopathy, focal temporal and frontal lobe epilepsies, severe myoclonic epilepsy of infancy (also known as Dravet's syndrome), intractable childhood epilepsy with generalized tonic-clonic seizures, Rasmussen encephalitis, malignant migrating partial seizures of infancy, West syndrome, Ohtahara syndrome, Lennox-Gastaut syndrome, Landau-Kleffner syndrome), neurodegenerative diseases (including Alzheimer's disease), neurodevelopmental disorders (including autism and tuberous sclerosis complex), neuromuscular disorders (including amyotropic lateral sclerosis, multiple sclerosis, periodic paralysis, and myotonia), pain (including acute, chronic, inflammatory and neuropathic pain), neural trauma, peripheral neuropathy, stroke, migraine, ataxia, irritable bowel syndrome, seizures, and paralysis.

In another embodiment, the compounds of the invention and their therapeutically acceptable salts, esters, tautomeric forms are potentially of use as medicaments for the treatment of irritable bowel syndrome.

### Definitions

As used herein, the terms “comprising” and “Including” are used in their open, non-limiting sense.

The terms “halo” and/or “halogen” refer to fluorine, chlorine, bromine or iodine.

The term “(C₁ to C₆)” alkyl refers to a saturated aliphatic hydrocarbon radical including straight chain and branched chain groups of 1 to 6 carbon atoms. Examples of (C₁ to C₆) alkyl groups include methyl, ethyl, propyl, 2-propyl, n-butyl, iso-butyl, tert-butyl, pentyl, and the like. The terms “Me” and “methyl” as used herein, mean a —CH₃ group. The terms “Et” and “ethyl,” as used herein, mean a —C₂H₅ group.

The term “(C₂ to C₉) alkenyl”, as used herein, means an alkyl moiety comprising 2 to 8 carbons having at least one carbon-carbon double bond. The carbon-carbon double bond in such a group may be anywhere along the 2 to 8 carbon chain that will result in a stable compound. Such groups include both the E and Z isomers of said alkenyl moiety. Examples of such groups include, but are not limited to, ethenyl, propenyl, butenyl, allyl, and pentenyl. The term “allyl,” as used herein, means a —CH₂CH═CH₂ group. The term, “C(R)═C(R),” as used herein, represents a carbon-carbon double bond in which each carbon is substituted by an R group, and includes E and Z isomers.

As used herein, the term “(C₂ to C₈) alkynyl” means an alkyl moiety comprising from 2 to 8 carbon atoms and having at least one carbon-carbon triple bond. The carbon-carbon triple bond in such a group may be anywhere along the 2 to 8 carbon chain that will result in a stable compound. Examples of such groups include, but are not limited to, ethyne, propyne, 1-butyne, 2-butyne, 1-pentyne, 2-pentyne, 1-hexyne, 2-hexyne, and 3-hexyne.

The term “(C₁ to C₈) alkoxy”, as used herein, means an O-alkyl group wherein said alkyl group contains from 1 to 8 carbon atoms and is straight, branched, or cyclic. Examples of such groups include, but are not limited to, methoxy, ethoxy, n-propyloxy, iso-propyloxy, n-butoxy, iso-butoxy, tert-butoxy, cyclopentyloxy, and cyclohexyloxy.

The term “(C₆ to C₁₀) aryl”, as used herein, means a group derived from an aromatic hydrocarbon containing from 6 to 10 carbon atoms. Examples of such groups include, but are not limited to, phenyl or naphthyl. The terms “Ph” and “phenyl,” as used herein, mean a —C₆H₅ group. The term “benzyl,” as used herein, means a —CH₂C₆H₅ group.

“(C₂ to C₉) heteroaryl”, as used herein, means an aromatic heterocyclic group having a total of from 5 to 10 atoms in its ring, and containing from 2 to 9 carbon atoms and from one to four heteroatoms each independently selected from O, S and N, and with the proviso that the ring of said group does not contain two adjacent O atoms or two adjacent S atoms. The heterocyclic groups include benzo-fused ring systems. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The (C₂ to C₉) heteroaryl groups may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached).

“(C₂ to C₉) cycloheteroalkyl”, as used herein, means a non-aromatic, monocyclic, bicyclic, tricyclic, spirocyclic, or tetracyclic group having a total of from 4 to 13 atoms in its ring system, and containing from 2 to 9 carbon atoms and from 1 to 4 heteroatoms each independently selected from O, S and N, and with the proviso that the ring of said group does not contain two adjacent O atoms or two adjacent S atoms. Furthermore, such C₂ to C₉ cycloheteroalkyl groups may contain an oxo substituent at any available atom that will result in a stable compound. For example, such a group may contain an oxo atom at an available carbon or nitrogen atom. Such a group may contain more than one oxo substituent if chemically feasible. In addition, it is to be understood that when such a C2 to C9 cycloheteroalkyl group contains a sulfur atom, said sulfur atom may be oxidized with one or two oxygen atoms to afford either a sulfoxide or sulfone. An example of a 4 membered cycloheteroalkyl group is azetidinyl (derived from azetidine). An example of a 5 membered cycloheteroalkyl group is pyrrolidinyl. An example of a 6 membered cycloheteroalkyl group is piperidinyl. An example of a 9 membered cycloheteroalkyl group is indolinyl. An example of a 10 membered cycloheteroalkyl group is 4H-quinolizinyl. Further examples of such C₂ to C₉ cycloheteroalkyl groups include, but are not limited to, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, quinolizinyl, 3-oxopiperazinyl, 4-methylpiperazinyl, 4-ethylpiperazinyl, and 1-oxo-2,8,diazaspiro[4.5]dec-8-yl.

The term “(C₃ to C₁₀) cycloalkyl group” means a saturated, monocyclic, fused, spirocyclic, or polycyclic ring structure having a total of from 3 to 10 carbon ring atoms. Examples of such groups include, but are not limited to, cyclopropyl, cyclobutyl, cyclopentyl, cyclopentenyl, cyclohexyl, cycloheptyl, and adamantyl.

The term “spirocyclic” as used herein has its conventional meaning, that is, any compound containing two or more rings wherein two of the rings have one ring carbon in common. The rings of a spirocyclic compound, as herein defined, independently have 3 to 20 ring atoms. Preferably, they have 3 to 10 ring atoms. Non-limiting examples of a spirocyclic compound include spiro[3.3]heptane, spiro[3.4]octane, and spiro[4.5]decane.

The term “(C₅ to C₆) cycloalkenyl” means an unsaturated, monocyclic, fused, spirocyclic ring structures having a total of from 5 to 8 carbon ring atoms. Examples of such groups include, but are not limited to, cyclopentenyl, cyclohexenyl.

The term cyano” refers to a —C≡N group.

An “aldehyde” group refers to a carbonyl group where R is hydrogen.

An “alkoxy” group refers to both an —O-alkyl and an —O-cycloalkyl group, as defined herein.

An “alkoxycarbonyl” refers to a —C(O)OR.

An “alkylaminoalkyl” group refers to an -alkyl-NR-alkyl group.

An “alkylsulfonyl” group refer to a —SO₂alkyl.

An “amino” group refers to an —NH₂ or an —NRR′ group.

An “aminoalkyl” group refers to an -alkyl-NRR′ group.

An “aminocarbonyl” refers to a —C(O)NRR′.

An “arylalkyl” group refers to -alkylaryl, where alkyl and aryl are defined herein.

An “aryloxy” group refers to both an —O-aryl and an —O-heteroaryl group, as defined herein.

An “aryloxycarbonyl” refers to —C(O)Oaryl.

An “arylsulfonyl” group refers to a —SO₂aryl.

A “C-amido” group refers to a —C(O)NRR′ group.

A “carbonyl” group refers to a —C(O)R.

A “C-carboxyl” group refers to a —C(O)OR groups.

A “carboxylic acid” group refers to a C-carboxyl group in which R is hydrogen.

A “dialkylaminoalkyl” group refers to an -(alkyl)N(alkyl)₂ group.

A “halo” or “halogen” group refers to fluorine, chlorine, bromine or iodine.

A “haloalkyl” group refers to an alkyl group substituted with one or more halogen atoms.

A “heteroalicycloxy” group refers to a heteroalicyclic-O group with heteroalicyclic as defined herein.

A “heteroaryloxyl” group refers to a heteroaryl-O group with heteroaryl as defined herein.

A “hydroxy” group refers to an —OH group.

An “N-amido” group refers to a —R′C(O)NR group.

An “N-carbamyl” group refers to a —ROC(O)NR-group.

A “nitro” group refers to a —NO₂ group.

An “N-Sulfonamido” group refers to a —NR—S(O)₂R group.

An “N-thiocarbamyl” group refers to a ROC(S)NR′ group.

An “O-carbamyl” group refers to a —OC(O)NRR′ group.

An “O-carboxyl” group refers to a RC(O)O group.

An “O-thiocarbamyl” group refers to a —OC(S)NRR′ group.

An “oxo” group refers to a carbonyl moiety such that alkyl substituted by oxo refers to a ketone group.

A “perfluoroalkyl group” refers to an alkyl group where all of the hydrogen atoms have been replaced with fluorine atoms.

A “phosphonyl” group refers to a —P(O)(OR)₂ group.

A “silyl” group refers to a —SiR₃ group.

An “S-sulfonamido” group refers to a —S(O)₂NR-group.

A “sulfinyl” group refers to a —S(O)R group.

A “sulfonyl” group refers to a —S(O)₂R group.

A “thiocarbonyl” group refers to a —C(═S)—R group.

A “trihalomethanecarbonyl” group refers to a Z₃CC(O) group, where Z is halogen.

A “trihalomethanesulfonamido” group refers to a Z₃CS(O)₂NR-group, where Z is halogen.

A “trihalomethanesulfonyl” group refers to a Z₃CS(O)₂ group, where Z is halogen.

A “trihalomethyl” group refers to a —CZ₃ group, where Z is halogen.

A “C-carboxyl” group refers to a —C(O)OR groups.

The term “substituted,” means that the specified group or moiety bears one or more substituents.

The term “unsubstituted,” means that the specified group bears no substituents. The term “optionally substituted” means that the specified group is unsubstituted or substituted by one or more substituents. It is to be understood that in the compounds of the present invention when a group is said to be “unsubstituted,” or is “substituted” with fewer groups than would fill the valencies of all the atoms in the compound, the remaining valencies on such a group are filled by hydrogen. For example, if a C₆ aryl group, also called “phenyl” herein, is substituted with one additional substituent, one of ordinary skill in the art would understand that such a group has 4 open positions left on carbon atoms of the C₆ aryl ring (6 initial positions, minus one to which the remainder of the compound of the present invention is bonded, minus an additional substituent, to leave 4). In such cases, the remaining 4 carbon atoms are each bound to one hydrogen atom to fill their valencies. Similarly, if a C₆ aryl group in the present compounds is said to be “disubstituted,” one of ordinary skill in the art would understand it to mean that the C₆ aryl has 3 carbon atoms remaining that are unsubstituted. Those three unsubstituted carbon atoms are each bound to one hydrogen atom to fill their valencies.

The term “solvate,” is used to describe a molecular complex between compounds of the present invention and solvent molecules. Examples of solvates include, but are not limited to, compounds of the invention in combination water, isopropanol, ethanol, methanol, dimethylsulfoxide (DMSO), ethyl acetate, acetic acid, ethanolamine, or mixtures thereof. The term “hydrate” can be used when said solvent is water. It is specifically contemplated that in the present invention one solvent molecule can be associated with one molecule of the compounds of the present invention, such as a hydrate. Furthermore, it is specifically contemplated that in the present invention, more than one solvent molecule may be associated with one molecule of the compounds of the present invention, such as a dihydrate. Additionally, it is specifically contemplated that in the present invention less than one solvent molecule may be associated with one molecule of the compounds of the present invention, such as a hemihydrate. Furthermore, solvates of the present invention are contemplated as solvates of compounds of the present invention that retain the biological effectiveness of the non-hydrate form of the compounds.

The term “pharmaceutically acceptable salt,” as used herein, means a salt of a compound of the present invention that retains the biological effectiveness of the free acids and bases of the specified derivative and that is not biologically or otherwise undesirable.

The term “pharmaceutically acceptable formulation,” as used herein, means a combination of a compound of the invention, or a salt or solvate thereof, and a carrier, diluent, and/or excipient(s) that are compatible with a compound of the present invention, and is not deleterious to the recipient thereof. Pharmaceutical formulations can be prepared by procedures known to those of ordinary skill in the art. For example, the compounds of the present invention can be formulated with common excipients, diluents, or carriers, and formed into tablets, capsules, and the like. Examples of excipients, diluents, and carriers that are suitable for such formulations include the following: fillers and extenders such as starch, sugars, mannitol, and silicic derivatives; binding agents such as carboxymethyl cellulose and other cellulose derivatives, alginates, gelatin, and polyvinyl pyrrolidone; moisturizing agents such as glycerol; disintegrating agents such as povidone, sodium starch glycolate, sodium carboxymethylcellulose, agar, calcium carbonate, and sodium bicarbonate; agents for retarding dissolution such as paraffin; resorption accelerators such as quaternary ammonium compounds; surface active agents such as cetyl alcohol, glycerol monostearate; adsorptive carriers such as kaolin and bentonite; and lubricants such as talc, calcium and magnesium stearate and solid polyethylene glycols. Final pharmaceutical forms may be pills, tablets, powders, lozenges, saches, cachets, or sterile packaged powders, and the like, depending on the type of excipient used. Additionally, it is specifically contemplated that pharmaceutically acceptable formulations of the present invention can contain more than one active ingredient. For example, such formulations may contain more than one compound according to the present invention. Alternatively, such formulations may contain one or more compounds of the present invention and one or more additional agents that reduce abnormal cell growth.

The term “modulating” as used herein, refers to blocking or enhancing one or more components of the activity, such as peak, late or persistent current, by a measurable amount and by block we mean that the sodium channel activity is decreased and by enhancing we mean that the sodium channel activity is increased.

The term “modulating amount” as used herein, refers to the amount of a compound of the present invention, or a salt or solvate thereof, required to block or enhance one or more components of the voltage-gated sodium channel activity, such as peak, late or persistent current, in vivo, such as in a mammal or in vitro. The amount of such compounds required to cause such modulation can be determined without undue experimentation using methods described herein and those known to those of ordinary skill in the art.

The term “inhibiting amount” as used herein, refers to the amount of a compound of the present invention, or a salt or solvate thereof, required to block or enhance one or more components of the voltage-gated sodium channel activity, such as peak, late or persistent current, in vivo, such as in a mammal, or in vitro. The amount of such compounds required to cause such blockage or enhancement can be determined without undue experimentation using methods described herein and those known to those of ordinary skill in the art.

The term “therapeutically effective amount,” as used herein, means an amount of a compound of the present invention, or a salt or solvate thereof, that, when administered to a mammal in need of such treatment, is sufficient to effect treatment, as defined herein. Thus, a therapeutically effective amount of a compound of the present invention, or a salt or solvate thereof, is a quantity sufficient to block or enhance one or more components of the voltage-gated sodium channel activity, such as peak, late or persistent current, such that the condition associated with pathophysiological voltage-gated sodium channel activity or other pathophysiological disorders of ion homeostasis that may be influenced by voltage-gated sodium channel activity is reduced or alleviated.

The terms “treat”, “treating”, and “treatment” with reference to voltage-gated sodium channel activity, in mammals, particularly a human, include: (i) preventing the disease or condition from occurring in a subject which may be predisposed to the condition, such that the treatment constitutes prophylactic treatment for the pathologic condition; (ii) modulating or inhibiting the disease or condition, i.e., arresting its development; (iii) relieving the disease or condition, i.e., causing regression of the disease or condition; or (iv) relieving and/or alleviating the disease or condition or the symptoms resulting from the disease or condition.

Unless indicated otherwise, all references herein to the inventive compounds include references to salts, solvates, and complexes thereof, including polymorphs, stereoisomers, tautomers, and isotopically labeled versions thereof. For example, compounds of the present invention can be pharmaceutically acceptable salts and/or pharmaceutically acceptable solvates.

The term “stereoisomers” refers to compounds that have identical chemical constitution, but differ with regard to the arrangement of their atoms or groups in space. In particular, the term “enantiomers” refers to two stereoisomers of a compound that are non-superimposable mirror images of one another. A pure enantiomer can be contaminated with up to 2% of the opposite enantiomer.

The terms “racemic” or “racemic mixture,” as used herein, refer to a 1:1 mixture of enantiomers of a particular compound. The term “diastereomers”, on the other hand, refers to the relationship between a pair of stereoisomers that comprise two or more asymmetric centers and are not mirror images of one another. In accordance with a convention used in the art, the symbol is used in structural formulas herein to depict the bond that is the point of attachment of the moiety or substituent to the core or backbone structure. In accordance with another convention, in some structural formulae herein the carbon atoms and their bound hydrogen atoms are not explicitly depicted, e.g.,

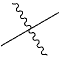

represents a methyl group,

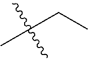 [SMILES: *CC]

represents an ethyl group,

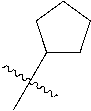 [SMILES: *C1CCCC1]

represents a cyclopentyl group, etc.

The compounds of the present invention may have asymmetric carbon atoms. The carbon carbon bonds of the compounds of the present invention may be depicted herein using a solid line (-), a solid wedge ( [symbol] ), or a dotted wedge ( [symbol] ). The use of a solid line to depict bonds to asymmetric carbon atoms is meant to indicate that all possible stereoisomers (e.g. specific enantiomers, racemic mixtures, etc.) at that carbon atom are included. The use of either a solid or dotted wedge to depict bonds to asymmetric carbon atoms is meant to indicate that only the stereoisomer shown is meant to be included. It is possible that compounds of the invention may contain more than one asymmetric carbon atom. In those compounds, the use of a solid line to depict bonds to asymmetric carbon atoms is meant to indicate that all possible stereoisomers are meant to be included. For example, unless stated otherwise, it is intended that the compounds of the present invention can exist as enantiomers and diastereomers or as racemates and mixtures thereof. The use of a solid line to depict bonds to one or more asymmetric carbon atoms in a compound of the invention and the use of a solid or dotted wedge to depict bonds to other asymmetric carbon atoms in the same compound is meant to indicate that a mixture of diastereomers is present.

Unless otherwise defined, a substituent “R” may reside on any atom of the ring system, assuming replacement of a depicted, implied, or expressly defined hydrogen from one of the ring atoms, so long as a stable structure is formed.

Conventional techniques for the preparation/isolation of individual enantiomers include chiral synthesis from a suitable optically pure precursor or resolution of the racemate using, for example, chiral high pressure liquid chromatography (HPLC). Alternatively, the racemate (or a racemic precursor) may be reacted with a suitable optically active compound, for example, an alcohol, or, in the case where the compound contains an acidic or basic moiety, an acid or base such as tartaric acid or 1-phenyl ethyl amine. The resulting diastereomeric mixture may be separated by chromatography and/or fractional crystallization and one or both of the diastereoisomers converted to the corresponding pure enantiomer(s) by means well known to one skilled in the art. Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture. Stereoisomeric conglomerates may be separated by conventional techniques known to those skilled in the art. See, e.g. “Stereochemistry of Organic Compounds” by E L Eliel (Wiley, New York, 1994), the disclosure of which is incorporated herein by reference in its entirety.

Where a compound of the invention contains an alkenyl or alkenylene group, geometric cis/trans (or Z/E) isomers are possible. Where the compound contains, for example, a keto or oxime group or an aromatic moiety, tautomeric isomerism (‘tautomerism’) can occur. Examples of tautomerism include keto and enol tautomers. A single compound may exhibit more than one type of isomerism. Included within the scope of the invention are all stereoisomers, geometric isomers and tautomeric forms of the inventive compounds, including compounds exhibiting more than one type of isomerism, and mixtures of one or more thereof. Cis/trans isomers may be separated by conventional techniques well known to those skilled in the art, for example, chromatography and fractional crystallization.

The compounds of the present invention may be administered as prodrugs. Thus certain derivatives of compounds of Formula I, which may have little or no pharmacological activity themselves can, when administered to a mammal, be converted into a compound of Formula I having the desired activity, for example, by hydrolytic cleavage. Such derivatives are referred to as “prodrugs”. Prodrugs can, for example, be produced by replacing appropriate functionalities present in the compounds of Formula I with certain moieties known to those skilled in the art. See, e.g. “Prodrugs as Novel Delivery Systems”, Vol. 14, ACS Symposium Series (T Higuchi and W Stella) and “Bioreversible Carriers in Drug Design”, Pergamon Press, 1987 (ed. E B Roche, American Pharmaceutical Association), the disclosures of which are incorporated herein by reference in their entireties. Some examples of such prodrugs include: an ester moiety in the place of a carboxylic acid functional group; an ether moiety or an amide moiety in place of an alcohol functional group; and an amide moiety in place of a primary or secondary amino functional group. Further examples of replacement groups are known to those of skill in the art. See, e.g. “Design of Prodrugs” by H Bundgaard (Elsevier, 1985), the disclosure of which is incorporated herein by reference in its entirety. It is also possible that certain compounds of Formula I may themselves act as prodrugs of other compounds of Formula I.

Salts of the present invention can be prepared according to methods known to those of skill in the art. Examples of salts include, but are not limited to, acetate, acrylate, benzenesulfonate, benzoate (such as chlorobenzoate, methylbenzoate, dinitrobenzoate, hydroxybenzoate, and methoxybenzoate), bicarbonate, bisulfate, bisulfite, bitartrate, borate, bromide, butyne-1,4-dioate, calcium edetate, camsylate, carbonate, chloride, caproate, caprylate, clavulanate, citrate, decanoate, dihydrochloride, dihydrogenphosphate, edetate, edisylate, estolate, esylate, ethylsuccinate, formate, fumarate, gluceptate, gluconate, glutamate, glycollate, glycollylarsanilate, heptanoate, hexyne-1,6-dioate, hexylresorcinate, hydrabamine, hydrobromide, hydrochloride, γ-hydroxybutyrate, iodide, isobutyrate, isothionate, lactate, lactobionate, laurate, malate, maleate, malonate, mandelate, mesylate, metaphosphate, methanesulfonate, methylsulfate, monohydrogenphosphate, mucate, napsylate, naphthalene-1-sulfonate, naphthalene-2-sulfonate, nitrate, oleate, oxalate, pamoate (embonate), palmitate, pantothenate, phenylacetates, phenylbutyrate, phenylpropionate, phthalate, phosphate/diphosphate, polygalacturonate, propanesulfonate, propionate, propiolate, pyrophosphate, pyrosulfate, salicylate, stearate, subacetate, suberate, succinate, sulfate, sulfonate, sulfite, tannate, tartrate, teoclate, tosylate, triethiodide, and valerate salts.

The compounds of the present invention that are basic in nature are capable of forming a wide variety of different salts with various inorganic and organic acids. Although such salts must be pharmaceutically acceptable for administration to animals, it is often desirable in practice to initially isolate the compound of the present invention from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention can be prepared by treating the base compound with a substantially equivalent amount of the selected mineral or organic acid in an aqueous solvent medium or in a suitable organic solvent, such as methanol or ethanol. Upon evaporation of the solvent, the desired solid salt is obtained. The desired acid salt can also be precipitated from a solution of the free base in an organic solvent by adding an appropriate mineral or organic acid to the solution.

Those compounds of the present invention that are acidic in nature are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the acidic compounds of the present invention. Such non-toxic base salts include those derived from such pharmacologically acceptable cations as sodium, potassium, calcium and magnesium, etc. These salts can be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

If the inventive compound is a base, the desired salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the inventive compound is an acid, the desired salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

In the case of agents that are solids, it is understood by those skilled in the art that the inventive compounds, agents and salts may exist in different crystal or polymorphic forms, all of which are intended to be within the scope of the present invention and specified formulas.

The invention also includes isotopically-labeled compounds of the invention, wherein one or more atoms is replaced by an atom having the same atomic number, but an atomic mass or mass number different from the atomic mass or mass number usually found in nature. Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, ³⁵Cl, and ³⁷Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S.

Certain isotopically-labeled compounds of the invention, for example, those incorporating a radioactive isotope, are useful in drug and/or substrate tissue distribution studies. The radioactive isotopes tritium, ³H, and carbon-14, ¹⁴C, are particularly useful for this purpose in view of their ease of incorporation and ready means of detection. Substitution with heavier isotopes such as deuterium, ²H, may afford certain therapeutic advantages resulting from greater metabolic stability, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Tomography (PET) studies for examining substrate receptor occupancy. Isotopically-labeled compounds of the invention can generally be prepared by conventional techniques known to those skilled in the art or by processes analogous to those described herein, using an appropriate isotopically-labeled reagent in place of the non-labeled reagent otherwise employed.

The term “deuterated” refers to the replacement of one or more hydrogen atoms with a corresponding number of deuterium atoms. Unless otherwise stated, when a particular position in a compound of this invention is designated specifically as “D”, “deuterium”, being “deuterated”, or “having deuterium” (the element deuterium is represented by the letter “D” in chemical structures and formulas and indicated with a lower case “d” in chemical names), the position is understood to have deuterium at an abundance that is at least 3000 times greater than the natural abundance of deuterium, which is 0.015% (i.e., the term “D”, “d” or “deuterium” indicates at least 45% incorporation of deuterium).

The term “isotopic enrichment factor”, as used herein, means the ratio between the isotopic abundance and the natural abundance of a specified isotope.

In some embodiments, a compound of this invention has an isotopic enrichment factor for each deuterium present at a site designated as a potential site of deuteration on the compound of at least 3500 (52.5% deuterium incorporation), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

The compounds of the present invention may be formulated into pharmaceutical compositions as described below in any pharmaceutical form recognizable to the skilled artisan as being suitable. Pharmaceutical compositions of the invention comprise a therapeutically effective amount of at least one compound of the present invention and an inert, pharmaceutically acceptable carrier or diluent.

To treat or prevent diseases or conditions mediated in part or whole by voltage-gated sodium channel activity, a pharmaceutical composition of the invention is administered in a suitable formulation prepared by combining a therapeutically effective amount (i.e., a voltage-gated sodium channel modulating, regulating, or inhibiting amount effective to achieve therapeutic efficacy) of at least one compound of the present invention (as an active ingredient) with one or more pharmaceutically suitable carriers, which may be selected, for example, from diluents, excipients and auxiliaries that facilitate processing of the active compounds into the final pharmaceutical preparations.

The pharmaceutical carriers employed may be either solid or liquid. Exemplary solid carriers are lactose, sucrose, talc, gelatin, agar, pectin, acacia, magnesium stearate, stearic acid and the like. Exemplary liquid carriers are syrup, peanut oil, olive oil, water and the like. Similarly, the inventive compositions may include time-delay or time-release material known in the art, such as glyceryl monostearate or glyceryl distearate alone or with a wax, ethylcellulose, hydroxypropylmethylcellulose, methylmethacrylate or the like. Further additives or excipients may be added to achieve the desired formulation properties. For example, a bioavailability enhancer, such as Labrasol, Gelucire or the like, or formulator, such as CMC (carboxy-methylcellulose), PG (propyleneglycol), or PEG (polyethyleneglycol), may be added. Gelucire®, a semi-solid vehicle that protects active ingredients from light, moisture and oxidation, may be added, e.g., when preparing a capsule formulation.

If a solid carrier is used, the preparation can be tableted, placed in a hard gelatin capsule in powder or pellet form, or formed into a troche or lozenge. The amount of solid carrier may vary, but generally will be from about 25 mg to about 1 g. If a liquid carrier is used, the preparation may be in the form of syrup, emulsion, soft gelatin capsule, sterile injectable solution or suspension in an ampoule or vial or non-aqueous liquid suspension. If a semi-solid carrier is used, the preparation may be in the form of hard and soft gelatin capsule formulations. The inventive compositions are prepared in unit-dosage form appropriate for the mode of administration, e.g. parenteral or oral administration.

To obtain a stable water-soluble dose form, a salt of a compound of the present invention may be dissolved in an aqueous solution of an organic or inorganic acid, such as a 0.3 M solution of succinic acid or citric acid. If a soluble salt form is not available, the agent may be dissolved in a suitable co-solvent or combinations of co-solvents. Examples of suitable co-solvents include alcohol, propylene glycol, polyethylene glycol 300, polysorbate 80, glycerin and the like in concentrations ranging from 0 to 60% of the total volume. In an exemplary embodiment, a compound of the present invention is dissolved in DMSO and diluted with water. The composition may also be in the form of a solution of a salt form of the active ingredient in an appropriate aqueous vehicle such as water or isotonic saline or dextrose solution.

Proper formulation is dependent upon the route of administration selected. For injection, the agents of the compounds of the present invention may be formulated into aqueous solutions, preferably in physiologically compatible buffers such as Hanks solution, Ringer's solution, or physiological saline buffer.

For transmucosal administration, penetrants appropriate to the barrier to be permeated are used in the formulation. Such penetrants are generally known in the art.

For oral administration, the compounds can be formulated by combining the active compounds with pharmaceutically acceptable carriers known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

Pharmaceutical preparations that can be used orally also include capsules made of hydroxypropyl methylcellulose (HPMC).

For administration intranasally or by inhalation, the compounds for use according to the present invention may be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount.

Capsules and cartridges of gelatin for use in an inhaler or insufflator and the like may be formulated containing a powder mix of the compound and a suitable powder base such as lactose or starch.

The compounds may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Alternatively, the active ingredient may be in powder form for constitution with a suitable vehicle, e.g. sterile pyrogen-free water, before use.

In addition to the formulations described above, the compounds of the present invention may also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt. A pharmaceutical carrier for hydrophobic compounds is a cosolvent system comprising benzyl alcohol, a non-polar surfactant, a water-miscible organic polymer, and an aqueous phase. The co-solvent system may be a VPD co-solvent system. VPD is a solution of 3% w/v benzyl alcohol, 8% w/v of the non-polar surfactant polysorbate 80, and 65% w/v polyethylene glycol 300, made up to volume in absolute ethanol. The VPD co-solvent system (VPD: 5W) contains VPD diluted 1:1 with a 5% dextrose in water solution. This co-solvent system dissolves hydrophobic compounds well, and itself produces low toxicity upon systemic administration. The proportions of a cosolvent system may be suitably varied without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity due to the toxic nature of DMSO. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for product stabilization may be employed.

The pharmaceutical compositions also may comprise suitable solid- or gel-phase carriers or excipients. These carriers and excipients may provide marked improvement in the bioavailability of poorly soluble drugs. Examples of such carriers or excipients include calcium carbonate, calcium phosphate, sugars, starches, cellulose derivatives, gelatin, and polymers such as polyethylene glycols. Furthermore, additives or excipients such as Gelucire®, Capryol®, Labrafil®, Labrasol®, Lauroglycol®, Plurol®, Peceol® Transcutol® and the like may be used.

Further, the pharmaceutical composition may be incorporated into a skin patch for delivery of the drug directly onto the skin.

It will be appreciated that the actual dosages of the agents of this invention will vary according to the particular agent being used, the particular composition formulated, the mode of administration, and the particular site, host, and disease being treated. Those skilled in the art using conventional dosage determination tests in view of the experimental data for a given compound may ascertain optimal dosages for a given set of conditions. For oral administration, an exemplary daily dose generally employed will be from about 0.001 to about 1000 mg/kg of body weight, with courses of treatment repeated at appropriate intervals.

Furthermore, the pharmaceutically acceptable formulations of the present invention may contain a compound of the present invention, or a salt or solvate thereof, in an amount of about 10 mg to about 2000 mg, or from about 10 mg to about 1500 mg, or from about 10 mg to about 1000 mg, or from about 10 mg to about 750 mg, or from about 10 mg to about 500 mg, or from about 25 mg to about 500 mg, or from about 50 to about 500 mg, or from about 100 mg to about 500 mg.

Additionally, the pharmaceutically acceptable formulations of the present invention may contain a compound of the present invention, or a salt or solvate thereof, in an amount from about 0.5 w/w % to about 95 w/w %, or from about 1 w/w % to about 95 w/w %, or from about 1 w/w % to about 75 w/w %, or from about 5 w/w % to about 75 w/w %, or from about 10 w/w % to about 75 w/w %, or from about 10 w/w % to about 50 w/w %.

The compounds of the present invention, or salts or solvates thereof, may be administered to a mammal, such as a human, suffering from a condition or disease mediated by voltage-gated sodium channel activity, either alone or as part of a pharmaceutically acceptable formulation, once a day, twice a day, three times a day, four times a day, or even more frequently.

The compounds of the present invention, or salts or solvates thereof, may be administered to a mammal, such as a human, suffering from voltage-gated sodium channel-mediated diseases or conditions in combination with at least one other agent used for treatment of voltage-gated sodium channel-mediated diseases or conditions, alone or as part of a pharmaceutically acceptable formulation, once a day, twice a day, three times a day, four times a day, or even more frequently.

Those of ordinary skill in the art will understand that with respect to the compounds of the present invention, the particular pharmaceutical formulation, the dosage, and the number of doses given per day to a mammal requiring such treatment, are all choices within the knowledge of one of ordinary skill in the art and can be determined without undue experimentation.

The compounds of the present invention are useful for modulating or inhibiting voltage-gated sodium channels.

Accordingly, these compounds are useful for the prevention and/or treatment of voltage-gated sodium channel-mediated diseases or conditions.

This invention also relates to a method for the treatment of voltage-gated sodium channel-mediated diseases or conditions including a human comprising administering to said mammal an amount of a compound of the Formula I, as defined above, or a salt or solvate thereof, that is effective in treating voltage-gated sodium channel-mediated diseases or conditions.

### Combination Therapy

Certain drugs are known to prolong the QT interval of the electrocardiogram (ECG). These drugs include the antiarrhythmics (Dronedarone, Sotalol, Quinidine, Procainamide, Disopyramide, Amiodarone, Flecainide, Encainide, Dofetilide, and Ibutilide), antimicrobials such as (Erythromycin, Clarithromycin, Moxifloxacin, Gatifloxacin, Ciprofloxacin, Levofloxacin, Fluconazole, and Ketoconazole), antipsychotics such as (Risperidone, Fluphenazine, Haloperidol, Droperidol, Pimozide, Chlorpromazine, Quetiapine, Clozapine, Olanzapine, Amisulpride, Thioridazine, and Ziprasidone), antidepressants such as (Citalopram, Amitriptyline, Clomipramine, Dosulepin, Doxepin, Fluoxetine, Sertraline, Imipramine, Lofepramine, Desipramine, Nortriptyline, Mianserin, Escitalopram, Venlafaxine, Bupropion, and Moclobemide), antihistamines such as (Diphenhydramine, Astemizole, Loratidine, and Terfanadine), antiemetics such as (Domperidone, Droperidol, and Ondansetron), antimalarials such as (Chloroquine, Hydroxychloroquine, and Quinine), antivirals (Nelfinavir), and others.

Compounds of Structural Formula I of the invention may be administered with drugs causing QT prolongation to eliminate the risk of adverse cardiac effects.

The QT interval is measured on an ECG from the start of the QRS complex to the end of the T wave, and represents the duration between the onset of depolarisation and the completion of repolarization of the myocardium. If the ion channels of the myocardium malfunction, most commonly the delayed potassium rectifier channels (IKr), an excess sodium influx or a decreased potassium efflux may result. This surplus of positively charge ions leads to an extended repolarization phase, thus resulting in a prolonged QT interval. Prolongation of the QT interval (QTc greater than 500 milliseconds) can lead to a life-threatening ventricular arrhythmia known as torsades de pointes (TdP) which can result in sudden cardiac death.

In the following Preparations and Examples, “Ac” means acetyl, “Me” means methyl, “Et” means ethyl, “Ph” means phenyl, “Py” means pyridine, “BOC”, “Boc” or“boc” means N-tert-butoxycarbonyl, “Ns” means 2-Nitrophenylsulfonyl, “DCM” (CH₂Cl₂) means dichloromethane or methylene chloride, “dba” means dibenzylideneacetone, “DCE” means dichloroethane or ethylene chloride, “DIAD” means diisopropylazadicarboxylate, “DIPEA” or “DIEA” means diisopropyl ethyl amine, “DMA” means N,N-dimethylacetamide, “DMF” means N—N-dimethyl formamide, “DMSO” means dimethylsulfoxide, “DPPP” means 1,3-bis(diphenylphosphino)propane, “HOAc” means acetic acid, “IPA” means isopropyl alcohol, “NMP” means 1-methyl 2-pyrrolidinone, “TEA” means triethyl amine, “TFA” means trifluoroacetic acid, “DCM” means dichloromethane, “EtOAc” means ethyl acetate, “MgSO₄” means magnesium sulphate, “Na₂SO₄” means sodium sulphate, “MeOH” means methanol, “Et₂O” means diethyl ether, “EtOH” means ethanol, “H₂O” means water, “HCl” means hydrochloric acid, “POCl₃” means phosphorus oxychloride, “SOCl₂” means thionylchloride, “K₂CO₃” means potassium carbonate, “THF” means tetrahydrofuran, “DBU” means 1,8-diazabicyclo[5.4.0]undec-7-ene, “LiHMDS” or “LHMDS” means lithium hexamethyldisilazide, “TBME” or “MTBE” means tert-butyl methyl ether, “LDA” means lithium diisopropylamide, “NBS” means N-bromosuccinimide, “NIS” means N-iodosuccinimide, “Xanthphos” means 4,5-bis(diphenylphosphino)-9,9-dimethylxanthene; “P(Ph₃)” means triphenylphosphine, “N” means Normal, “M” means molar, “mL” means millilitre, “mmol” means millimoles, “μmol” means micromoles, “eq.” means equivalent, “° C.” means degrees Celsius, “Pa” means pascals, “Xanthphos” means 4,5-bis(diphenylphosphino)-9,9-dimethylxanthene, “rt” means room temperature.

### Methods of Preparation.

Compounds of the present invention may be prepared using the reaction routes and synthetic schemes described below, employing the techniques available in the art using starting materials that are readily available. The preparation of certain embodiments of the present invention is described in detail in the following examples, but those of ordinary skill in the art will recognize that the preparations described may be readily adapted to prepare other embodiments of the present invention. For example, the synthesis of non-exemplified compounds according to the invention may be performed by modifications apparent to those skilled in the art, e.g. by appropriately protecting interfering groups, by changing to other suitable reagents known in the art, or by making routine modifications of reaction conditions. Alternatively, other reactions referred to herein or known in the art will be recognized as having adaptability for preparing other compounds of the invention.

Scheme 1 depicts a method useful for the synthesis of compounds of structural Formula I wherein A is C—R³. Compound 1-1 (X═Cl, Br) can be coupled with a boronic acid or boronic ester R¹B(OR)₂ using a catalyst such as [1,1′-bis(diphenylphosphino)ferrocene] palladium(II) dichloride in the presence of a base such as K₂CO₃ in a solvent such as dimethoxyethane to form 1-2 which can be treated with a halogenation reagent such as bromine or N-bromosuccinimide (NBS), or iodine or N-iodosuccinimide (NIS) to form compound 1-3 (Y═Br, I). Treatment of 1-3 with a boronic acid or boronic ester R²B(OR)₂ using a catalyst such as tetrakis(triphenylphosphine)palladium in the presence of a base such as K₂CO₃ in a solvent such as dioxane can provide a compound of Structural Formula I. Alternatively, compound 1-4 (X═Cl, Br) can react with boronic acid or boronic ester R²B(OR)₂ using a catalyst such as tetrakis(triphenylphosphine) palladium in the presence of a base such as K₂CO₃ in a solvent such as dioxane to provide compound 1-5 which can react with a second boronic acid or boronic ester R¹B(OR)₂ using a catalyst such as [1,1′-bis(diphenylphosphino)ferrocene] palladium(II) dichloride in the presence of a base such as K₂CO₃ in a solvent such as dimethoxyethane to provide a compound of structural Formula I.

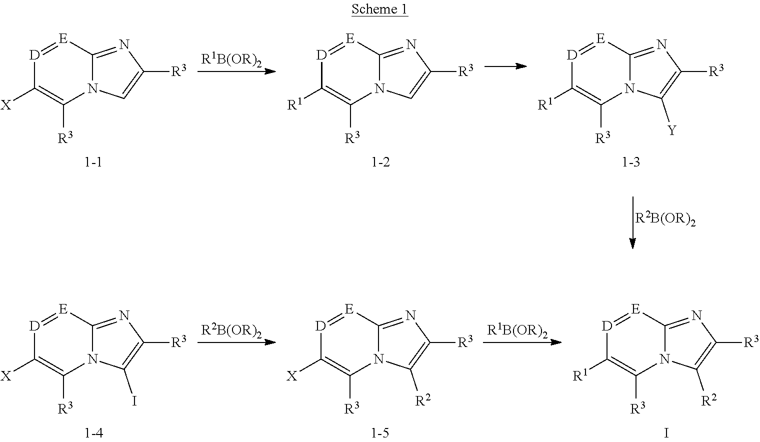

Scheme 2 depicts a method useful for the synthesis of compounds of structural Formula I wherein A is N. Compound 2-1 (X═Cl, Br) can be coupled with a boronic acid or boronic ester R¹B(OR)₂ using a catalyst such as [1,1′-bis(diphenylphosphino)ferrocene] palladium(II) dichloride in the presence of a base such as K₂CO₃ in a solvent such as dimethoxyethane to form 2-2 which can be treated with a halogenation reagent such as bromine or N-bromosuccinimide (NBS), or iodine or N-iodosuccinimide (NIS) to form compound 2-3 (Y═Br, I). Treatment of 2-3 with a boronic acid or boronic ester R²B(OR)₂ using a catalyst such as tetrakis(triphenylphosphine)palladium in the presence of a base such as K₂CO₃ in a solvent such as dioxane can provide a compound of Structural Formula I. Alternatively, compound 2-4 (X═Cl, Br) can react with boronic acid or boronic ester R²B(OR)₂ using a catalyst such as tetrakis(triphenylphosphine) palladium in the presence of a base such as K₂CO₃ in a solvent such as dioxane to provide compound 2-5 which can react with a second boronic acid or boronic ester R¹B(OR)₂ using a catalyst such as [1,1′-bis(diphenylphosphino)ferrocene] palladium(II) dichloride in the presence of a base such as K₂CO₃ in a solvent such as dimethoxyethane to provide a compound of structural Formula I.

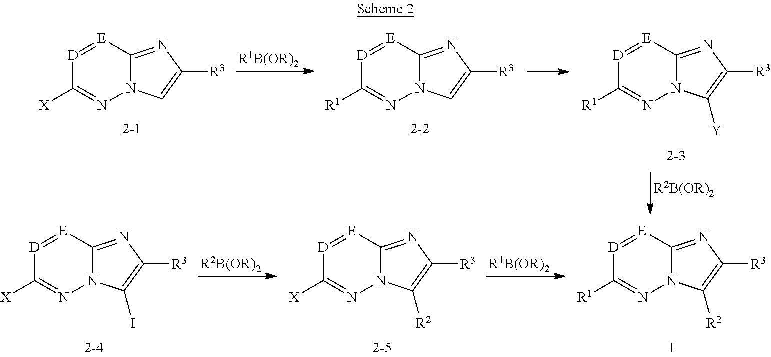

Reaction Schemes 3-5 illustrate methods of synthesis of borane reagents 3-4, 4-4, and 5-4 useful in preparing deuterated intermediates and final compounds of the invention as described in Schemes 1 and 2 above, to introduce R¹ and/or R² substituents.

Scheme 3 depicts a method useful for the synthesis of deuterated boronic acid or ester 3-4. Reaction of phenol 3-1 (X═Br or I) with deuterated alkyl halide 3-2 (X′═Br or I) in the presence of a base such as K₂CO₃ in a solvent such as N,N-dimethylformamide can afford compound 3-3. Compound 3-3 can be converted to a boronic acid or ester 3-4 using standard borylation reaction conditions well known to those skilled in the art. For example, metal-halogen exchange of compound 3-3 with organolithium reagent such as n-Butyllithium followed by treatment with trialkyl borate B(OR)₃ can provide boronic ester 3-4, which can be hydrolyzed to afford free boronic acid 3-4 (R═H).

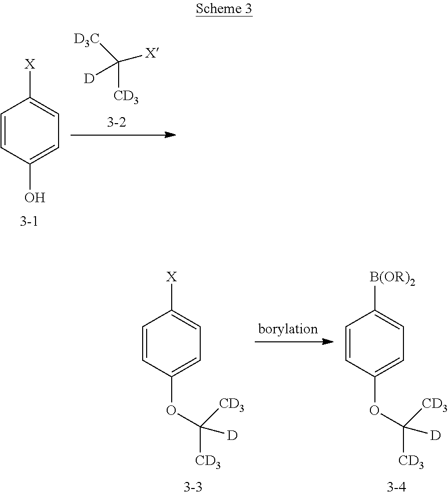

Scheme 4 depicts a method useful for the synthesis of deuterated boronic acid or ester 4-4. Reaction of phenol 4-1 (X═Br or I) with deuterated alkyl bromide 4-2 using catalyst such as nickel(II) acetylacetonate in the presence of a base such as NaHCO₃ in a solvent such as toluene can afford compound 4-3 [Hodous, B. L. US patent application, publication number US2016/0031892, 4 Feb. 2016]. The above patent is herein incorporated by reference in its entirety for all purposes. Compound 4-3 can be converted to a boronic acid or ester 4-4 using standard borylation reaction conditions well known to those skilled in the art. For example, metal-halogen exchange of compound 4-3 with organolithium reagent such as n-Butyllithium followed by treatment with trialkyl borate B(OR)₃ can provide boronic ester 4-4, which can be hydrolyzed to afford free boronic acid 4-4 (R═H).

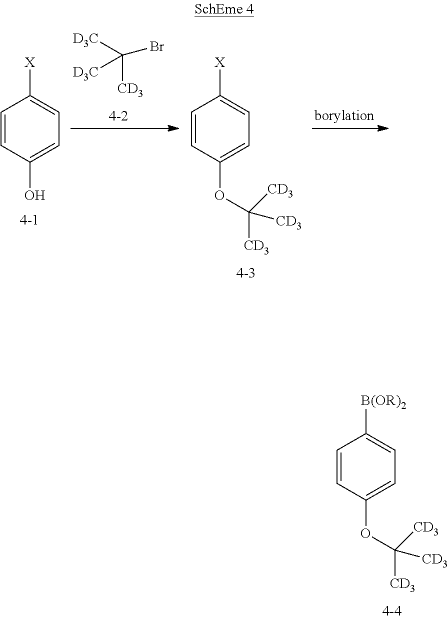

Scheme 5 depicts a method useful for the synthesis of deuterated boronic acid or ester 5-4. Metal-halogen exchange of compound 5-1 (X═Br or I) with organolithium reagent such as n-Butyllithium followed by treatment with compound 5-2 in a solvent such as tetrahydrofuran can provide compound 5-3. Compound 5-3 can be converted to a boronic acid or ester 5-4 using standard borylation reaction conditions well known to those skilled in the art. For example, coupling of compound 5-3 with diboronyl reagent such as bis(pinacolato)diboron using a catalyst such as [1,1′-bis(diphenylphosphino)ferrocene]palladium(II) dichloride in the presence of a base such as potassium acetate in a solvent such as dioxane can provide boronic ester 5-4.

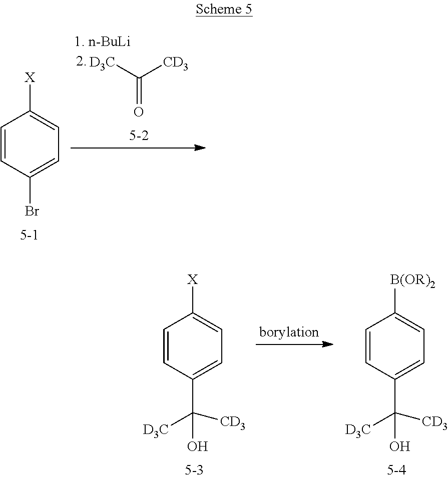

### EXAMPLES

### Example C1: 2-(4-{3-[4-(propan-2-yloxy)phenyl]imidazo[1,2-a]pyridin-6-yl}phenyl)propan-2-ol

### Step 1: 6-bromo-3-[4-(propan-2-yloxy)phenyl]imidazo[1,2-a]pyridine

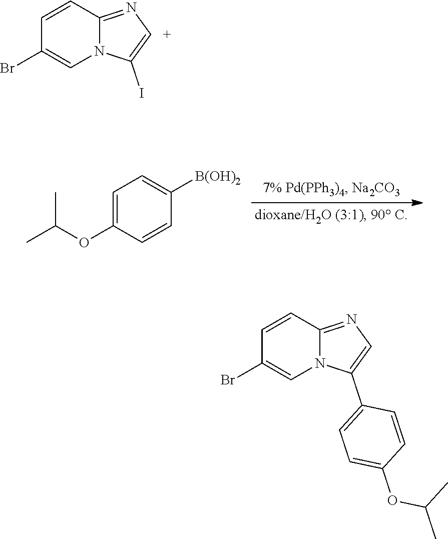 [SMILES: BrC1=CN2C(I)=[C+]N=C2C=C1 ; CC(C)Oc1ccc(B(O)O)cc1 ; CC(C)Oc1ccc(-c2cnc3ccc(Br)cn23)cc1]

To a solution of 6-bromo-3-iodoimidazo[1,2-a]pyridine (0.3 g, 0.93 mmol) in dioxane (9 mL) were added (4-isopropoxyphenyl)boronic acid (0.167 g, 0.93 mmol), tetrakis(triphenylphosphine) palladium(0) (0.075 g, 0.065 mmol), sodium carbonate (0.3 g, 2.8 mmol) and water (3 mL). The resulting reaction mixture was degassed with nitrogen for 10 min, then heated to 90° C. for 5 h. Then the reaction mixture was diluted with ethyl acetate and washed with water. The organic phase was dried over Na₂SO₄, filtered, and concentrated in vacuo. The residue was purified by SiO₂ column chromatography (hexanes/EtOAc from 4:1 to 1:2) to give 0.26 g (84%) of the product as a white solid. ¹H NMR (500 MHz, CDCl₃) δ 8.36 (s, 1H), 7.62 (s, 1H), 7.57 (d, 1H), 7.42 (d, 2H), 7.23 (d, 1H), 7.03 (d, 2H), 4.62 (sep, 1H), 1.39 (d, 6H). LC/MS m/z: 331.07 (⁷⁹Br, M+H)⁺, 333.14 (⁸¹Br, M+H)⁺.

### Step 2: 2-(4-{3-[4-(propan-2-yloxy)phenyl]imidazo[1,2-a]pyridin-6-yl}phenyl)propan-2-ol

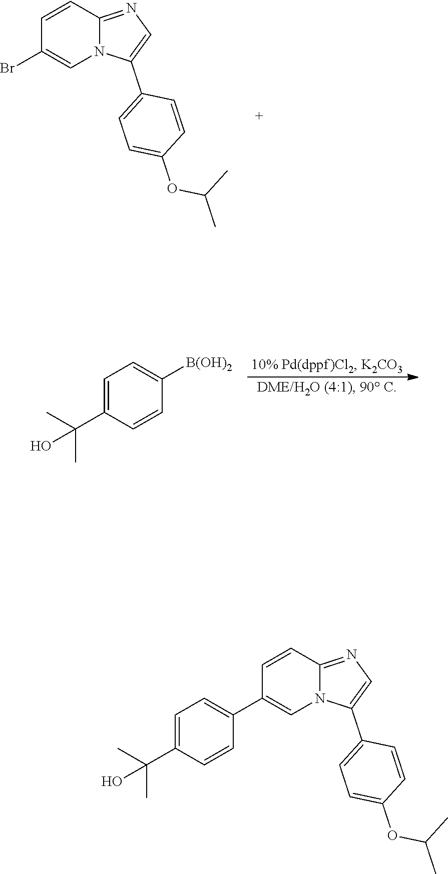 [SMILES: CC(C)Oc1ccc(-c2cnc3ccc(Br)cn23)cc1 ; CC(C)(O)c1ccc(B(O)O)cc1 ; "10% Pd(dppf)Cl2, K2CO3" ; "DME/H2O (4:1), 90° C." ; CC(C)Oc1ccc(-c2cnc3ccc(-c4ccc(C(C)(C)O)cc4)cn23)cc1]

To a solution of 6-bromo-3-[4-(propan-2-yloxy)phenyl]imidazo[1,2-a]pyridine (20 mg, 0.06 mmol) in 1,2-dimethoxyethane (0.8 mL) were added [4-(2-hydroxypropan-2-yl)phenyl]boronic acid (22 mg, 0.12 mmol), [1,1′-bis(diphenylphosphino)ferrocene]palladium(II) dichloride (4.4 mg, 0.006 mmol), potassium carbonate (25 mg, 0.18 mmol) and water (0.2 mL). The resulting reaction mixture was degassed with nitrogen for 10 min, then heated to 90° C. for 5 h. Then the reaction mixture was diluted with ethyl acetate and washed with water. The organic phase was dried over Na₂SO₄, filtered, and concentrated in vacuo. The residue was purified by SiO₂ column chromatography (hexanes/EtOAc from 1:1 to 1:10, then pure EtOAc) to give 12 mg (52%) of the product as a colorless oil. ¹H NMR (300 MHz, CDCl₃) δ8.42 (s, 1H), 7.82 (d, 1H), 7.66 (s, 1H), 7.59 (d, 2H), 7.48-7.51 (m, 3H), 7.46 (d, 2H), 7.05 (d, 2H), 4.62-4.64 (m, 1H), 2.20-2.45 (br s, 1H), 1.62 (s, 6H), 1.40 (d, 6H). LC/MS m/z: 387.27 (M+H)⁺.

### Example C2: 3,6-bis[4-(propan-2-yloxy)phenyl]imidazo[1,2-a]pyridine

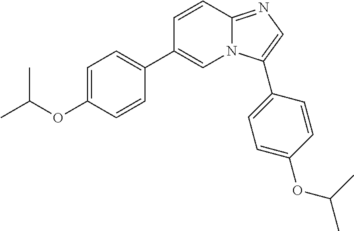 [SMILES: CC(C)Oc1ccc(-c2ccc3ncc(-c4ccc(OC(C)C)cc4)n3c2)cc1]

To a solution of 6-bromo-3-iodoimidazo[1,2-a]pyridine (0.3 g, 0.93 mmol) in dioxane (9 mL) and water (3 mL) was added (4-isopropoxyphenyl)boronic acid (0.334 g, 1.86 mmol) and sodium carbonate (0.6 g, 5.6 mmol). The reaction mixture was purged with nitrogen, then Pd(dppf)Cl₂ (0.05 g, 0.06 mmol) was added. The resulting reaction mixture was heated to 90° C. for 12h, brought to room temperature and was extracted with ethyl acetate. Dried over Na₂SO₄, filtered, and concentrated in vacuo. The residue was purified by SiO₂ column chromatography (hexanes/EtOAc from 4:1 to 1:2) to give 0.24 g (62%) of the title compound as a beige solid. ¹H NMR (300 MHz, CDCl₃) δ 8.37 (s, 1H), 7.70 (d, 1H), 7.64 (s, 1H), 7.49 (d, 2H), 7.44 (d, 2H), 7.41 (d, 1H), 7.05 (d, 2H), 6.97 (d, 2H), 4.56-4.67 (m, 2H), 1.40 (d, 6H), 1.37 (d, 6H). LC/MS m/z: 387.28 (M+H)⁺.

Examples C3 to C26 were prepared in the same manner as described above for example C1, 2-(4-{3-[4-(propan-2-yloxy)phenyl]imidazo[1,2-a]pyridin-6-yl}phenyl)propan-2-ol using 6-bromo-3-[4-(propan-2-yloxy)phenyl]imidazo[1,2-a]pyridine and appropriate commercial boronic acid or boronic acid pinacol ester performing the reaction either under conventional heating or in a microwave reactor.

| Ex. | Starting materials | Product/Name | Analytical Data |
|---|---|---|---|
| C3 | 4-(cyclopropoxy) phenylboronic acid | 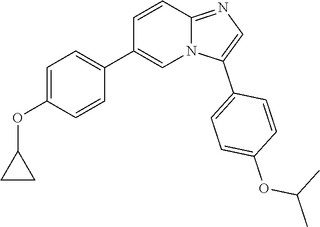 [SMILES: CC(C)Oc1ccc(-c2cnc3ccc(-c4ccc(OC5CC5)cc4)cn23)cc1] | LC/MS m/z: 385.31 (M + H)⁺ |
| | | 6-(4-cyclopropoxyphenyl)-3- | |
| | | [4-(propan-2-yloxy)phenyl] | |
| | | imidazo[1,2-a]pyridine | |
| C4 | 4-(difluoromethoxy) phenyl boronic acid | 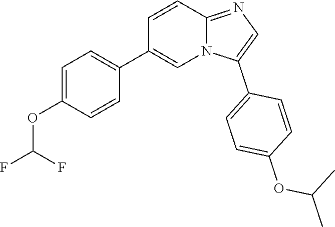 [SMILES: CC(C)Oc1ccc(-c2cnc3ccc(-c4ccc(OC(F)F)cc4)cn23)cc1] | LC/MS m/z: 395.22 (M + H)⁺ |
| | | 6-[4-(difluoromethoxy) | |
| | | phenyl]-3-[4-(propan-2-yloxy) | |
| | | phenyl]imidazo[1,2- | |
| | | a]pyridine | |
| C5 | 4-(difluoromethyl) phenyl boronic acid | 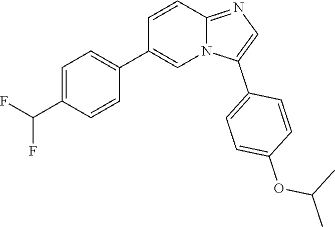 [SMILES: CC(C)Oc1ccc(-c2cnc3ccc(-c4ccc(C(F)F)cc4)cn23)cc1] | LC/MS m/z: 379.23 (M + H)⁺ |
| | | 6-[4-(difluoromethyl)phenyl]- | |
| | | 3-[4-(propan-2-yloxy)phenyl] | |
| | | imidazo[1,2-a]pyridine | |
| C6 | 4-hydroxyphenyl boronic acid | 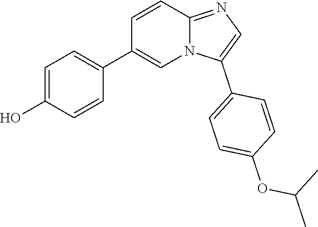 [SMILES: CC(C)Oc1ccc(-c2cnc3ccc(-c4ccc(O)cc4)cn23)cc1] | LC/MS m/z: 345.27 (M + H)⁺ |
| | | 4-{3-[4-(propan-2-yloxy) | |
| | | phenyl]imidazo[1,2-a]pyridin- | |
| | | 6-yl}phenol | |
| C7 | 4-fluorophenyl boronic acid | 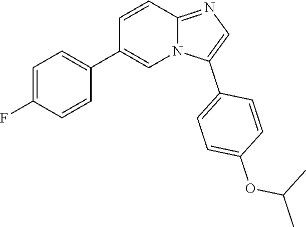 [SMILES: CC(C)Oc1ccc(-c2cnc3ccc(-c4ccc(F)cc4)cn23)cc1] | LC/MS m/z: 347.30 (M + H)⁺ |
| | | 6-(4-fluorophenyl)-3-[4- | |
| | | (propan-2-yloxy)phenyl] | |
| | | imidazo[1,2-a]pyridine | |
| C8 | 1H-indol-5-ylboronic acid | 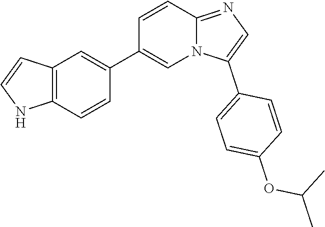 [SMILES: CC(C)Oc1ccc(-c2cnc3ccc(-c4ccc5[nH]ccc5c4)cn23)cc1] | LC/MS m/z: 368.28 (M + H)⁺ |
| | | 5-{3-[4-(propan-2-yloxy) | |
| | | phenyl]imidazo[1,2-a]pyridin- | |
| | | 6-yl}-1H-indole | |
| C9 | 4-(1-hydroxycyclo butyl)phenyl boronic acid | 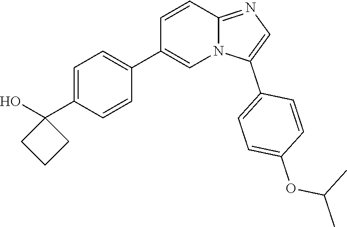 [SMILES: CC(C)Oc1ccc(-c2cnc3ccc(-c4ccc(C5(O)CCC5)cc4)cn23)cc1] | LC/MS m/z: 399.28 (M + H)⁺ |
| | | 1-(4-{3-[4-(propan-2-yloxy) | |
| | | phenyl]imidazo[1,2-a]pyridin- | |
| | | 6-yl}phenyl)cyclobutan-1-ol | |
| C10 | 1H-indazol-5- ylboronic acid | 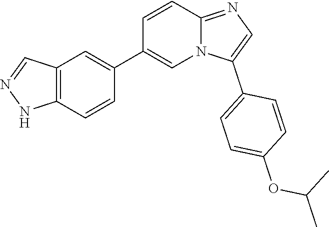 [SMILES: CC(C)Oc1ccc(-c2cnc3ccc(-c4ccc5[nH]ncc5c4)cn23)cc1] | LC/MS m/z: 369.33 (M + H)⁺ |
| | | 5-{3-[4-(propan-2-yloxy) | |
| | | phenyl]imidazo[1,2-a]pyridin- | |
| | | 6-yl}-1H-indazole | |
| C11 | 1H-indol-6-boronic acid | 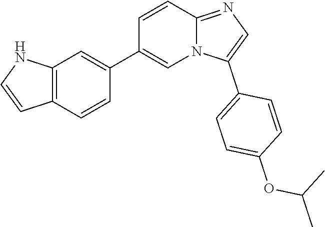 [SMILES: CC(C)Oc1ccc(-c2cnc3ccc(-c4ccc5cc[nH]c5c4)cn23)cc1] | LC/MS m/z: 368.36 (M + H)⁺ |
| | | 6-{3-[4-(propan-2-yloxy) | |
| | | phenyl]imidazo[1,2-a]pyridin- | |
| | | 6-yl}-1H-indole | |
| C12 | 2-isopropoxypyridine- 5-boronic acid | 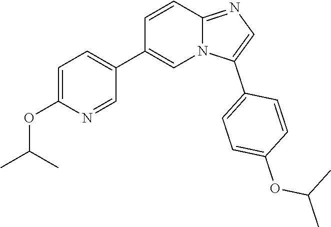 [SMILES: CC(C)Oc1ccc(-c2cnc3ccc(-c4ccc(OC(C)C)nc4)cn23)cc1] | LC/MS m/z: 388.28 (M + H)⁺ |
| | | 2-(propan-2-yloxy)-5-{3-[4- | |
| | | (propan-2-yloxy)phenyl] | |
| | | imidazo[1,2-a]pyridin-6-yl} | |
| | | pyridine | |
| C13 | (4-acetylphenyl) boronic acid | 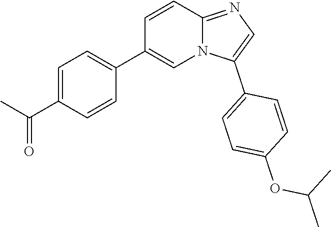 [SMILES: CC(=O)c1ccc(-c2ccc3ncc(-c4ccc(OC(C)C)cc4)n3c2)cc1] | ¹H NMR (300 MHz, CDCl₃) δ 8.48 (s, 1H), 8.06 (d, 2H), 7.93 (d, 1H), 7.70 (s, 1H), 7.66-7.58 (m, 3H), 7.49 (d, 2H), 7.08 (d, 2H), 4.69- 4.61 (m, 1H), 2.65 (s, 3H), 1.41 (d, 6H)., LC/MS m/z: 371.33 (M + H)⁺ |
| | | 1-(4-{3-[4-(propan-2-yloxy) | |
| | | phenyl]imidazo[1,2-a]pyridin- | |
| | | 6-yl}phenyl)ethan-1-one | |
| C14 | 5-(4,4,5,5- tetramethyl-1,3,2- dioxaborolan-2- yl)pyridin-2-amine | 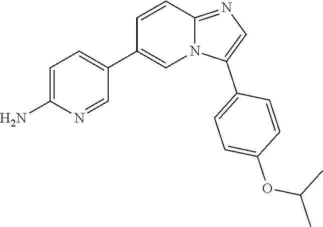 [SMILES: CC(C)Oc1ccc(-c2cnc3ccc(-c4ccc(N)nc4)cn23)cc1] | LC/MS m/z: 345.32 (M + H)⁺ |
| | | 5-{3-[4-(propan-2-yloxy) | |
| | | phenyl]imidazo[1,2-a]pyridin- | |
| | | 6-yl}pyridin-2-amine | |
| C15 | 4-(1-aminocyclo propyl)phenyl boronic acid | 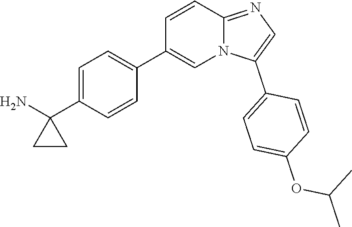 [SMILES: CC(C)Oc1ccc(-c2cnc3ccc(-c4ccc(C5(N)CC5)cc4)cn23)cc1] | LC/MS m/z: 384.35 (M + H)⁺ |
| | | 1-(4-{3-[4-(propan-2-yloxy) | |
| | | phenyl]imidazo[1,2-a]pyridin- | |
| | | 6-yl}phenyl)cyclopropan-1- | |
| | | amine | |
| C16 | (4-carbamoylphenyl) boronic acid | 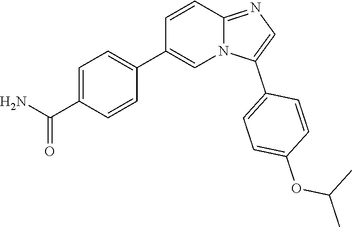 [SMILES: CC(C)Oc1ccc(-c2cnc3ccc(-c4ccc(C(N)=O)cc4)cn23)cc1] | LC/MS m/z: 372.23 (M + H)⁺ |
| | | 4-{3-[4-(propan-2-yloxy) | |
| | | phenyl]imidazo[1,2-a]pyridin- | |
| | | 6-yl}benzamide | |
| C17 | 1-cyclopropyl-4- (4,4,5,5-tetramethyl- 1,3,2-dioxaborolan-2- yl)pyrazole | 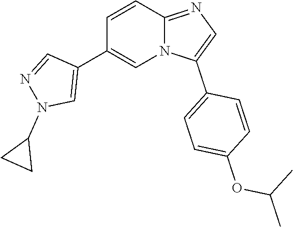 [SMILES: CC(C)Oc1ccc(-c2cnc3ccc(-c4cnn(C5CC5)c4)cn23)cc1] | LC/MS m/z: 359.26 (M + H)⁺ |
| | | 1-cyclopropyl-4-{3-[4- | |
| | | (propan-2-yloxy)phenyl] | |
| | | imidazo[1,2-a]pyridin-6-yl}- | |
| | | 1H-pyrazole | |
| C18 | 4-isopropoxy-2- methylphenyl boronic acid | 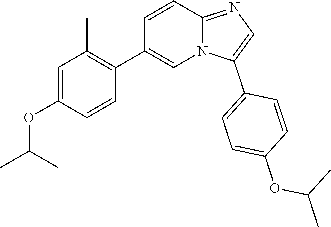 [SMILES: Cc1cc(OC(C)C)ccc1-c1ccc2ncc(-c3ccc(OC(C)C)cc3)n2c1] | LC/MS m/z: 401.39 (M + H)⁺ |
| | | 6-[2-methyl-4-(propan-2- | |
| | | yloxy)phenyl]-3-[4-(propan-2- | |
| | | yloxy)phenyl]imidazo[1,2- | |
| | | a]pyridine | |
| C19 | 4-isopropoxy-3- methylphenyl boronic acid | 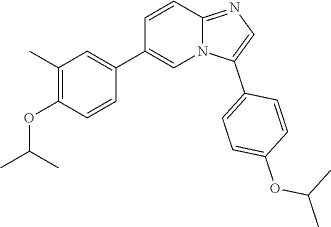 [SMILES: Cc1cc(-c2ccc3ncc(-c4ccc(OC(C)C)cc4)n3c2)ccc1OC(C)C] | ¹H NMR (300 MHz, CDCl₃) δ 8.36 (s, 1H), 8.13 (d, 1H), 7.76 (d, 1H), 7.69 (s, 1H), 7.48 (d, 2H), 7.29 (s, 1H), 7.26 (d, 1H), 7.09 (d, 2H), 6.92 (d, 1H), 4.51-4.73 (m, 2H), 2.27 (s, 3H), 1.41 (d, 6H), 1.38 (d, 6H)., LC/MS m/z: 401.34 (M + H)⁺ |
| | | 6-[3-methyl-4-(propan-2 | |
| | | yloxy)phenyl]-3-[4-(propan-2- | |
| | | yloxy)phenyl]imidazo[1,2-a] | |
| | | pyridine | |
| C20 | {4-[(propan-2-yloxy) carbonyl]phenyl} boronic acid | 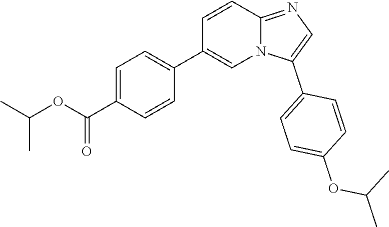 [SMILES: CC(C)OC(=O)c1ccc(-c2ccc3ncc(-c4ccc(OC(C)C)cc4)n3c2)cc1] | LC/MS m/z: 415.30 (M + H)⁺ |
| | | propan-2-yl 4-{3-[4-(propan- | |
| | | 2-yloxy)phenyl]imidazo[1,2-a] | |
| | | pyridin-6-yl}benzoate | |
| C21 | [4-(tert-butoxy) phenyl]boronic acid | 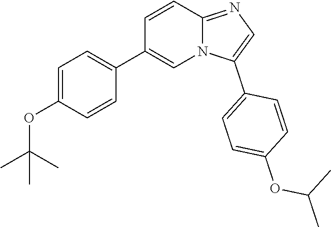 [SMILES: CC(C)Oc1ccc(-c2cnc3ccc(-c4ccc(OC(C)(C)C)cc4)cn23)cc1] | ¹H NMR (300 MHz, CDCl₃) δ 8.39 (s, 1H), 7.94 (d, 1H), 7.68 (s, 1H), 7.62 (d, 1H), 7.49 (d, 2H), 7.43 (d, 2H), 7.09 (d, 2H), 7.06 (d, 2H), 4.60-4.71 (m, 1H), 1.42 (d, 6H), 1.39 (s, 9H)., LC/MS m/z: 401.30 (M + H)⁺ |
| | | 6-[4-(tert-butoxy)phenyl]-3-[4- | |
| | | (propan-2-yloxy)phenyl] | |
| | | imidazo[1,2-a]pyridine | |
| C22 | 1-methylindole-5- boronic acid | 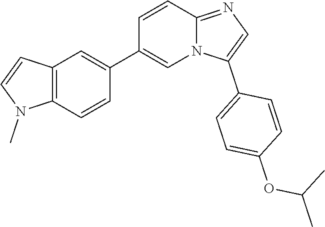 [SMILES: CC(C)Oc1ccc(-c2cnc3ccc(-c4ccc5c(ccn5C)c4)cn23)cc1] | ¹H NMR (300 MHz, CDCl₃) δ 8.45 (s, 1H), 7.85 (d, 1H), 7.77 (s, 1H), 7.66 (s, 1H), 7.64 (d, 1H), 7.51 (d, 2H), 7.40 (br s, 2H), 7.12 (d, 1H), 7.06 (d, 2H), 6.55 (d, 1H), 4.61- 4.69 (m, 1H), 3.84 (s, 3H), 1.41 (d, 6H)., LC/MS m/z: 382.35 (M + H)⁺ |
| | | 1-methyl-5-{3-[4-(propan-2- | |
| | | yloxy)phenyl]imidazo[1,2-a] | |
| | | pyridin-6-yl}-1H-indole | |
| C23 | [2-fluoro-4-(propan-2- yloxy)phenyl]boronic acid | 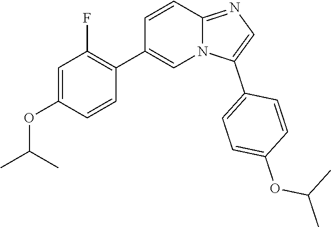 [SMILES: CC(C)Oc1ccc(-c2cnc3ccc(-c4ccc(OC(C)C)cc4F)cn23)cc1] | LC/MS m/z: 405.30 (M + H)⁺ |
| | | 6-[2-fluoro-4-(propan-2- | |
| | | yloxy)phenyl]-3-[4-(propan-2- | |
| | | yloxy)phenyl]imidazo[1,2-a] | |
| | | pyridine | |
| C24 | [3-fluoro-4-(propan-2- yloxy)phenyl]boronic acid | 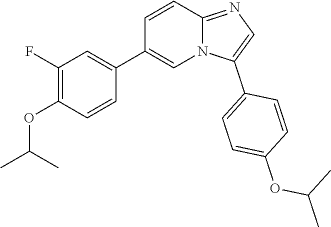 [SMILES: CC(C)Oc1ccc(-c2cnc3ccc(-c4ccc(OC(C)C)c(F)c4)cn23)cc1] | ¹H NMR (300 MHz, CDCl₃) δ 8.36 (s, 1H), 7.99 (d, 1H), 7.69 (s, 1H), 7.59 (d, 1H), 7.48 (d, 2H), 7.28 (s, 1H), 7.23 (d, 1H), 7.08 (d, 2H), 7.05 (d, 1H), 4.53-4.72 (m, 2H), 1.41 (d, 6H), 1.39 (d, 6H)., LC/MS m/z: 405.26 (M + H)⁺ |
| | | 6-[3-fluoro-4-(propan-2-yloxy) | |
| | | phenyl]-3-[4-(propan-2-yloxy) | |
| | | phenyl]imidazo[1,2-a]pyridine | |
| C25 | 4-(2-morpholino ethoxy)phenyl boronic acid | 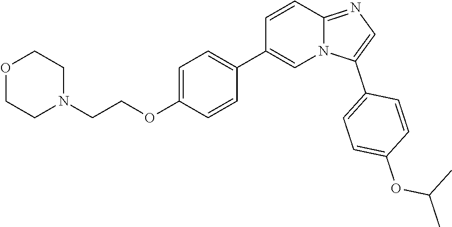 [SMILES: CC(C)Oc1ccc(-c2cnc3ccc(-c4ccc(OCCN5CCOCC5)cc4)cn23)cc1] | LC/MS m/z: 458.32 (M + H)⁺ |
| | | 4-[2-(4-{3-[4-(propan-2-yloxy) | |
| | | phenyl]imidazo[1,2-a]pyridin- | |
| | | 6-yl}phenoxy)ethyl] | |
| | | morpholine | |
| C26 | N-(propan-2-yl)-4- (4,4,5,5-tetramethyl- 1,3,2-dioxaborolan-2- yl)aniline | 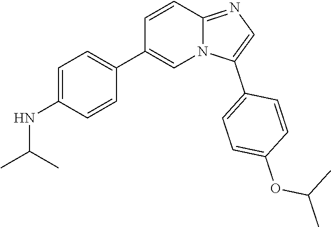 [SMILES: CC(C)Nc1ccc(-c2ccc3ncc(-c4ccc(OC(C)C)cc4)n3c2)cc1] | ¹H NMR (300 MHz, CDCl₃) δ 8.32 (s, 1H), 8.19 (d, 1H), 7.82 (dd, 1H), 7.66 (s, 1H), 7.45 (d, 2H), 7.31 (d, 2H), 7.07 (d, 2H), 6.65 (d, 2H), 4.69-4.59 (m, 1H), 3.73-3.60 (m, 1H), 1.40 (d, 6H), 1.23 (d, 6H). LC/MS m/z: 386.32 (M + H)⁺ |
| | | N-(propan-2-yl)-4-{3-[4- | |
| | | (propan-2-yloxy)phenyl] | |
| | | imidazo[1,2-a]pyridin-6-yl} | |
| | | aniline | |

### Preparation of Boronic Acid Pinacol Esters for Examples C27-C29

### tert-butyldimethyl{1-[4-(4,4,5,5-tetramethyl-1,3,2-dioxaborolan-2-yl)phenyl]cyclopropoxy}silane

### Step 1: [1-(4-bromophenyl)cyclopropoxy](tert-butyl)dimethylsilane

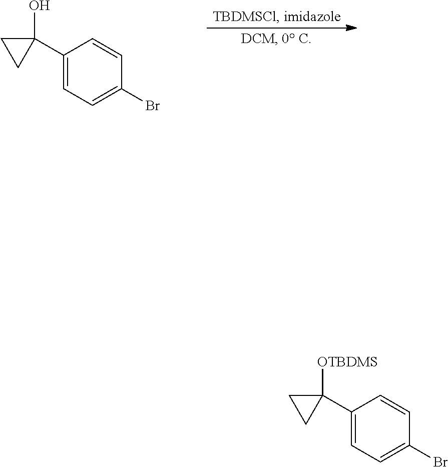 [SMILES: OC1(c2ccc(Br)cc2)CC1 ; "DCM, 0° C." ; "TBDMSCl, imidazole" ; CC(C)(C)[Si](C)(C)OC1(c2ccc(Br)cc2)CC1]

To a solution of 1-(4-bromophenyl)cyclopropan-1-ol (90 mg, 0.42 mmol) in DCM (1.5 mL) cooled to 0° C. was added imidazole (57 mg, 0.84 mmol), followed by TBDMSCl (76 mg, 0.5 mmol). The reaction mixture was brought to r.t. gradually and stirred for 2h. Then DCM (10 mL) was added and the reaction mixture was washed with water. The organic layer was dried over Na₂SO₄, filtered, and concentrated in vacuo. The residue was dissolved in DCM and passed through a short pad of silica gel eluting with DCM. Then volatiles were removed in vacuo to give 0.14 g (quantitative yield) of the product as a colorless oil, which was used in the next step.

### Step 2: tert-butyldimethyl{1-[4-(4,4,5,5-tetramethyl-1,3,2-dioxaborolan-2-yl)phenyl]cyclopropoxy}silane

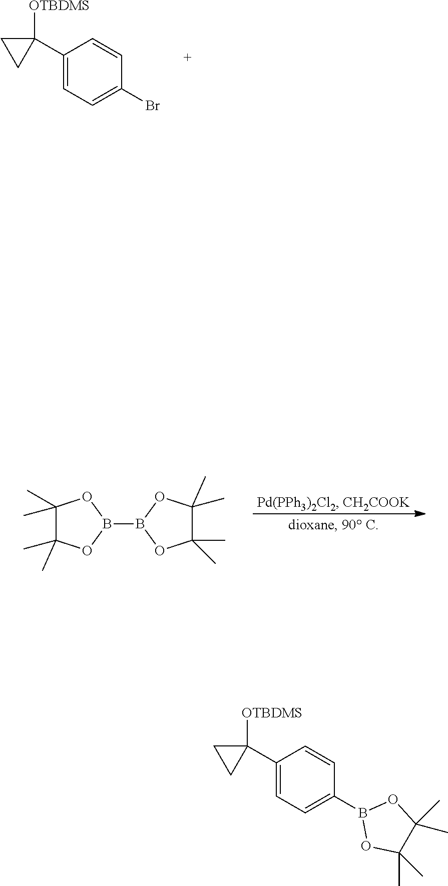

To a degassed mixture of [1-(4-bromophenyl)cyclopropoxy](tert-butyl)dimethylsilane (0.14 g, 0.42 mmol), bis(pinacolato)diboron (0.16 g, 0.63 mmol) and potassium acetate (0.13 g, 1.3 mmol) in anhydrous dioxane (1.6 mL) was added bis(triphenylphosphine)palladium(II) dichloride (0.03 g, 0.042 mmol). The resulting reaction mixture was stirred under N₂ atmosphere at 90° C. for 3h. Then the reaction mixture was diluted with ethyl acetate and washed with water. The organic phase was dried over Na₂SO₄, filtered, and concentrated in vacuo. The residue was purified by flash chromatography on SiO₂ (hexanes/EtOAc 8:1) to give 0.11 g (69%) of the product as a colorless oil which solidified upon standing. ¹H NMR (300 MHz, CDCl₃) δ 7.75 (d, 2H), 7.31 (d, 2H), 1.35 (s, 12H), 1.23-1.17 (m, 2H), 1.04-0.99 (m, 2H), 0.89 (s, 9H), 0.00 (s, 6H).

### {[3-(4-Bromophenyl)oxetan-3-yl]oxy}(tert-butyl)dimethylsilane

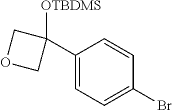 [SMILES: CC(C)(C)[Si](C)(C)OC1(c2ccc(Br)cc2)COC1]

The title compound was prepared from 3-(4-bromophenyl)oxetan-3-olin the same manner as described above for [1-(4-bromophenyl)cyclopropoxy](tert-butyl)dimethylsilane.

### tert-Butyldimethyl({3-[4-(4,4,5,5-tetramethyl-1,3,2-dioxaborolan-2-yl)phenyl]oxetan-3-yl}oxy)silane

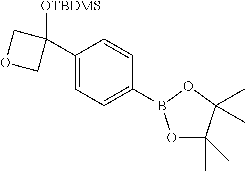 [SMILES: CC1(C)OB(c2ccc(C3(O[Si](C)(C)C(C)(C)C)COC3)cc2)OC1(C)C]

The title compound was prepared from {[3-(4-bromophenyl)oxetan-3-yl]oxy}(tert-butyl)dimethylsilane in the same manner as described above for tert-butyldimethyl{1-[4-(4,4,5,5-tetramethyl-1,3,2-dioxaborolan-2-yl)phenyl]cyclopropoxy}silane. ¹H NMR (300 MHz, CDCl₃) δ 7.87 (d, 2H), 7.62 (d, 2H), 5.00 (d, 2H), 4.84 (d, 2H), 1.37 (s, 12H), 0.96 (s, 9H), 0.00 (s, 6H).

### tert-Butyl 3-(4-bromophenyl)-3-[(tert-butyldimethylsilyl)oxy]azetidine-1-carboxylate

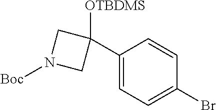 [SMILES: CC(C)(C)OC(=O)N1CC(O[Si](C)(C)C(C)(C)C)(c2ccc(Br)cc2)C1]

The title compound was prepared from tert-butyl 3-(4-bromophenyl)-3-hydroxyazetidine-1-carboxylate in the same manner as described above for [1-(4-bromophenyl)cyclopropoxy](tert-butyl)dimethylsilane.

### tert-Butyl 3-[(tert-butyldimethylsilyl)oxy]-3-[4-(4,4,5,5-tetramethyl-1,3,2-dioxaborolan-2-yl)phenyl]azetidine-1-carboxylate

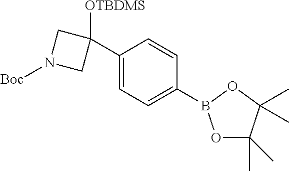 [SMILES: CC(C)(C)OC(=O)N1CC(O[Si](C)(C)C(C)(C)C)(c2ccc(B3OC(C)(C)C(C)(C)O3)cc2)C1]

The title compound was prepared from tert-butyl 3-(4-bromophenyl)-3-[(tert-butyldimethylsilyl)oxy]azetidine-1-carboxylate in the same manner as described above for tert-butyldimethyl{1-[4-(4,4,5,5-tetramethyl-1,3,2-dioxaborolan-2-yl)phenyl]cyclopropoxy}silane.

### Example C27: 1-(4-{3-[4-(propan-2-yloxy)phenyl]imidazo[1,2-a]pyridin-6-yl}phenyl)cyclopropan-1-ol

### Step 1: 6-(4-{1-[(tert-butyldimethylsilyl)oxy]cyclopropyl}phenyl)-3-[4-(propan-2-yloxy)phenyl]imidazo[1,2-a]pyridine

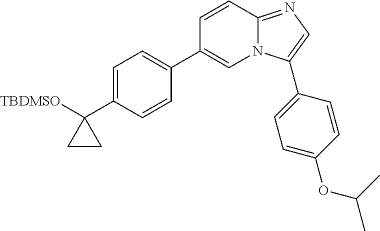 [SMILES: CC(C)Oc1ccc(-c2cnc3ccc(-c4ccc(C5(O[Si](C)(C)C(C)(C)C)CC5)cc4)cn23)cc1]

The title compound was prepared from 6-bromo-3-[4-(propan-2-yloxy)phenyl]imidazo[1,2-a]pyridine and tert-butyldimethyl{1-[4-(4,4,5,5-tetramethyl-1,3,2-dioxaborolan-2-yl)phenyl]cyclopropoxy}silane in the same manner as described above for 2-(4-{3-[4-(propan-2-yloxy)phenyl]imidazo[1,2-a]pyridin-6-yl}phenyl)propan-2-ol (Example C1). LC/MS W/z 499.36 (M+H)⁺.

### Step 2: 1-(4-{3-[4-(propan-2-yloxy)phenyl]imidazo[1,2-a]pyridin-6-yl}phenyl)cyclopropan-1-ol

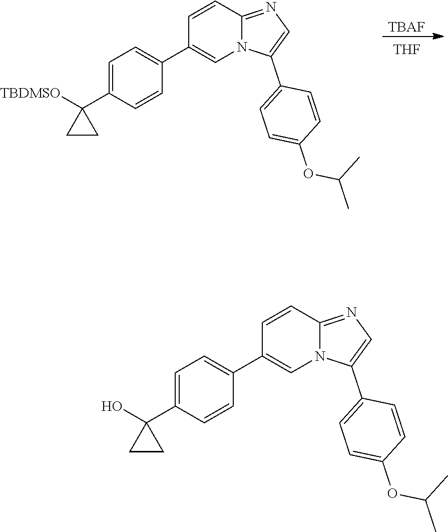 [SMILES: CC(C)Oc1ccc(-c2cnc3ccc(-c4ccc(C5(O[Si](C)(C)C(C)(C)C)CC5)cc4)cn23)cc1 ; "TBAF" ; "THF" ; CC(C)Oc1ccc(-c2cnc3ccc(-c4ccc(C5(O)CC5)cc4)cn23)cc1]

To a solution of 6-(4-{1-[(tert-butyldimethylsilyl)oxy]cyclopropyl}phenyl)-3-[4-(propan-2-yloxy)phenyl]imidazo[1,2-a]pyridine (12 mg, 0.024 mmol) in THF (0.5 mL) was added TBAF·3H₂O (20 mg, 0.063 mmol). The reaction mixture was stirred at r.t. for 2h, then concentrated in vacuo. The residue was dissolved in EtOAc and washed with water. The organic layer was dried over Na₂SO₄, filtered, and concentrated in vacuo. The residue was purified by SiO₂ column chromatography (hexanes/EtOAc from 1:1 to 1:10, then pure EtOAc) to give 5.6 mg (60%) of the title compound as a white solid. ¹H NMR (300 MHz, CDCl₃) δ 8.39 (s, 1H), 8.02 (d, 1H), 7.69-7.63 (m, 2H), 7.46-7.36 (m, 6H), 7.06 (d, 2H), 4.63 (sop, 1H), 3.31 (br. s, 1H), 1.39 (d, 6H), 1.37-1.33 (m, 2H), 1.09-1.05 (m, 2H). LC/MS m/z: 385.29 (M+H)⁺.

### Example C28: 3-(4-{3-[4-(propan-2-yloxy)phenyl]imidazo[1,2-a]pyridin-6-yl}phenyl)oxetan-3-ol

### Step 1: 6-(4-{3-[(tert-butyldimethylsilyl)oxy]oxetan-3-yl}phenyl)-3-[4-(propan-2-yloxy)phenyl] imidazo [1,2-a]pyridine

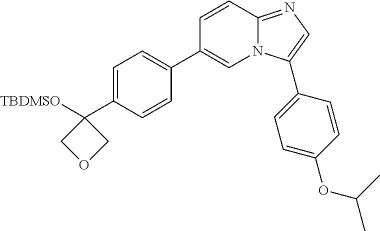 [SMILES: CC(C)Oc1ccc(-c2cnc3ccc(-c4ccc(C5(O[Si](C)(C)C(C)(C)C)COC5)cc4)cn23)cc1]

The title compound was prepared from 6-bromo-3-[4-(propan-2-yloxy)phenyl]imidazo[1,2-a]pyridine and tert-butyldimethyl({3-[4-(4,4,5,5-tetramethyl-1,3,2-dioxaborolan-2-yl)phenyl]oxetan-3-yl}oxy)silane in the same manner as described above for 2-(4-{3-[4-(propan-2-yloxy)phenyl]imidazo[1,2-a]pyridin-6-yl}phenyl)propan-2-ol (Example C1). LC/MS m/z: 515.38 (M+H)⁺.

### Step 2: 3-(4-{3-[4-(propan-2-yloxy)phenyl]imidazo[1,2-a]pyridin-6-yl}phenyl)oxetan-3-ol

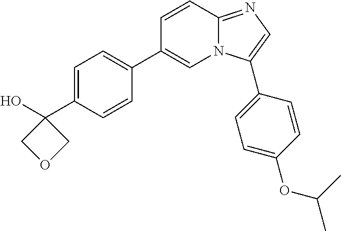 [SMILES: CC(C)Oc1ccc(-c2cnc3ccc(-c4ccc(C5(O)COC5)cc4)cn23)cc1]

The title compound was prepared from 6-(4-{3-[(tert-butyldimethylsilyl)oxy]oxetan-3-yl}phenyl)-3-[4-(propan-2-yloxy)phenyl]imidazo[1,2-a]pyridine in the same manner as described above for 1-(4-{3-[4-(propan-2-yloxy)phenyl]imidazo[1,2-a]pyridin-6-yl}phenyl)cyclopropan-1-ol. LC/MS m/z: 401.31 (M+H)⁺.

### Example C29: 3-(4-{3-[4-(propan-2-yloxy)phenyl]imidazo[1,2-a]pyridin-6-yl}phenyl)azetidin-3-ol

### Step 1: tert-butyl 3-[(tert-butyidimethylsilyl)oxy]-3-(4-{3-[4-(propan-2-yloxy)phenyl]imidazo[1,2-a]pyridin-6-yl}phenyl)azetidine-1-carboxylate

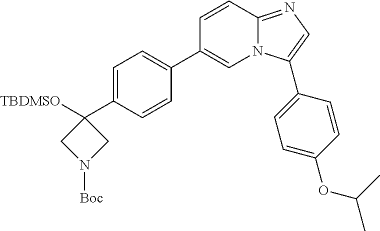 [SMILES: CC(C)Oc1ccc(-c2cnc3ccc(-c4ccc(C5(O[Si](C)(C)C(C)(C)C)CN(C(=O)OC(C)(C)C)C5)cc4)cn23)cc1]

The title compound was prepared from 6-bromo-3-[4-(propan-2-yloxy)phenyl]imidazo[1,2-a]pyridine and tert-butyl 3-[(tert-butyldimethylsilyl)oxy]-3-[4-(4,4,5,5-tetramethyl-1,3,2-dioxaborolan-2-yl)phenyl]azetidine-1-carboxylate in the same manner as described above for 2-(4-{3-[4-(propan-2-yloxy)phenyl]imidazo[1,2-a]pyridin-6-yl}phenyl)propan-2-ol (Example C1). LC/MS m/z: 614.42 (M+H)⁺.

### Step 2: 3-[(tert-butyldimethylsilyl)oxy]-3-(4-{3-[4-(propan-2-yloxy)phenyl]imidazo[1,2-a]pyridin-6-yl}phenyl)azetidine

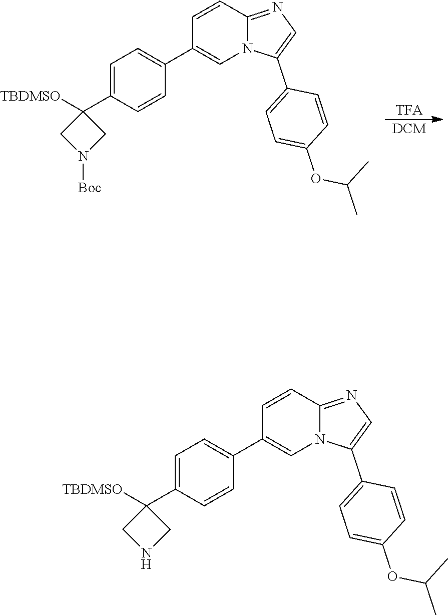

To a solution of tert-butyl 3-[(tert-butyldimethylsilyl)oxy]-3-(4-{3-[4-(propan-2-yloxy)phenyl]imidazo[1,2-a]pyridin-6-yl}phenyl)azetidine-1-carboxylate (29 mg, 0.047 mmol) in DCM (1 mL) was added trifluoroacetic acid (0.25 mL). The reaction mixture was stirred at r.t. overnight. Then, the solvent was evaporated, and the residue was dissolved in DCM and washed with saturated aq. NaHCO₃ solution. The organic phase was dried over Na₂SO₄, filtered, and concentrated in vacuo to give 20 mg (83%) of the product as a colorless oil, which was used in the next step without further purification. LC/MS m/z: 514.44 (M+H)⁺.

### Step 3: 3-(4-{3-[4-(propan-2-yloxy)phenyl]imidazo[1,2-a]pyridin-6-yl}phenyl)azetidin-3-ol

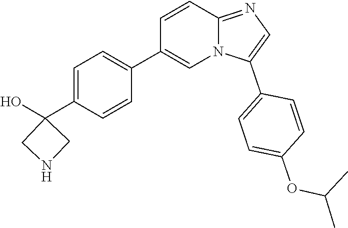 [SMILES: CC(C)Oc1ccc(-c2cnc3ccc(-c4ccc(C5(O)CNC5)cc4)cn23)cc1]

The title compound was prepared from 3-[(tert-butyldimethylsilyl)oxy]-3-(4-{3-[4-(propan-2-yloxy)phenyl]imidazo[1,2-a]pyridin-6-yl}phenyl)azetidine in the same manner as described above for 1-(4-{3-[4-(propan-2-yloxy)phenyl]imidazo[1,2-a]pyridin-6-yl}phenyl)cyclopropan-1-ol. LC/MS m/z: 400.41 (M+H)⁺.

### Example C30: 3-(4-cyclopropylphenyl)-6-[4-(propan-2-yloxy)phenyl]imidazo[1,2-a]pyridine

### Step 1: 3-bromo-6-[4-(propan-2-yloxy)phenyl]imidazo[1,2-a]pyridine

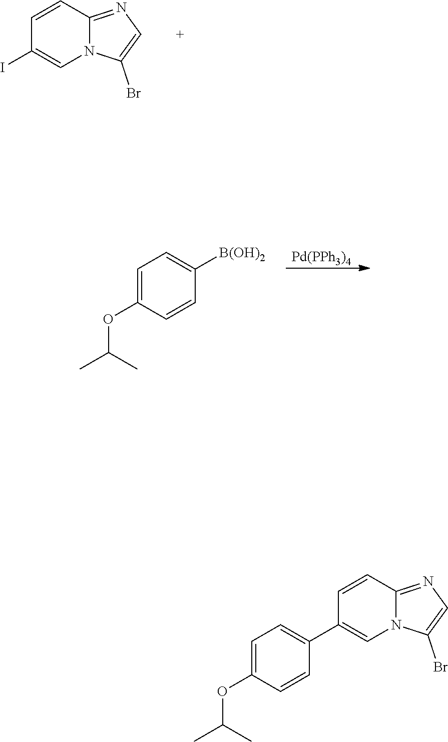 [SMILES: Brc1cnc2ccc(I)cn12 ; CC(C)Oc1ccc(B(O)O)cc1 ; CC(C)Oc1ccc(-c2ccc3ncc(Br)n3c2)cc1 ; "Pd(PPh3)4"]

3-bromo-6-iodoimidazo[1,2-a]pyridine (100 mg, 0.3 mmol) was added to a microwave reactor vial followed by 4-isopropoxyphenyl boronic acid (62 mg, 0.34 mmol), tetrakis(triphenylphosphine) palladium(0) (34 mg, 0.03 mmol), sodium carbonate (94 mg, 0.9 mmol), 3 mL of 1,4-dioxane, and 1 mL of water. The mixture was capped tightly and degassed by bubbling nitrogen through the septum for 5 minutes. The resulting solution was heated to 90° C. for 15 minutes in a microwave reactor and allowed to cool. The mixture was then diluted with ethyl acetate, washed with water (2×) and brine (1×), dried over sodium sulfate, and evaporated. The crude oil was purified by flash chromatography on silica (1:1 hexanes:ethyl acetate, isocratic) to provide the title compound (82 mg) as a light yellow oil that slowly solidified. LC/MS m/z: 331.24 (⁷⁹Br, M+H)⁺, 333.28 (⁸¹Br, M+H)⁺.

### Step 2:3-(4-cyclopropylphenyl)-6-[4-(propan-2-yloxy)phenyl]imidazo[1,2-a]pyridine

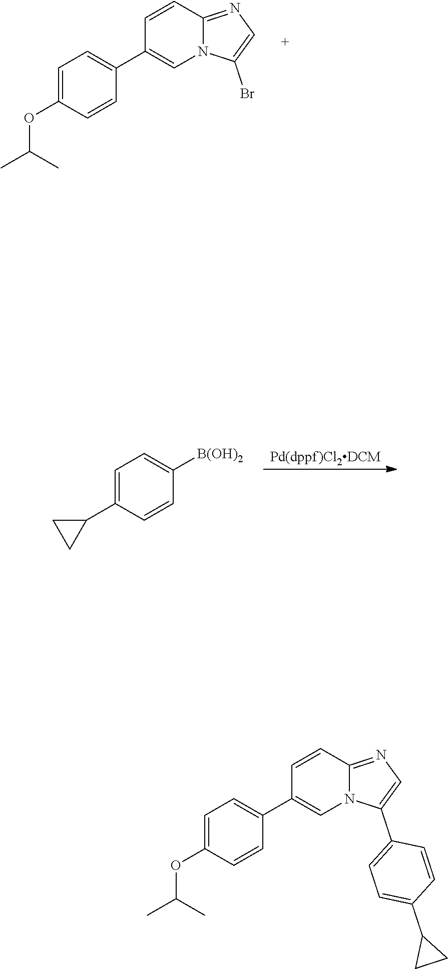 [SMILES: CC(C)Oc1ccc(-c2ccc3ncc(Br)n3c2)cc1 ; OB(O)c1ccc(C2CC2)cc1 ; "Pd(dppf)Cl2·DCM" ; CC(C)Oc1ccc(-c2ccc3ncc(-c4ccc(C5CC5)cc4)n3c2)cc1]

To a solution of 3-bromo-6-[4-(propan-2-yloxy)phenyl]imidazo[1,2-a]pyridine (20 mg, 0.06 mmol) in 2 mL THF:Water 3:1 was added 4-cyclopropylboronic acid (12 mg, 0.072 mmol), potassium carbonate (5 mg, 0.18 mmol), and Pd(dppf)Cl₂ dichloromethane complex (5 mg, 0.006 mmol). The mixture was capped tightly and degassed by bubbling nitrogen through the septum for 5 minutes. The resulting solution was heated to 100° C. for 15 minutes in a microwave reactor and allowed to cool. The mixture was then diluted with ethyl acetate, washed with water (2×) and brine (1×), dried over sodium sulfate, and evaporated. The crude oil was purified by flash chromatography on silica (3:7 hexanes:ethyl acetate, isocratic) to provide 13 mg of the title compound as a light yellow oil. LC/MS m/z: 369.26 (M+H)⁺

Examples C31 to C43 were prepared in the same manner as described above for example C30, 3-(4-cyclopropylphenyl)-6-[4-(propan-2-yloxy)phenyl]imidazo[1,2-a]pyridine, using 3-bromo-6-[4-(propan-2-yloxy)phenyl]imidazo[1,2-a]pyridine, the appropriate commercial boronic acid or boronic acid pinacol ester, and the appropriate catalyst (Method A: Pd(PPh₃)₄; Method B: Pd(dppf)Cl₂·DCM; Method C: Pd(dba)₂ (0.1 eq.)/PCy₃ (0.15 eq.))

| | | Starting | | |
|---|---|---|---|---|
| Ex. | Method | materials | Product/Name | Analytical Data |
| C31 | A | (4- cyclopropoxy phenyl)boronic acid | 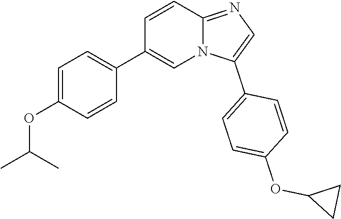 [SMILES: CC(C)Oc1ccc(-c2ccc3ncc(-c4ccc(OC5CC5)cc4)n3c2)cc1] | ¹H NMR (300 MHz, CDCl₃) δ ppm 8.39 (s, 1H), 8.08 (d, 1H), 7.72 (d, 2H), 7.56-7.39 (m, 5H), 7.25 (s, 1H), 7.00 (d, 2H), 4.62 (sep, 1H), 3.88-3.80 (m, 1H), 1.38 (d, 6H), 0.87 (br:s, 4H)., LC/MS m/z: 385.31 (M + H)⁺ |
| | | | 3-(4-cyclopropoxy phenyl)-6- | |
| | | | [4-(propan-2-yloxy)phenyl] | |
| | | | imidazo[1,2-a]pyridine | |
| C32 | A | (4-(tert-butoxy) phenyl)boronic acid | 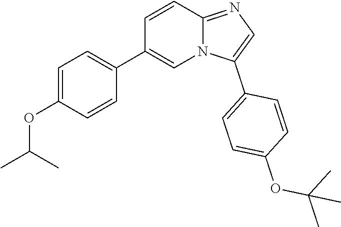 [SMILES: CC(C)Oc1ccc(-c2ccc3ncc(-c4ccc(OC(C)(C)C)cc4)n3c2)cc1] | ¹H NMR (300 MHz, CDCl₃) δ ppm 8.41 (s, 1H), 8.02 (d, 1H), 7.72 (s, 1H), 7.66 (d, 1H), 7.47 (dd, 4H), 7.19 (d, 2H), 7.00 (d, 2H), 4.62 (sept, 1H), 1.46 (s, 9H), 1.39 (d, 6H)., LC/MS m/z: 401.35 (M + H)⁺ |
| | | | 3-[4-(tert-butoxy)phenyl]-6- | |
| | | | [4-(propan-2-yloxy)phenyl] | |
| | | | imidazo[1,2-a]pyridine | |
| C33 | A | (4-(cyclopropyl methoxy)phenyl) boronic acid | 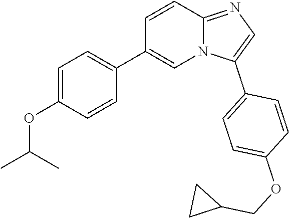 [SMILES: CC(C)Oc1ccc(-c2ccc3ncc(-c4ccc(OCC5CC5)cc4)n3c2)cc1] | ¹H NMR (300 MHz, CDCl₃) δ ppm 8.37 (s, 1H), 8.05 (d, 1H), 7.73- 7.66 (m, 2H), 7.47 (dd, 4H), 7.11 (d, 2H), 6.99 (d, 2H), 4.62 (sept, 1H), 3.91 (d, 2H), 1.39 (d, 6H), 0.77-0.66 (m, 2H), 0.46-0.37 (m, 2H)., LC/MS m/z: 399.31 (M + H)⁺ |
| | | | 3-[4-(cyclopropylmethoxy) | |
| | | | phenyl]-6-[4-(propan-2-yloxy) | |
| | | | phenyl]imidazo[1,2 a]pyridine | |
| C34 | A | (2,3-dihydro benzofuran-5- yl)boronic acid | 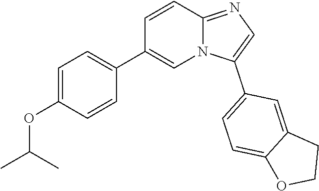 [SMILES: CC(C)Oc1ccc(-c2ccc3ncc(-c4ccc5c(c4)CCO5)n3c2)cc1] | LC/MS m/z: 371.35 (M + H)⁺ |
| | | | 3-(2,3-dihydro-1-benzofuran- | |
| | | | 5-yl)-6-[4-(propan-2-yloxy) | |
| | | | phenyl]imidazo[1,2- | |
| | | | a]pyridine | |
| C35 | A | (3-isopropoxy phenyl)boronic acid | 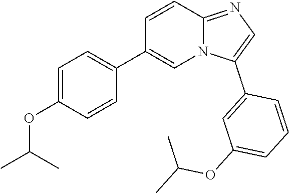 [SMILES: CC(C)Oc1ccc(-c2ccc3ncc(-c4cccc(OC(C)C)c4)n3c2)cc1] | LC/MS m/z: 387.34 (M + H)⁺, 428.47 (M + H + CH₃CN)⁺ |
| | | | 3-[3-(propan-2-yloxy)phenyl]- | |
| | | | 6-[4-(propan-2-yloxy)phenyl] | |
| | | | imidazo[1,2-a]pyridine | |
| C36 | B | (4- (difluoromethyl) phenyl)boronic acid | 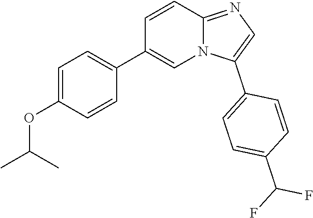 [SMILES: CC(C)Oc1ccc(-c2ccc3ncc(-c4ccc(C(F)F)cc4)n3c2)cc1] | LC/MS m/z: 379.31 (M + H)⁺, 420.51 (M + H + CH₃CN)⁺ |
| | | | 3-[4-(difluoromethyl)phenyl]- | |
| | | | 6-[4-(propan-2-yloxy)phenyl] | |
| | | | imidazo[1,2-a]pyridine | |
| C37 | A | (6-isopropoxy pyridin-3- yl)boronic acid | 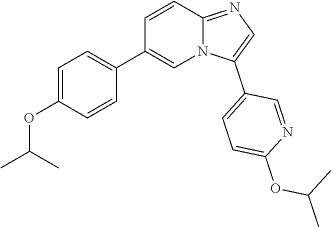 [SMILES: CC(C)Oc1ccc(-c2ccc3ncc(-c4ccc(OC(C)C)nc4)n3c2)cc1] | LC/MS m/z: 388.32 (M + H)⁺, 429.34 (M + H + CH₃CN)⁺ |
| | | | 2-(propan-2-yloxy)-5-{6-[4- | |
| | | | (propan-2-yloxy)phenyl] | |
| | | | imidazo[1,2-a]pyridin-3- | |
| | | | yl}pyridine | |
| C38 | B | (4-(2-hydroxy propan-2-yl phenyl)boronic acid | 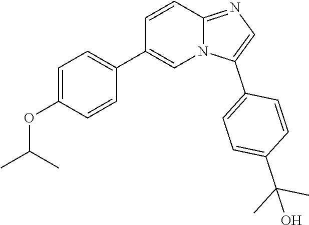 [SMILES: CC(C)Oc1ccc(-c2ccc3ncc(-c4ccc(C(C)(C)O)cc4)n3c2)cc1] | LC/MS m/z: 387.28 (M + H)⁺, 428.55 (M + H + CH₃CN)⁺ |
| | | | 2-(4-{6-[4-(propan-2-yloxy) | |
| | | | phenyl]imidazo[1,2-a]pyridin- | |
| | | | 3-yl}phenyl)propan-2-ol | |
| C39 | B | 1H-indol-5- ylboronic acid | 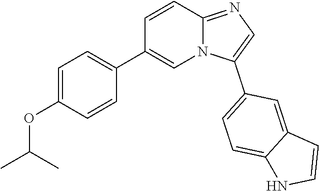 [SMILES: CC(C)Oc1ccc(-c2ccc3ncc(-c4ccc5[nH]ccc5c4)n3c2)cc1] | LC/MS m/z: 368.24 (M + H)⁺ |
| | | | 5-{6-[4-(propan- | |
| | | | 2yloxy)phenyl]imidazo[1,2- | |
| | | | a]pyridin-3-yl}-1H-indole | |
| C40 | C | (4-(difluoro methoxy)phenyl) boronic acid | 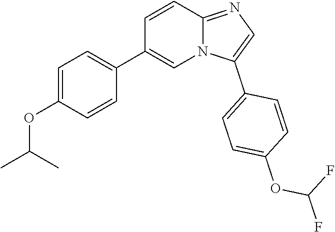 [SMILES: CC(C)Oc1ccc(-c2ccc3ncc(-c4ccc(OC(F)F)cc4)n3c2)cc1] | LC/MS m/z: 395.26 (M + H)⁺, 436.22 (M + H + CH₃CN)⁺ |
| | | | 3-[4-(difluoromethoxy) | |
| | | | phenyl]-6-[4-(propan-2- | |
| | | | yloxy)phenyl] | |
| | | | imidazo[1,2a]pyridine | |
| C41 | A | (4- acetylphenyl) boronic acid | 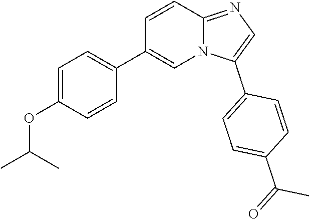 [SMILES: CC(=O)c1ccc(-c2cnc3ccc(-c4ccc(OC(C)C)cc4)cn23)cc1] | LC/MS m/z: 371.28 (M + H)⁺ |
| | | | 1-(4-{6-[4-(propan-2-yloxy) | |
| | | | phenyl]imidazo[1,2-a]pyridin- | |
| | | | 3-yl}phenyl)ethan-1-one | |
| C42 | B | [4-(2-methoxy ethoxy)phenyl] boronic acid | 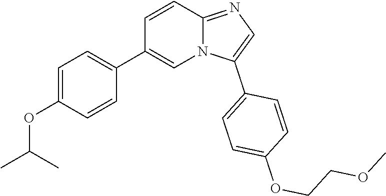 [SMILES: COCCOc1ccc(-c2cnc3ccc(-c4ccc(OC(C)C)cc4)cn23)cc1] | LC/MS m/z: 403.31 (M + H)⁺ |
| | | | 3-[4-(2-methoxyethoxy) | |
| | | | phenyl]-6-[4-(propan-2- | |
| | | | yloxy)phenyl]imidazo[1,2- | |
| | | | a]pyridine | |
| C43 | B | 4-(2- morpholino ethoxy)phenyl boronic acid | 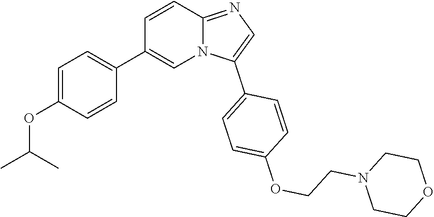 [SMILES: CC(C)Oc1ccc(-c2ccc3ncc(-c4ccc(OCCN5CCOCC5)cc4)n3c2)cc1] | LC/MS m/z: 458.10 (M + H)⁺ |
| | | | 4-[2-(4-{6-[4-(propan-2- | |
| | | | yloxy)phenyl]imidazo[1,2- | |
| | | | alpyridin-3-yl}phenoxy) | |
| | | | ethyl]morpholine | |

### Example C44: 1-(4-{6-[4-(propan-2-yloxy)phenyl]imidazo[1,2-a]pyridin-3-yl}phenyl)cyclopropan-1-ol

### Step 1: 3-(4-{1-[(tert-butyldimethylsilyl)oxy]cyclopropyl}phenyl)-6-[4-(propan-2-yloxy)phenyl]imidazo[1,2-a]pyridine

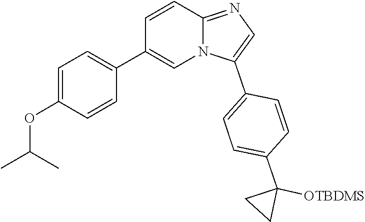 [SMILES: CC(C)Oc1ccc(-c2ccc3ncc(-c4ccc(C5(O[Si](C)(C)C(C)(C)C)CC5)cc4)n3c2)cc1]

The title compound was prepared from 3-bromo-6-[4-(propan-2-yloxy)phenyl]imidazo[1,2-a]pyridine and tert-butyldimethyl{1-[4-(4,4,5,5-tetramethyl-1,3,2-dioxaborolan-2-yl)phenyl]cyclopropoxy}silane in the same manner as described above for 3-(4-cyclopropylphenyl)-6-[4-(propan-2-yloxy)phenyl]imidazo[1,2-a]pyridine (Example C30). LC/MS m/z: 499.33 (M+H)⁺.

### Step 2: 1-(4-{6-[4-(propan-2-yloxy)phenyl]imidazo[1,2-a]pyridin-3-yl}phenyl)cyclopropan-1-ol

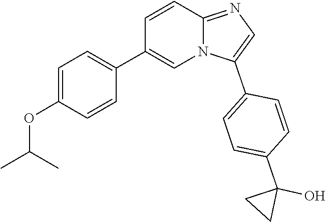 [SMILES: CC(C)Oc1ccc(-c2ccc3ncc(-c4ccc(C5(O)CC5)cc4)n3c2)cc1]

The title compound was prepared from 3-(4-{1-[(tert-butyldimethylsilyl)oxy]cyclopropyl}phenyl)-6-[4-(propan-2-yloxy)phenyl]imidazo[1,2-a]pyridine in the same manner as described above for 1-(4-{3-[4-(propan-2-yloxy)phenyl]imidazo[1,2-a]pyridin-6-yl}phenyl)cyclopropan-1-ol. ¹H NMR (300 MHz, CDCl₃) δ 8.43-8.37 (m, 2H), 7.97 (d, 1H), 7.63 (s, 1H), 7.55-7.48 (m, 4H), 7.41 (d, 2H), 6.99 (d, 2H), 4.60 (sep, 1H), 1.96 (br. s, 1H), 1.47-1.42 (m, 2H), 1.36 (d, 6H), 1.17-1.13 (m, 2H). LC/MS m/z: 385.35 (M+H)⁺.

### Example C45: 4-{6-[4-(propan-2-yloxy)phenyl]imidazo[1,2-a]pyridin-3-yl}benzoic Acid

### Step 1: ethyl 4-{6-[4-(propan-2-yloxy)phenyl]imidazo[1,2-a]pyridin-3-yl}benzoate

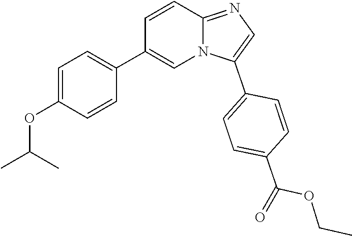 [SMILES: CCOC(=O)c1ccc(-c2cnc3ccc(-c4ccc(OC(C)C)cc4)cn23)cc1]

The title compound was prepared from 3-bromo-6-[4-(propan-2-yloxy)phenyl]imidazo[1,2-a]pyridine and [4-(ethoxycarbonyl)phenyl)oronic acid in the same manner as described above for 3-(4-cyclopropylphenyl)-6-[4-(propan-2-yloxy)phenyl]imidazo[1,2-a]pyridine (Example C30). LC/MS m/z: 401.31 (M+H)⁺.

### Step 2: 4-{6-[4-(propan-2-yloxy)phenyl]imidazo[1,2-a]pyridin-3-yl}benzoic Acid

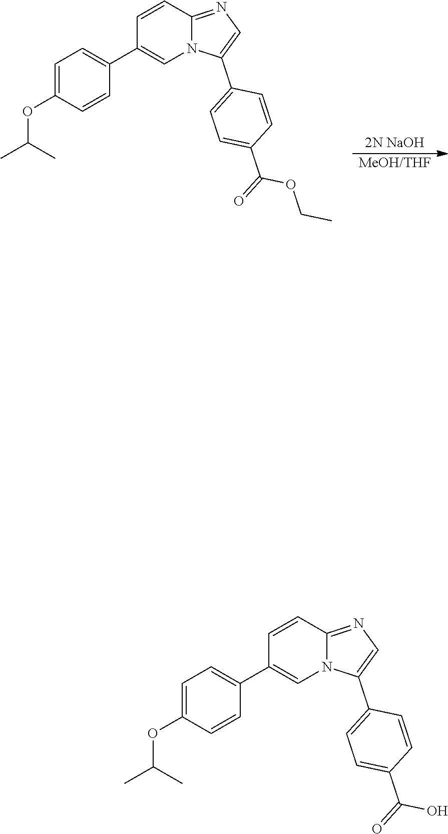

To a solution of ethyl 4-{6-[4-(propan-2-yloxy)phenyl]imidazo[1,2-a]pyridin-3-yl}benzoate (9.7 mg, 0.024 mmol) in a mixture of MeOH/THF (1:1) (0.5 mL) was added 2N NaOH (0.25 mL). The reaction mixture was stirred at r.t. for 4h, then concentrated in vacuo. The residue was acidified with 1N HCl, and then purified by preparative HPLC to afford 2 mg (22%) of the product as a white solid. LC/MS m/z: 373.20 (M+H)⁺.

### Example C46: 3,6-bis(4-cyclopropoxyphenyl)imidazo[1,2-a]pyridine

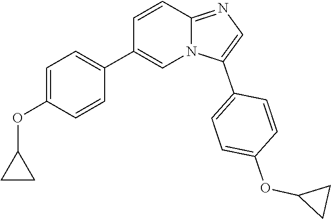 [SMILES: c1cc(-c2ccc3ncc(-c4ccc(OC5CC5)cc4)n3c2)ccc1OC1CC1]

The title compound was prepared from 6-bromo-3-iodoimidazo[1,2-a]pyridine and 4-(cyclopropoxy)phenylboronic acid in the same manner as described above for 3,6-bis[4-(propan-2-yloxy)phenyl]imidazo[1,2-a]pyridine (Example C2). LC/MS m/z: 383.33 (M+H)⁺.

### Example C47: 1-(4-{3-[4-(1-hydroxycyclobutyl)phenyl]imidazo[1,2-a]pyridin-6-yl}phenyl)cyclobutan-1-ol

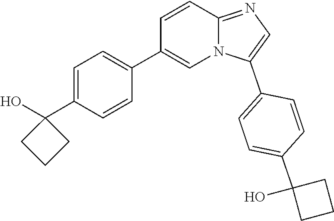 [SMILES: OC1(c2ccc(-c3ccc4ncc(-c5ccc(C6(O)CCC6)cc5)n4c3)cc2)CCC1]

The title compound was prepared from 6-bromo-3-iodoimidazo[1,2-a]pyridine and 4-(1-hydroxycyclobutyl)phenylboronic acid in the same manner as described above for 3,6-bis[4-(propan-2-yloxy)phenyl]imidazo[1,2-a]pyridine (Example C2). LC/MS m/z: 411.34 (M+H)⁺.

### Example C48: 7-methyl-3,6-bis[4-(propan-2-yloxy)phenyl]imidazo[1,2-a]pyridine

### Step 1: 6-bromo-3-iodo-7-methylimidazo[1,2-a]pyridine

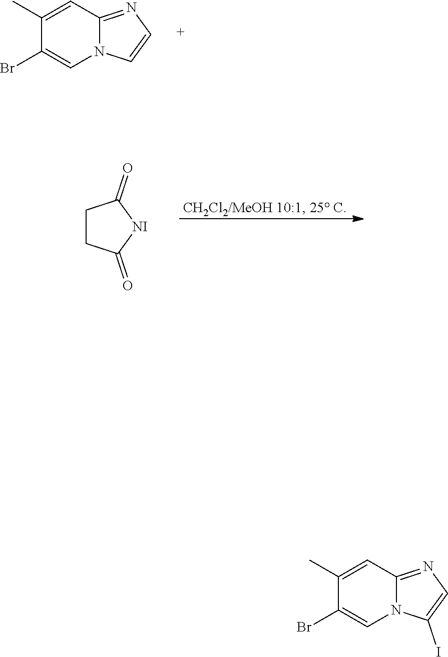

To a solution of 6-bromo-7-methylimidazo[1,2-a]pyridine (100 mg, 0.47 mmol) in CH₂Cl₂ (1 mL), was added 1-Iodopyrrolidine-2,5-dione (84 mg, 0.47 mmol) and MeOH (0.1 mL). The resulting mixture was stirred at room temperature for 2 h. The reaction was poured into water and extracted with ethyl acetate. The organic phase was dried over Na₂SO₄, filtered, and concentrated in vacuo. The residue was purified by SiO₂ column chromatography (hexane/EtOAc=1:1) to give 122 mg (77%) of the product as a white solid. LC/MS m/z: 337.00 (M+H)⁺.

### Step 2: 7-methyl-3,6-bis[4-(propan-2-yloxy)phenyl]imidazo[1,2-a]pyridine

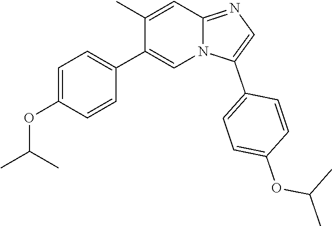 [SMILES: Cc1cc2ncc(-c3ccc(OC(C)C)cc3)n2cc1-c1ccc(OC(C)C)cc1]

The title compound was prepared from 6-bromo-3-iodo-7-methylimidazo[1,2-a]pyridine and [4-(propan-2-yloxy)phenyl]boronic acid in the same manner as described above for 3,6-bis[4-(propan-2-yloxy)phenyl]imidazo[1,2-a]pyridine (Example C2). LC/MS m/z: 401.33 (M+H)⁺.

### Example C49: 7-methoxy-3,6-bis[4-(propan-2-yloxy)phenyl]imidazo[1,2-a]pyridine

### Step 1: 6-bromo-3-iodo-7-methoxyimidazo[1,2-a]pyridine

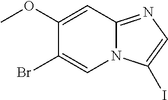

The title compound was prepared from 6-bromo-7-methoxyimidazo[1,2-a]pyridine in the same manner as described above for 6-bromo-3-iodo-7-methylimidazo[1,2-a]pyridine (Example C48, step 1). LC/MS m/z: 394.01 (M+H+CH₃CN)⁺.

### Step 2: 7-methoxy-3,6-bis[4-(propan-2-yloxy)phenyl]imidazo[1,2-a]pyridine

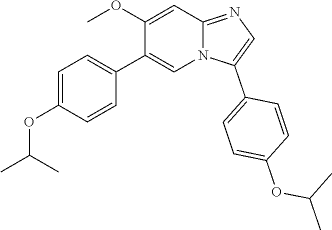 [SMILES: COc1cc2ncc(-c3ccc(OC(C)C)cc3)n2cc1-c1ccc(OC(C)C)cc1]

The title compound was prepared from 6-bromo-3-iodo-7-methoxyimidazo[1,2-a]pyridine and [4-(propan-2-yloxy)phenyl]boronic acid in the same manner as described above for 3,6-bis[4-(propan-2-yloxy)phenyl]imidazo[1,2-a]pyridine (Example C2). ¹H NMR (300 MHz, CDCl₃) δ 8.06 (s, 1H), 7.68 (s, 1H), 7.53 (s, 1H), 7.41 (d, 2H), 7.34 (d, 2H), 7.06 (d, 2H), 6.96 (d, 2H), 4.54-4.68 (m, 2H), 4.05 (s, 3H), 1.39 (d, 6H), 1.37 (d, 6H). LC/MS m/z: 417.27 (M+H)⁺.

### Example C50: 8-methyl-3,6-bis[4-(propan-2-yloxy)phenyl]imidazo[1,2-a]pyridine

### Step 1: 6-bromo-3-iodo-8-methylimidazo[1,2-a]pyridine

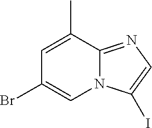

The title compound was prepared from 6-bromo-8-methylimidazo[1,2-a]pyridine in the same manner as described above for 6-bromo-3-iodo-7-methylimidazo[1,2-a]pyridine (Example C48, step 1). LC/MS m/z: 337.05 (⁷⁹Br, M+H)⁺, 339.04 (⁸¹Br, M+H)⁺, 378.22 (⁷⁹Br, M+H+CH₃CN)⁺, 380.00 (⁸¹Br, M+H+CH₃CN)⁺.

### Step 2: 8-methyl-3,6-bis[4-(propan-2-yloxy)phenyl]imidazo[1,2-a]pyridine

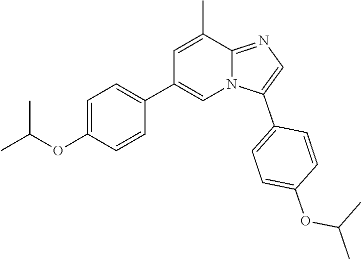 [SMILES: Cc1cc(-c2ccc(OC(C)C)cc2)cn2c(-c3ccc(OC(C)C)cc3)cnc12]

The title compound was prepared from 6-bromo-3-iodo-8-methylimidazo[1,2-a]pyridine and [4-(propan-2-yloxy)phenyl]boronic acid in the same manner as described above for 3,6-bis[4-(propan-2-yloxy)phenyl]imidazo[1,2-a]pyridine (Example C2). LC/MS m/z: 401.33 (M+H)⁺.

### Example C51: 5-methyl-3,6-bis[4-(propan-2-yloxy)phenyl]imidazo[1,2-a]pyridine

### Step 1: 6-bromo-3-iodo-5-methylimidazo[1,2-a]pyridine

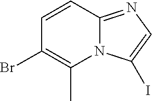

The title compound was prepared from 6-bromo-7-methylimidazo[1,2-a]pyridine in the same manner as described above for 6-bromo-3-iodo-8-methylimidazo[1,2-a]pyridine (Example C50, step 1). LC/MS m/z: 337.00 (M+H)⁺.

### Step 2: 5-methyl-3,6-bis[4-(propan-2-yloxy)phenyl]imidazo[1,2-a]pyridine

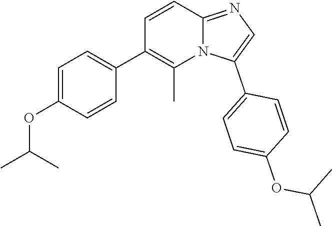 [SMILES: Cc1c(-c2ccc(OC(C)C)cc2)ccc2ncc(-c3ccc(OC(C)C)cc3)n12]

The title compound was prepared from 6-bromo-3-iodo-7-methoxyimidazo[1,2-a]pyridine and [4-(propan-2-yloxy)phenyl]boronic acid in the same manner as described above for 3,6-bis[4-(propan-2-yloxy)phenyl]imidazo[1,2-a]pyridine (Example C2). ¹H NMR (300 MHz, CDCl₃) δ8.14 (d, 1H), 7.63 (s, 1H), 7.56 (d, 1H), 7.37 (d, 2H), 7.18 (d, 2H), 6.97 (d, 2H), 6.94 (d, 2H), 4.53-4.69 (m, 2H), 2.19 (s, 3H), 1.39 (d, 6H), 1.37 (d, 6H). LC/MS m/z: 401.29 (M+H)⁺.

### Preparation of Intermediates for Examples C52 to C59

### 6-bromoimidazo[1,2-a]pyridine-7-carbonitrile

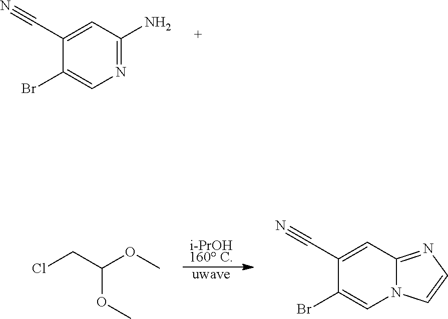

To a solution of 2-amino-5-bromoisonicotinonitrile (150 mg, 0.76 mmol) in i-PrOH (2 mL) is added 0.6 mL (1.5 eq) of 2-chloro-1,1-dimethoxyethane. The solution is capped tightly and heated in a microwave reactor to 160° C. for 30 minutes. The mixture is cooled and evaporated in vacuo, the residue dissolved in ethyl acetate, washed with saturated aq. NaHCO₃, and evaporated in vacuo to give 0.47 g of the title compound, pure enough for further use. LC/MS m/z: 221.10 (M+H)⁺

### 6-bromo-3-iodoimidazo[1,2-a]pyridine-7-carbonitrile

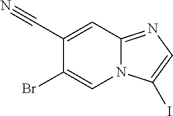 [SMILES: N#Cc1cc2ncc(I)n2cc1Br]

The title compound was prepared from 6-bromoimidazo[1,2-a]pyridine-7-carbonitrile and NIS in the same manner as described for 6-bromo-3-iodo-7-methylimidazo[1,2-a]pyridine. LC/MS m/z: 348.01 (M+H)⁺.

### 6-bromo-3-(4-(tert-butoxy)phenyl)-7-methylimidazo[1,2-a]pyridine

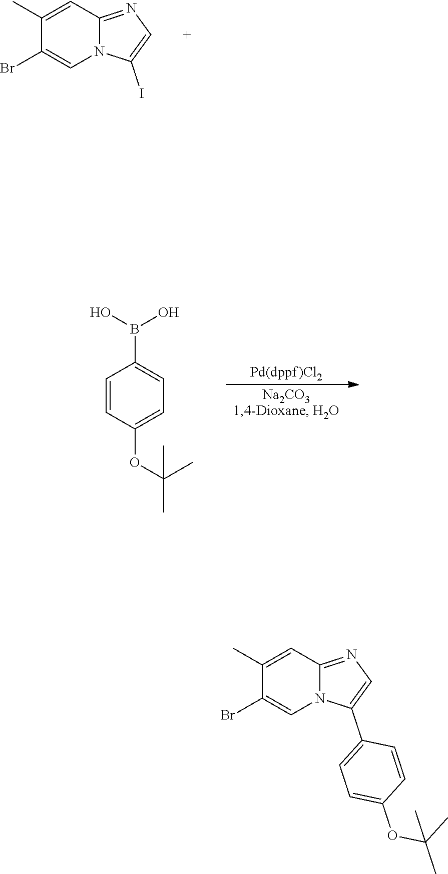 [SMILES: Cc1cc2ncc(I)n2cc1Br ; CC(C)(C)Oc1ccc(B(O)O)cc1 ; "Na2CO31,4-Dioxane, H2O" ; "Pd(dppf)Cl2" ; Cc1cc2ncc(-c3ccc(OC(C)(C)C)cc3)n2cc1Br]

To a solution of 6-bromo-3-iodo-7-methylimidazo[1,2-a]pyridine (33.6 mg, 0.1 mmol) in 1,4-dioxane (1 mL) was added 4-tert-butoxyphenylboronic acid (19.4 mg, 0.1 mmol), sodium carbonate (21.2 mg, 0.2 mmol) and water (0.5 mL). Resulting reaction mixture was purged with nitrogen and Pd(dppf)Cl₂ (5 mg) was added. Reaction mixture was stirred at 80° C. for 4 hours and brought to RT, diluted with ethyl acetate. Extracted with ethyl acetate, followed by drying over Na₂SO₄ and evaporation to yield the crude product. The crude material is purified on silica gel flash chromatography using ethyl acetate/hexane as eluent to give 24 mg (66%) of the title compound. LC/MS m/z: 359.22 (M+H)⁺

### 6-bromo-3-(4-isopropoxyphenyl)-7-methylimidazo[1,2-a]pyridine

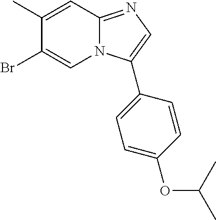 [SMILES: Cc1cc2ncc(-c3ccc(OC(C)C)cc3)n2cc1Br]

The title compound was prepared from 6-bromo-3-iodo-7-methylimidazo[1,2-a]pyridine and 4-isopropoxyboronic acid in the same manner as described for 6-bromo-3-(4-(tert-butoxy)phenyl)-7-methylimidazo[1,2-a]pyridine. LC/MS m/z: 345.20 (M+H)⁺

### 2-(4-(6-bromo-7-methylimidazo[1,2-a]pyridin-3-yl)phenyl)propan-2-ol

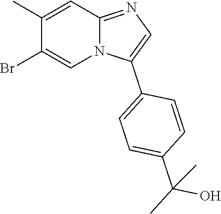 [SMILES: Cc1cc2ncc(-c3ccc(C(C)(C)O)cc3)n2cc1Br]

The title compound was prepared from 6-bromo-3-iodo-7-methylimidazo[1,2-a]pyridine and (4-(2-hydroxypropan-2-yl)phenyl)boronic acid in the same manner as described for 6-bromo-3-(4-(tert-butoxy)phenyl)-7-methylimidazo[1,2-a]pyridine. LC/MS m/z: 345.10 (M+H)⁺

### 6-bromo-3-(4-(trifluoromethoxy)phenyl)imidazo[1,2-a]pyridine

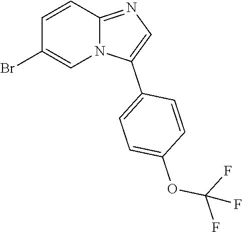 [SMILES: FC(F)(F)Oc1ccc(-c2cnc3ccc(Br)cn23)cc1]

The title compound was prepared from 6-bromo-3-iodoimidazo[1,2-a]pyridine and (4-(trifluoromethoxy)phenyl)boronic acid in the same manner as described for 6-bromo-3-(4-(tert-butoxy)phenyl)-7-methylimidazo[1,2-a]pyridine. LC/MS m/z: 357.13 (⁷⁹Br, M+H)⁺, 359.32 (⁸¹Br, M+H)⁺

### 6-bromo-3-(3,5-dimethoxyphenyl)imidazo[1,2-a]pyridine

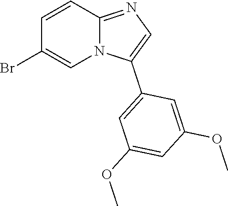 [SMILES: COc1cc(OC)cc(-c2cnc3ccc(Br)cn23)c1]

The title compound was prepared from 6-bromo-3-iodoimidazo[1,2-a]pyridine and (3,5-dimethoxyphenyl)boronic acid in the same manner as described for 6-bromo-3-(4-(tert-butoxy)phenyl)-7-methylimidazo[1,2-a]pyridine. LC/MS m/z: 333.12 (⁷⁹Br, M+H)⁺, 335.16 (⁸¹Br, M+H)⁺

Examples C52 to C61 were prepared in the same manner as described above for 6-bromo-3-(4-(tert-butoxy)phenyl)-7-methylimidazo[1,2-a]pyridine using the appropriate aryl halide and commercially available boronic acids. In the case of compounds which have identical substitutions of the aryl halide, 2 equivalents of the boronic acid are used.

| Ex. | Starting materials | Product/Name | Analytical Data |
|---|---|---|---|
| C52 | 6-bromo-3- iodoimidazo[1,2- a]pyridine-7-carbonitrile 4- isopropoxyphenylboronic acid (2 Equivalents) | 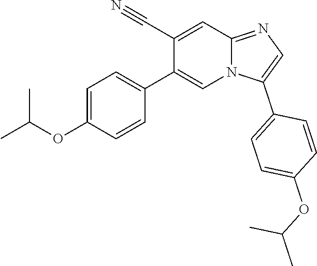 [SMILES: CC(C)Oc1ccc(-c2cn3c(-c4ccc(OC(C)C)cc4)cnc3cc2C#N)cc1] | LC/MS m/z: 412.29 (M + H)⁺ |
| | | 3,6-bis(4- | |
| | | isopropoxyphenyl)imidazo[1,2-a] | |
| | | pyridine-7-carbonitrile | |
| C53 | 6-bromo-3-(4- isopropoxyphenyl)-7- methylimidazo[1,2- a]pyridine (4-(2-hydroxypropan-2- yl)phenyl)boronic acid | 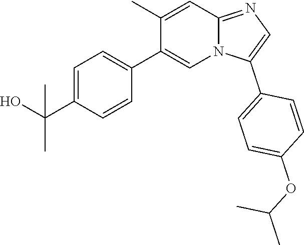 [SMILES: Cc1cc2ncc(-c3ccc(OC(C)C)cc3)n2cc1-c1ccc(C(C)(C)O)cc1] | ¹H NMR (500 MHz, DMSO-d6) δ 8.11 (s, 1H), 7.62 (s, 1H), 7.58 (s, 1H), 7.56 (d, 2H), 7.55 (d, 2H), 7.42 (d, 2H), 7.05 (d, 2H), 4.63- 4.68 (m, 1H), 2.26 (s, 3H), 1.46 (s, 6H), 1.27 (d, 6H). LC/MS m/z: 401.31 (M + H)⁺ |
| | | 2-(4-(3-(4-isopropoxyphenyl)- | |
| | | 7-methylimidazo[1,2- | |
| | | a]pyridin-6-yl)phenyl)propan- | |
| | | 2-ol | |
| C54 | 6-bromo-3-(4-(tert- butoxy)phenyl)-7- methylimidazo[1,2- a]pyridine (4-(2-hydroxypropan-2- yl)phenyl)boronic acid | 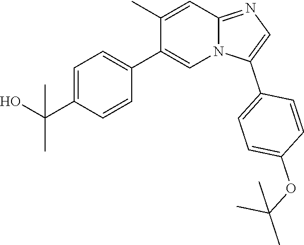 [SMILES: Cc1cc2ncc(-c3ccc(OC(C)(C)C)cc3)n2cc1-c1ccc(C(C)(C)O)cc1] | ¹H NMR (500 MHz, DMSO-d6) δ 8.17 (s, 1H), 7.67 (s, 1H), 7.58 (d, 2H), 7.56 (s, 1H), 7.54 (d, 2H), 7.37 (d, 2H), 7.10 (d, 2H), 2.26 (s, 3H), 1.46 (s, 6H), 1.33 (s, 9H). LC/MS m/z: 415.34 (M + H)⁺ |
| | | 2-(4-(3-(4-(tert- | |
| | | butoxy)phenyl)-7- | |
| | | methylimidazo[1,2-a]pyridin- | |
| | | 6-yl)phenyl)propan-2-ol | |
| C55 | 6-bromo-3-(4-(tert- butoxy)phenyl)-7- methylimidazo[1,2- a]pyridine 4- isopropoxyphenylboronic acid | 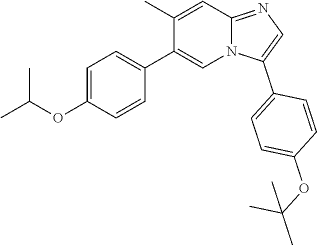 [SMILES: Cc1cc2ncc(-c3ccc(OC(C)(C)C)cc3)n2cc1-c1ccc(OC(C)C)cc1] | ¹H NMR (500 MHz, DMSO-d6) δ 8.13 (s, 1H), 7.65 (s, 1H), 7.57 (d, 2H), 7.54 (s, 1H), 7.33 (d, 2H), 7.11 (d, 2H), 6.97 (d, 2H), 4.62- 4.67 (m, 1H), 2.24 (s, 3H), 1.33 (s, 9H), 1.28 (d, 6H). LC/MS m/z: 415.36 (M + H)⁺ |
| | | 3-(4-(tert-butoxy)phenyl)-6-(4- | |
| | | isopropoxyphenyl)-7- | |
| | | methylimidazo[1,2-a]pyridine | |
| C56 | 2-(4-(6-bromo-7- methylimidazo[1,2- a]pyridin-3- yl)phenyl)propan-2-ol 4- isopropoxyphenylboronic acid | 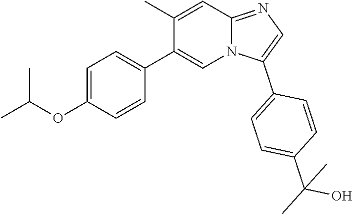 [SMILES: Cc1cc2ncc(-c3ccc(C(C)(C)O)cc3)n2cc1-c1ccc(OC(C)C)cc1] | ¹H NMR (500 MHz, DMSO-d6) δ 8.29 (s, 1H), 8.15 (s, 1H), 7.88 (S, 1H), 7.38 (d, 2H), 7.34 (d, 2H), 7.13 (d, 2H), 7.01 (d, 2H), 4.64- 4.69 (m, 1H), 2.38 (s, 3H), 1.46 (s, 6H), 1.28 (d, 6H). LC/MS m/z: 401.36 (M + H)⁺ |
| | | 2-(4-(6-(4-isopropoxyphenyl)- | |
| | | 7-methylimidazo[1,2- | |
| | | a]pyridin-3-yl)phenyl)propan- | |
| | | 2-ol | |
| C57 | 2-(4-(6-bromo-7- methylimidazo[1,2- a]pyridin-3- yl)phenyl)propan-2-ol (4-(2-hydroxypropan-2- yl)phenyl)boronic acid | 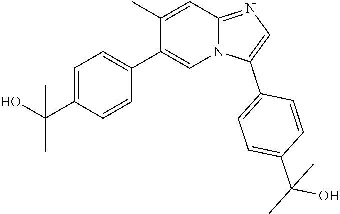 [SMILES: Cc1cc2ncc(-c3ccc(C(C)(C)O)cc3)n2cc1-c1ccc(C(C)(C)O)cc1] | LC/MS m/z: 401.37 (M + H)⁺ |
| | | 2,2′-((7-methylimidazo[1,2- | |
| | | a]pyridine-3,6-diyl)bis(4,1- | |
| | | phenylene))bis(propan-2-ol) | |
| C58 | 6-bromo-3-(4- (trifluoromethoxy)phenyl) imidazo[1,2-a]pyridine (3,5- dimethoxyphenyl) boronic acid | 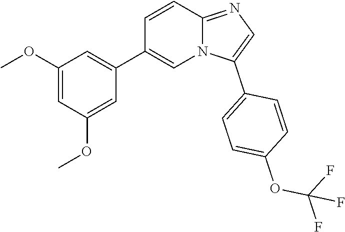 [SMILES: COc1cc(OC)cc(-c2ccc3ncc(-c4ccc(OC(F)(F)F)cc4)n3c2)c1] | LC/MS m/z: 415.20 (M + H)⁺ |
| | | 6-(3,5-dimethoxyphenyl)-3-(4- | |
| | | (trifluoromethoxy)phenyl) | |
| | | imidazo[1,2-a]pyridine | |
| C59 | 6-bromo-3-(3,5- dimethoxyphenyl)imidazo [1,2-a]pyridine (4- (trifluoromethoxy)phenyl) boronic acid | 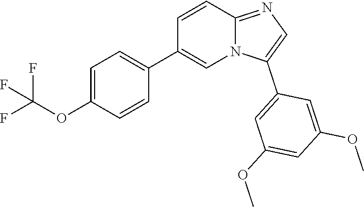 [SMILES: COc1cc(OC)cc(-c2cnc3ccc(-c4ccc(OC(F)(F)F)cc4)cn23)c1] | LC/MS m/z: 415.23 (M + H)⁺ |
| | | 3-(3,5-dimethoxyphenyl)-6-(4- | |
| | | (trifluoromethoxy)phenyl) | |
| | | imidazo[1,2-a]pyridine | |
| C60 | 3-bromo-6- (trifluoromethyl)imidazo [1,2-a]pyridine (4- (trifluoromethoxy)phenyl) boronic acid | 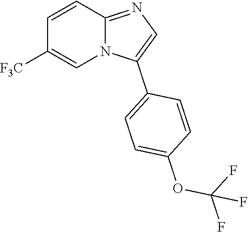 [SMILES: FC(F)(F)Oc1ccc(-c2cnc3ccc(C(F)(F)F)cn23)cc1] | LC/MS m/z: 347.14 (M + H)⁺ |
| | | 3-(4- | |
| | | (trifluoromethoxy)phenyl)-6- | |
| | | (trifluoromethyl)imidazo[1,2- | |
| | | a]pyridine | |
| C61 | 3-bromo-6- (trifluoromethyl)imidazo [1,2-a]pyridine 4- isopropoxyphenylboronic acid | 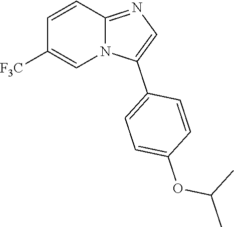 [SMILES: CC(C)Oc1ccc(-c2cnc3ccc(C(F)(F)F)cn23)cc1] | LC/MS m/z: 321.32 (M + H)⁺ |
| | | 3-(4-isopropoxyphenyl)-6- | |
| | | (trifluoromethyl)imidazo[1,2- | |
| | | a]pyridine | |

### Example C62: ethyl 3,6-bis[4-(propan-2-yloxy)phenyl]imidazo[1,2-a]pyridine-2-carboxylate

### Step 1: ethyl 3,6-dibromoimidazo[1,2-a]pyridine-2-carboxylate

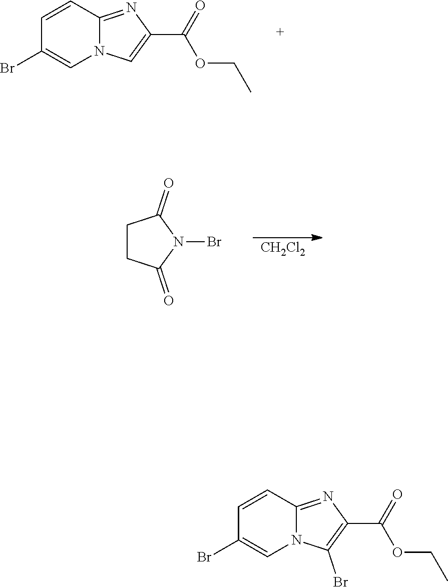

To a solution of ethyl 6-bromoimidazo[1,2-a]pyridine-2-carboxylate (200 mg, 0.74 mmol) in CH₂Cl₂ (3 mL), was added 1-bromopyrrolidine-2,5-dione (145 mg, 0.81 mmol) at 10° C. The resulting mixture was brought to room temperature and stirred for 3 hr. The reaction was poured into water and extracted with ethyl acetate. The organic phase was dried over Na₂SO₄, filtered, and concentrated in vacuo. The residue was purified by SiO₂ column chromatography (hexanes/EtOAc) to give 184 mg (71%) of the product as a beige solid. LC/MS m/z: 346.95 (⁷⁹Br, M+H)⁺, 348.97 (⁸¹Br, M+H)⁺.

### Step 2: ethyl 3,6-bis[4-(propan-2-yloxy)phenyl]imidazo[1,2-a]pyridine-2-carboxylate

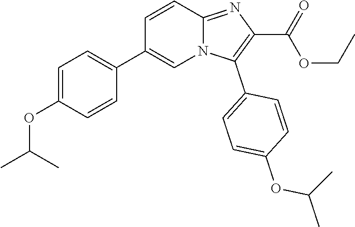 [SMILES: CCOC(=O)c1nc2ccc(-c3ccc(OC(C)C)cc3)cn2c1-c1ccc(OC(C)C)cc1]

The title compound was prepared from ethyl 3,6-dibromoimidazo[1,2-a]pyridine-2-carboxylate and (4-isopropoxyphenyl)boronic acid in the same manner as described above for 3,6-bis[4-(propan-2-yloxy)phenyl]imidazo[1,2-a]pyridine (Example C2). LC/MS m/z: 459.27 (M+H)⁺.

### Example C63: 3,6-bis[4-(propan-2-yloxy)phenyl]imidazo[1,2-a]pyridine-2-carboxylic Acid

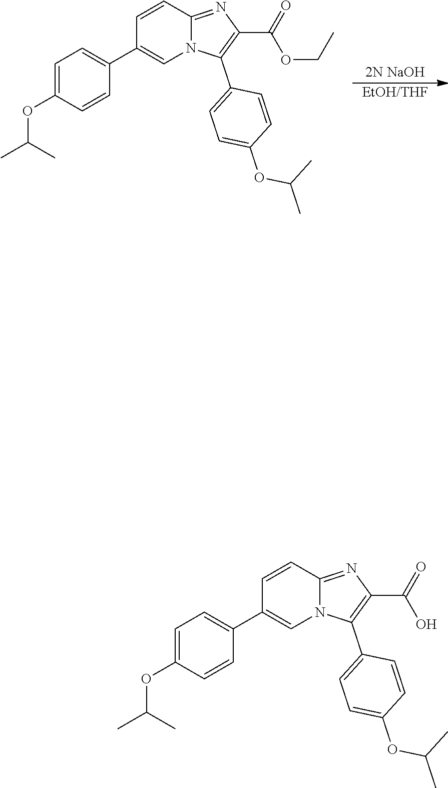

To a solution of ethyl 3,6-bis[4-(propan-2-yloxy)phenyl]imidazo[1,2-a]pyridine-2-carboxylate (30 mg, 0.065 mmol) in a mixture of EtOH/THF (1:1) (1 mL) was added 2N NaOH (0.5 mL). The reaction mixture was stirred at r.t. for 4h, then concentrated in vacuo. The residue was acidified with 1N HCl, and then purified by preparative HPLC to afford 19.6 mg (70%) of the product as a white solid. LC/MS m/z: 431.25 (M+H)⁺.

### Example C64: 3,6-bis[4-(propan-2-yloxy)phenyl]imidazo[1,2-a]pyridine-2-carboxamide

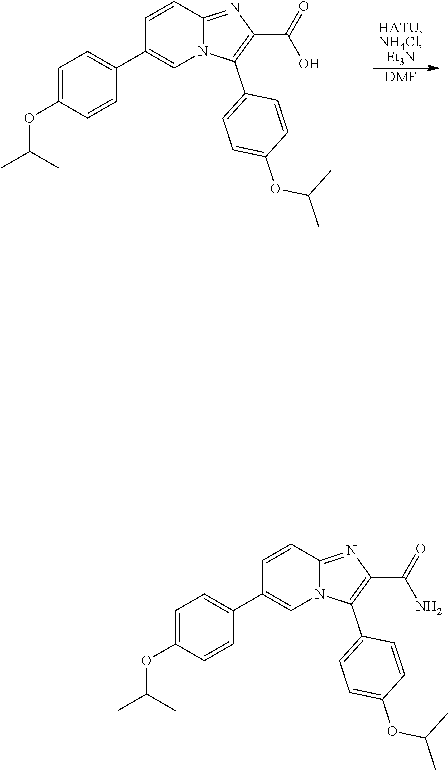

To a solution of 3,6-bis[4-(propan-2-yloxy)phenyl]imidazo[1,2-a]pyridine-2-carboxylic acid (16 mg, 0.037 mmol) in DMF (0.5 mL) cooled to 10° C. were added Et₃N (11 mg, 0.11 mmol) and HATU (21 mg, 0.055 mmol). The reaction mixture was stirred at r.t. for 0.5 h, and then NH₄Cl (8 mg, 0.15 mmol) was added. The reaction mixture was stirred at r.t. overnight, then diluted with DCM. The organic phase was washed with saturated NaHCO₃ solution and water. The organic layer was dried over Na₂SO₄, filtered, and concentrated in vacuo. The residue was purified by preparative HPLC to afford 6 mg (37%) of the product as a white solid. LC/MS m/z: 430.28 (M+H)⁺.

### Example C65: 3,6-bis[4-(propan-2-yloxy)phenyl]imidazo[1,2-a]pyridine-2-carbonitrile

### Step 1: 6-bromoimidazo[1,2-a]pyridine-2-carbonitrile

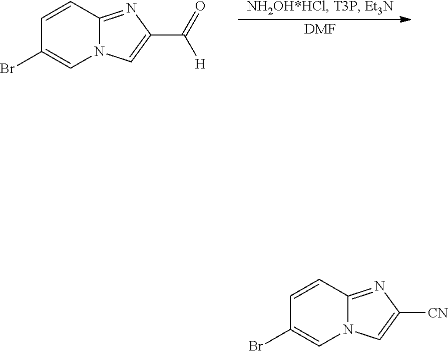

To a mixture of 6-bromoimidazo[1,2-a]pyridine-2-carbaldehyde (0.1 g, 0.44 mmol), hydroxylamine hydrochloride (34 mg, 0.49 mmol), and Et₃N (49 mg, 0.49 mmol) in DMF (0.5 mL) was added propylphosphonic anhydride solution in DMF (50 wt. %, 0.29 mL, 0.49 mmol). The reaction mixture was stirred at 100° C. for 3h. Then, the reaction mixture was cooled to r.t., diluted with EtOAc and washed with saturated NaHCO₃ solution and water. The organic layer was dried over Na₂SO₄, filtered, and concentrated in vacuo. The residue was purified by SiO₂ column chromatography (hexanes/EtOAc from 7:3 to 1:1) to give 35 mg (35%) of the product as a white solid. LC/MS m/z: 222.08 (⁷⁹Br, M+H)⁺, 224.13 (⁸¹Br, M+H)⁺.

### Step 2: 3,6-dibromoimidazo[1,2-a]pyridine-2-carbonitrile

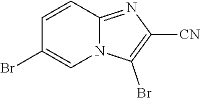 [SMILES: N#Cc1nc2ccc(Br)cn2c1Br]

The title compound was prepared from 6-bromoimidazo[1,2-a]pyridine-2-carbonitrile in the same manner as described above for ethyl 3,6-dibromoimidazo[1,2-a]pyridine-2-carboxylate. LC/MS m/z: 340.92 (⁷⁹Br, M+CH₃CN+H)⁺, 342.97 (⁸¹Br, M+CH₃CN+H)⁺.

### Step 3: 3,6-bis[4-(propan-2-yloxy)phenyl]imidazo[1,2-a]pyridine-2-carbonitrile

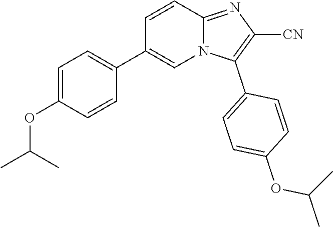 [SMILES: CC(C)Oc1ccc(-c2ccc3nc(C#N)c(-c4ccc(OC(C)C)cc4)n3c2)cc1]

The title compound was prepared from 3,6-dibromoimidazo[1,2-a]pyridine-2-carbonitrile and (4-isopropoxyphenyl)boronic acid in the same manner as described above for 3,6-bis[4-(propan-2-yloxy)phenyl]imidazo[1,2-a]pyridine. LC/MS m/z: 412.21 (M+H)⁺.

### Example C66: N-(methylsulfonyl)-3-(4-(trifluoromethoxy)phenyl)imidazo[1,2-a]pyridine-7-carboxamide

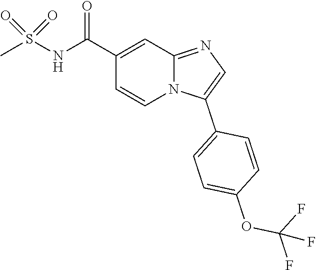 [SMILES: CS(=O)(=O)NC(=O)c1ccn2c(-c3ccc(OC(F)(F)F)cc3)cnc2c1]

### Step 1: 3-bromo-N-(methylsulfonyl)imidazo[1,2-a]pyridine-7-carboxamide

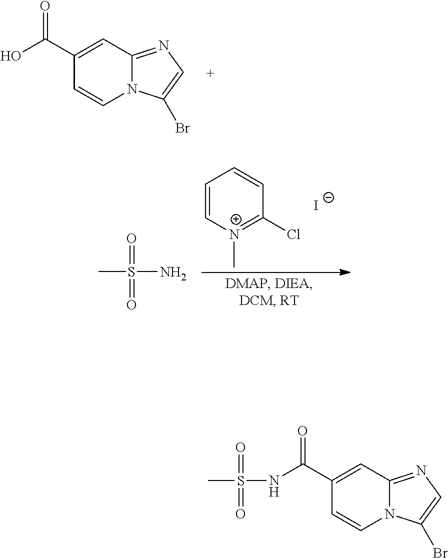

To a solution of 3-bromoimidazo[1,2-a]pyridine-7-carboxylic acid (0.241 g, 1 mmol) in DCM (3 mL) were added 2-chloro-1-methylpyridinium iodide (0.306 g, 1.2 mmol) followed by DIEA (0.52 mL, 3 mmol). Resulting reaction mixture was stirred at RT for 15 min. Then methanesulfonamide (0.285 g, 3 mmol) followed by catalytic amount of DMAP (0.005 g) was added, and reaction mixture was stirred for overnight. Then the reaction mixture was diluted with DCM (5 mL) and washed with water. The organic phase was dried over Na₂SO₄, filtered, and concentrated in vacuo. The residue was purified by SiO₂ column chromatography (DCM/MeOH) to give 0.082 g of the title compound (Yield: 25%). LC/MS m/z: 319.93 (M+H)⁺

### Step 2: N-(methylsulfonyl)-3-(4-(trifluoromethoxy)phenyl)imidazo[1,2-a]pyridine-7-carboxamide

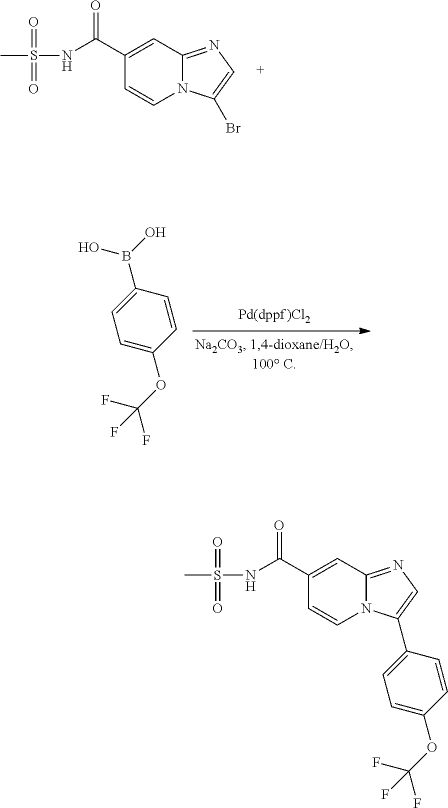 [SMILES: CS(=O)(=O)NC(=O)c1ccn2c(Br)cnc2c1 ; OB(O)c1ccc(OC(F)(F)F)cc1 ; CS(=O)(=O)NC(=O)c1ccn2c(-c3ccc(OC(F)(F)F)cc3)cnc2c1]

To a solution of 3-bromo-N-(methylsulfonyl)imidazo[1,2-a]pyridine-7-carboxamide (0.080 g, 0.25 mmol) in 1,4-dioxane (1 mL) were added 4-(trifluoromethoxy)phenylboronic acid (0.052 g, 0.25 mlmol), Na₂CO₃ (0.080 g, 0.75 mmol), water (0.5 mL) followed by catalytic amount of Pd(dppf)Cl₂·DCM (10 mg, 0.012 mmol). The resulting reaction mixture was purged with nitrogen, and then heated at 100° C. for overnight. Reaction was brought to RT, solvent was evaporated under vacuum, and extracted with ethyl acetate. The organic phase was dried over Na₂SO₄, filtered, and evaporated under vacuum to give a crude residue, which was purified using preparative HPLC to give 0.0024 g of the title compound (Yield: 1.8%). LC/MS m/z: 400.22 (M+H)⁺

### Example C67: N-(methylsulfonyl)-3,6-bis(4-(trifluoromethoxy)phenyl)imidazo[1,2-a]pyridine-7-carboxamide

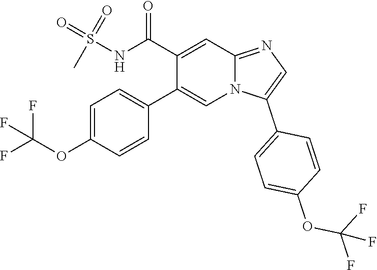 [SMILES: CS(=O)(=O)NC(=O)c1cc2ncc(-c3ccc(OC(F)(F)F)cc3)n2cc1-c1ccc(OC(F)(F)F)cc1]

### Step 1: Methyl 6-bromoimidazo[1,2-a]pyridine-7-carboxylate

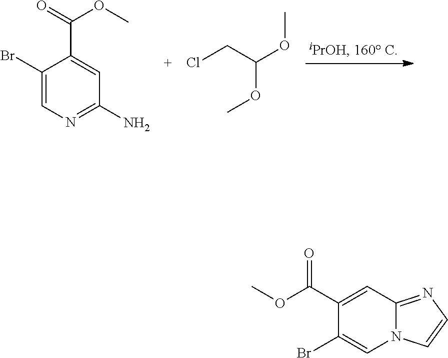

To a solution of methyl 2-amino-5-bromoisonicotinate (0.400 g, 1.72 mmol) in ⁱPrOH (2 mL) was added chloroacetaldehyde dimethyl acetal (1 mL). Resulting reaction mixture was microwaved at 160° C. for 2h. Solvent was evaporated under vacuum to give hydrochloride salt, which was then treated with ammonia in methanol, and methanol was evaporated to give 0.320 g of the title compound (Yield: 73%). LC/MS m/z: 256.12 (M+H)⁺

### Step 2: Methyl 6-bromo-3-iodoimidazo[1,2-a]pyridine-7-carboxylate

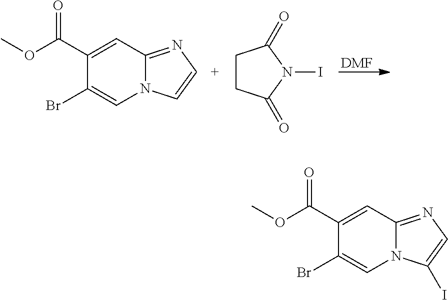

To a solution of methyl 6-bromoimidazo[1,2-a]pyridine-7-carboxylate (0.058 g, 0.23 mmol) in DMF (1 mL), was added N-iodosuccinimide (0.056 g, 0.25 mmol). The resulting reaction mixture was stirred at room temperature for overnight. The reaction was poured into water and extracted with ethyl acetate. The organic phase was dried over Na₂SO₄, filtered, and concentrated under vacuum. The residue was purified by SiO₂ column chromatography (hexane/EtOAc=1:1) to give 0.065 g of the title compound (Yield: 74%). LC/MS m/z: 380.89 (M+H)⁺.

### Step 3: Methyl 3,6-bis(4-(trifluoromethoxy)phenyl)imidazo[1,2-a]pyridine-7-carboxylate

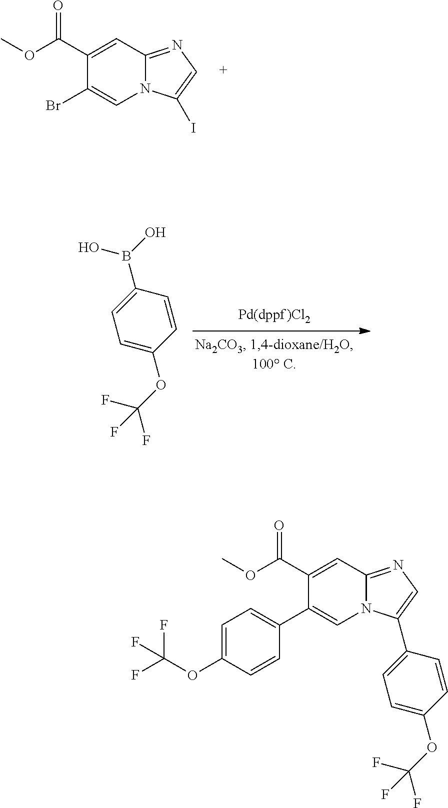 [SMILES: COC(=O)c1cc2ncc(I)n2cc1Br ; OB(O)c1ccc(OC(F)(F)F)cc1 ; COC(=O)c1cc2ncc(-c3ccc(OC(F)(F)F)cc3)n2cc1-c1ccc(OC(F)(F)F)cc1]

To a solution of methyl 6-bromo-3-iodoimidazo[1,2-a]pyridine-7-carboxylate (0.050 g, 0.13 mmol) in 1,4-dioxane (2 mL) were added 4-(trifluoromethoxy)phenylboronic acid (0.054 g, 0.26 mmol), Na₂COM (0.084 g, 0.8 mmol), water (1 mL) followed by catalytic amount of Pd(dppf)Cl₂·DCM (20 mg, 0.024 mmol). The resulting reaction mixture was purged with nitrogen, and then heated at 100° C. for overnight. Reaction was brought to RT, solvent was evaporated under vacuum, and extracted with ethyl acetate. The organic phase was dried over Na₂SO₄, filtered, and evaporated under vacuum to give a crude residue, which was purified on SiO₂ column chromatography using (hexane/EtOAc=1:1) to give 0.053 g of the title compound (Yield: 82%). LC/MS m/Z 497.18 (M+H)⁺

### Step 4: 3,6-bis(4-(trifluoromethoxy)phenyl)imidazo[1,2-a]pyridine-7-carboxylic Acid

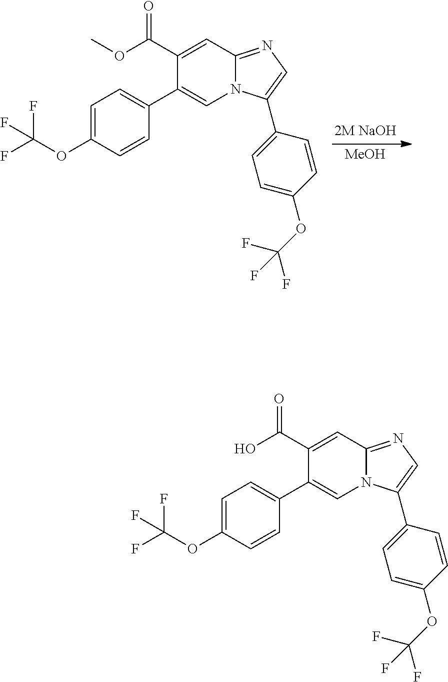 [SMILES: "MeOH" ; COC(=O)c1cc2ncc(-c3ccc(OC(F)(F)F)cc3)n2cc1-c1ccc(OC(F)(F)F)cc1 ; O=C(O)c1cc2ncc(-c3ccc(OC(F)(F)F)cc3)n2cc1-c1ccc(OC(F)(F)F)cc1]

To a solution of methyl 3,6-bis(4-(trifluoromethoxy)phenyl)imidazo[1,2-a]pyridine-7-carboxylate (0.053 g, 0.106 mmol) in MeOH (1 mL) was added 2N NaOH (0.25 mL). The reaction mixture was stirred at r.t. for overnight, then solvent was removed under vacuum. The residue was acidified with 1N HCl to give 0.041 g of the title compound (Yield: 80%). LC/MS m/z: 483.20 (M+H)⁺.

### Step 5: N-(methylsulfonyl)-3,6-bis(4-(trifluoromethoxy)phenyl)imidazo[1,2-a]pyridine-7-carboxamide

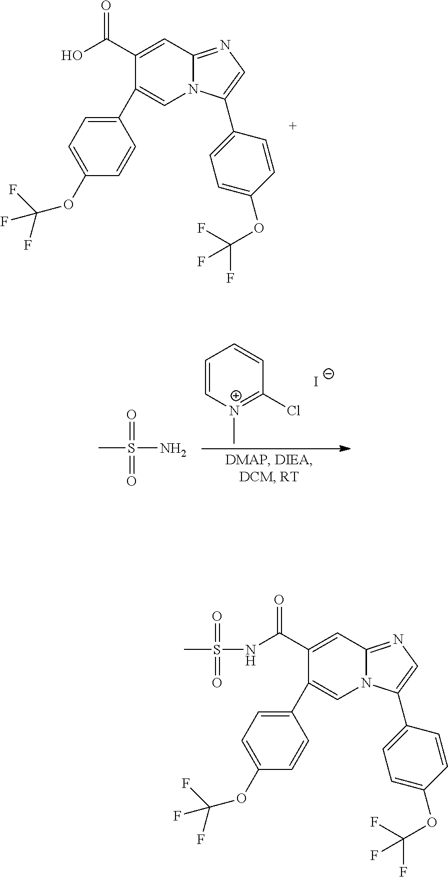

To a solution of 3,6-bis(4-(trifluoromethoxy)phenyl)imidazo[1,2-a]pyridine-7-carboxylic acid (0.040 g, 0.082 mmol) in DCM (1 mL) were added 2-chloro-1-methylpyridinium iodide (0.036 g, 0.141 mmol) followed by DIEA (0.065 mL, 0.37 mmol). Resulting reaction mixture was stirred at RT for 15 min. Then methanesulfonamide (0.035 g, 0.368 mmol) followed by catalytic amount of DMAP (0.005 g) was added, and reaction mixture was stirred for overnight. Then the reaction mixture was evaporated and crude residue was purified using preparative to give 0.018 g of the title compound (Yield: 33%). LC/MS m/z: 560.16 (M+H)⁺

### Example C68: N-(methylsulfonyl)-6-phenyl-3-(4-(trifluoromethoxy)phenyl)imidazo[1,2-a]pyridine-7-carboxamide

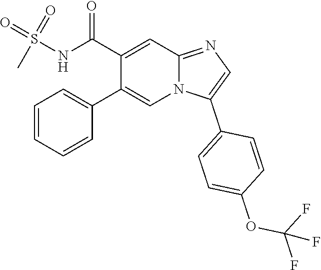 [SMILES: CS(=O)(=O)NC(=O)c1cc2ncc(-c3ccc(OC(F)(F)F)cc3)n2cc1-c1ccccc1]

### Step 1: Methyl 6-phenylimidazo[1,2-a]pyridine-7-carboxylate

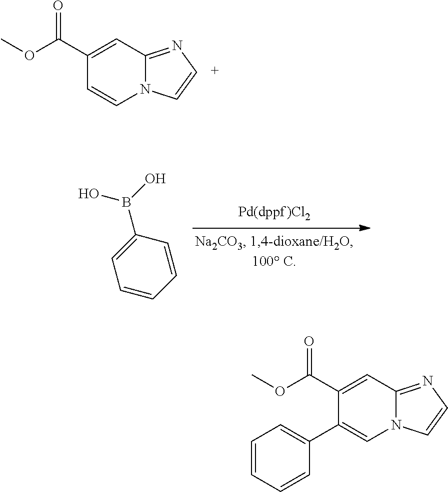 [SMILES: COC(=O)c1ccn2ccnc2c1 ; OB(O)c1ccccc1 ; COC(=O)c1cc2nccn2cc1-c1ccccc1]

To a solution of methyl 6-bromoimidazo[1,2-a]pyridine-7-carboxylate (0.255 g, 1 mmol) in 1,4-dioxane (3 mL) were added phenylboronic acid (0.130 g, 1.06 mmol), Na₂CO₃ (0.318 g, 3 mmol), water (1 mL) followed by catalytic amount of Pd(dppf)Cl₂·DCM (20 mg, 0.024 mmol). The resulting reaction mixture was purged with nitrogen, and then heated at 100° C. for overnight. Reaction was brought to RT, solvent was evaporated under vacuum, and extracted with ethyl acetate. The organic phase was dried over Na₂SO₄, filtered, and evaporated under vacuum to give a crude residue, which was purified on SiO₂ column chromatography using (hexane/EtOAc=1:1) to give 0.215 g of the title compound (Yield: 85%). LC/MS m/z: 253.03 (M+H)⁺

### Step 2: Methyl 3-iodo-6-phenylimidazo[1,2-a]pyridine-7-carboxylate

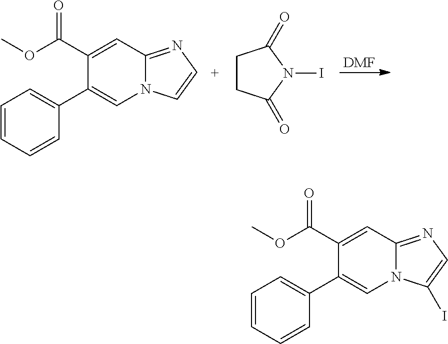

To a solution of methyl 6-phenylimidazo[1,2-a]pyridine-7-carboxylate (0.200 g, 0.79 mmol) in DMF (2 mL), was added N-iodosuccinimide (0.220 g, 0.97 mmol). The resulting reaction mixture was stirred at room temperature overnight. The reaction was poured into water and extracted with ethyl acetate. The organic phase was dried over Na₂SO₄, filtered, and concentrated under vacuum. The residue was purified by SiO₂ column chromatography (hexane/EtOAc=1:1) to give 0.184 g of the title compound (Yield: 61%). LC/MS m/z: 379.14 (M+H)⁺.

### Step 3: Methyl 6-phenyl-3-(4-(trifluoromethoxy)phenyl)imidazo[1,2-a]pyridine-7-carboxylate

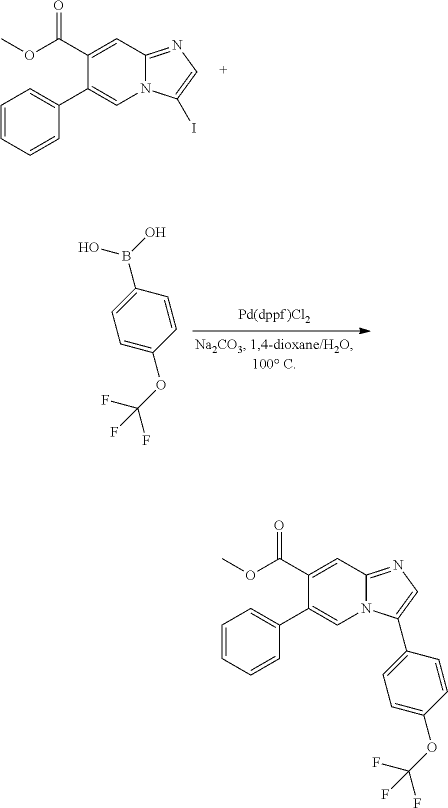 [SMILES: COC(=O)c1cc2ncc(I)n2cc1-c1ccccc1 ; OB(O)c1ccc(OC(F)(F)F)cc1 ; COC(=O)c1cc2ncc(-c3ccc(OC(F)(F)F)cc3)n2cc1-c1ccccc1]

To a solution of methyl 3-iodo-6-phenylimidazo[1,2-a]pyridine-7-carboxylate (0.045 g, 0.12 mmol) in 1,4-dioxane (1 mL) were added 4-(trifluoromethoxy)phenylboronic acid (0.031 g, 0.15 mmol), Na₂COM (0.038 g, 0.36 mmol), water (1 mL) followed by catalytic amount of Pd(dppf)Cl₂·DCM (10 mg, 0.012 mmol). The resulting reaction mixture was purged with nitrogen, and then heated at 100° C. for overnight. Reaction was brought to RT, solvent was evaporated under vacuum, and extracted with ethyl acetate. The organic phase was dried over Na₂SO₄, filtered, and evaporated under vacuum to give a crude residue, which was purified on SiO₂ column chromatography using (hexane/EtOAc=1:1) to give 0.045 g of the title compound (Yield: 91%). LC/MS m/z: 413.24 (M+H)⁺

### Step 4: 6-phenyl-3-(4-(trifluoromethoxy)phenyl)imidazo[1,2-a]pyridine-7-carboxylic Acid

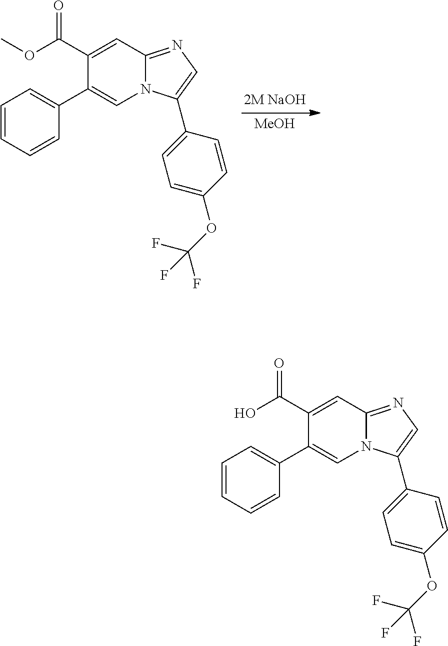 [SMILES: "MeOH" ; COC(=O)c1cc2ncc(-c3ccc(OC(F)(F)F)cc3)n2cc1-c1ccccc1 ; O=C(O)c1cc2ncc(-c3ccc(OC(F)(F)F)cc3)n2cc1-c1ccccc1]

To a solution of methyl 6-phenyl-3-(4-(trifluoromethoxy)phenyl)imidazo[1,2-a]pyridine-7-carboxylate (0.045 g, 0.109 mmol) in MeOH (1 mL) was added 2N NaOH (0.25 mL). The reaction mixture was stirred at r.t. for overnight, then solvent was removed under vacuum. The residue was acidified with 1N HCl to give 0.020 g of the title compound (Yield: 80%). LC/MS m/Z 399.21 (M+H)⁺.

### Step 5: N-(methylsulfonyl)-6-phenyl-3-(4-(trifluoromethoxy)phenyl)imidazo[1,2-a]pyridine-7-carboxamide

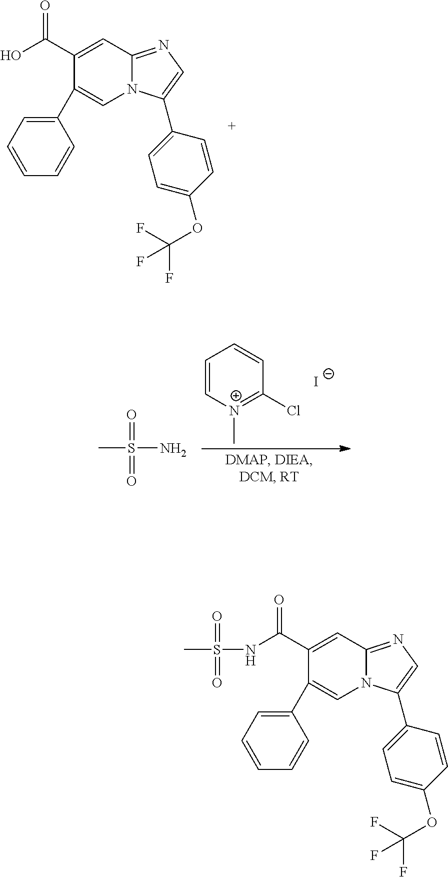

To a solution of 6-phenyl-3-(4-(trifluoromethoxy)phenyl)imidazo[1,2-a]pyridine-7-carboxylic acid (0.020 g, 0.05 mmol) in DCM (1 mL) were added 2-chloro-1-methylpyridinium iodide (0.020 g, 0.075 mmol) followed by DIEA (0.027 mL, 0.15 mmol). Resulting reaction mixture was stirred at RT for 15 min. Then methanesulfonamide (0.015 g, 0.15 mmol) followed by catalytic amount of DMAP (0.005 g) was added, and reaction mixture was stirred for overnight. Then the reaction mixture was evaporated and crude residue was purified using preparative to give 0.0037 g of the title compound (Yield: 12%). LC/MS m/z: 476.06 (M+H)⁺

### Example C69: 3-(4-isopropoxyphenyl)-N-(methylsulfonyl)-6-phenylimidazo[1,2-a]pyridine-7-carboxamide

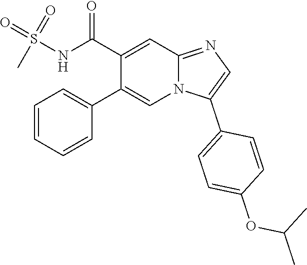 [SMILES: CC(C)Oc1ccc(-c2cnc3cc(C(=O)NS(C)(=O)=O)c(-c4ccccc4)cn23)cc1]

The title compound was prepared from Methyl 6-phenyl-3-(4-(trifluoromethoxy)phenyl)imidazo[1,2-a]pyridine-7-carboxylate and 4-isopropoxyphenylboronic acid in the same manner as described above for N-(methylsulfonyl)-6-phenyl-3-(4-(trifluoromethoxy)phenyl)imidazo[1,2-a]pyridine-7-carboxamide using steps 3 to 5. LC/MS m/z: 450.21 (M+H)⁺.

### Example C70: 6-(4-isopropoxyphenyl)-3-(6-isopropoxypyridin-3-yl)-7-methylimidazo[1,2-a]pyridine

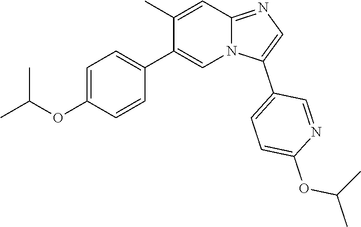 [SMILES: Cc1cc2ncc(-c3ccc(OC(C)C)nc3)n2cc1-c1ccc(OC(C)C)cc1]

### Step 1: 6-(4-isopropoxyphenyl)-7-methylimidazo[1,2-a]pyridine

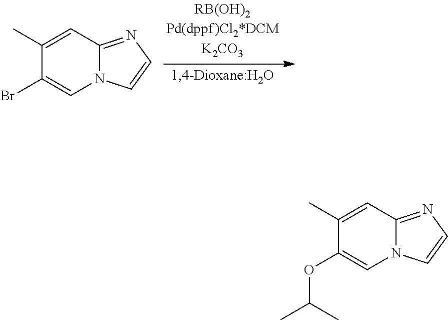

To a solution of 6-bromo-7-methylimidazo[1,2-a]pyridine (0.3 g, 1.42 mmol) in dioxane (4 mL) and water (1 mL) is added potassium carbonate (0.584 g, 4.26 mmol), 4-isoproproxyphenylboronic acid (0.3 g, 1.71 mmol), and Pd(dppf)Cl₂ dichloromethane complex (0.114 g, 0.14 mmol). The vial is capped tightly and degassed by bubbling nitrogen for 5 minutes, then heated on a microwave reactor at 100° C. for 1 hour. The mixture is diluted with ethyl acetate, washed with water and brine, and the organics dried over sodium sulfate. The residue is purified by flash chromatography on silica using ethyl acetate as eluent to give 0.251 g of the title compound as a brown oil. LC/MS m/z: 267.17 (M+H)⁺

### Step 2: 3-iodo-6-(4-isopropoxyphenyl)-7-methylimidazo[1,2-a]pyridine

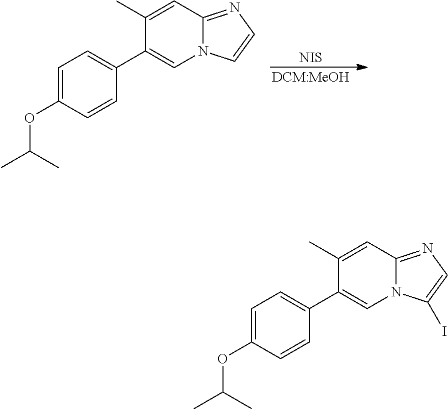

To a solution of 6-(4-isopropoxyphenyl)-7-methylimidazo[1,2-a]pyridine (0.251 g, 0.94 mmol) in DCM (10 mL) and MeOH (1 mL), was added N-iodosuccinimide (0.254 g, 1.13 mmol). The resulting reaction mixture was stirred at room temperature for overnight. The reaction was poured into water and extracted with ethyl acetate. The organic phase was dried over Na₂SO₄, filtered, and concentrated under vacuum. The residue was purified by SiO₂ column chromatography to give 87 mg of the title compound. LC/MS m/z: 393.17 (M+H)⁺

### Step 3: 6-(4-isopropoxyphenyl)-3-(6-isopropoxypyridin-3-yl)-7-methylimidazo[1,2-a]pyridine

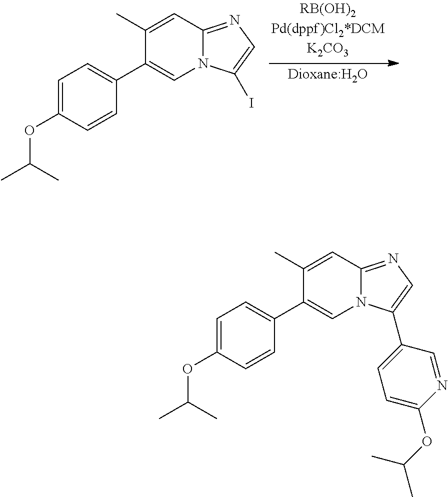 [SMILES: Cc1cc2ncc(I)n2cc1-c1ccc(OC(C)C)cc1 ; Cc1cc2ncc(-c3ccc(OC(C)C)nc3)n2cc1-c1ccc(OC(C)C)cc1]

To a solution of 3-iodo-6-(4-isopropoxyphenyl)-7-methylimidazo[1,2-a]pyridine (30 mg, 0.077 mmol) in dioxane (1.5 mL) and water (0.5 mL) is added potassium carbonate (32 mg, 0.231 mmol), (6-isopropoxypyridin-3-yl)boronic acid (17 mg, 0.092 mmol), and Pd(dppf)Cl₂ dichloromethane complex (9 mg, 0.0077 mmol). The vial is capped tightly and degassed by bubbling nitrogen for 5 minutes, then heated on a microwave reactor at 100° C. for 1 hour. The mixture is diluted with ethyl acetate, washed with water and brine, and the organics dried over sodium sulfate. The residue is purified by flash chromatography on silica using 3:7 hexanes:ethyl acetate as eluent to give 3.1 mg of the title compound as a clear oil. LC/MS m/z: 402.39 (M+H)⁺

### Example C71: 3-(6-isopropoxy-5-methylpyridin-3-yl)-6-(4-isopropoxyphenyl)imidazo[1,2-a]pyridine

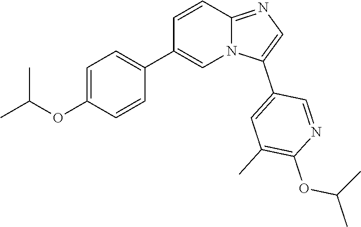 [SMILES: Cc1cc(-c2cnc3ccc(-c4ccc(OC(C)C)cc4)cn23)cnc1OC(C)C]

### Step 1: 6-(4-isopropoxyphenyl)imidazo[1,2-a]pyridine

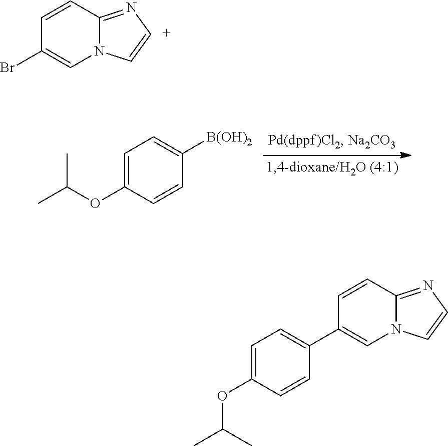 [SMILES: BrC1=CN2C=[C+]N=C2C=C1 ; CC(C)Oc1ccc(B(O)O)cc1 ; CC(C)Oc1ccc(-c2ccc3nccn3c2)cc1]

To a solution of 6-bromoimidazo[1,2-a]pyridine (1.97 g, 10 mmol) in 30 mL 1,4-dioxane:water (4:1) was added 4-isopropoxyphenylboronic acid (2.16 g, 12 mmol), sodium carbonate (3.180 g, 30 mmol), and Pd(dppf)Cl₂ dichloromethane complex (82 mg, 0.1 mmol). The reaction mixture was heated to 100° C. under N₂ atmosphere overnight. Cooled to RT, reaction mixture was then diluted with ethyl acetate, washed with water (2×) and brine (1×), dried over sodium sulfate, and evaporated. The crude residue was purified by flash chromatography on a silica gel column to provide 1.8 g of the title compound (Yield: 71%). LC/MS m/z: 253.26 (M+H)⁺

### Step 2: 3-iodo-6-(4-isopropoxyphenyl)imidazo[1,2-a]pyridine

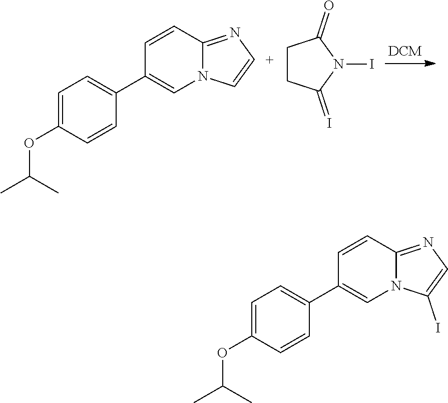

To a solution of 6-(4-isopropoxyphenyl)imidazo[1,2-a]pyridine (1.8 g, 7.14 mmol) in DCM (30 mL) and MeOH (0.4 mL), was added N-iodosuccinimide (1.928 g, 8.57 mmol). The resulting reaction mixture was stirred at room temperature overnight. The reaction mixture was poured into water and extracted with ethyl acetate. The organic phase was dried over Na₂SO₄, filtered, and concentrated under vacuum. The residue was purified by SiO₂ column chromatography to give 2.365 g of the title compound (Yield: 87%). LC/MS m/z: 379.18 (M+H)⁺.

### Step 3: 3-(6-isopropoxy-5-methylpyridin-3-yl)-6-(4-isopropoxyphenyl)imidazo[1,2-a]pyridine

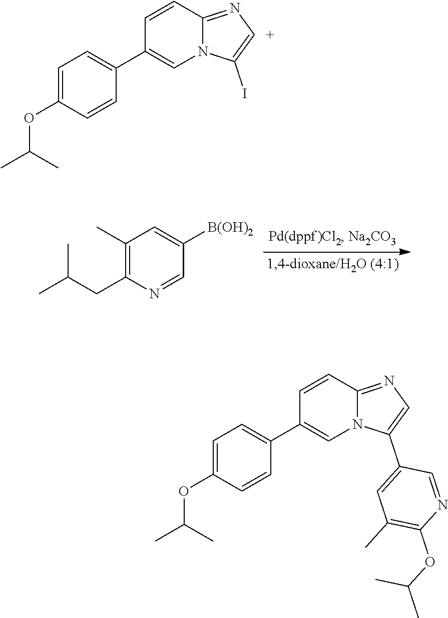 [SMILES: CC(C)Oc1ccc(C2=CN3C(I)=[C+]N=C3C=C2)cc1 ; Cc1cc(B(O)O)cnc1CC(C)C ; Cc1cc(-c2cnc3ccc(-c4ccc(OC(C)C)cc4)cn23)cnc1OC(C)C]

To a solution of 3-iodo-6-(4-isopropoxyphenyl)imidazo[1,2-a]pyridine (0.100 g, 0.264 mmol) in 4 mL 1,4-dioxane:water (4:1) was added (6-isopropoxy-5-methylpyridin-3-yl)boronic acid (0.056 g, 0.29 mmol), sodium carbonate (0.084 g, 0.792 mmol), and Pd(dppf)Cl₂ dichloromethane complex (8 mg, 0.01 mmol). The reaction mixture was heated to 100° C. under N₂ atmosphere for overnight. Cooled to RT, the reaction mixture was then diluted with ethyl acetate, washed with water (2×) and brine (1×), dried over sodium sulfate, and evaporated. The crude residue was purified by flash chromatography on a silica gel column to provide 0.085 g of the title compound (Yield: 81%). LC/MS m/z: 402.36 (M+H)⁺

### Example C72: 6-(4-isopropoxyphenyl)-3-(4-phenoxyphenyl)imidazo[1,2-a]pyridine

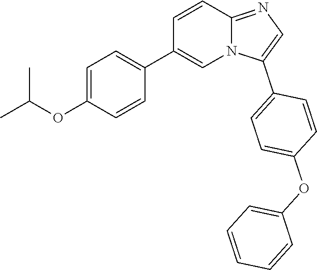 [SMILES: CC(C)Oc1ccc(-c2ccc3ncc(-c4ccc(Oc5ccccc5)cc4)n3c2)cc1]

The title compound was prepared from 3-iodo-6-(4-isopropoxyphenyl)imidazo[1,2-a]pyridine in the same manner as described above for 3-(6-isopropoxy-5-methylpyridin-3-yl)-6-(4-isopropoxyphenyl)imidazo[1,2-a]pyridine (example C71). LC/MS m/z: 420.50 (M+H)⁺

### Example C73: 6-(4-isopropoxyphenyl)-3-(4-isopropylphenyl)imidazo[1,2-a]pyridine

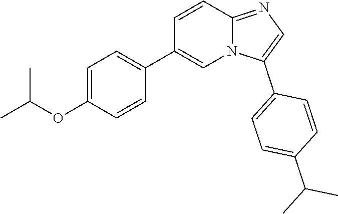 [SMILES: CC(C)Oc1ccc(-c2ccc3ncc(-c4ccc(C(C)C)cc4)n3c2)cc1]

The title compound was prepared from 3-iodo-6-(4-isopropoxyphenyl)imidazo[1,2-a]pyridine in the same manner as described above for 3-(6-isopropoxy-5-methylpyridin-3-yl)-6-(4-isopropoxyphenyl)imidazo[1,2-a]pyridine (example C71). LC/MS m/z: 371.40 (M+H)⁺

### Example C74: 3-(2-fluoro-6-isopropoxypyridin-3-yl)-6-(4-isopropoxyphenyl)imidazo[1,2-a]pyridine

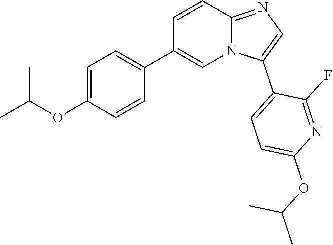 [SMILES: CC(C)Oc1ccc(-c2ccc3ncc(-c4ccc(OC(C)C)nc4F)n3c2)cc1]

The title compound was prepared from 3-iodo-6-(4-isopropoxyphenyl)imidazo[1,2-a]pyridine in the same manner as described above for 3-(6-isopropoxy-5-methylpyridin-3-yl)-6-(4-isopropoxyphenyl)imidazo[1,2-a]pyridine (example C71). LC/MS m/z: 406.34 (M+H)⁺

### Example C75: 6-(4-isopropoxyphenyl)-3-(2-isopropoxypyrimidin-5-yl)imidazo[1,2-a]pyridine

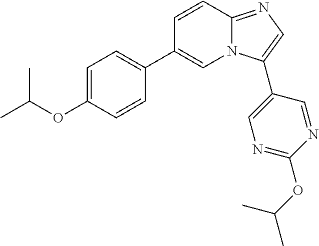 [SMILES: CC(C)Oc1ccc(-c2ccc3ncc(-c4cnc(OC(C)C)nc4)n3c2)cc1]

The title compound was prepared from 3-iodo-6-(4-isopropoxyphenyl)imidazo[1,2-a]pyridine in the same manner as described above for 3-(6-isopropoxy-5-methylpyridin-3-yl)-6-(4-isopropoxyphenyl)imidazo[1,2-a]pyridine (example C71). LC/MS m/z: 389.35 (M+H)⁺

### Example C76: 6-(4-isopropoxyphenyl)-3-(2-isopropoxypyridin-4-yl)imidazo[1,2-a]pyridine

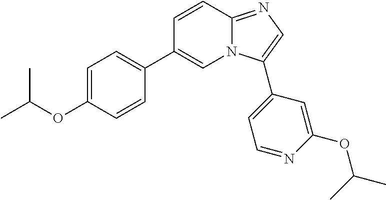 [SMILES: CC(C)Oc1ccc(-c2ccc3ncc(-c4ccnc(OC(C)C)c4)n3c2)cc1]

The title compound was prepared from 3-iodo-6-(4-isopropoxyphenyl)imidazo[1,2-a]pyridine in the same manner as described above for 3-(6-isopropoxy-5-methylpyridin-3-yl)-6-(4-isopropoxyphenyl)imidazo[1,2-a]pyridine (example C71). LC/MS m/z: 388.32 (M+H)⁺

### Example C77: 6-(4-isopropoxyphenyl)-3-(5-isopropoxypyridin-3-yl)imidazo[1,2-a]pyridine

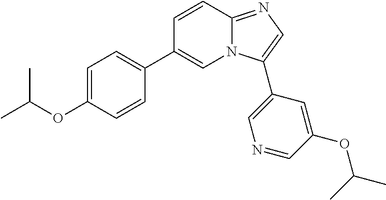 [SMILES: CC(C)Oc1ccc(-c2ccc3ncc(-c4cncc(OC(C)C)c4)n3c2)cc1]

The title compound was prepared from 3-iodo-6-(4-isopropoxyphenyl)imidazo[1,2-a]pyridine in the same manner as described above for 3-(6-isopropoxy-5-methylpyridin-3-yl)-6-(4-isopropoxyphenyl)imidazo[1,2-a]pyridine (example C71). LC/MS m/z: 388.33 (M+H)⁺

### Example C78: 3-(4-(tert-butoxy)phenyl)-6-(6-isopropoxypyridin-3-yl)-7-methylimidazo[1,2-a]pyridine

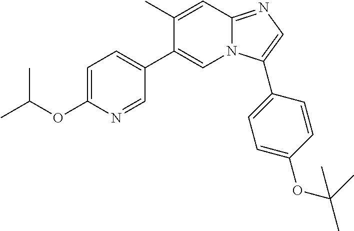 [SMILES: Cc1cc2ncc(-c3ccc(OC(C)(C)C)cc3)n2cc1-c1ccc(OC(C)C)nc1]

### Step 1: 6-(6-isopropoxypyridin-3-yl)-7-methylimidazo[1,2-a]pyridine

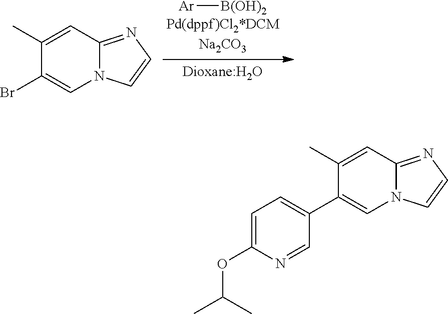

To a solution of 6-bromo-7-methylimidazo[1,2-a] pyridine (0.422 g, 2 mmol) in 1,4-dioxane (8 mL) and water (4 mL) is added sodium carbonate (0.636 g, 6 mmol), (6-isopropoxypyridin-3-yl) boronic acid (0.440 g, 2.4 mmol), and Pd(dppf)Cl₂ dichloromethane complex (0.076 g, 0.09 mmol). The reaction mixture was degassed, then heated at 100° C. for 6 hours. The mixture is diluted with ethyl acetate, washed with water and brine, and the organics dried over sodium sulfate. The residue is purified by silica gel flash chromatography using ethyl acetate as eluent to give 0.462 g (yield: 86%) of the title compound. LC/MS m/z: 268.24 (M+H)⁺

### Step 2: 3-iodo-6-(6-isopropoxypyridin-3-yl)-7-methylimidazo[1,2-a] pyridine

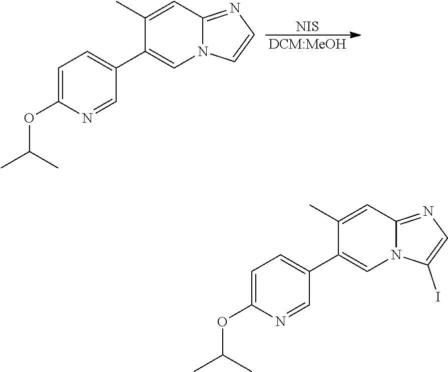

To a solution of 6-(6-isopropoxypyridin-3-yl)-7-methylimidazo[1,2-a] pyridine (0.450 g, 1.685 mmol) in DCM (8 mL) and MeOH (1 mL), was added N-iodosuccinimide (0.455 g, 2.02 mmol). The resulting reaction mixture was stirred at room temperature for overnight. The reaction was poured into water and extracted with ethyl acetate. The organic phase was dried over Na₂SO₄, filtered, and concentrated under vacuum. The residue was purified by SiO₂ column chromatography to give 0.285 g (yield: 43%) of the title compound. LC/MS m/z: 393.27 (M+H)⁺

### Step 3: 3-(4-(tert-butoxy)phenyl)-6-(6-isopropoxypyridin-3-yl)-7-methylimidazo[1,2-a] pyridine

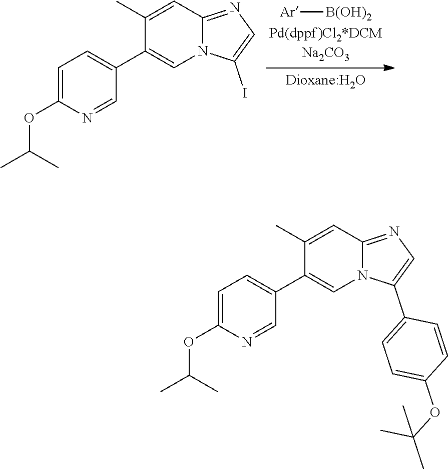

To a solution of 3-iodo-6-(6-isopropoxypyridin-3-yl)-7-methylimidazo[1,2-a] pyridine (0.100 g, 0.254 mmol) in 1,4-dioxane (4 mL) and water (2 mL) was added sodium carbonate (0.081 g, 0.76 mmol), 4-(tert-butoxy) phenyl boronic acid (0.059 g, 0.305 mmol), and Pd(dppf)Cl₂ dichloromethane complex (10 mg, 0.0077 mmol). The vial is capped tightly and degassed by bubbling nitrogen for 5 minutes, then heated in a microwave reactor at 100° C. for 1 hour. The mixture is diluted with ethyl acetate, washed with water and brine, and the organics dried over sodium sulfate. The residue is purified by SiO₂ flash chromatography using 3:7 hexanes:ethyl acetate as eluent to give the title compound. LC/MS m/z: 416.37 (M+H)⁺

### Example C79: 3-(4-(tert-butoxy)phenyl)-6-(3-fluoro-4-isopropoxyphenyl)-7-methylimidazo[1,2-a]pyridine

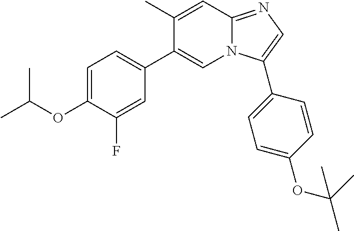 [SMILES: Cc1cc2ncc(-c3ccc(OC(C)(C)C)cc3)n2cc1-c1ccc(OC(C)C)c(F)c1]

### Step 1: 6-(3-fluoro-4-isopropoxyphenyl)-7-methylimidazo[1,2-a]pyridine

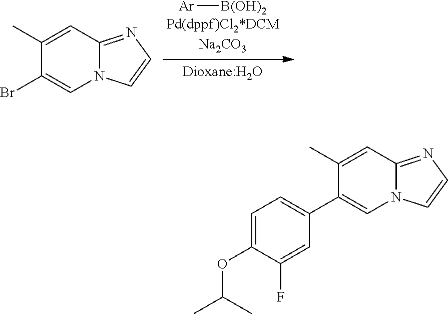

To a solution of 6-bromo-7-methylimidazo[1,2-a] pyridine (0.633 g, 3 mmol) in 1,4-dioxane (12 mL) and water (6 mL) is added sodium carbonate (0.954 g, 9 mmol), 3-fluoro-4-isoproproxyphenylboronic acid (0.653 g, 3.3 mmol), and Pd(dppf)Cl₂ dichloromethane complex (0.114 g, 0.14 mmol). The reaction mixture was degassed, then heated at 100° C. for 6 hours. The mixture is diluted with ethyl acetate, washed with water and brine, and the organics dried over sodium sulfate. The residue is purified by silica gel flash chromatography using ethyl acetate as eluent to give 0.470 g (yield: 55%) of the title compound. LC/MS m/z: 285.32 (M+H)⁺

### Step 2: 6-(3-fluoro-4-isopropoxyphenyl)-3-iodo-7-methylimidazo[1,2-a] pyridine

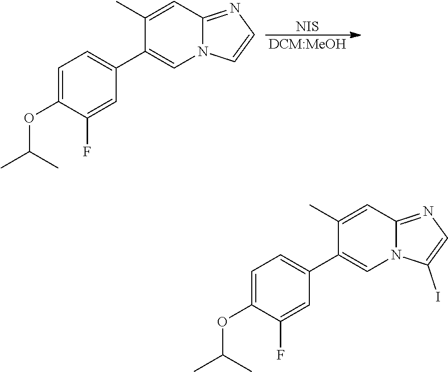

To a solution of 6-(3-fluoro-4-isopropoxyphenyl)-7-methylimidazo[1,2-a] pyridine (0.460 g, 1.61 mmol) in DCM (8 mL) and MeOH (1 mL), was added N-iodosuccinimide (0.437 g, 1.94 mmol). The resulting reaction mixture was stirred at room temperature for overnight. The reaction was poured into water and extracted with ethyl acetate. The organic phase was dried over Na₂SO₄, filtered, and concentrated under vacuum. The residue was purified by SiO₂ column chromatography to give 0.320 g (yield: 48%) of the title compound. LC/MS m/z: 411.14 (M+H)⁺

### Step 3: 3-(4-(tert-butoxy) phenyl)-6-(3-fluoro-4-isopropoxyphenyl)-7-methylimidazo[1,2-a] pyridine

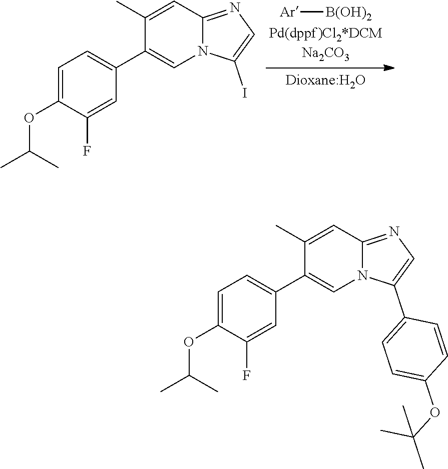

To a solution of 6-(3-fluoro-4-isopropoxyphenyl)-3-iodo-7-methylimidazo[1,2-a] pyridine (0.100 g, 0.24 mmol) in 1,4-dioxane (4 mL) and water (2 mL) was added sodium carbonate (0.081 g, 0.76 mmol), 4-(tert-butoxy) phenyl boronic acid (0.059 g, 0.305 mmol), and Pd(dppf)Cl₂ dichloromethane complex (10 mg, 0.0077 mmol). The vial is capped tightly and degassed by bubbling nitrogen for 5 minutes, then heated on a microwave reactor at 100° C. for 1 hour. The mixture is diluted with ethyl acetate, washed with water and brine, and the organics dried over sodium sulfate. The residue is purified by SiO₂ flash chromatography using 3:7 hexanes:ethyl acetate as eluent to give 0.032 g of the title compound. LC/MS m/z: 433.34 (M+H)⁺

### Example C80: 6-(3-fluoro-4-isopropoxyphenyl)-3-(4-isopropoxyphenyl)-7-methylimidazo[1,2-a]pyridine

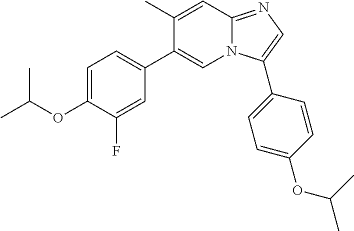 [SMILES: Cc1cc2ncc(-c3ccc(OC(C)C)cc3)n2cc1-c1ccc(OC(C)C)c(F)c1]

The title compound was prepared from 6-(3-fluoro-4-isopropoxyphenyl)-3-iodo-7-methylimidazo[1,2-a]pyridine in the same manner as described above for 3-(4-(tert-butoxy) phenyl)-6-(3-fluoro-4-isopropoxyphenyl)-7-methylimidazo[1,2-a] pyridine (example C79). LC/MS m/z: 419.53 (M+H)⁺

### Example C81: 3-(4-(tert-butoxy)phenyl)-6-(3-fluoro-4-isopropoxyphenyl)imidazo[1,2-a] pyridine

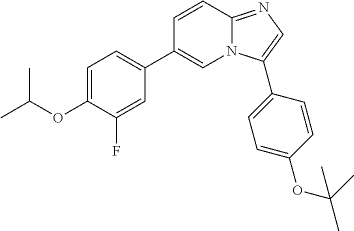 [SMILES: CC(C)Oc1ccc(-c2ccc3ncc(-c4ccc(OC(C)(C)C)cc4)n3c2)cc1F]

### Step 1: 6-bromo-3-(4-(tert-butoxy)phenyl)imidazo[1,2-a] pyridine

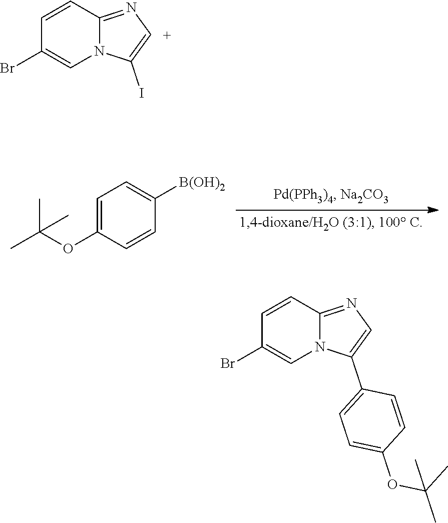 [SMILES: BrC1=CN2C(I)=[C+]N=C2C=C1 ; CC(C)(C)Oc1ccc(B(O)O)cc1 ; CC(C)(C)Oc1ccc(-c2cnc3ccc(Br)cn23)cc1]

To a solution of 6-bromo-3-iodoimidazo[1,2-a] pyridine (0.032 g, 0.1 mmol) in dioxane (2 mL) was added (4-tert-butoxyphenyl) boronic acid (0.019 g, 0.1 mmol), tetrakis(triphenylphosphine)palladium (0.007 g, 0.0065 mmol), sodium carbonate (0.030 g, 0.28 mmol) and water (1 mL). The resulting reaction mixture was degassed with nitrogen for 10 min, then heated to 100° C. for 5 h. Then the reaction mixture was diluted with ethyl acetate and washed with water. The organic phase was dried over Na₂SO₄, filtered, and concentrated in vacuo. The residue was purified by SiO₂ column chromatography (hexanes/EtOAc from 4:1 to 12) to give 0.025 g (yield: 72%) of the title compound. LC/MS m/z: 345.16 (M+H)⁺

### Step 2: 3-(4-(tert-butoxy)phenyl)-6-(3-fluoro-4-isopropoxyphenyl)imidazo[1,2-a]pyridine

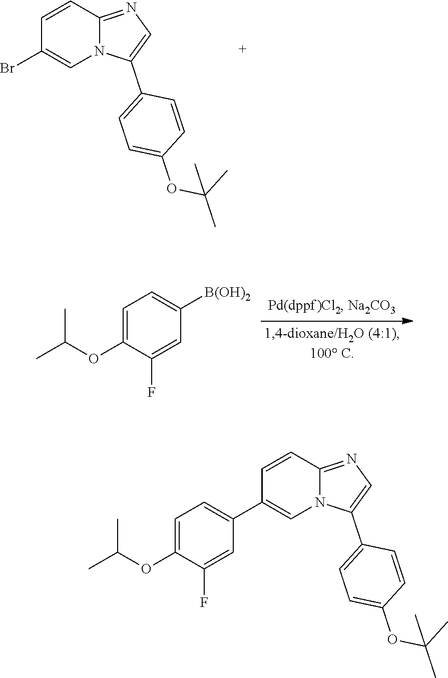 [SMILES: CC(C)(C)Oc1ccc(-c2cnc3ccc(Br)cn23)cc1 ; "+" ; CC(C)Oc1ccc(B(O)O)cc1F ; CC(C)Oc1ccc(-c2ccc3ncc(-c4ccc(OC(C)(C)C)cc4)n3c2)cc1F]

To a solution of 6-bromo-3-(4-(tert-butoxy)phenyl)imidazo[1,2-a] pyridine (0.025 g, 0.072 mmol) in 1,4-dioxane (1 mL) was added 3-fluoro-4-isoproproxyphenylboronic acid (0.019 g, 0.1 mmol), [1,1′-bis(diphenylphosphino)ferrocene]palladium(II) dichloride (4.0 mg, 0.006 mmol), sodium carbonate (0.031 g, 0.3 mmol) and water (0.5 mL). The resulting reaction mixture was degassed with nitrogen for 10 min, then heated to 100° C. for 6 h. Then the reaction mixture was diluted with ethyl acetate and washed with water. The organic phase was dried over Na₂SO₄, filtered, and concentrated in vacuo. The residue was purified by SiO₂ column chromatography (hexanes/EtOAc from 1:1 to 1:10, then pure EtOAc) to give the title compound. LC/MS m/z: 419.40 (M+H)⁺

### Example C82: 3,6-bis(4-(tert-butoxy)phenyl)imidazo[1,2-a]pyridine

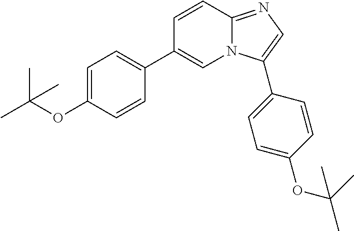 [SMILES: CC(C)(C)Oc1ccc(-c2ccc3ncc(-c4ccc(OC(C)(C)C)cc4)n3c2)cc1]

The title compound was prepared from 6-bromo-3-iodoimidazo[1,2-a]pyridine in the same manner as described above for 3-(6-isopropoxy-5-methylpyridin-3-yl)-6-(4-isopropoxyphenyl)imidazo[1,2-a]pyridine (example C71) using 2 equivalents of boronic acid. LC/MS m/z: 415.32 [M+H]⁺

### Example C83: 3-(4-(tert-butoxy)phenyl)-6-(6-isopropoxypyridin-3-yl)imidazo[1,2-a]pyridine

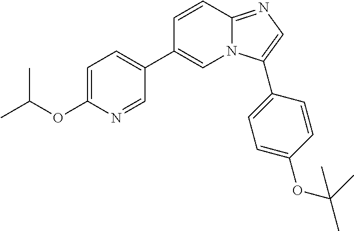 [SMILES: CC(C)Oc1ccc(-c2ccc3ncc(-c4ccc(OC(C)(C)C)cc4)n3c2)cn1]

The title compound was prepared from 6-bromo-3-(4-(tert-butoxy)phenyl)imidazo[1,2-a]pyridine in the same manner as described above for 3-(6-isopropoxy-5-methylpyridin-3-yl)-6-(4-isopropoxyphenyl)imidazo[1,2-a]pyridine (example C71). LC/MS m/z: 402.36 [M+H]⁺

### Example C84: 3-(6-(tert-butoxy)pyridin-3-yl)-6-(4-isopropoxyphenyl)-7-methylimidazo[1,2-a]pyridine

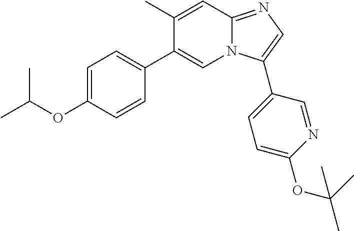 [SMILES: Cc1cc2ncc(-c3ccc(OC(C)(C)C)nc3)n2cc1-c1ccc(OC(C)C)cc1]

The title compound was prepared from 3-iodo-6-(4-isopropoxyphenyl)-7-methylimidazo[1,2-a]pyridine in the same manner as described above for 3-(6-isopropoxy-5-methylpyridin-3-yl)-6-(4-isopropoxyphenyl)imidazo[1,2-a]pyridine (example C71). LC/MS m/z: 416.37 [M+H]⁺

### Example C85: 3-(6-(tert-butoxy)pyridin-3-yl)-6-(3-fluoro-4-isopropoxyphenyl)-7-methylimidazo[1,2-a]pyridine

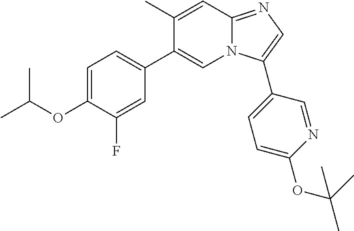 [SMILES: Cc1cc2ncc(-c3ccc(OC(C)(C)C)nc3)n2cc1-c1ccc(OC(C)C)c(F)c1]

The title compound was prepared from 6-(3-fluoro-4-isopropoxyphenyl)-3-iodo-7-methylimidazo[1,2-a]pyridine in the same manner as described above for 3-(4-(tert-butoxy) phenyl)-6-(3-fluoro-4-isopropoxyphenyl)-7-methylimidazo[1,2-a] pyridine (example C79). LC/MS m/z: 434.33 (M+H)⁺

### Example C86: 3-(4-(tert-butoxy)phenyl)-6-(5-fluoro-6-(2,2,2-trifluoroethoxy)pyridin-3-yl)imidazo[1,2-a]pyridine

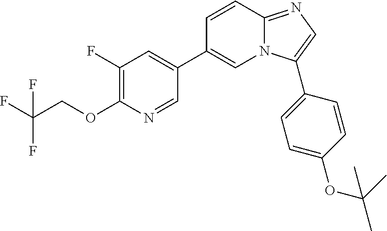 [SMILES: CC(C)(C)Oc1ccc(-c2cnc3ccc(-c4cnc(OCC(F)(F)F)c(F)c4)cn23)cc1]

The title compound was prepared from 6-bromo-3-(4-(tert-butoxy)phenyl)imidazo[1,2-a] pyridine in the same manner as described above for 3-(4-(tert-butoxy)phenyl)-6-(3-fluoro-4-isopropoxyphenyl)imidazo[1,2-a]pyridine (example C81). LC/MS m/z: 460.24 (M+H)⁺

### Example C87: 3-(4-(tert-butyl)phenyl)-6-(4-isopropoxyphenyl)imidazo[1,2-a]pyridine

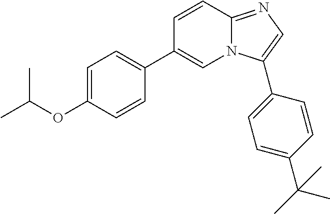 [SMILES: CC(C)Oc1ccc(-c2ccc3ncc(-c4ccc(C(C)(C)C)cc4)n3c2)cc1]

The title compound was prepared from 3-iodo-6-(4-isopropoxyphenyl)imidazo[1,2-a]pyridine in the same manner as described above for 3-(6-isopropoxy-5-methylpyridin-3-yl)-6-(4-isopropoxyphenyl)imidazo[1,2-a]pyridine (example C71). LC/MS m/z: 385.38 (M+H)⁺

### Example C88: 6-(4-isopropoxyphenyl)-3-(6-phenoxypyridin-3-yl)imidazo[1,2-a]pyridine

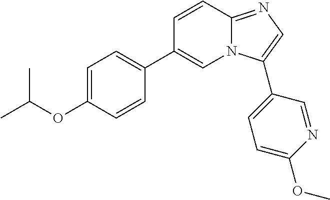 [SMILES: COc1ccc(-c2cnc3ccc(-c4ccc(OC(C)C)cc4)cn23)cn1]

The title compound was prepared from 3-iodo-6-(4-isopropoxyphenyl)imidazo[1,2-a]pyridine in the same manner as described above for 3-(6-isopropoxy-5-methylpyridin-3-yl)-6-(4-isopropoxyphenyl)imidazo[1,2-a]pyridine (example C71). LC/MS m/z: 422.29 (M+H)⁺

### Example C89: 3-(6-(tert-butyl)pyridin-3-yl)-6-(4-isopropoxyphenyl)imidazo[1,2-a]pyridine

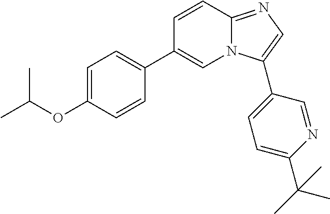 [SMILES: CC(C)Oc1ccc(-c2ccc3ncc(-c4ccc(C(C)(C)C)nc4)n3c2)cc1]

The title compound was prepared from 3-iodo-6-(4-isopropoxyphenyl)imidazo[1,2-a]pyridine in the same manner as described above for 3-(6-isopropoxy-5-methylpyridin-3-yl)-6-(4-isopropoxyphenyl)imidazo[1,2-a]pyridine (example C71). LC/MS m/z: 386.49 (M+H)⁺

### Example C90: 3-(6-(tert-butoxy)pyridin-3-yl)-6-(4-isopropoxyphenyl)imidazo[1,2-a]pyridine

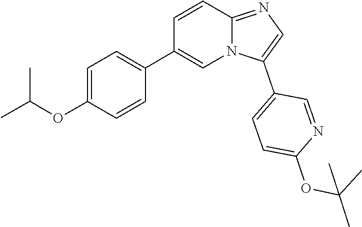 [SMILES: CC(C)Oc1ccc(-c2ccc3ncc(-c4ccc(OC(C)(C)C)nc4)n3c2)cc1]

The title compound was prepared from 3-iodo-6-(4-isopropoxyphenyl)imidazo[1,2-a]pyridine in the same manner as described above for 3-(6-isopropoxy-5-methylpyridin-3-yl)-6-(4-isopropoxyphenyl)imidazo[1,2-a]pyridine (example C71). LC/MS f/r 402.37 (M+H)⁺

### Example C91: 3-(4-(tert-butoxy)phenyl)-7-chloro-6-(4-isopropoxyphenyl)imidazo[1,2-a]pyridine

### Step 1: 7-chloro-6-(4-isopropoxyphenyl)imidazo[1,2-a]pyridine

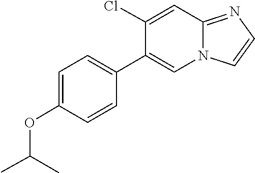 [SMILES: CC(C)Oc1ccc(-c2cn3ccnc3cc2Cl)cc1]

The title compound was prepared from 6-bromo-7-chloroimidazo[1,2-a]pyridine and 4-isopropoxyphenylboronic acid in the same manner as described above for 6-(6-isopropoxypyridin-3-yl)-7-methylimidazo[1,2-a] pyridine (Example C78, step 1).

### Step 2: 7-chloro-3-iodo-6-(4-isopropoxyphenyl)imidazo[1,2-a]pyridine

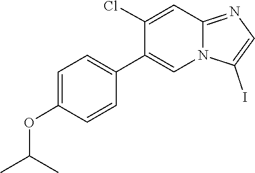 [SMILES: CC(C)Oc1ccc(-c2cn3c(I)cnc3cc2Cl)cc1]

The title compound was prepared from 7-chloro-6-(4-isopropoxyphenyl)imidazo[1,2-a]pyridine in the same manner as described above for 3-iodo-6-(6-isopropoxypyridin-3-yl)-7-methylimidazo[1,2-a]pyridine (Example C78, step 2).

### Step 3: 3-(4-(tert-butoxy)phenyl)-7-chloro-6-(4-isopropoxyphenyl)imidazo[1,2-a]pyridine

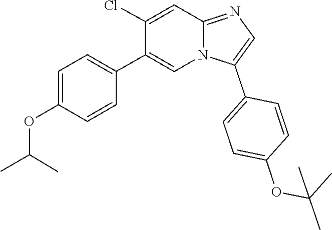 [SMILES: CC(C)Oc1ccc(-c2cn3c(-c4ccc(OC(C)(C)C)cc4)cnc3cc2Cl)cc1]

The title compound was prepared from 7-chloro-3-iodo-6-(4-isopropoxyphenyl)imidazo[1,2-a]pyridine and 4-tert-butoxyphenylboronic acid in the same manner as described above for 3-(4-(tert-butoxy)phenyl)-6-(6-isopropoxypyridin-3-yl)-7-methylimidazo[1,2-a] pyridine (Example C78, step 3). LC/MS m/z: 435.2 (M+H)⁺

### Example D10: 3,6-bis[4-(propan-2-yloxy)phenyl]imidazo[1,2-b]pyridazine

### Step 1: 6-chloro-3-[4-(propan-2-yloxy)phenyl]imidazo[1,2-b]pyridazine

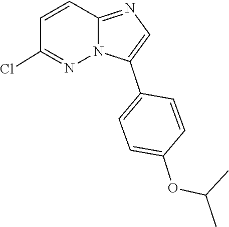 [SMILES: CC(C)Oc1ccc(-c2cnc3ccc(Cl)nn23)cc1]

The title compound was prepared from 6-chloro-3-iodoimidazo[1,2-b]pyridazine and (4-isopropoxyphenyl)boronic acid in the same manner as described above for 6-bromo-3-[4-(propan-2-yloxy)phenyl]imidazo[1,2-a]pyridine. LC/MS m/z: 288.13 (³⁵Cl, M+H)⁺, 290.15 (³⁷Cl, M+H)⁺.

### Step 2: 3,6-bis[4-(propan-2-yloxy)phenyl]imidazo[1,2-b]pyridazine

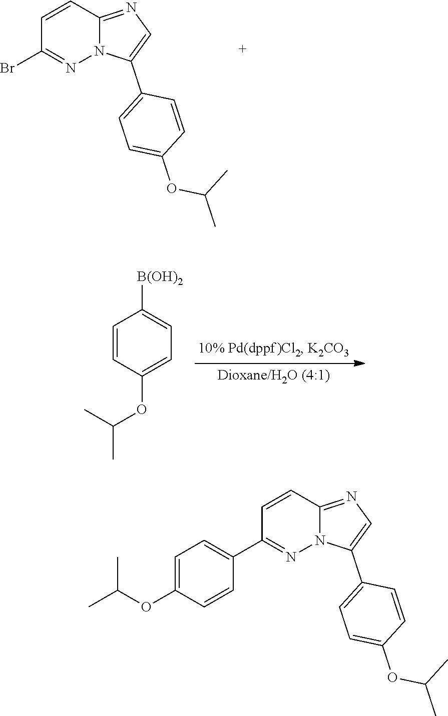 [SMILES: CC(C)Oc1ccc(-c2cnc3ccc(Br)nn23)cc1 ; "+" ; CC(C)Oc1ccc(B(O)O)cc1 ; CC(C)Oc1ccc(-c2ccc3ncc(-c4ccc(OC(C)C)cc4)n3n2)cc1]

To a solution of 6-chloro-3-[4-(propan-2-yloxy)phenyl]imidazo[1,2-b]pyridazine (25 mg, 0.087 mmol) in dioxane (0.6 mL) and water (0.15 mL) was added (4-isopropoxyphenyl)boronic acid (19 mg, 1.2 eq.) and potassium carbonate (36 mg g, 3 eq.). The reaction mixture was purged with nitrogen, then Pd(dppf)Cl₂ (7 mg, 0.1 eq.) was added. The resulting reaction mixture was heated to 100° C. for 10 min, then to 120° C. for 15 min under microwave irradiation. After cooling the reaction was poured into water and extracted with ethyl acetate. Dried over Na₂SO₄, filtered, and concentrated in vacuo. The residue was purified by preparative HPLC to give 4.5 mg (13%) of the title compound as an yellow oil. LC/MS m/z: 388.25 (M+H)⁺.

### Example D23: 3-(4-(tert-butoxy)phenyl)-6-(4-isopropoxyphenyl)-7-methylimidazo[1,2-b]pyridazine

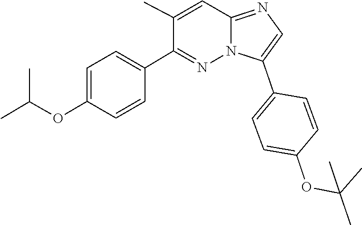 [SMILES: Cc1cc2ncc(-c3ccc(OC(C)(C)C)cc3)n2nc1-c1ccc(OC(C)C)cc1]

The title compound was prepared 3-(4-(tert-butoxy)phenyl)-6-chloro-7-methylimidazo[1,2-b]pyridazine and (4-isopropoxyphenyl)boronic acid in the same manner as described above for 3,6-bis[4-(propan-2-yloxy)phenyl]imidazo[1,2-b]pyridazine (example D10). LC/MS m/z: 416.56 [M+H]⁺.

### Example E1: 3-(4-(tert-butoxy)phenyl)-6-(4-(isopropoxy-d₇)phenyl)-7-methylimidazo[1,2-a]pyridine

### Step 1: 4-(3-(4-(tert-butoxy)phenyl)-7-methylimidazo[1,2-a]pyridin-6-yl)phenol

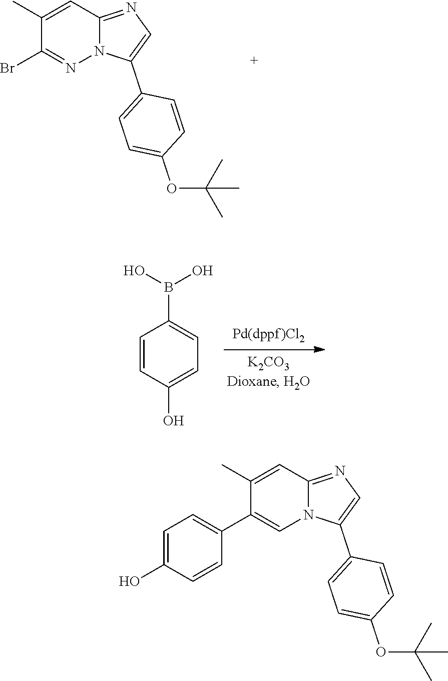 [SMILES: Cc1cc2ncc(-c3ccc(OC(C)(C)C)cc3)n2nc1Br ; OB(O)c1ccc(O)cc1 ; Cc1cc2ncc(-c3ccc(OC(C)(C)C)cc3)n2cc1-c1ccc(O)cc1]

To a microwave reactor vial was added 6-bromo-3-(4-(tert-butoxy)phenyl)-7-methylimidazo[1,2-a]pyridine (25 mg, 0.07 mmol), 4-hydroxyphenylboronic acid (11 mg, 0.083 mmol, 1.2 Eq), Pd(dppf)Cl₂ (6 mg, 10 mol %), and potassium carbonate (30 mg, 0.210 mmol, 3 Eq). 1.5 mL 1,4-dioxane and 0.5 mL water were then added, and the mixture degassed by bubbling nitrogen for 5 minutes. The vial is then tightly capped and subjected to microwave irradiation at 80° C. for 1 hour. The resulting mixture is diluted with ethyl acetate and the aqueous layer extracted 3× with ethyl acetate, followed by drying over Na₂SO₄ and evaporation to yield the crude product. The crude material is purified using silica gel flash chromatography with 100% ethyl acetate as eluent to give 20 mg of the title compound as a light brown oil. LC/MS m/z: 373.35 (M+H)⁺

### Step 2: 3-(4-(tert-butoxy)phenyl)-6-(4-(isopropoxy-d₇)phenyl)-7-methylimidazo[1,2-a]pyridine

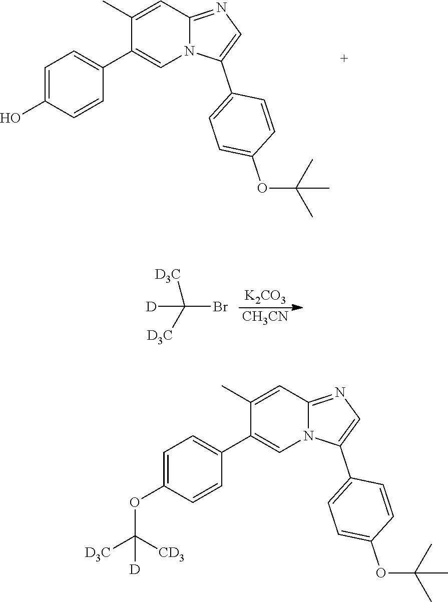

To a solution of 4-(3-(4-(tert-butoxy)phenyl)-7-methylimidazo[1,2-a]pyridin-6-yl)phenol (142 mg, 0.38 mmol) in acetonitrile was added potassium carbonate (0.105 g, 0.76 mmol, 2 Eq). The mixture is stirred at reflux for 30 minutes, at which time d7-isopropyl bromide (57 uL, 1.5 Eq) is added in one portion. The mixture is stirred at reflux overnight, then evaporated to dryness. The residue is dissolved in ethyl acetate, washed twice with water, dried over Na₂SO₄ and evaporated to give the crude solid, which is purified using silica gel flash chromatography with 3:7 hexanes:ethyl acetate as eluent to give 42 mg of the title compound. LC/MS m/z: 422.33 (M+H)⁺

Following schemes 1-5 and procedures above using the appropriate starting materials, the following examples can be made:

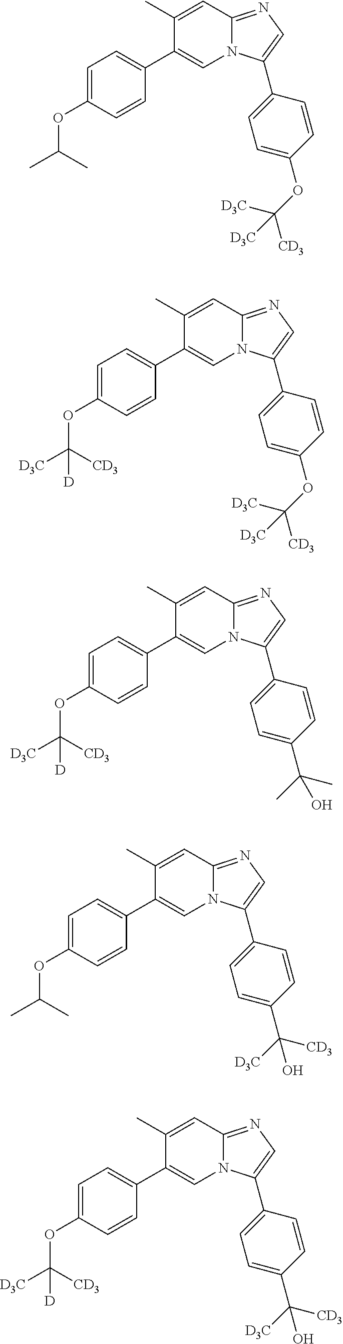

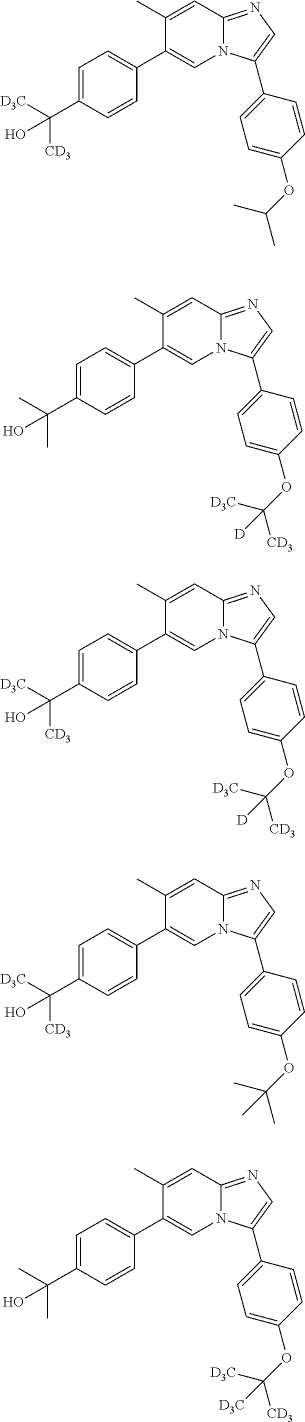

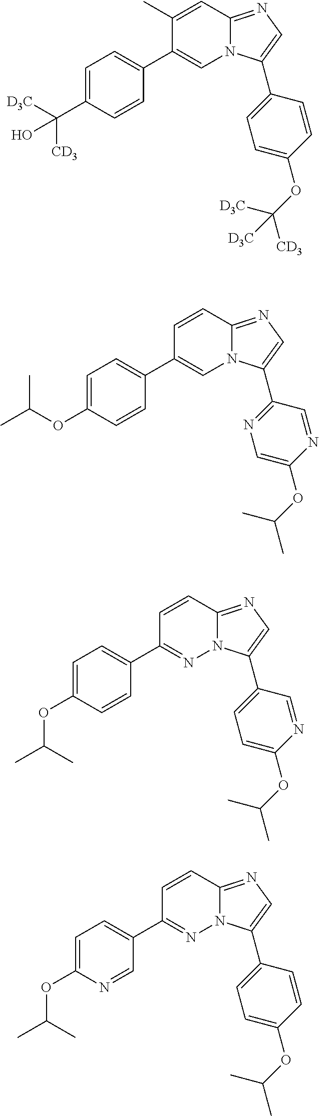

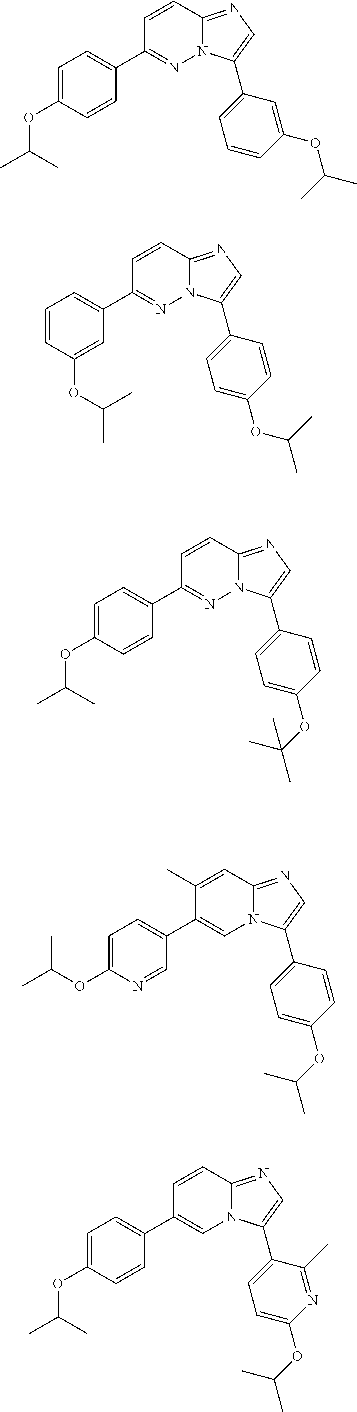

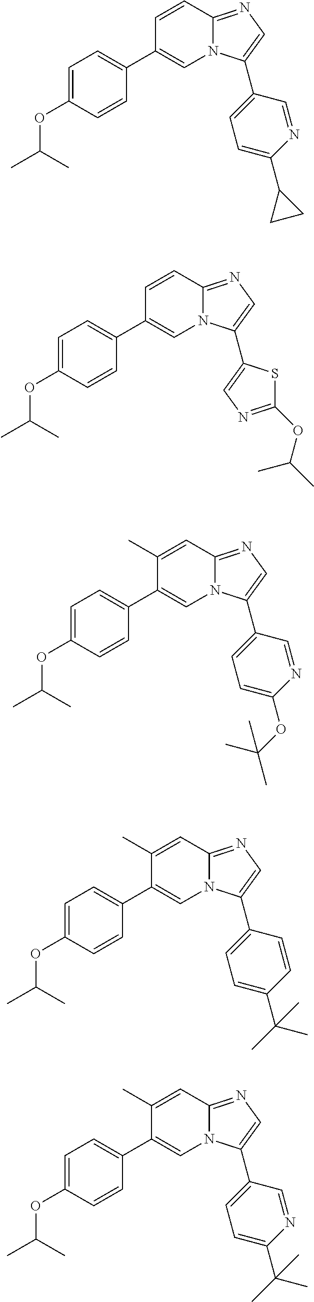

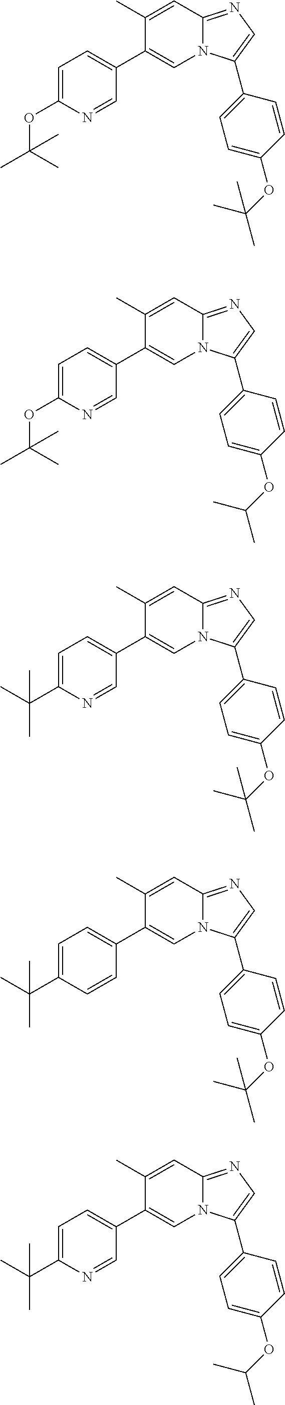 [SMILES: Cc1cc2ncc(-c3ccc(OC(C)(C)C)cc3)n2cc1-c1ccc(OC(C)(C)C)nc1 ; Cc1cc2ncc(-c3ccc(OC(C)C)cc3)n2cc1-c1ccc(OC(C)(C)C)nc1 ; Cc1cc2ncc(-c3ccc(OC(C)(C)C)cc3)n2cc1-c1ccc(C(C)(C)C)nc1 ; Cc1cc2ncc(-c3ccc(OC(C)(C)C)cc3)n2cc1-c1ccc(C(C)(C)C)cc1 ; Cc1cc2ncc(-c3ccc(OC(C)C)cc3)n2cc1-c1ccc(C(C)(C)C)nc1]

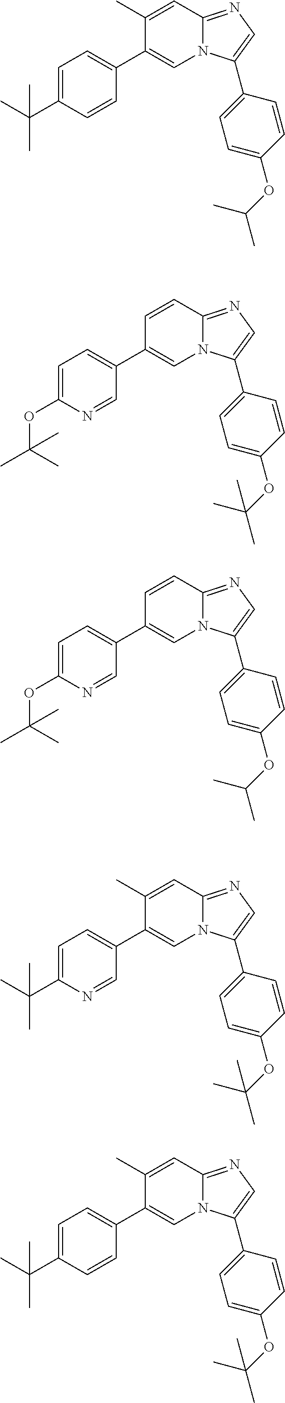

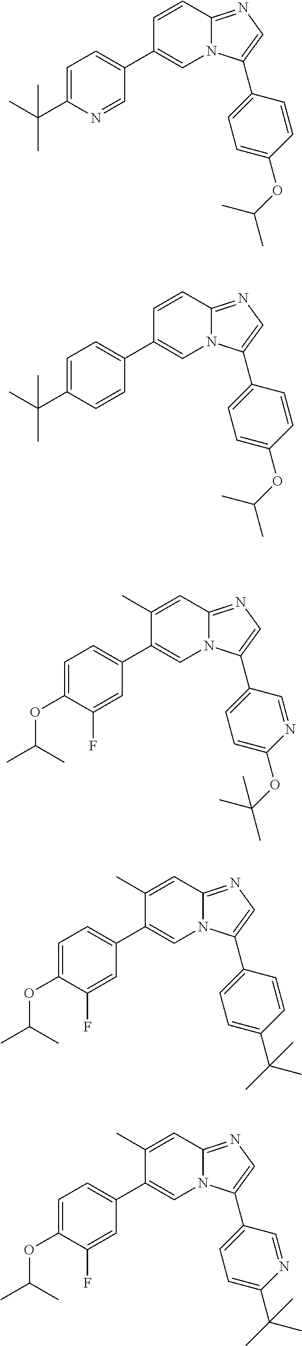

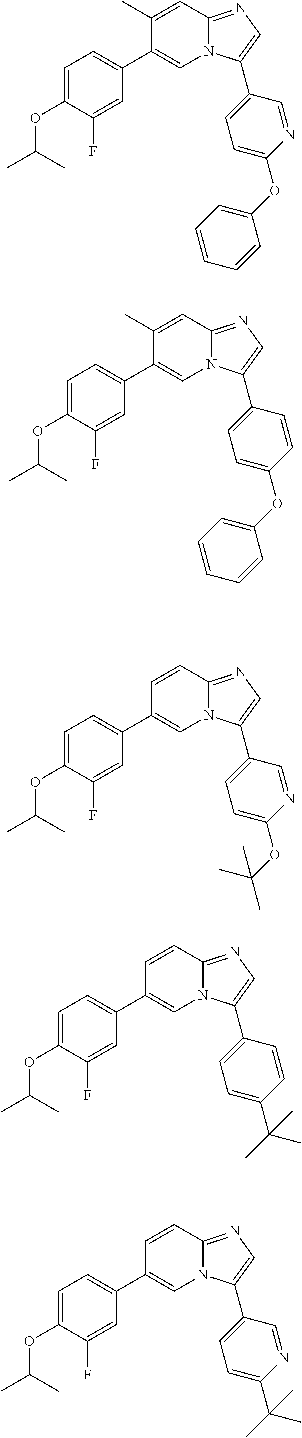

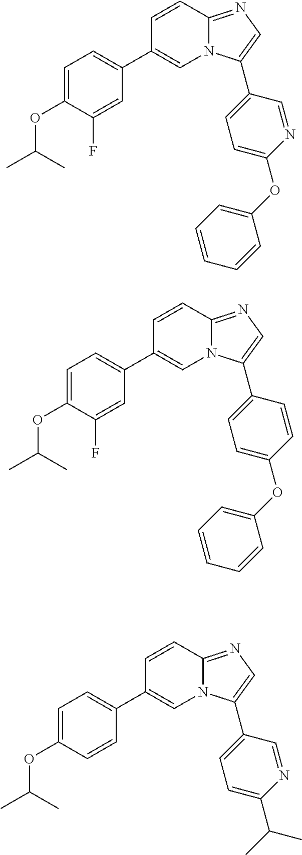

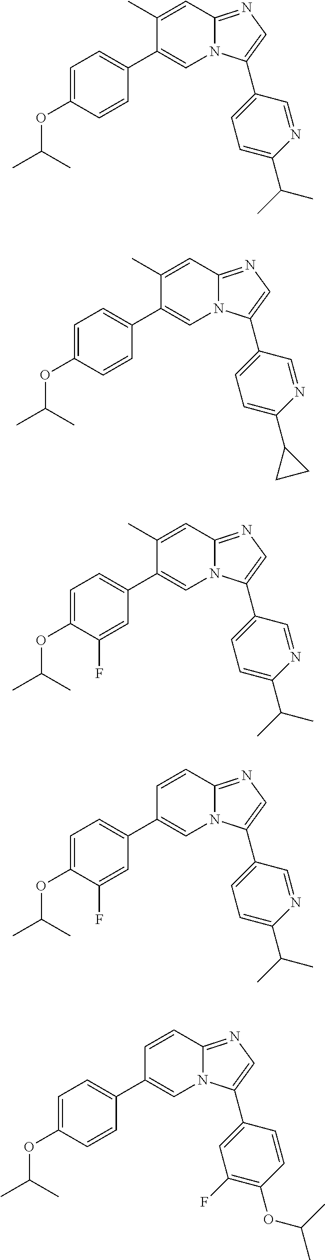

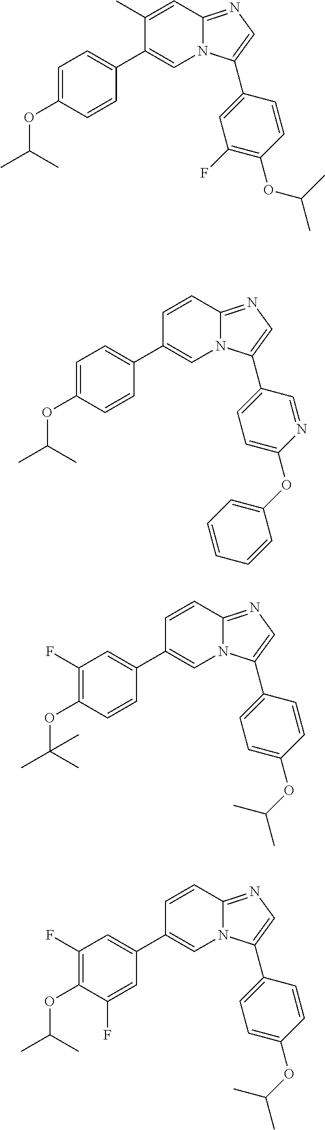

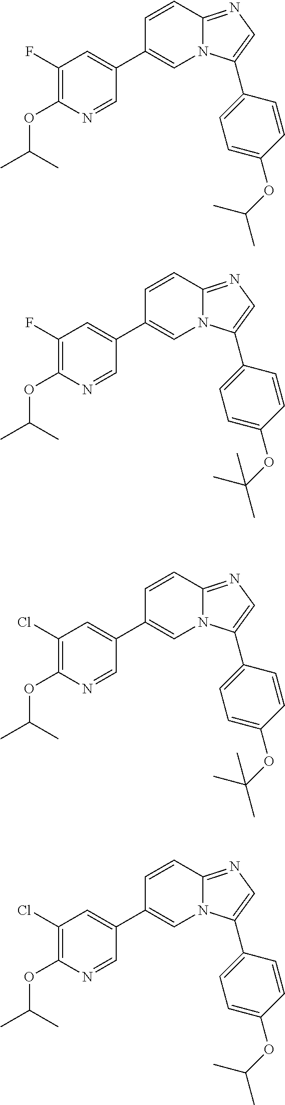

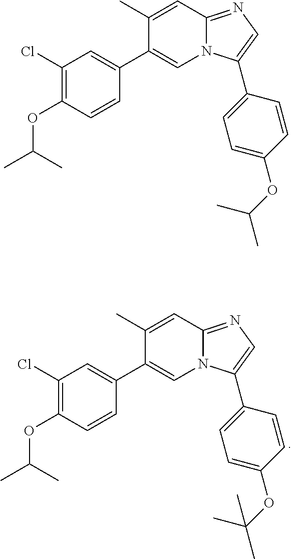 [SMILES: Cc1cc2ncc(-c3ccc(OC(C)C)cc3)n2cc1-c1ccc(OC(C)C)c(Cl)c1 ; Cc1cc2ncc(-c3ccc(OC(C)(C)C)cc3)n2cc1-c1ccc(OC(C)C)c(Cl)c1 ; "."]

### In Vitro Na+ Current Inhibition Assays

In vitro Na+ Current Inhibition Assays were performed by Eurofins Discovery (St Charles, MO). All compound functional activity determinations were determined through electrophysiological analyses of Na+ currents in HEK293 cells expressing a stably transfected Na+ channel (human Nav1.1, 1.2, 1.3, 1.5, 1.6, 1.7, or 1.8). After harvesting with Accutase cells are maintained in serum free medium at room temperature prior to electrophysiological recording utilizing the QPatch HT automated whole-cell patch-clamp platform (Sophion Bioscience, Woburn MA). Stock solution aliquots of the test compound are prepared in DMSO at 300× the final assay concentrations, and stored at −20° C. until the day of assay. On the day of the assay, an aliquot of the stock solution is thawed and diluted into external solution (137 mM NaCl, 4 mM KCl, 1 mM MgCl2, 1.8 mM CaCl2, 10 mM HEPES, 10 mM Glucose (pH 7.3, titrated with 10M NaOH) to make final test concentrations. A final concentration of 0.33% DMSO is maintained for each concentration of the assay compounds. Internal solution for recordings of Na+ consist consists of 100 mM CsF, 45 mM CsCl, 5 mM NaCl, 10 mM HEPES, 5 mM EGTA (pH 7.3, titrated with 1M CsOH).

### Human Nav1.5 Peak and Late Current Assays:

### Nav1.5 Peak Current Assay Protocol:

Onset and steady state block of peak Nav1.5 current was measured using a pulse pattern, repeated every 5 sec, consisting of a hyperpolarizing pulse to −120 mV for a 200 ms duration, depolarization to -15 mV amplitude for a 40 ms duration, followed by step to 40 mV for 200 ms and finally a 100 ms ramp (1.2 V/s) to a holding potential of −80 mV. Peak current was measured during the step to −15 mV.

Each concentration of compound was applied for 5 minutes. The assay was performed at room temperature and Tetracaine was used concurrently as a positive control. Only current amplitudes in excess of 200 pA at the control stage were analyzed. The amplitude of the sodium current was calculated by measuring the difference between the peak inward current on stepping to −15 mV (i.e. peak of the current) and the leak current. The sodium current was assessed in vehicle control conditions and then at the end of each five (5) minute compound application. Individual cell result was normalized to the vehicle control amplitude and the mean±SEM calculated for each compound concentration.

### Nav1.5 Late Current Assay:

Onset and steady state induction of late Nav1.5 current was measured using a pulse pattern, repeated every 5 sec, consisting of a hyperpolarizing pulse to −120 mV for a 200 ms duration, depolarization to −15 mV amplitude for a 40 ms duration, followed by step to 40 mV for 200 ms and finally a 100 ms ramp (1.2 V/s) to a holding potential of −80 mV. Each concentration of compound was applied for 5 minutes. The assay was performed at room temperature. After establishing vehicle control condition, all further addition of external solutions contain 30 nM ATX-II to activate late currents. Each concentration of compound was also co-applied with 30 nM ATXII to estimate the Nav1.5 late current inhibition. The assay was performed at room temperature. ATXII was used as the agonist positive control and was tested concurrently with the test compound. Ranolazine was used as the positive control and was tested concurrently with the test compound.

Nav1.5 late current was measured as the total charge flowing through the Nav1.5 channels (area under the curve) 5 ms after the Nav1.5 peak current till the end of the test pulse and as charge at their maxima during the ramp. Individual cell results were normalized to their respective vehicle control and the results were averaged. The sodium current was assessed in 30 nM ATXII as vehicle control conditions and then at the end of each five (5) minute compound application in the presence of 30 nM ATXII. Individual cell result was normalized to 30 nM ATXII vehicle control amplitude and the mean±SEM calculated for each compound concentration.

Two pulse assay protocol for human Nav1.1, 1.2, 1.3, 1.5, 1.6, 1.7, and 1.8 channels. Mammalian cultured cells expressing the indicated human Nav1.1, 1.2, 1.3, 1.5, 1.6, 1.7 or 1.8 channels were assayed using the The QPatch HT (Sophion Bioscience, Woburn MA) automated system with an intracellular solution (mM) of 100 CsF, 45 CsCl, 5 NaCl, 10 HEPES, 5 EGTA (pH 7.3, titrated with 1M CsOH) and extracellular solution (mM) of 137 NaCl, 4 KCl, 1 MgCl2, 1.8 CaCl2, 10 HEPES, 10 Glucose (pH 7.3, titrated with 10M NaOH). Onset and steady state block of the sodium channel was measured using a pulse pattern, repeated every 20 seconds (s) from a holding potential of −120 mV for 100 ms, and then pulsed to 0 mV for 500 ms to inactivate the Na channels and facilitate inactivated state-dependent binding of the test compounds. The cell membranes were then stepped back to −120 mV for 10 ms to recover from inactivation into resting or closed state (channels with test compound bound to them will not recover from inactivation and will not open) before stepping to 0 mV for 50 ms (pulse 2) to measure Na channels that are available to open. Peak current was measured during the step to 0 mV at pulse 1 and 2 to measure tonic and state-dependent inhibition. Compound activities were assessed by dividing post current amplitude (at the end of each five (5) minute compound application) by the vehicle control amplitude and the mean calculated for each compound concentration. Tetracaine was used as a positive control and was tested concurrently with the test compounds. Current amplitudes in excess of 200 pA at the control stage were analyzed. The amplitude of the current was calculated by measuring the difference between the peak inward current on stepping to 0 mV (i.e. peak of the current) and remaining current at the end of the step. The current was assessed in vehicle control conditions and then at the end of each five (5) minute compound application.

Results from the assays related to percent inhibition of hNav1.5 Late and Peak Current at 5 uM are summarized in Table 1. Results from the assays related to percent inhibition of hNav1.1, hNav1.2, hNav1.3, hNav1.5, hNav1.6, hNav1.7, and hNav1.8 peak currents at pulse 1 and 2 at 5 uM and 2.5 uM are summarized in Tables 2-4. In these tables, “A” indicates inhibition of less than 50%; “B” indicates inhibition of between about 50% to about 90%; “C” indicates inhibition of greater than 90%.

TABLE 1
Example compounds and their observed inhibitory activities
against hNav1.5 Late and Peak Current; nd: not determined

| | hNav1.5 Late | hNav1.5 Peak |
|---|---|---|
| Ex. | (5 uM) | (5 uM) |
| A32 | A | nd |
| C4 | C | nd |
| C12 | C | B |
| C15 | A | nd |
| C16 | A | nd |
| C17 | A | nd |
| C32 | C | B |
| C35 | C | nd |
| C37 | C | A |
| C40 | A | nd |
| C49 | A | nd |
| C52 | A | nd |
| C55 | B | A |
| C56 | A | nd |
| D12 | B | nd |
| D21 | B | nd |
| C70 | B | A |

TABLE 2
Example compounds and their observed inhibitory activities
against hNav1.6 and hNav1.8 peak currents at pulse 1 and 2

| | hNav1.6, | hNav1.6, | hNav1.8, | hNav1.8, |
|---|---|---|---|---|
| | Pulse 1 | Pulse 2 | Pulse 1 | Pulse 2 |
| Ex. | (2.5 uM) | (2.5 uM) | (5 uM) | (5 uM) |
| C37 | nd | nd | A | B |
| C58 | A | C | B | B |
| C55 | nd | nd | A | A |
| C59 | nd | nd | A | A |
| C60 | nd | nd | A | A |
| C61 | nd | nd | A | A |
| C66 | nd | nd | A | A |
| C67 | nd | nd | A | A |
| C68 | nd | nd | A | A |
| C91 | A | A | nd | nd |

TABLE 3
Example compounds and their observed inhibitory activities against
hNav1.5, hNav1.6, and hNav1.7 peak currents at pulse 1 and 2

| | hNav1.5, Pulse 1 | hNav1.5, Pulse 2 | hNav1.6, Pulse 1 | hNav1.6, Pulse 2 | hNav1.7, Pulse 1 | hNav1.7, Pulse 2 |
|---|---|---|---|---|---|---|
| Ex. | (5 uM) | (5 uM) | (5 uM) | (5 uM) | (5 uM) | (5 uM) |
| C2 | nd | nd | A | C | nd | nd |
| C12 | nd | nd | A | C | A | C |
| C18 | nd | nd | A | B | nd | nd |
| C21 | nd | nd | A | C | nd | nd |
| C23 | nd | nd | A | A | nd | nd |
| C24 | A | C | A | C | A | B |
| C32 | nd | nd | A | C | nd | nd |
| C37 | nd | nd | A | C | A | C |
| C48 | nd | nd | A | B | nd | nd |
| C50 | nd | nd | A | A | nd | nd |
| C51 | nd | nd | A | A | nd | nd |
| C55 | nd | nd | A | C | A | B |
| C60 | nd | nd | A | A | nd | nd |
| C61 | nd | nd | A | A | nd | nd |
| C66 | A | A | nd | nd | A | A |
| C67 | A | A | nd | nd | A | A |
| C68 | A | A | nd | nd | A | A |
| C70 | nd | nd | A | A | nd | nd |
| C71 | A | C | A | C | A | C |
| C72 | A | B | A | C | A | C |
| C73 | A | C | A | C | A | B |
| C74 | nd | nd | A | B | nd | nd |
| C75 | nd | nd | A | B | nd | nd |
| C76 | nd | nd | A | C | nd | nd |
| C77 | nd | nd | A | B | nd | nd |
| C78 | nd | nd | A | C | nd | nd |
| C79 | nd | nd | A | B | nd | nd |
| C80 | nd | nd | A | A | nd | nd |
| C81 | nd | nd | A | C | nd | nd |
| C82 | nd | nd | A | C | nd | nd |
| C83 | nd | nd | A | B | nd | nd |
| C84 | nd | nd | A | B | nd | nd |
| C85 | nd | nd | A | B | nd | nd |
| C86 | nd | nd | A | C | nd | nd |
| C87 | A | C | A | C | A | C |
| C88 | nd | nd | A | C | nd | nd |
| C89 | A | B | A | C | A | C |
| C90 | A | C | A | C | A | C |
| D10 | nd | nd | A | C | nd | nd |
| D23 | nd | nd | A | B | nd | nd |

TABLE 4
Example compounds C12, C72, and C73 and its observed inhibitory activities against
hNav1.1, hNav1.2, and hNav1.3 peak currents at pulse 1 and 2

| | hNav1.1, Pulse 1 | hNav1.1, Pulse 2 | hNav1.2, Pulse 1 | hNav1.2, Pulse 2 | hNav1.3, Pulse 1 | hNav1.3, Pulse 2 |
|---|---|---|---|---|---|---|
| Ex. | (5 uM) | (5 uM) | (5 uM) | (5 uM) | (5 uM) | (5 uM) |
| C12 | A | C | A | C | A | C |
| C72 | nd | nd | A | C | nd | nd |
| C73 | nd | nd | A | C | nd | nd |

What is claimed is:

1. A method of modulating at least one voltage-gated sodium channels in a mammal, including the Na_{V}1.1, 1.2, 1.3, 1.4, 1.5, 1.6, 1.7, 1.8, and 1.9 channels, wherein the method comprises administering to the mammal in need thereof a therapeutically effective amount of a compound of Structural Formula I 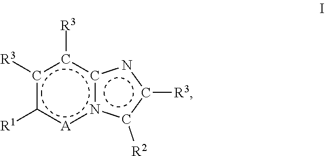
or a pharmaceutically acceptable salt, and a pharmaceutically acceptable carrier, diluent, or vehicle thereof, wherein:
A is independently N or C—R³;
R¹ is selected from (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₂ to C₉) heterocycloalkyl, (C₆ to C₁₀) aryl, and (C₂ to C₉) heteroaryl, wherein
each of the said (C₁ to C₆) alkyl, (C₂ to C₆) alkenyl, (C₂ to C₆) alkynyl, (C₅ to C₁₀) cycloalkyl, (C₅ to C₁₀) cycloalkenyl, (C₂ to C₉) heterocycloalkyl, (C₆ to C₁₀) aryl, and (C₂ to C₉) heteroaryl is optionally substituted with at least one R⁴ group, and wherein said (C₂ to C₉) heteroaryl is C-attached;
R² is selected from (C₂ to C₆) alkenyl, (C₂ to C₆) alkynyl, (C₃ to C₁₀) cycloalkyl, (C₅ to C₁₀) cycloalkenyl, (C₂ to C₉) heterocycloalkyl, (C₆ to C₁₀) aryl, and (C₂ to C₉) heteroaryl, wherein
each of the said (C₂ to C₆) alkenyl, (C₂ to C₆) alkynyl, (C₃ to C₁₀) cycloalkyl, (C₅ to C₁₀) cycloalkenyl, (C₂ to C₉) heterocycloalkyl, (C₆ to C₁₀) aryl, and (C₂ to C₉) heteroaryl is optionally substituted with at least one R⁴ group;
and wherein at least one of R¹ and R² is selected from (C₃ to C₁₀) cycloalkyl, (C₅ to C₁₀) cycloalkenyl, (C₂ to C₉) heterocycloalkyl, (C₆ to C₁₀) aryl, and (C₂ to C₉) heteroaryl;
each of the R³ is independently selected from hydrogen, deuterium, halogen, cyano, —CD₃, OH, CF₃, (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, (C₂ to C₉) heteroaryl, —C(O)R⁵, —C(O)NR^{6a}R^{6b}, —C(O)NR^{6a}S(O)ₘR⁵, —C(O)NR^{6a}S(O)ₘNR^{6a}R^{6b}, —NR^{6a}R^{6b}, —S(O)ₘR⁵, —S(O)ₘNR^{6a}R^{6b}, —NR^{6a}S(O)ₘR⁵, —(CH₂)ₙC(O)OR⁵, —(CH₂)ₙC(O)NR^{6a}R^{6b}, —OC(O)R⁵, —NR^{6a}C(O)R⁵, and —NR^{6c}C(O)NR^{6a}R^{6b}, wherein
each of the said (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, and (C₂ to C₉) heteroaryl is optionally substituted with at least one R⁴ group;
R⁴ is independently selected from hydrogen, deuterium, halogen, cyano, OH, CF₃, (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(Cato C₁₀) aryl, (C₅ to C₁₀) cycloakyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, (C₂ to C₉) heteroaryl, —C(O)R⁵, —C(O)NR^{6a}R^{6b}, —C(O)NR^{6a}S(O)ₘR⁵, —C(O)NR^{6a}S(O)ₘNR^{6a}R^{6b}, —NR^{6a}R^{6b}, —S(O)ₘR⁵, —S(O)ₘNR^{6a}R^{6b}, —NR^{6a}S(O)ₘR⁵, —(CH₂)ₙC(O)OR⁵, —(CH₂)ₙC(O)NR^{6a}R^{6b}, —OC(O)R⁵, —NR^{6a}C(O)R⁵, and —NR^{6c}C(O)NR^{6a}R^{6b}, wherein
each of the said (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₅ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, and (C₂ to C₉) heteroaryl is optionally substituted with at least one R⁷ group;
each of the R⁵ is independently selected from hydrogen, deuterium, (C₁ to C₆) alkyl, (C₂ to C₉) alkenyl, (C₂ to C₆) alkynyl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl, wherein
each of the said (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁷ group;
each of the R^{6a}, R^{6b}, and R^{6c} are independently selected from hydrogen, deuterium, (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl,
or R^{6a} and R^{6b} may be taken together with the nitrogen atom to which they are attached to form a 4 to 8 membered cycloheteroalkyl ring, wherein
said 4 to 8 membered cycloheteroalkyl ring has 1 to 3 ring heteroatoms selected from the group consisting of N, O, and S, and wherein
the said 4 to 8 membered cycloheteroalkyl ring is optionally substituted with at least one R⁷ group;
R⁷ is independently selected from hydrogen, deuterium, halogen, OH, CF₃, (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, (C₂ to C₉) heteroaryl, —C(O)R⁸, —C(O)NR^{9a}R^{9b}, —NR^{9a}R^{9b}, —S(O)ₘR⁸, —S(O)ₘNR^{9a}R^{9b}, —NR^{9a}S(O)ₘR⁸, —(CH₂)ₙC(O)OR⁸, —(CH₂)ₙC(O)N(R^{9a}R^{9b}), —OC(O)R⁸, —NR^{9a}C(O)R⁸, and —NR⁹C(O)N(R^{9a}R^{9b}), wherein
each of the said (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, and (C₂ to C₉) heteroaryl is optionally substituted with at least one R¹⁰ group;
each of the R⁶ is independently selected from hydrogen, deuterium, (C₁ to C₆) alkyl, (C₂ to C₉) alkenyl, (C₂ to C₆) alkynyl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl, wherein
each of the said (C₁ to C₆) alkyl, (C₂ to C₆) alkenyl, (C₂ to C₆) alkynyl, (C₅ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R¹⁰ group;
each of the R^{9a}, R^{9b}, and R^{9c} are independently selected from hydrogen, (C₁ to C₆) alkyl, (C₂ to C₆) alkenyl, (C₂ to C₆) alkynyl, (C₅ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl,
or R^{9a} and R^{9b} may be taken together with the nitrogen atom to which they are attached to form a 4 to 8 membered cycloheteroalkyl ring, wherein
said 4 to 8 membered cycloheteroalkyl ring has 1 to 3 ring heteroatoms selected from the group consisting of N, O, and S, and wherein
the said 4 to 8 membered cycloheteroalkyl ring is optionally substituted with hydrogen, deuterium, halogen, cyano, —CD₃, OH, CF₃, (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, and (C₂ to C₉) heteroaryl;
R¹⁰ is independently selected from hydrogen, deuterium, halogen, cyano, OH, CF₃, (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloakyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, and (C₂ to C₉) heteroaryl, wherein
each of the said (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, and (C₂ to C₉) heteroaryl is optionally substituted with hydrogen, deuterium, halogen, cyano, —CD₃, OH, CF₃, (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, and (C₂ to C₉) heteroaryl;
m is 0, 1, 2, 3, or 4;
and n is 0, 1, 2, 3, 4, 5, or 6.

2. The method of claim 1, wherein:
R¹ and R² are independently selected from the group consisting of 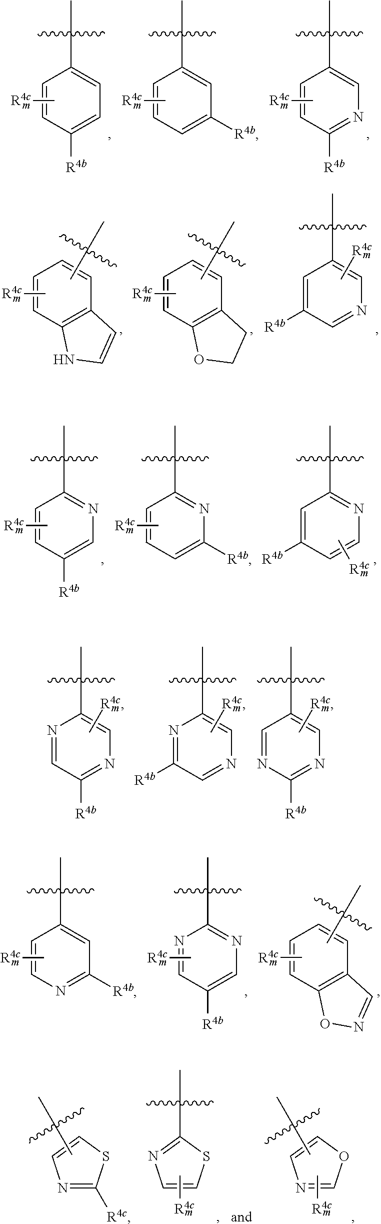
wherein
R^{4b} is selected from (C₁ to C₆) alkyl, (C₂ to C₆) alkenyl, (C₂ to C₆) alkynyl, —O—(C₁ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₆) cycloheteroalkyl, (C₆ to C₁₀) aryl, (C₂ to C₉) heteroaryl, and —C(O)R⁵, wherein
each of the said (C₁ to C₆) alkyl, (C₂ to C₆) alkenyl, (C₂ to C₆) alkynyl, —O—(C₁ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₆) cycloheteroalkyl, (C₆ to C₁₀) aryl, and (C₂ to C₉) heteroaryl is optionally substituted with at least one R⁷ group;
R^{4c} is independently selected from hydrogen, deuterium, halogen, cyano, OH, CF₃, (C₁ to C₆) alkyl, (C₂ to C₆) alkenyl, (C₂ to C₆) alkynyl, —O—(C₁ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, (C₆ to C₁₀) aryl, (C₂ to C₉) heteroaryl, —C(O)R⁵, —NR^{6a}R^{6b}, —S(O)ₘR⁵, —S(O)ₘNR^{6a}R^{6b}, —NR^{6a}S(O)ₘR⁵, —OC(O)R⁵, —NR^{6a}C(O)R⁵, and —NR^{6c}C(O)NR^{6a}R^{6b}, wherein
each of the said (C₁ to C₆) alkyl, (C₂ to C₆) alkenyl, (C₂ to C₆) alkynyl, —O—(C₁ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, (C₆ to C₁₀) aryl, and (C₂ to C₉) heteroaryl is optionally substituted with at least one R⁷ group.

3. The method of claim 2, wherein:
R¹ and R² are independently selected from the group consisting of

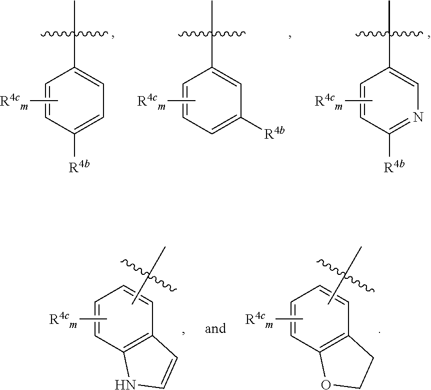

4. The method of claim 2, wherein:
R^{4b} is selected from the group consisting of
halogen, methoxy, isopropoxy, cyclopropoxy, tert-butoxy, cyclopropylmethoxy, (2-hydroxypropan)-2-yl, (1-hydroxycyclopropan)-1-yl, (1-hydroxycyclobutan)-1-yl, difluoromethoxy, trifluoromethoxy, trifluoromethyl, trifluoroethoxy, methoxyethoxy, phenoxy, benzyloxy, difluoromethyl, cyclopropyl, acetyl, n-propyloxy, n-butyloxy, isobutyloxy, sea-butyloxy, cyclobutyloxy, cyclopentyloxy, (1-hydroxycyclopentan)-1-yl, hydroxyethoxy, ethoxyethoxy, isopropoxyethoxy, isopropyl, n-propyl, n-butyl, isobutyl, sec-butyl, cyclobutyl, cyclopentyl, ter-butyl, propanoyl, isobutanoyl, sec-butanoyl, tert-butanoyl, cyclopropanoyl, cyclobutanoyl, and cyclopentanoyl.

5. The method of claim 3, wherein:
R^{4b} is selected from the group consisting of
halogen, methoxy, isopropoxy, cyclopropoxy, tert-butoxy, cyclopropylmethoxy, (2-hydroxypropan)-2-yl, (1-hydroxycyclopropan)-1-yl, (1-hydroxycyclobutan)-1-yl, difluoromethoxy, trifluoromethoxy, trifluoromethyl, trifluoroethoxy, methoxyethoxy, phenoxy, benzyloxy, difluoromethyl, cyclopropyl, acetyl, n-propyloxy, n-butyloxy, isobutyloxy, sec-butyloxy, cyclobutyloxy, cyclopentyloxy, (1-hydroxycyclopentan)-1-yl, hydroxyethoxy, ethoxyethoxy, isopropoxyethoxy, isopropyl, n-propyl, n-butyl, isobutyl, sec-butyl, cyclobutyl, cyclopentyl, ter-butyl, propanoyl, isobutanoyl, sec-butanoyl, tert-butanoyl, cyclopropanoyl, cyclobutanoyl, and cyclopentanoyl.

6. The method of claim 1, wherein: A is C—R³.

7. The method of claim 1, wherein: A is N.

8. A method of modulating at least one voltage-gated sodium channel in a mammal, including the Na_{V}1.1, 1.2, 1.3, 1.4, 1.5, 1.6, 1.7, 1.8, and 1.9 channels, wherein the method comprises administering to the mammal in need thereof a therapeutically effective amount of a compound selected from the group consisting of

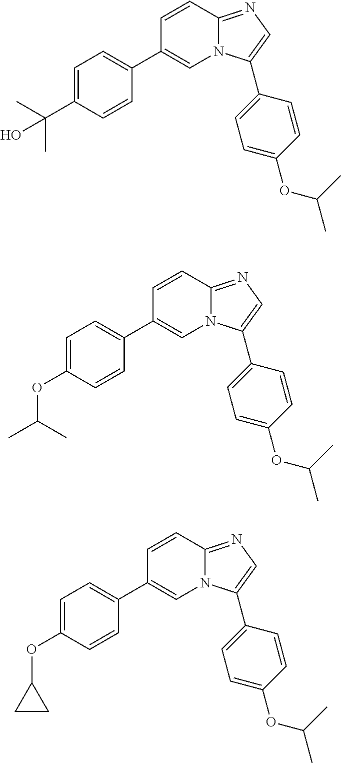

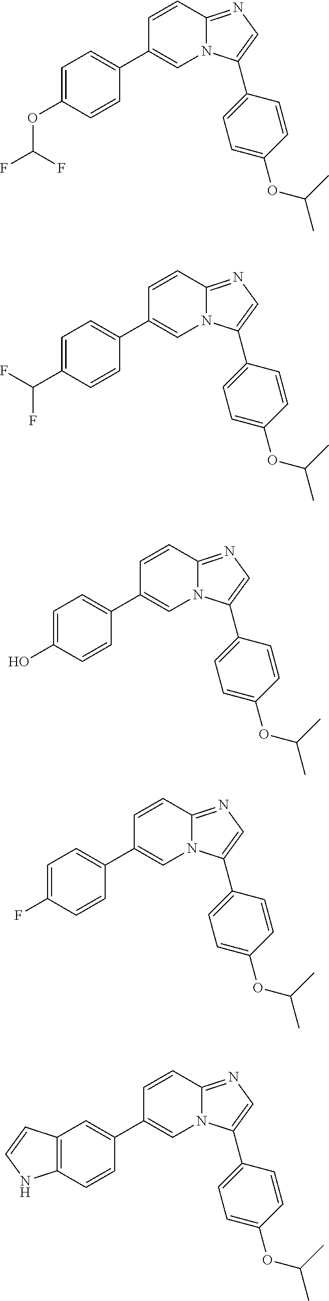 [SMILES: CC(C)Oc1ccc(-c2cnc3ccc(-c4ccc(OC(F)F)cc4)cn23)cc1 ; CC(C)Oc1ccc(-c2cnc3ccc(-c4ccc(C(F)F)cc4)cn23)cc1 ; CC(C)Oc1ccc(-c2cnc3ccc(-c4ccc(O)cc4)cn23)cc1 ; CC(C)Oc1ccc(-c2cnc3ccc(-c4ccc(F)cc4)cn23)cc1 ; CC(C)Oc1ccc(-c2cnc3ccc(-c4ccc5[nH]ccc5c4)cn23)cc1]

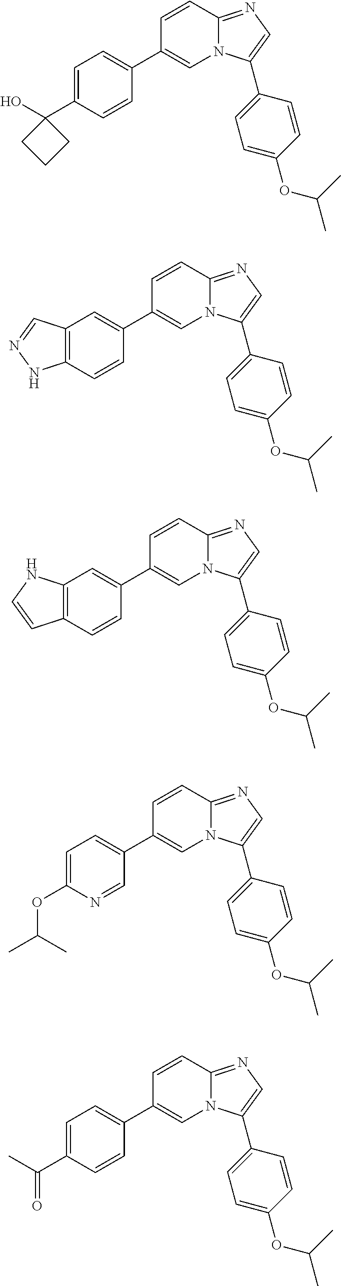

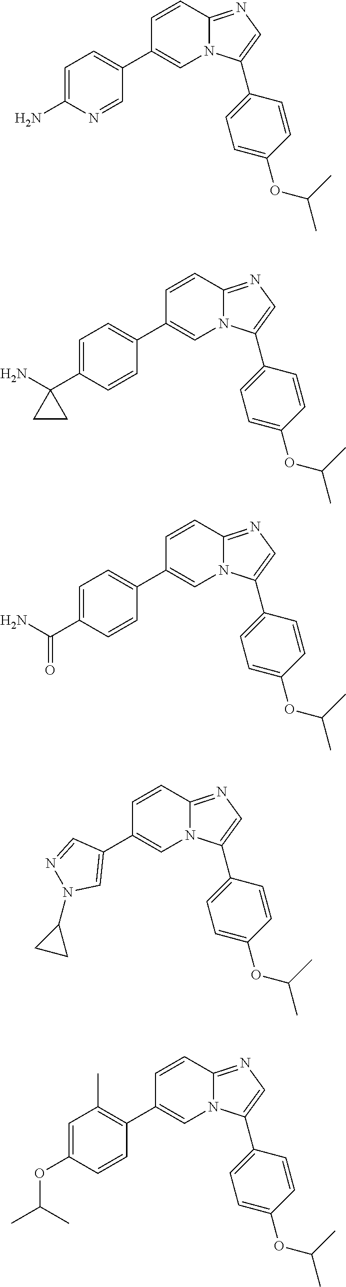

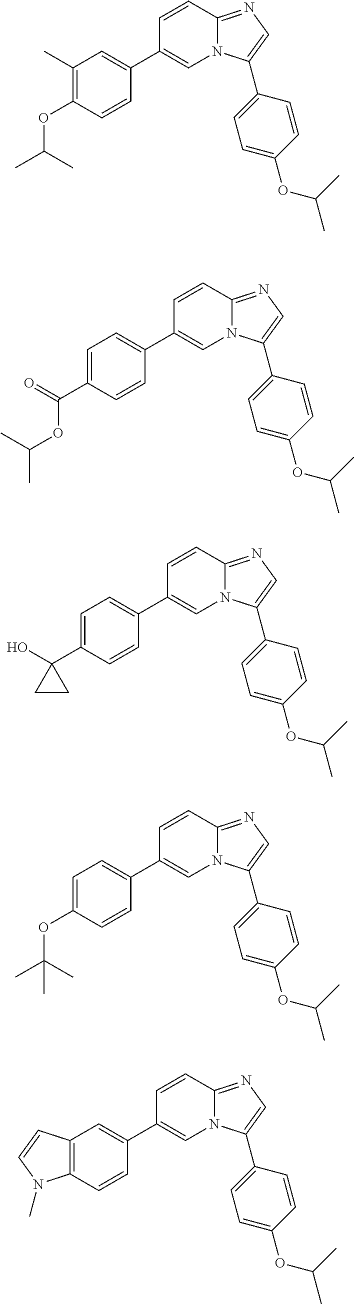

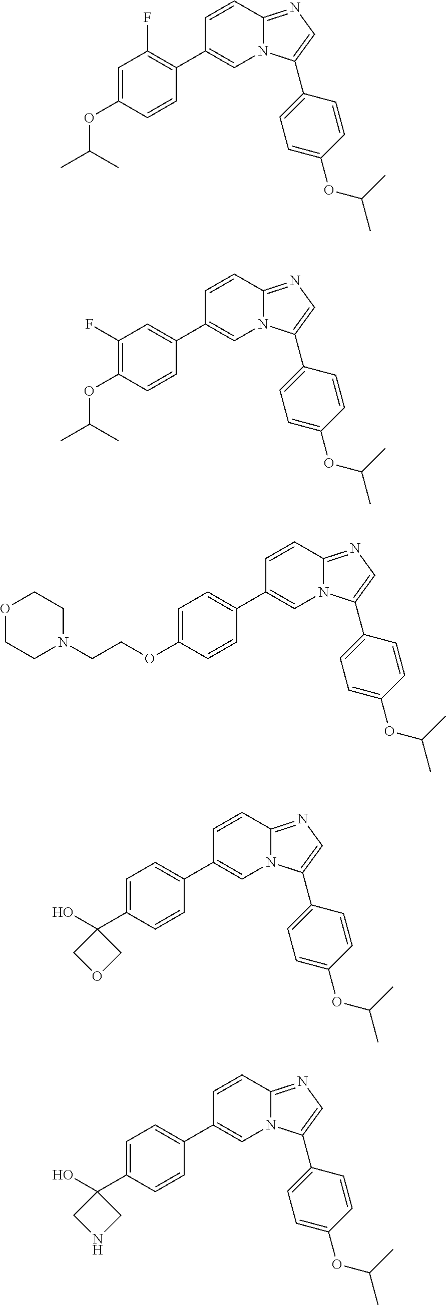

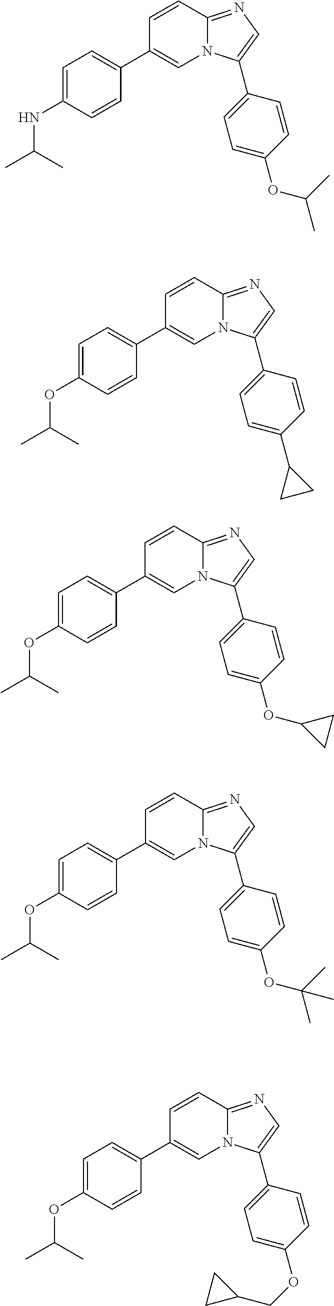

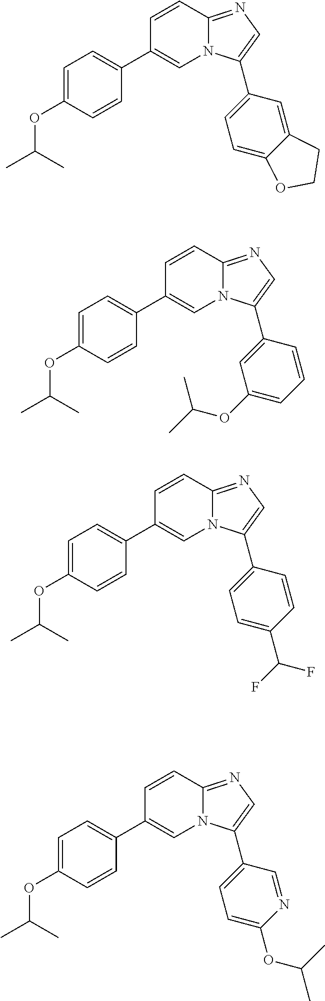

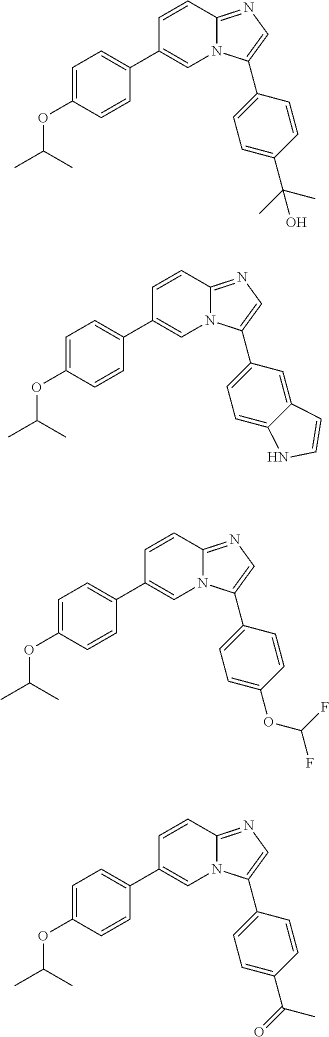

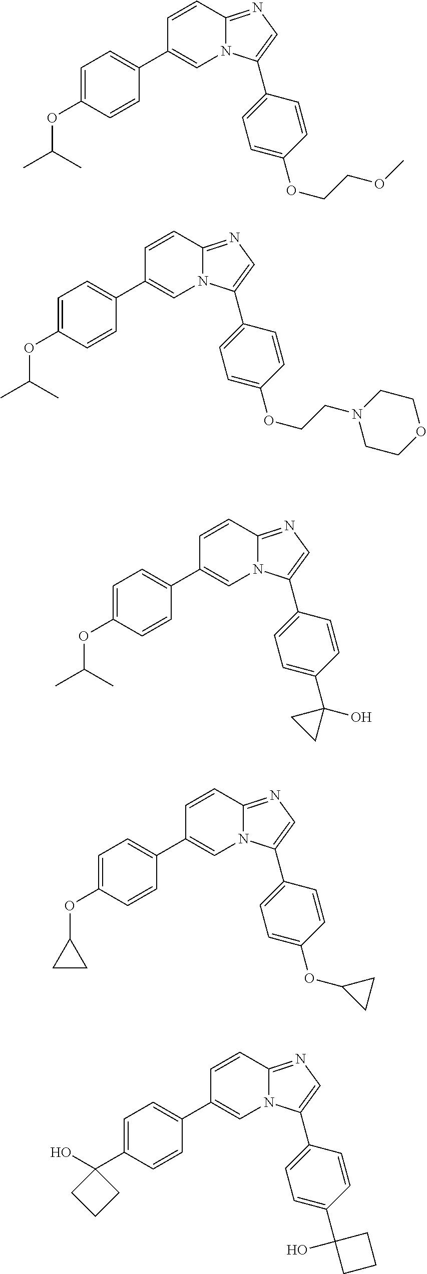

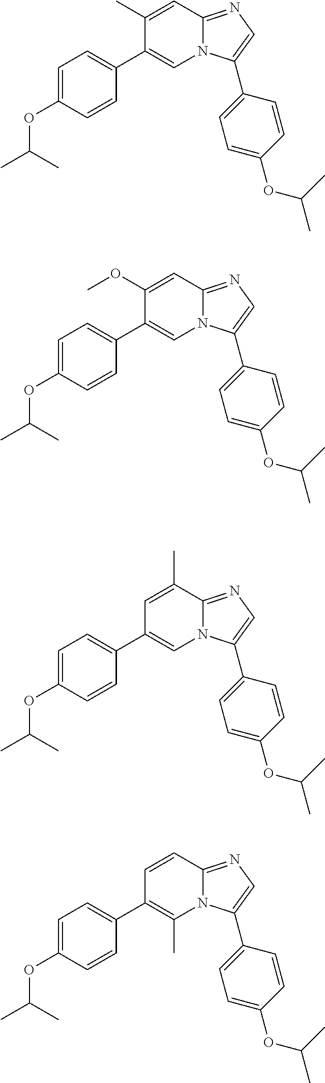

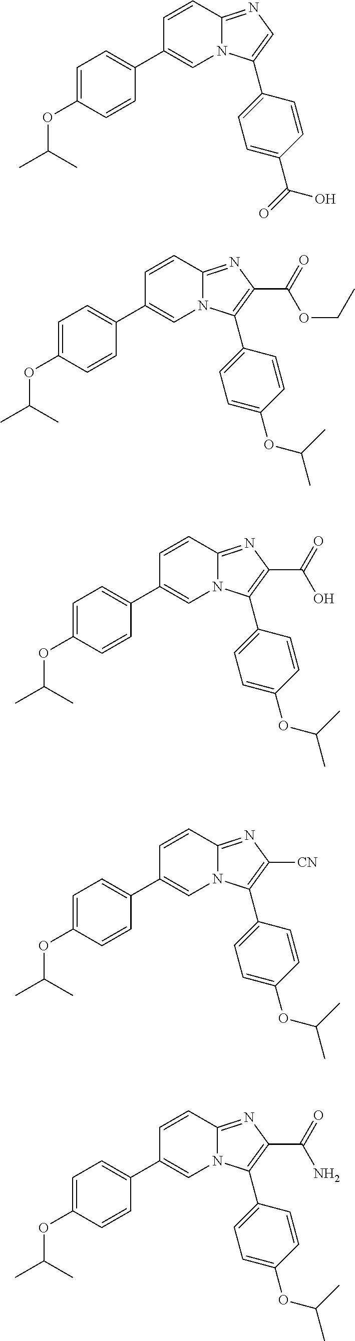

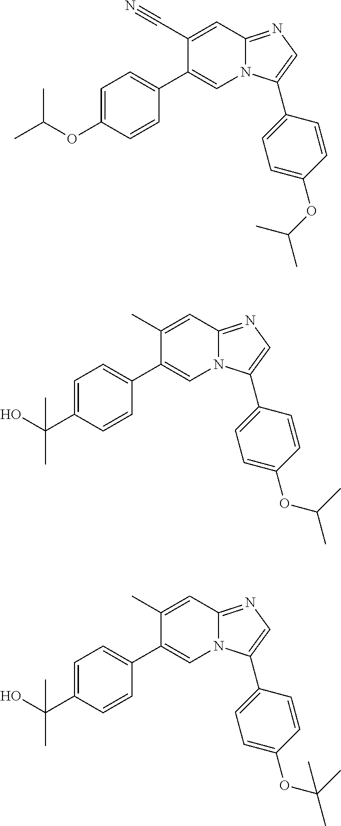 [SMILES: CC(C)Oc1ccc(-c2cn3c(-c4ccc(OC(C)C)cc4)cnc3cc2C#N)cc1 ; Cc1cc2ncc(-c3ccc(OC(C)C)cc3)n2cc1-c1ccc(C(C)(C)O)cc1 ; Cc1cc2ncc(-c3ccc(OC(C)(C)C)cc3)n2cc1-c1ccc(C(C)(C)O)cc1]

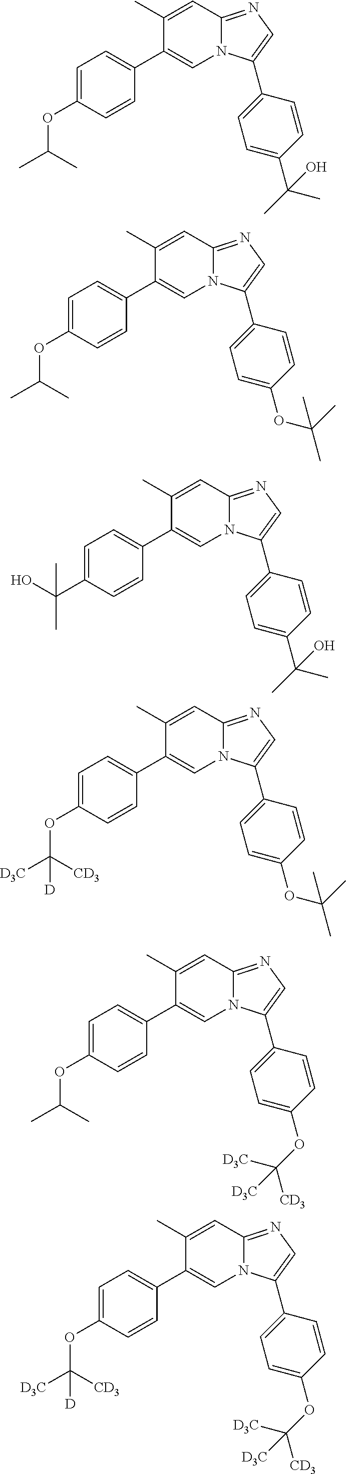

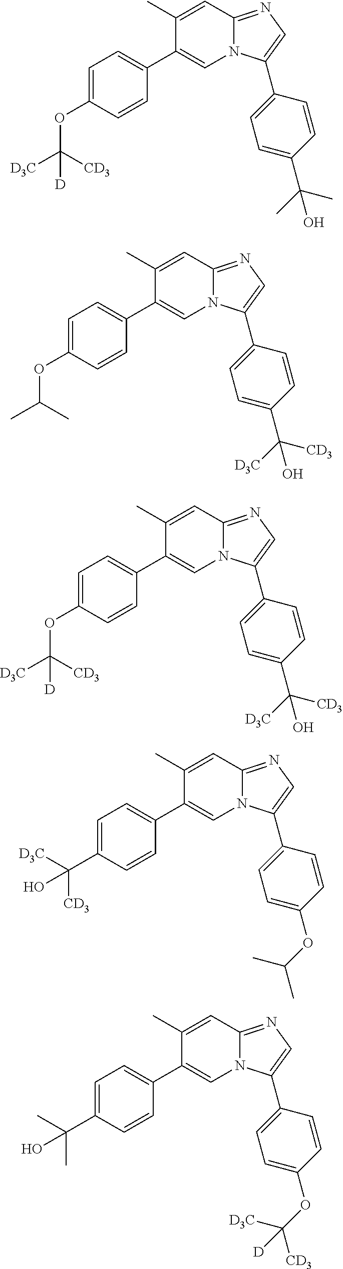

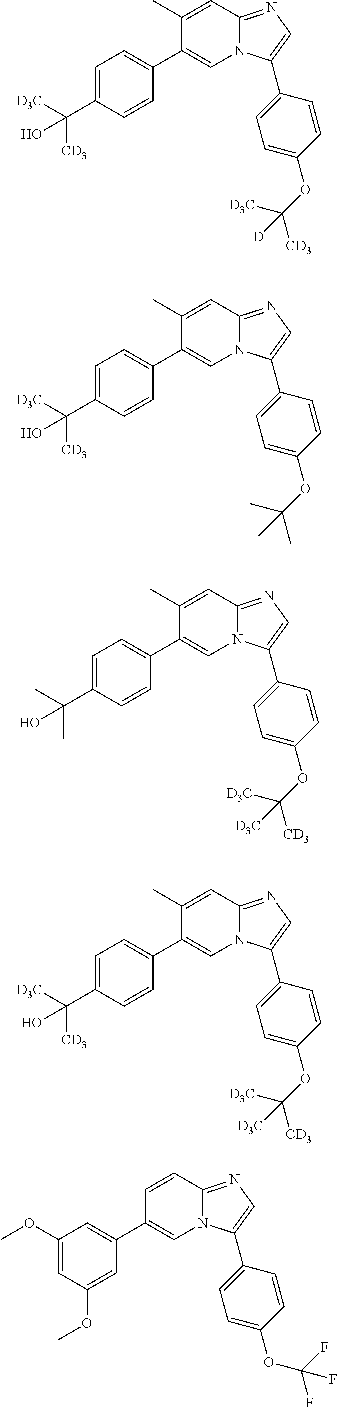

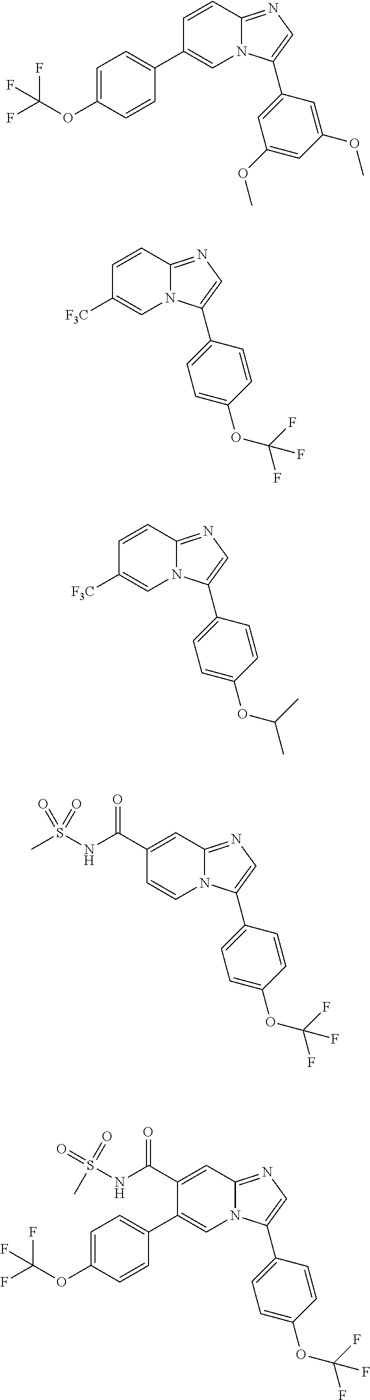

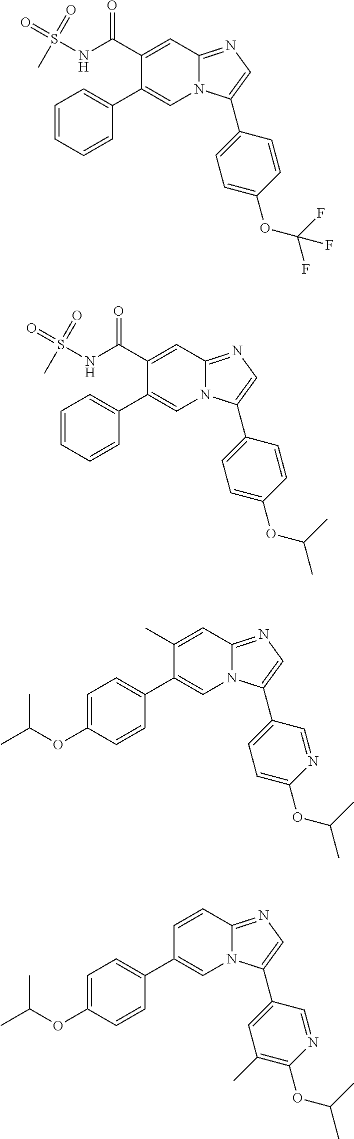

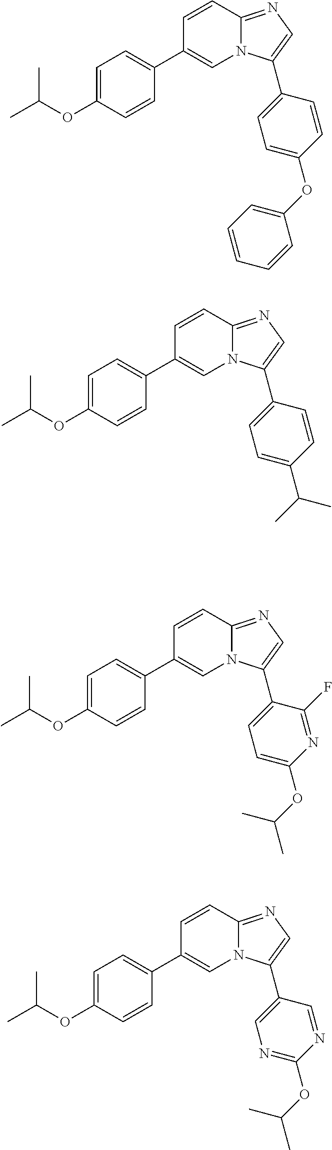

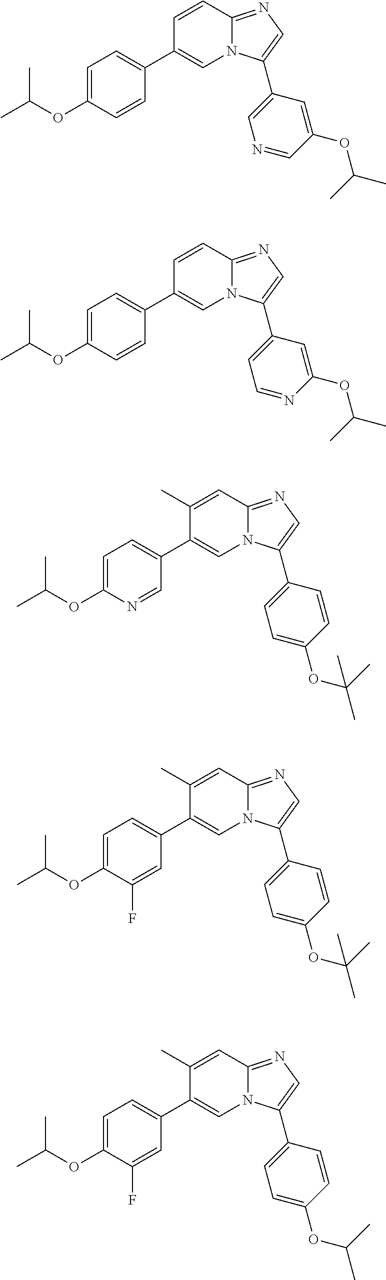

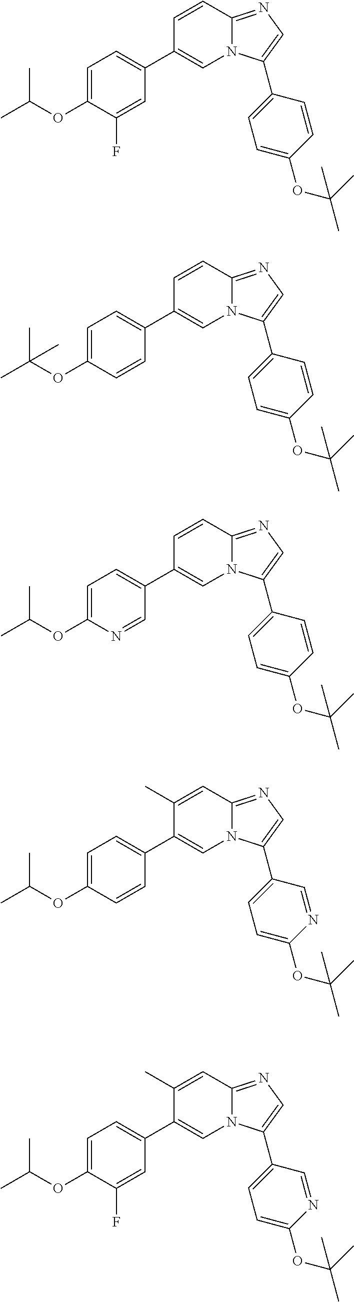

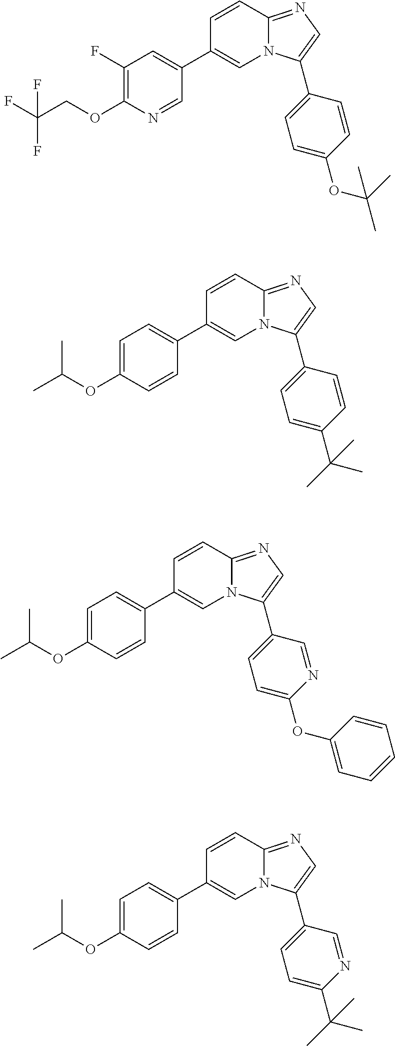

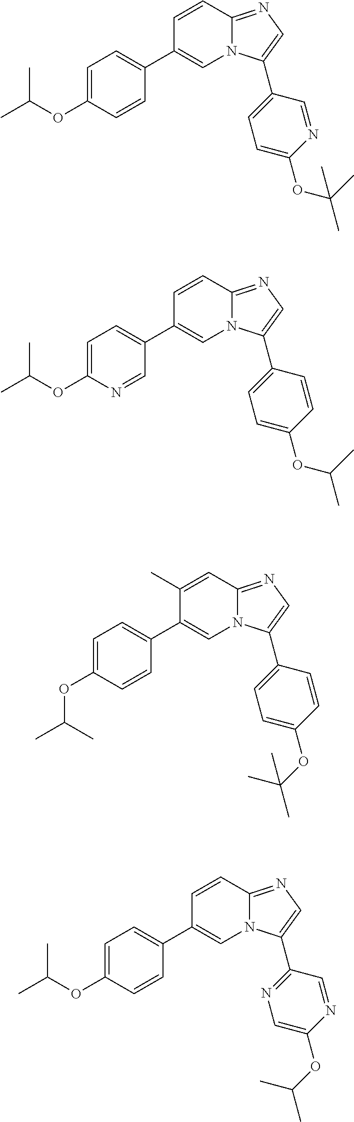

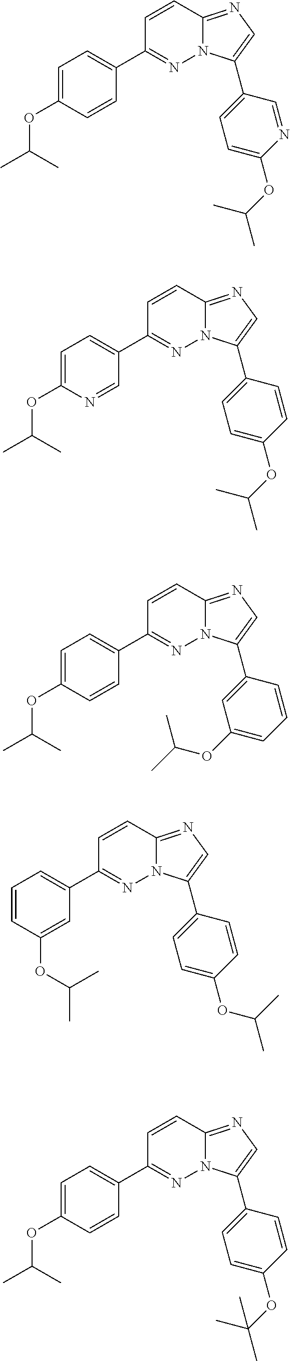

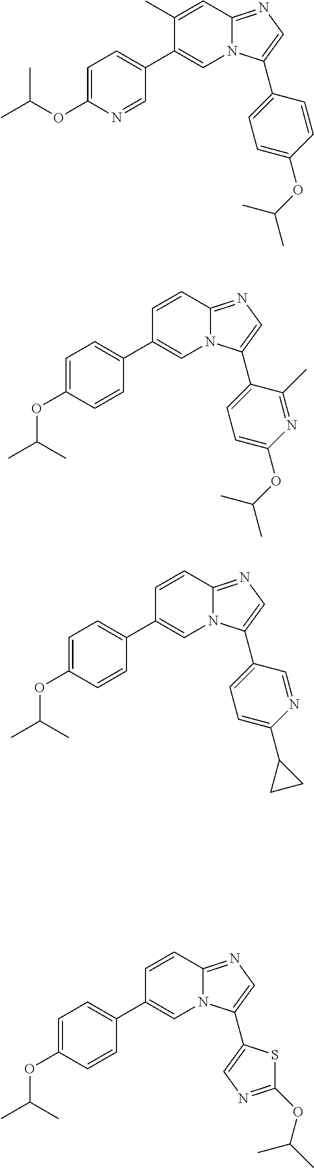

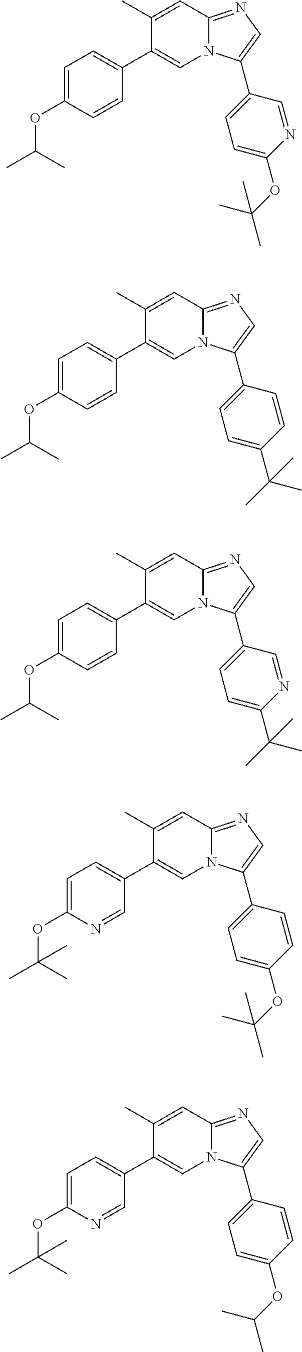

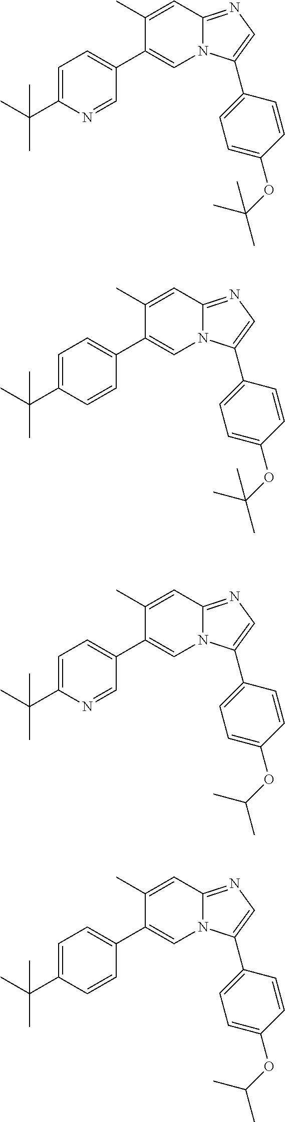 [SMILES: Cc1cc2ncc(-c3ccc(OC(C)(C)C)cc3)n2cc1-c1ccc(C(C)(C)C)nc1 ; Cc1cc2ncc(-c3ccc(OC(C)(C)C)cc3)n2cc1-c1ccc(C(C)(C)C)cc1 ; Cc1cc2ncc(-c3ccc(OC(C)C)cc3)n2cc1-c1ccc(C(C)(C)C)nc1 ; Cc1cc2ncc(-c3ccc(OC(C)C)cc3)n2cc1-c1ccc(C(C)(C)C)cc1]

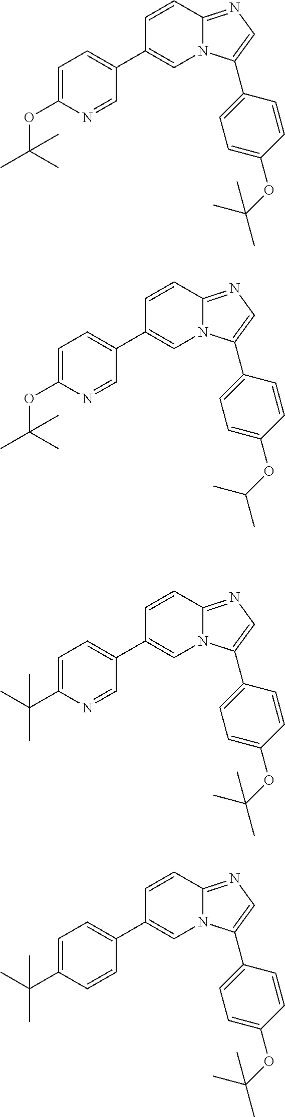 [SMILES: CC(C)(C)Oc1ccc(-c2cnc3ccc(-c4ccc(OC(C)(C)C)nc4)cn23)cc1 ; CC(C)Oc1ccc(-c2cnc3ccc(-c4ccc(OC(C)(C)C)nc4)cn23)cc1 ; CC(C)(C)Oc1ccc(-c2cnc3ccc(-c4ccc(C(C)(C)C)nc4)cn23)cc1 ; CC(C)(C)Oc1ccc(-c2cnc3ccc(-c4ccc(C(C)(C)C)cc4)cn23)cc1]

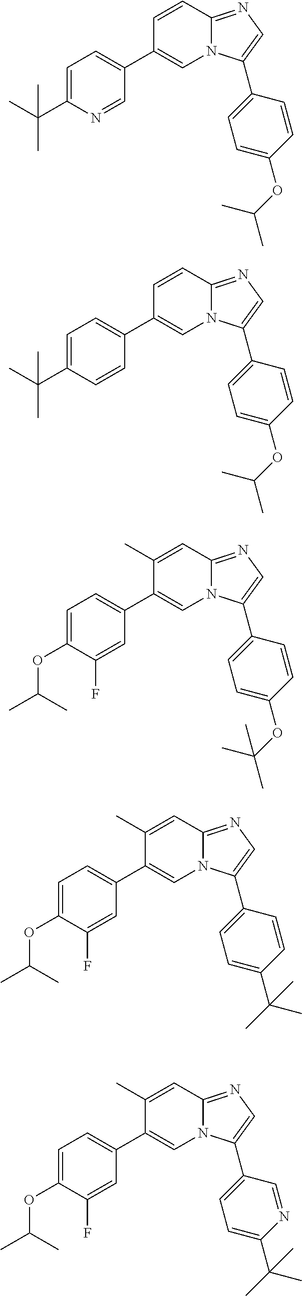

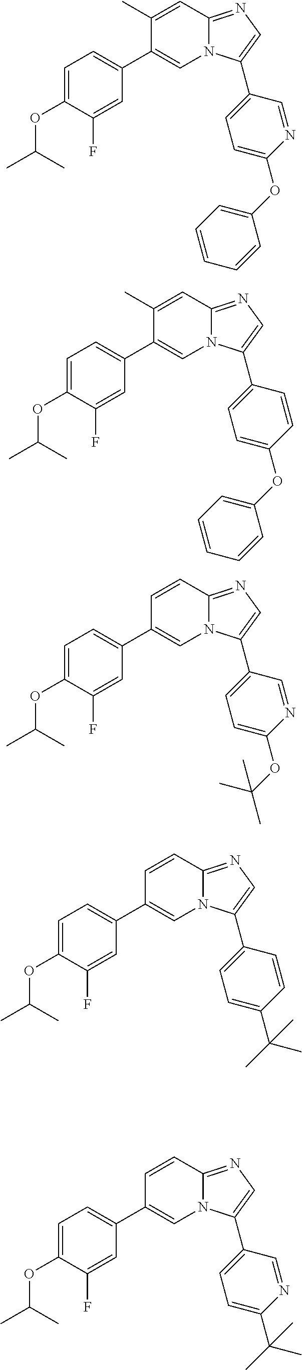

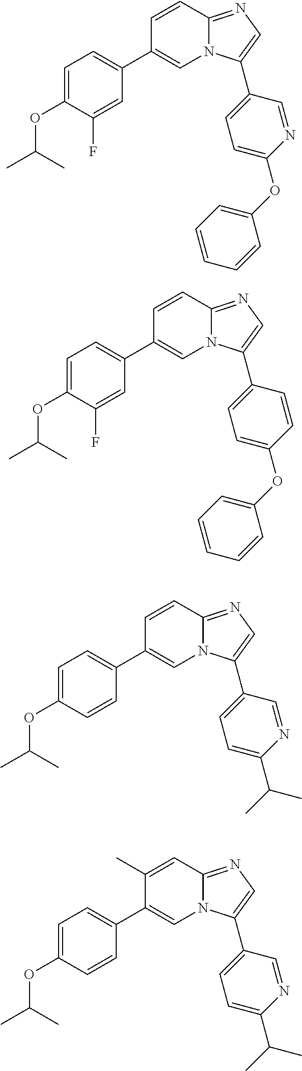

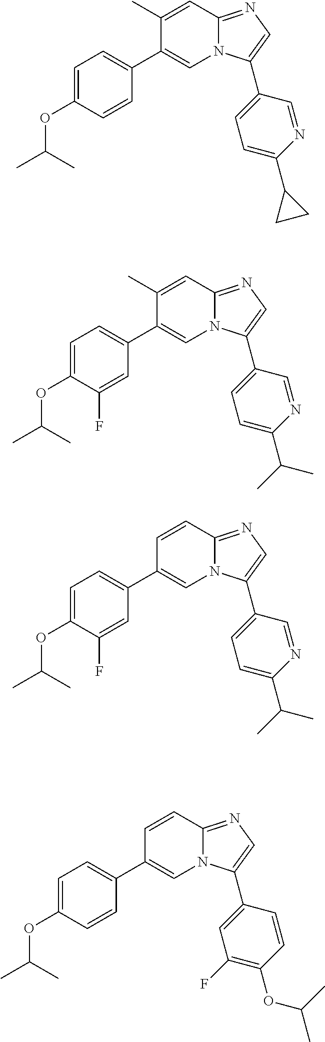

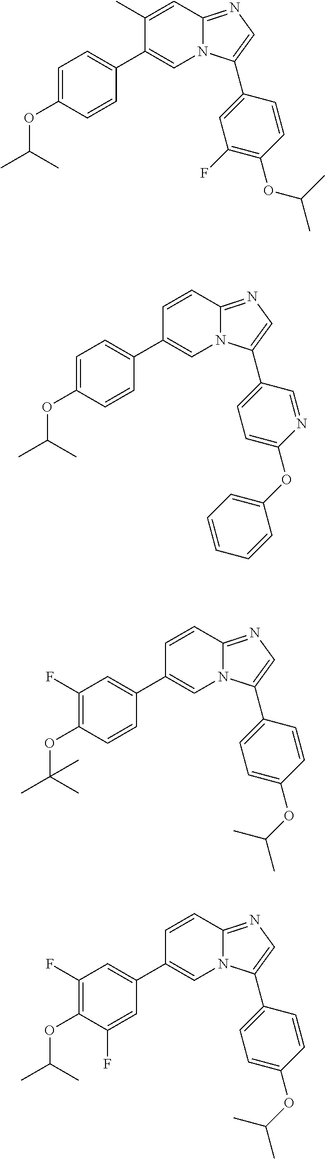

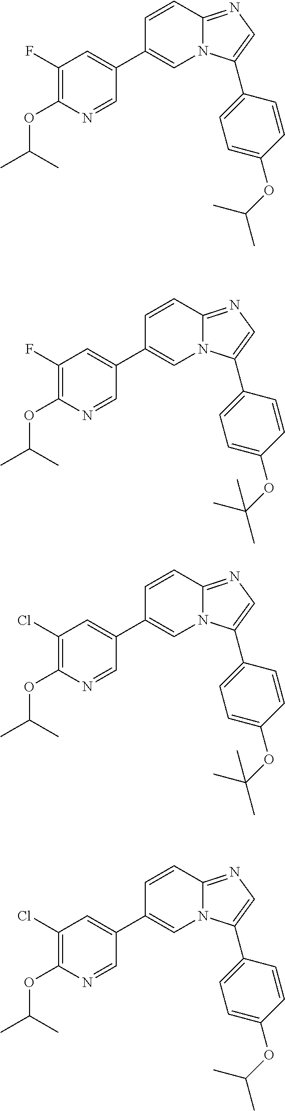

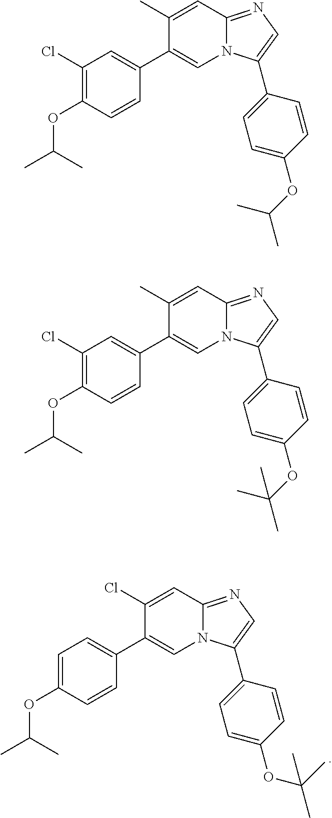 [SMILES: Cc1cc2ncc(-c3ccc(OC(C)C)cc3)n2cc1-c1ccc(OC(C)C)c(Cl)c1 ; Cc1cc2ncc(-c3ccc(OC(C)(C)C)cc3)n2cc1-c1ccc(OC(C)C)c(Cl)c1 ; CC(C)Oc1ccc(-c2cn3c(-c4ccc(OC(C)(C)C)cc4)cnc3cc2Cl)cc1 ; "."]

9. The method of claim 8, wherein the compound is selected from the group consisting of

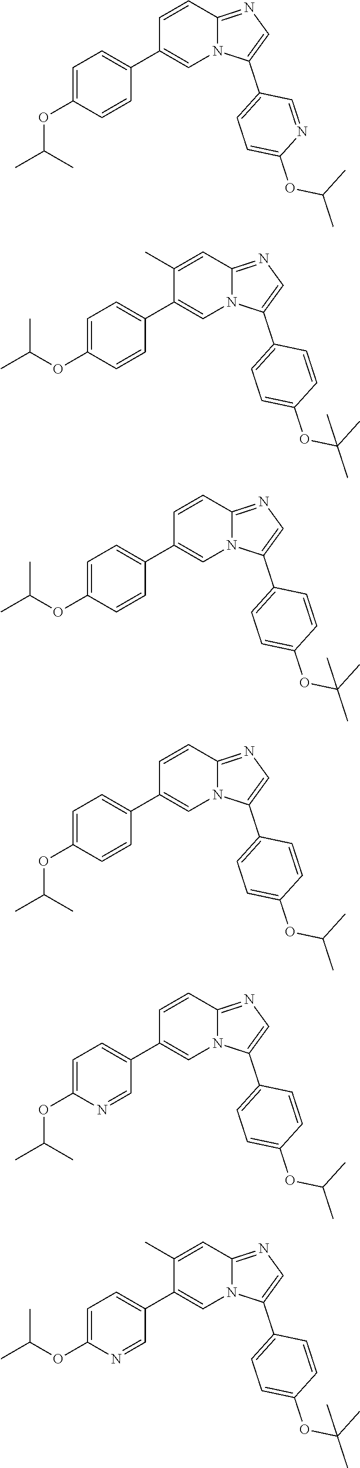

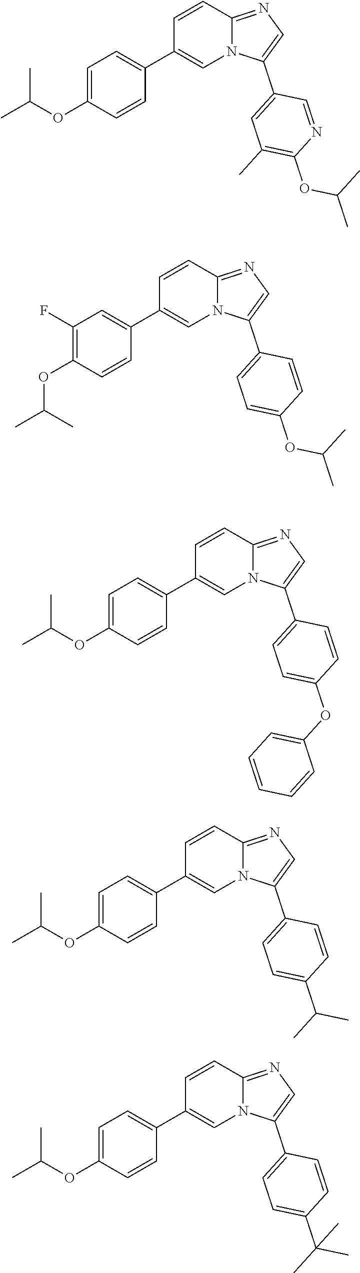

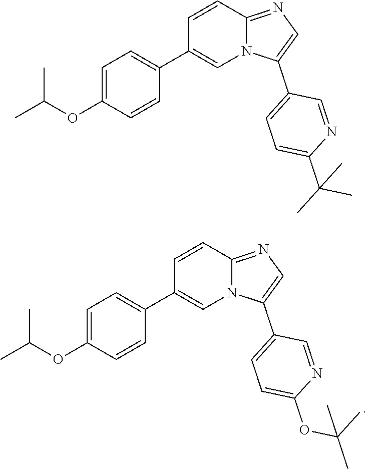

10. A method of treating a disease state in a mammal that is amenable to treatment by a sodium channel modulator, comprising administration of a therapeutically effective amount of a compound of Structural Formula I 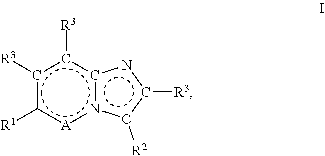
wherein A, R¹, R², and R³ have the definitions shown in claim 1.

11. The method of claim 10, wherein the disease state is selected from a cardiovascular disease, pain, a neurological disorder, an irritable bowel syndrome, or cancer.

12. The method of claim 11, wherein the disease state is a cardiovascular disease.

13. The method of claim 11, wherein the disease state is a neurological disorder.

14. The method of claim 12 wherein the cardiovascular disease is selected from the group consisting of ventricular tachycardia, ventricular fibrillation, ventricular arrhythmia, atrial arrhythmia, stable angina, unstable angina, Prinzmetal's angina, ischemia, recurrent ischemia, cerebrovascular ischemia, stroke, renal ischemia, ischemia and reperfusion injury, heart failure, congestive heart failure, systolic heart failure, diastolic heart failure, acute heart failure, myocardial infarction, reperfusion injury, intermittent claudication, peripheral artery disease, acute coronary syndrome, hypertrophic cardiomyopathy, and inherited arrhythmia syndromes.

15. The method of claim 14 wherein the inherited arrhythmia is selected from Brugada syndrome and long QT syndrome.

16. The method of claim 13, wherein a neurological disorder is selected from the group consisting of epilepsy or an epilepsy syndrome epileptic encephalopathy, focal temporal and frontal lobe epilepsies, severe myoclonic epilepsy of infancy, Dravet's syndrome, intractable childhood epilepsy with generalized tonic-clonic seizures, Rasmussen encephalitis, malignant migrating partial seizures of infancy, West syndrome, Ohtahara syndrome, Lennox-Gastaut syndrome, Landau-Kleffner syndrome, neurodegenerative diseases, dementia, Alzheimer's disease, neurodevelopmental disorders, autism, tuberous sclerosis complex, neuromuscular disorders, amyotropic lateral sclerosis, multiple sclerosis, periodic paralysis, myotonia), neural trauma, peripheral neuropathy, stroke, migraine, ataxia, seizures, and paralysis.

17. The method of claim 16 wherein the epilepsy syndrome is selected from benign neonatal-infantile familial seizures, simple febrile seizures, infantile spasms, generalized epilepsy with febrile seizures plus (GEFS+).

18. The method of claim 11 wherein the disease state is pain.

19. The method of claim 18 wherein the pain is selected from acute, chronic, inflammatory and neuropathic pain.

20. The method of claim 11 wherein the disease state is irritable bowel syndrome.

21. The method of claim 11 wherein the disease state is cancer.

22. The method of claim 21, wherein cancer is selected from the groups consisting of breast cancer, lung cancer, prostate cancer, pancreatic cancer, colon cancer, stomach cancer, ovary cancer, cervix cancer, bladder cancer, oral squamous cell cancer, endometrium cancer, connective tissue cancer, skin cancer, astrocytoma, lymphoma, neuroblastoma, mesothelioma, myeloma, hepatocellular carcinoma, leukemia, and osteosarcoma.

23. The method of claim 1, wherein a compound of Structural Formula I is administered with a compound that causes QT prolongation.

24. A compound of Structural Formulae Ia and Ib 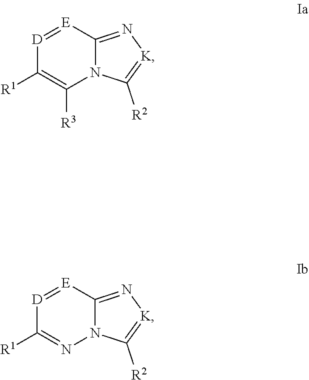
or a pharmaceutically acceptable salt, and a pharmaceutically acceptable carrier, diluent, or vehicle thereof, wherein:
D and E are independently selected from C—R³;
K is selected from CH and CD;
R¹ is selected from (Cato C₁₀) aryl and (C₂ to C₉) heteroaryl, wherein each of the said (Cato C₁₀) aryl and (C₂ to C₉) heteroaryl is substituted with at least one R^{4a} group, and wherein said (C2 to C₉) heteroaryl is C-attached;
with the proviso that R¹ is not selected from the group consisting of substituted 3-carbamoyl-2-phenyl-1-benzofuran-5-yl, substituted 1,3,4-oxadiazolyl, substituted 1,3,4-triazolyl, substituted 1,3,4-thiadiazolyl, substituted oxazoyl, substituted thiazoyl, substituted 1H-pyrazol-4-yl, substituted 1H-pyrazol-5-yl, optionally substituted 1-phenyl-1H-imidazol-5-yl, 4-{[(2-aminoethyl)amino]methyl}phenyl, (2-amino-1,3-benzoxazol)-5-yl; (2-amino-1,3-benzoxazol)-4-yl, 2-chloropyridyl-3-yl, 2-methylpyridinyl-4-yl, 2-fluoropyridyl-4-yl, 6-aminopyridyl-3-yl, 6-methoxypyridyl-3-yl, pyridyl-4-yl-N-oxide, 3,4-difluorphenyl, substituted 1H-pyrrol-3-yl, 6-methylpyridyl-3-yl, 2-methoxypyridyl-3-yl, 6-cyanopyridyl-3-yl, pyridyl-4-yl, 4-(methylsulfonyl)phenyl, thien-3-yl, and fur-3-yl;
R² is selected from the group consisting of 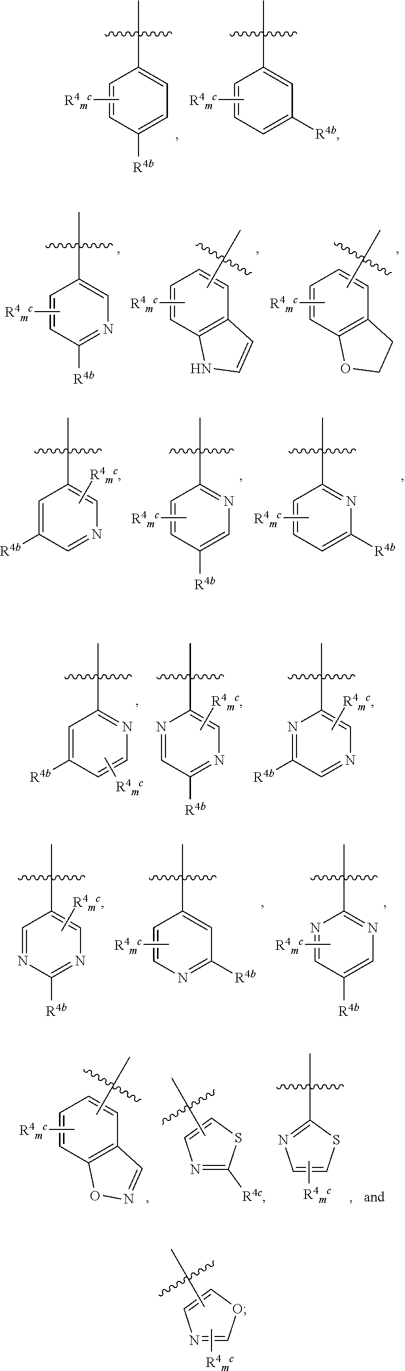
each of the R³ is independently selected from H, deuterium, halogen, cyano, CF₃, (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₆) cycloheteroalkyl, —O—(C₃ to C₁₀) cycloalkyl, —O—(C₅ to C₁₀) cycloalkenyl, —O—(C₂ to C₉) cycloheteroalkyl, C(O)R⁵, —S(O)ₘR⁵, —S(O)ₘNR^{6a}R^{6b}, —NR^{6a}S(O)ₘR⁵, —(CH₂)ₙC(O)OR⁵, —OC(O)R⁵, and —NR^{6c}C(O)NR^{5a}R^{5b}, wherein
each of the said (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, and —O—(C₂ to C₉) cycloheteroalkyl is optionally substituted with at least one R⁷ group;
R^{4a} is independently selected from halogen, cyano, OH, CF₃, (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, (C₂ to C₉) heteroaryl, —C(O)R⁵, —NR^{6a}R^{6b}, —S(O)ₘR⁵, —S(O)ₘNR^{6a}R^{6b}, —NR^{6a}S(O)ₘR⁵, —OC(O)R⁵, and —NR^{6c}C(O)NR^{6a}R^{6b}, wherein
each of the said (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₅ to C₁₀) cycloalkyl, (C₅ to C₁₀) cycloalkenyl, (C₂ to C₆) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, and (C₂ to C₆) heteroaryl is optionally substituted with at least one R⁷ group;
R^{4b} is selected from the group consisting of
(C₂ to C₆) alkyl, (C₂ to C₆) alkenyl, (C₂ to C₆) alkynyl, —O—(C₂ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, —O—(C₅ to C₁₀) cycloalkenyl, (C₆ to C₁₀) aryl, isopropoxy, cyclopropoxy, tert-butoxy, cyclopropylmethoxy, (2-hydroxypropan)-2-yl, (1-hydroxycyclopropan)-1-yl, (1-hydroxycyclobutan)-1-yl, trifluoromethoxy, trifluoromethyl, difluoromethoxy, trifluoroethoxy, methoxyethoxy, phenoxy, benzyloxy, difluoromethyl, cyclopropyl, n-propyloxy, n-butyloxy, isobutyloxy, sea-butyloxy, cyclobutyloxy, cyclopentyloxy, (1-hydroxycyclopentan)-1-yl, hydroxyethoxy, ethoxyethoxy, isopropoxyethoxy, isopropyl, n-propyl, n-butyl, sec-butyl, tert-butyl, cyclobutyl, cyclopentyl, propanoyl, isobutanoyl, sec-butanoyl, tert-butanoyl, cyclopropanoyl, cyclobutanoyl, and cyclopentanoyl;
wherein each of the said (C₂ to C₆) alkyl, (C₂ to C₆) alkenyl, (C₂ to C₆) alkynyl, —O—(C₂ to C₆) alkyl, —O—(C₃ to C₁₀) cycloalkyl, —O—(C₆ to C₁₀) aryl, (C₃ to C₁₀) cycloalkyl, (C₅ to C₁₀) cycloalkenyl, —O—(C₅ to C₁₀) cycloalkenyl, and (C₆ to C₁₀) aryl is optionally substituted with at least one R⁷ group;
R^{4c} is independently selected from hydrogen, deuterium, halogen, cyano, OH, CF₃, (C₁ to C₆) alkyl, (C₂ to C₆) alkenyl, (C₂ to C₆) alkynyl, —O—(C₂ to C₆) alkyl, —O—(C₂ to C₆) alkenyl, —O—(C₂ to C₆) alkynyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₅ to C₁₀) cycloalkenyl, —O—(C₂ to C₆) cycloheteroalkyl, (C₆ to C₁₀) aryl, (C₂ to C₉) heteroaryl, —C(O)R⁵, —NR^{6a}R^{6b}, —S(O)ₘR⁵, —S(O)ₘNR^{6a}R^{6b}, —NR^{6a}S(O)ₘR⁵, —OC(O)R⁵, —NR^{6a}C(O)R⁵, and —NR^{6c}C(O)NR^{6a}R^{6b}, wherein
each of the said (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₃ to C₁₀) cycloalkyl, —O—(C₆ to C₁₀) aryl, (C₃ to C₁₀) cycloalkyl, (C₅ to C₁₀) cycloalkenyl, (C₂ to C₉) cycloheteroalkyl, —O—(C₅ to C₁₀) cycloalkenyl, —O—(C₂ to C₉) cycloheteroalkyl, (C₆ to C₁₀) aryl, and (C₂ to C₉) heteroaryl is optionally substituted with at least one R⁷ group;
each of the R⁵ is independently selected from hydrogen, deuterium, (C₁ to C₆) alkyl, (C₂ to C₉) alkenyl, (C₂ to C₆) alkynyl, (C₃ to C₁₀) cycloalkyl, (C₅ to C₁₀) cycloalkenyl, (C₆ to C₁₀) aryl, and (C₂ to C₉) heteroaryl, wherein
each of the said (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₆ to C₁₀) aryl, and (C₂ to C₆) heteroaryl is optionally substituted with at least one R⁷ group;
each of the R^{6a}, R^{6b}, and R^{6c} are independently selected from hydrogen, deuterium, (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₂ to C₆) cycloheteroalkyl, (C₆ to C₁₀) aryl, and (C₂ to C₆) heteroaryl,
or R₆ₐ and R^{6b} may be taken together with the nitrogen atom to which they are attached to form a 4 to 8 membered cycloheteroalkyl ring, wherein
said 4 to 8 membered cycloheteroalkyl ring has 1 to 3 ring heteroatoms selected from the group consisting of N, O, and S, and wherein
the said 4 to 8 membered cycloheteroalkyl ring is optionally substituted with at least one R⁷ group;
R⁷ is independently selected from hydrogen, deuterium, OH, CF₃, (C₁ to C₆) alkyl, (C₂ to C₉) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₅ to C₁₀) cycloalkyl, (C₅ to C₁₀) cycloalkenyl, (C₂ to C₆) cycloheteroalkyl, —O—(C₃ to C₁₀) cycloalkyl, —O—(C₅ to C₁₀) cycloalkenyl, (C₆ to C₁₀) aryl, (C₂ to C₆) heteroaryl, —C(O)R⁸, —C(O)NR^{9a}R^{9b}, —NR^{9a}R^{9b}, —S(O)ₘR⁵, —S(O)ₘNR^{9a}R^{9b}, —NR^{9a}S(O)ₘR⁸, —(CH₂)ₙC(O)OR⁸, —(CH₂)ₙC(O)N(R^{9a}R^{9b}), —OC(O)R⁸, —NR^{9a}C(O)R⁸, and —NR^{9a}C(O)N(R^{9a}R^{9b}), wherein
each of the said (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₅ to C₁₀) cycloalkyl, (C₅ to C₁₀) cycloalkenyl, (C₂ to C₆) cycloheteroalkyl, —O—(C₃ to C₁₀) cycloalkyl, —O—(C₅ to C₁₀) cycloalkenyl, (Ceto C₁₀) aryl, and (C₂ to C₆) heteroaryl is optionally substituted with at least one R¹⁰ group;
each of the R⁶ is independently selected from hydrogen, deuterium, (C₁ to C₆) alkyl, (C₂ to C₉) alkenyl, (C₂ to C₆) alkynyl, (C₃ to C₁₀) cycloalkyl, (C₅ to C₁₀) cycloalkenyl, (C₂ to C₉) cycloheteroalkyl, (C₆ to C₁₀) aryl, and (C₂ to C₆) heteroaryl, wherein
each of the said (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₂ to C₆) cycloheteroalkyl, (C₆ to C₁₀) aryl, and (C₂ to C₆) heteroaryl is optionally substituted with at least one R¹⁰ group;
each of the R^{9a}, R^{9b}, and R^{9c} are independently selected from hydrogen, deuterium, (C₁ to C₆) alkyl, (C₂ to C₆) alkenyl, (C₂ to C₆) alkynyl, (C₃ to C₁₀) cycloalkyl, (C₅ to C₁₀) cycloalkenyl, (C₂ to C₆) cycloheteroalkyl, (C₆ to C₁₀) aryl, and (C₂ to C₆) heteroaryl,
or R^{9a} and R^{9b} may be taken together with the nitrogen atom to which they are attached to form a 4 to 8 membered cycloheteroalkyl ring, wherein
said 4 to 8 membered cycloheteroalkyl ring has 1 to 3 ring heteroatoms selected from the group consisting of N, O, and S, and wherein
the said 4 to 8 membered cycloheteroalkyl ring is optionally substituted;
R¹⁰ is independently selected from hydrogen, deuterium, halogen, cyano, OH, CF₃, (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloakyl, (C₅ to C₁₀) cycloalkenyl, (C₂ to C₆) cycloheteroalkyl, —O—(C₃ to C₁₀) cycloalkyl, —O—(C₅ to C₁₀) cycloalkenyl, —O—(C₂ to C₆) cycloheteroalkyl, (C₆ to C₁₀) aryl, and (C₂ to C₆) heteroaryl, wherein
each of the said (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(C₆ to C₁₀) aryl, (C₃ to C₁₀) cycloalkyl, (C₅ to C₁₀) cycloalkenyl, (C₂ to C₆) cycloheteroalkyl, —O—(C₃ to C₁₀) cycloalkyl, —O—(C₅ to C₁₀) cycloalkenyl, —O—(C₂ to C₉) cycloheteroalkyl, (C₆ to C₁₀) aryl, and (C₂ to C₆) heteroaryl is optionally substituted with hydrogen, deuterium, halogen, cyano, —CD₃, OH, CF₃, (C₁ to C₆) alkyl, (C₂ to C₆) alkenyl, (C₂ to C₆) alkynyl, —O—(C₁ to C₆) alkyl, —O—(C₂ to C₆) alkenyl, —O—(C₂ to C₆) alkynyl, —O—(Cato C₁₀) aryl, (C₃ to C₁₀) cycloalkyl, (C₅ to C₁₀) cycloalkenyl, (C₂ to C₉) cycloheteroalkyl, —O—(C₃ to C₁₀) cycloalkyl, —O—(C₅ to C₁₀) cycloalkenyl, —O—(C₂ to CY) cycloheteroalkyl, (C₆ to C₁₀) aryl, and (C₂ to C₉) heteroaryl;
m is 0, 1, or 2;
and n is 1, 2, 3, 4, 5, or 6;
with the proviso that
the following compounds are excluded:

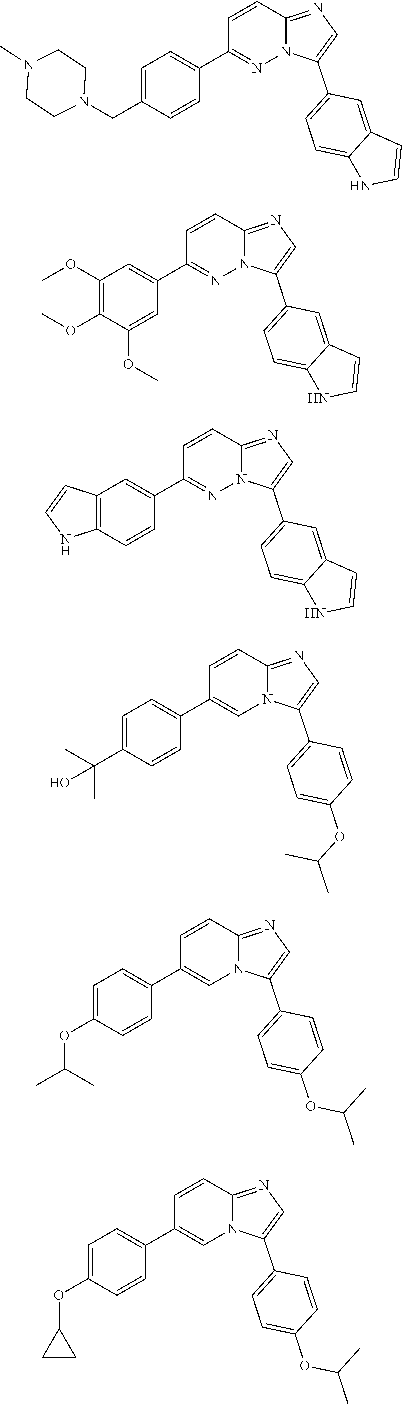 [SMILES: CN1CCN(Cc2ccc(-c3ccc4ncc(-c5ccc6[nH]ccc6c5)n4n3)cc2)CC1 ; COc1cc(-c2ccc3ncc(-c4ccc5[nH]ccc5c4)n3n2)cc(OC)c1OC ; c1cc2cc(-c3ccc4ncc(-c5ccc6[nH]ccc6c5)n4n3)ccc2[nH]1 ; CC(C)Oc1ccc(-c2cnc3ccc(-c4ccc(C(C)(C)O)cc4)cn23)cc1 ; CC(C)Oc1ccc(-c2ccc3ncc(-c4ccc(OC(C)C)cc4)n3c2)cc1 ; CC(C)Oc1ccc(-c2cnc3ccc(-c4ccc(OC5CC5)cc4)cn23)cc1]

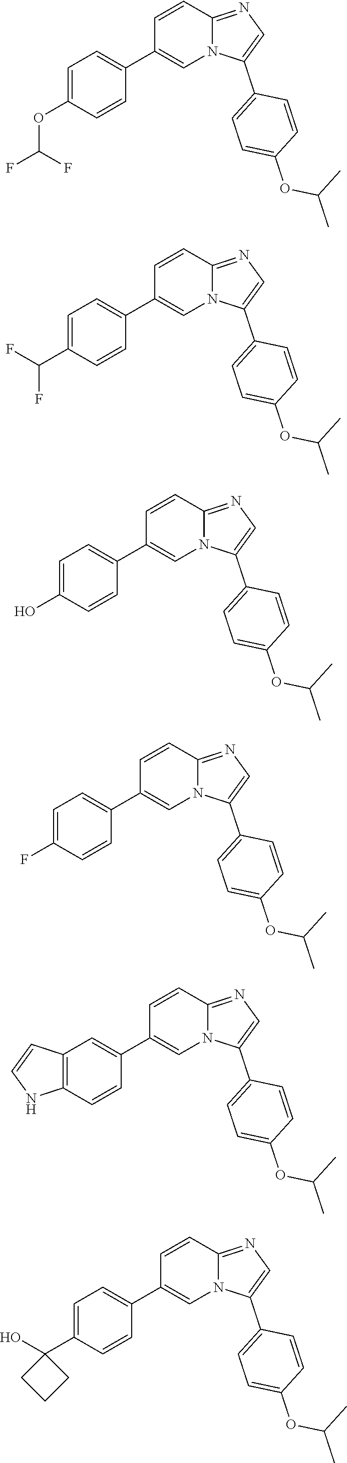 [SMILES: CC(C)Oc1ccc(-c2cnc3ccc(-c4ccc(OC(F)F)cc4)cn23)cc1 ; CC(C)Oc1ccc(-c2cnc3ccc(-c4ccc(C(F)F)cc4)cn23)cc1 ; CC(C)Oc1ccc(-c2cnc3ccc(-c4ccc(O)cc4)cn23)cc1 ; CC(C)Oc1ccc(-c2cnc3ccc(-c4ccc(F)cc4)cn23)cc1 ; CC(C)Oc1ccc(-c2cnc3ccc(-c4ccc5[nH]ccc5c4)cn23)cc1 ; CC(C)Oc1ccc(-c2cnc3ccc(-c4ccc(C5(O)CCC5)cc4)cn23)cc1]

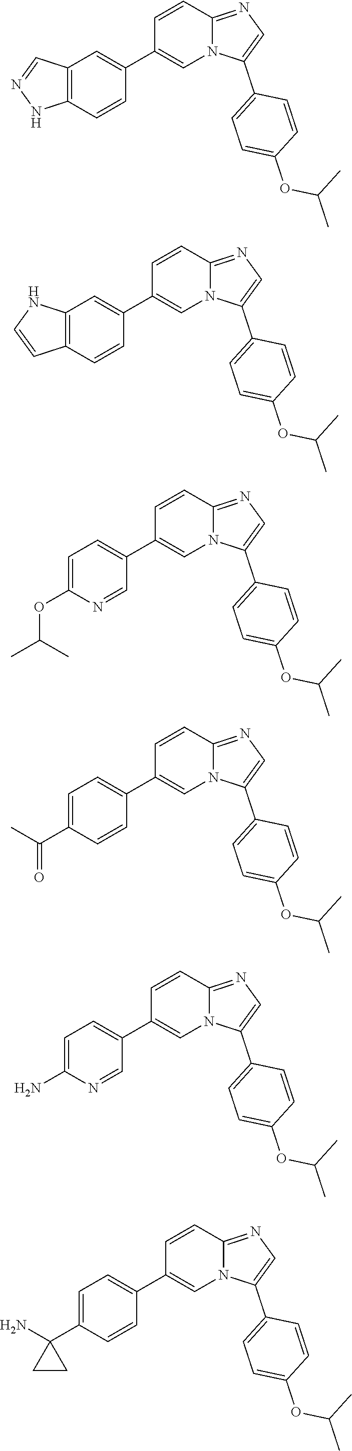

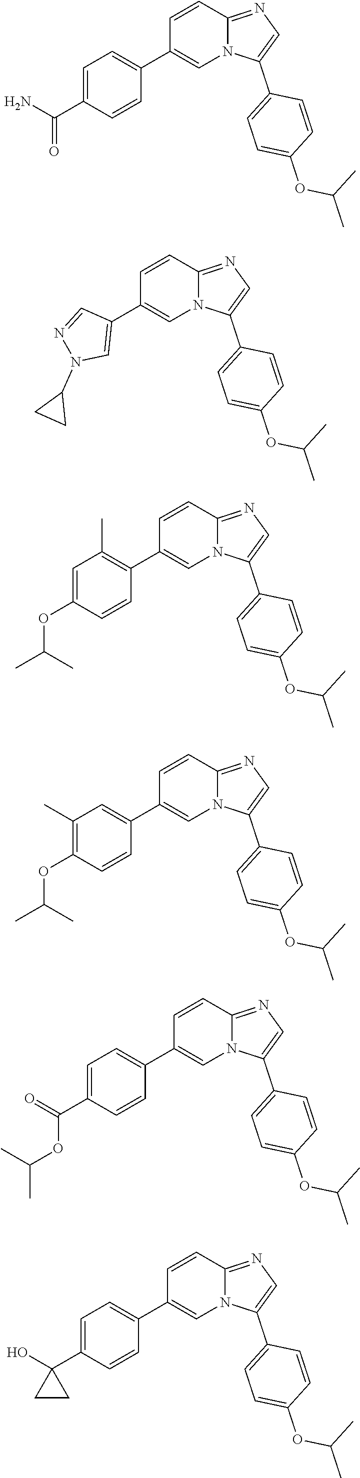

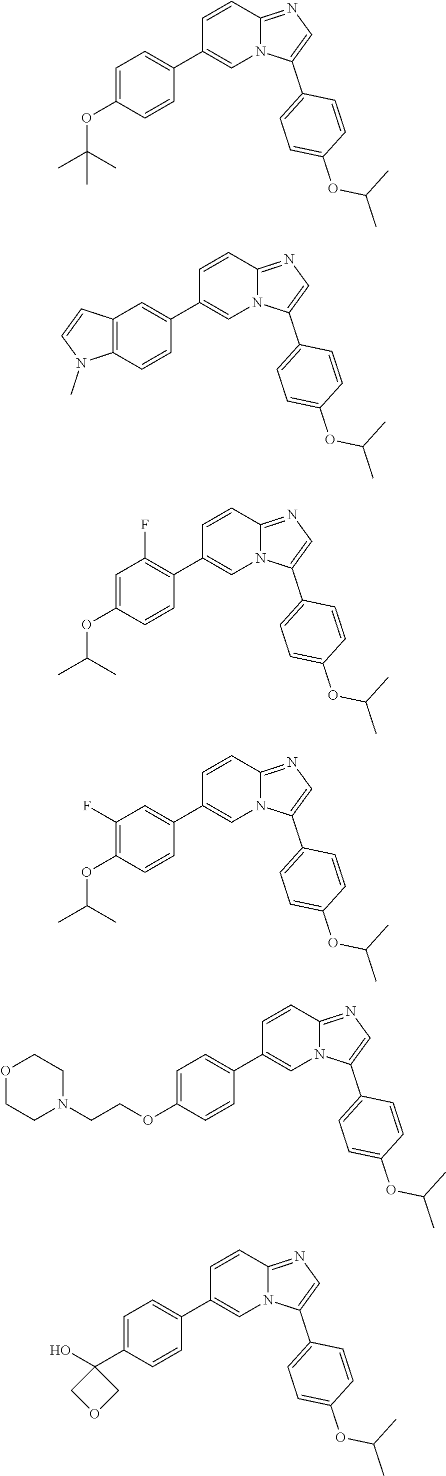

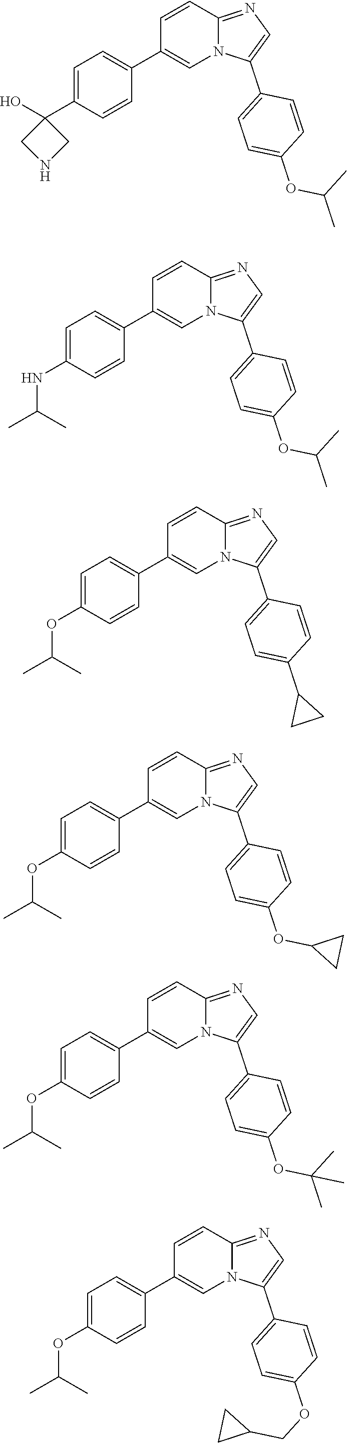

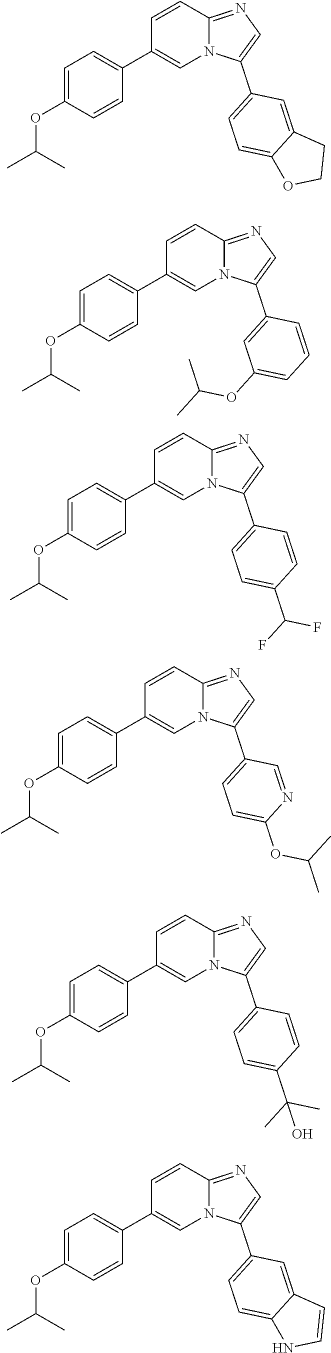

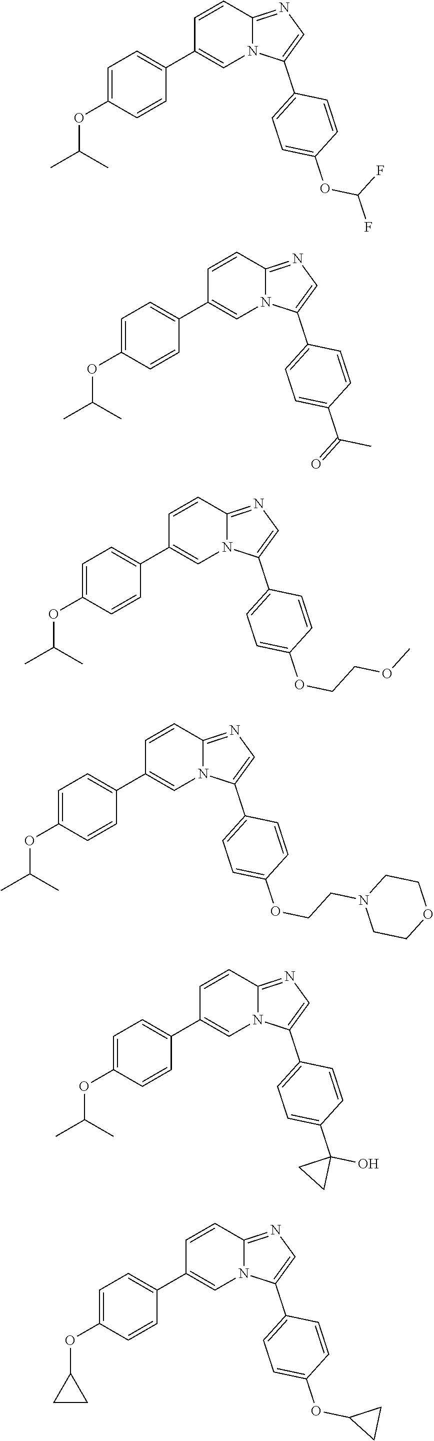

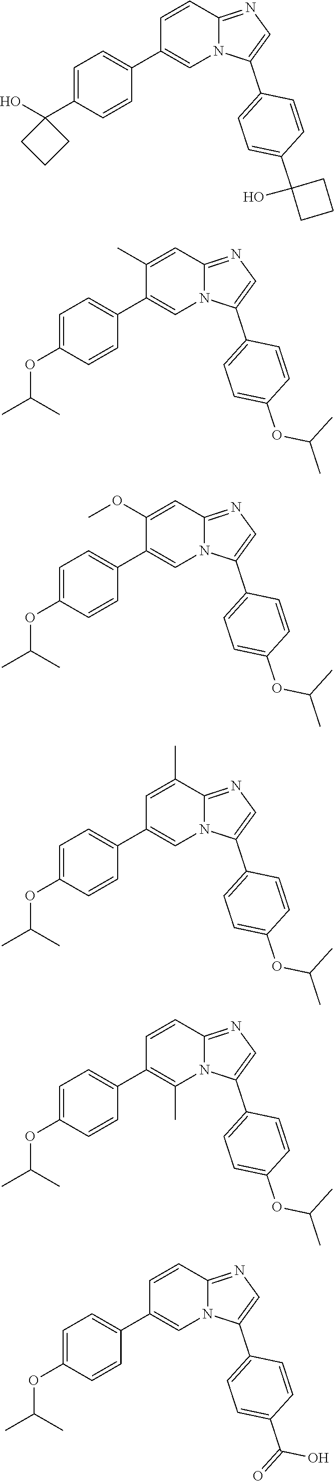

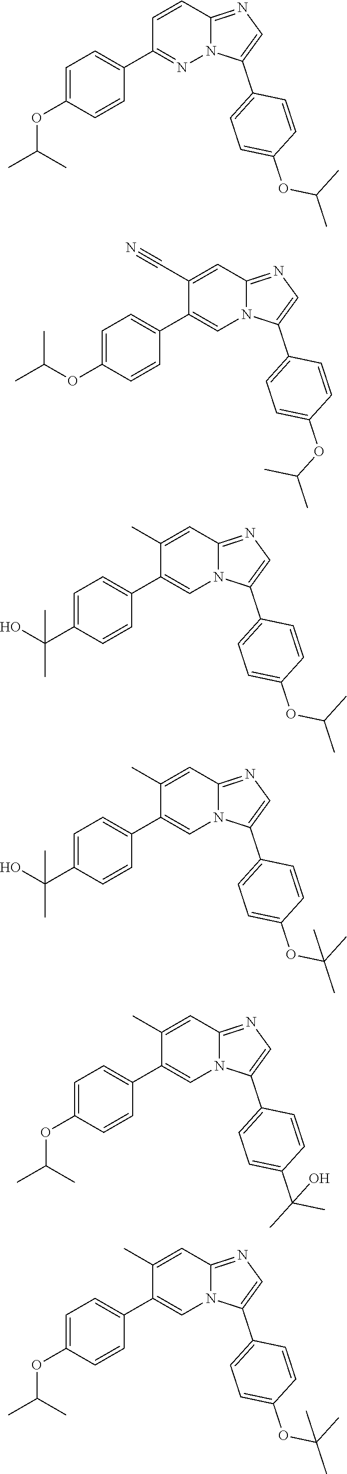

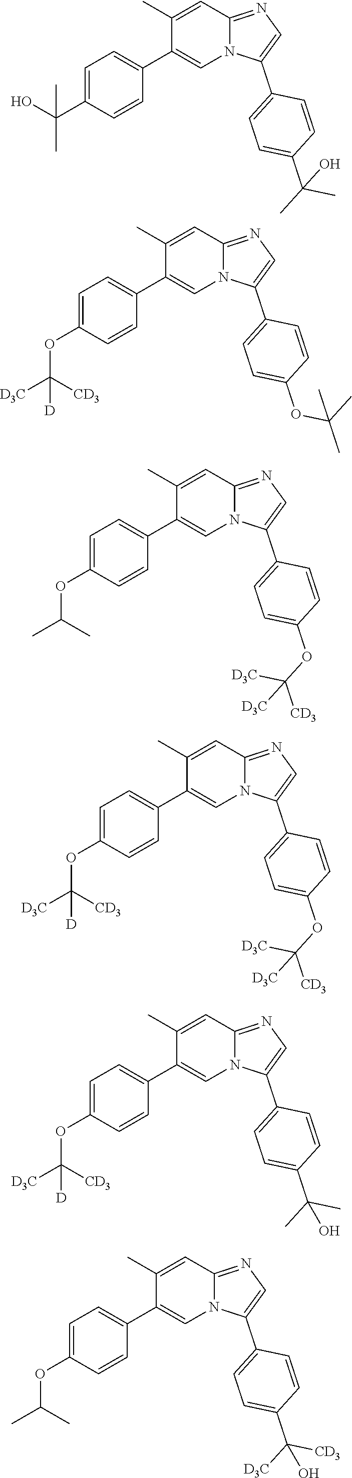

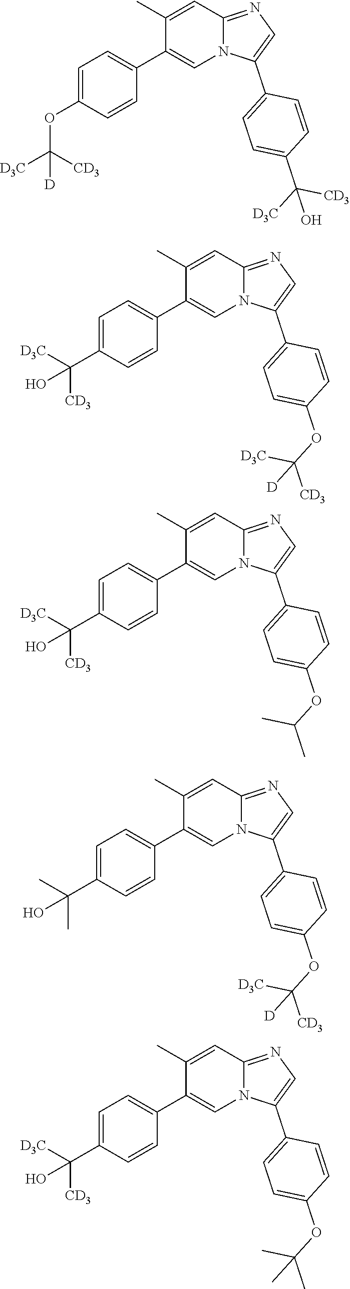

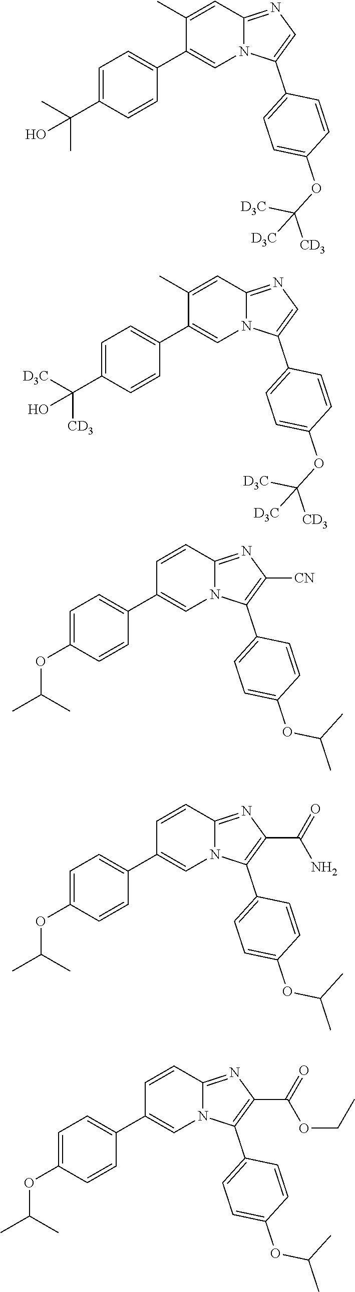

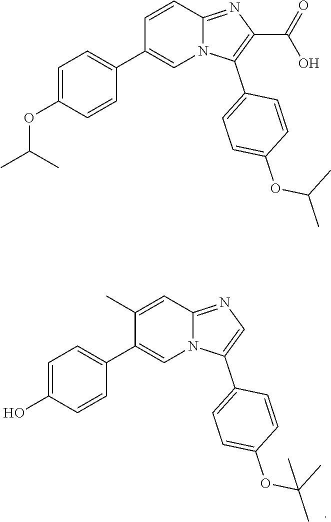 [SMILES: CC(C)Oc1ccc(-c2ccc3nc(C(=O)O)c(-c4ccc(OC(C)C)cc4)n3c2)cc1 ; Cc1cc2ncc(-c3ccc(OC(C)(C)C)cc3)n2cc1-c1ccc(O)cc1]

25. The compound of claim 24, wherein R² is selected from the group consisting of

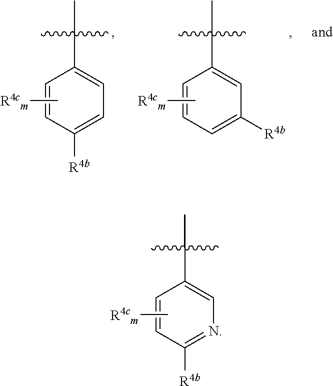

26. The compound of claim 25, wherein R² is selected from the group consisting of

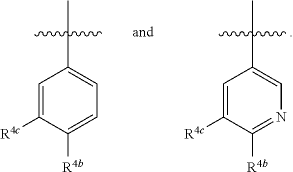

27. The compound of claim 26, wherein R^{4b} is isopropoxy, tert-butoxy, phenoxy, isopropyl, or tert-butyl.

28. The compound of claim 25, wherein a compound is selected from

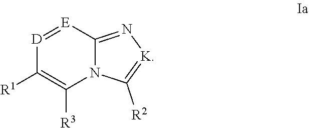

29. The compound of claim 28, wherein D is C—R³ and R³ is hydrogen, deuterium, C—CH₃, or C-CD₃.

30. The compound of claim 25, wherein a compound is selected from

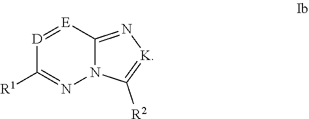

31. The compound of claim 30, wherein D is C—R³ and R³ is hydrogen, deuterium, C—CH₃, or C-CD₃.

32. The compound of claim 25, wherein R¹ is selected from the group consisting of

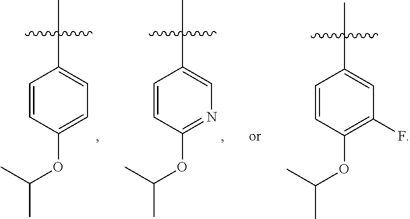 [SMILES: *c1ccc(OC(C)C)cc1 ; *c1ccc(OC(C)C)nc1 ; *c1ccc(OC(C)C)c(F)c1]

33. A compound selected from the group consisting of

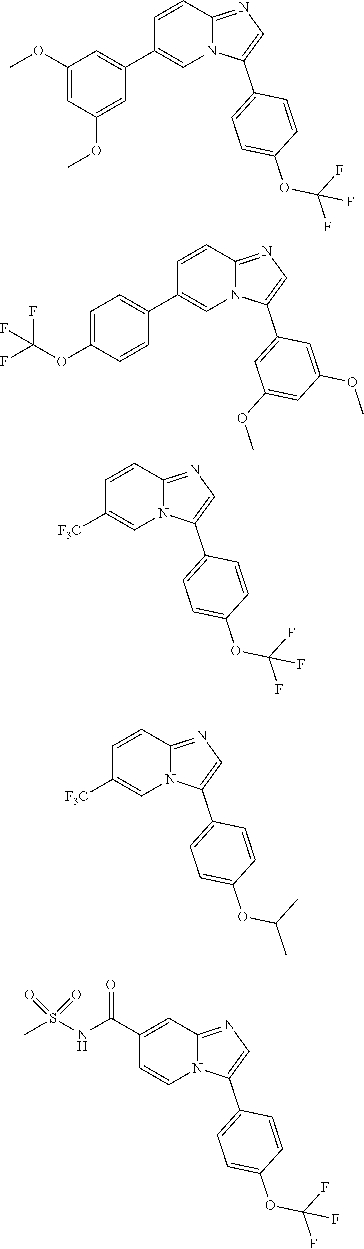

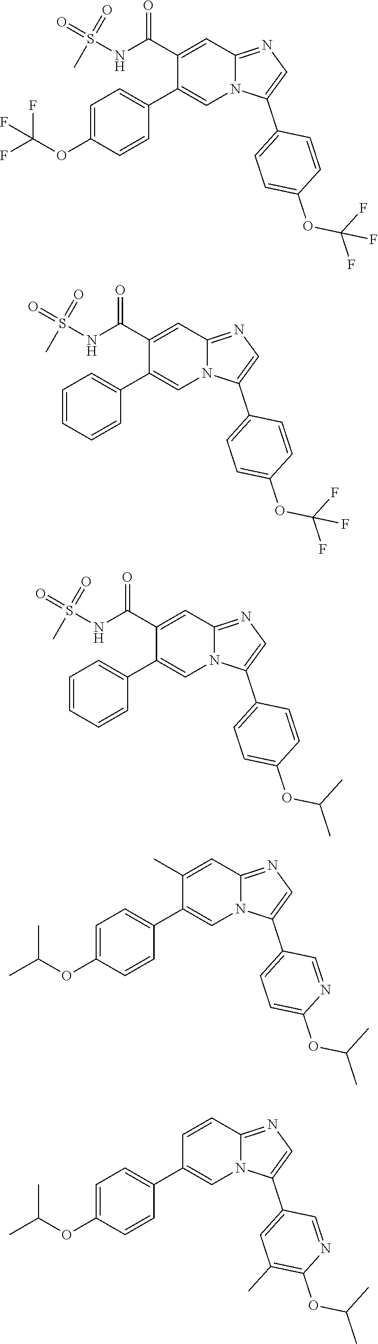

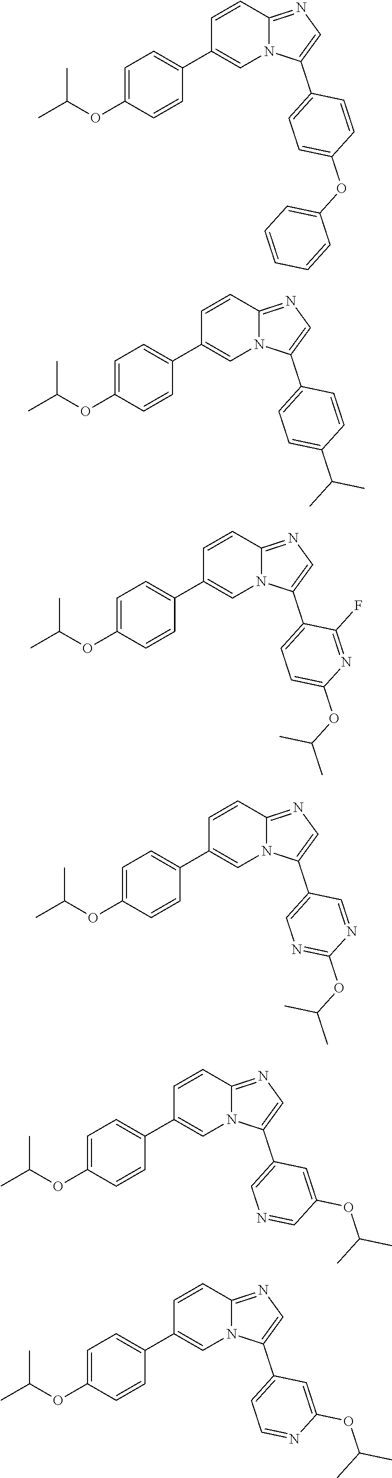

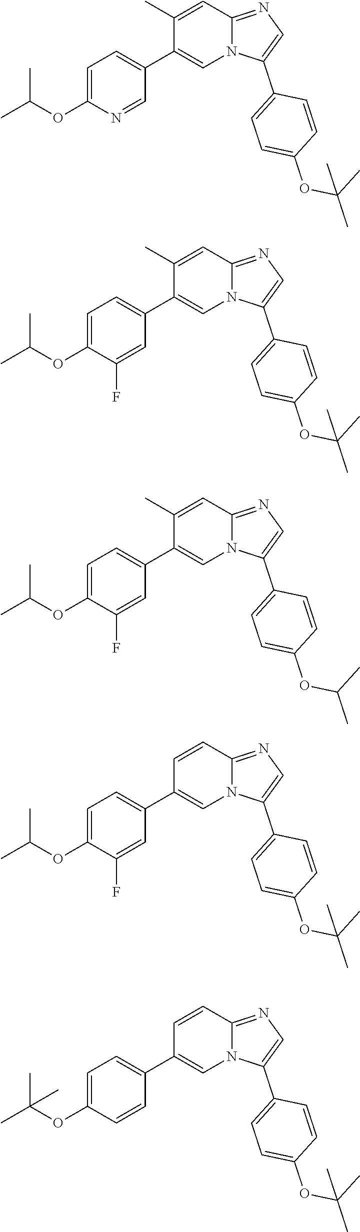

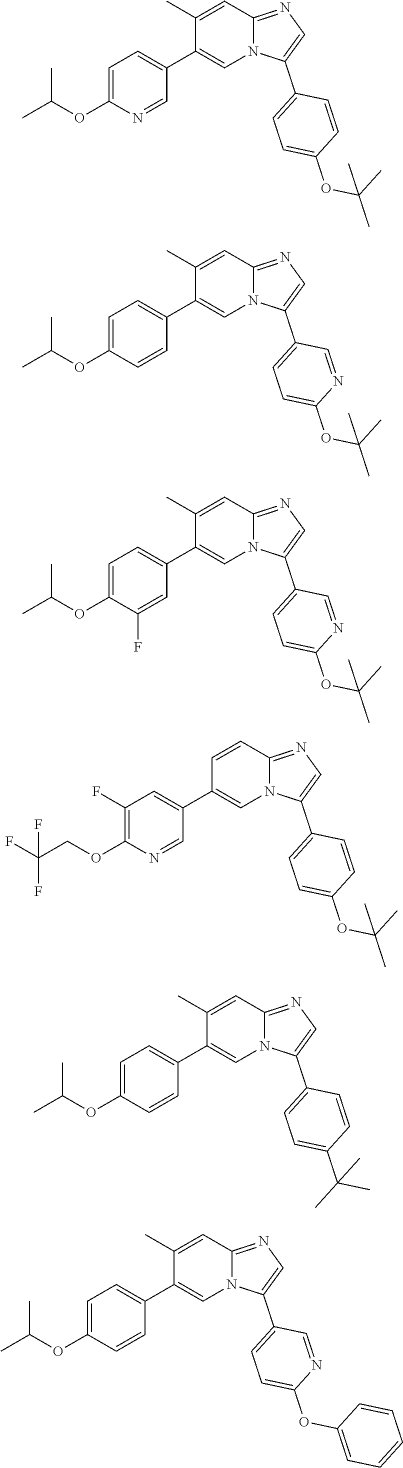

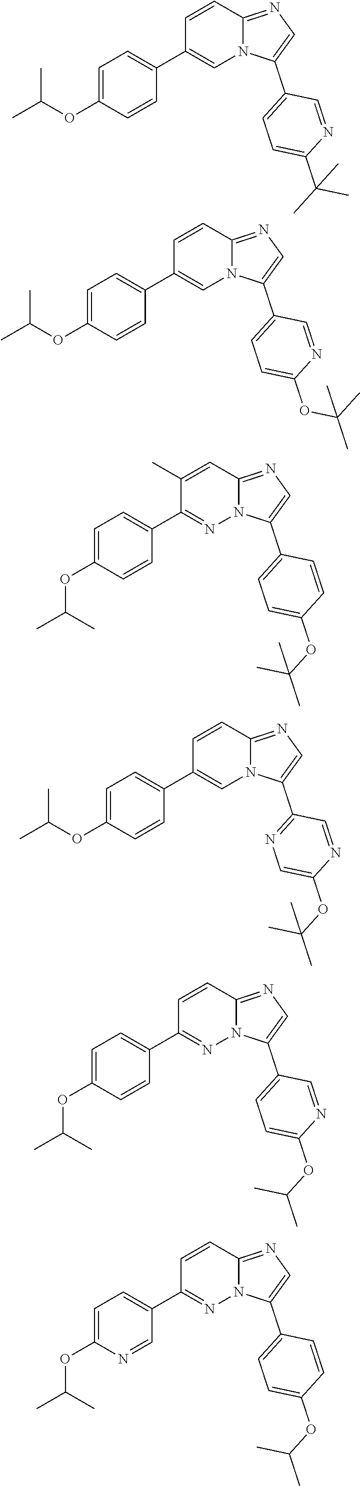

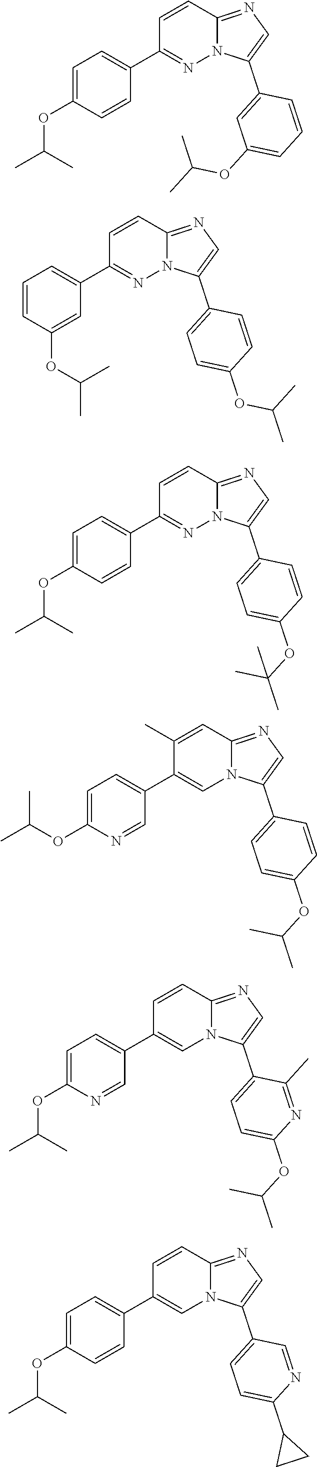

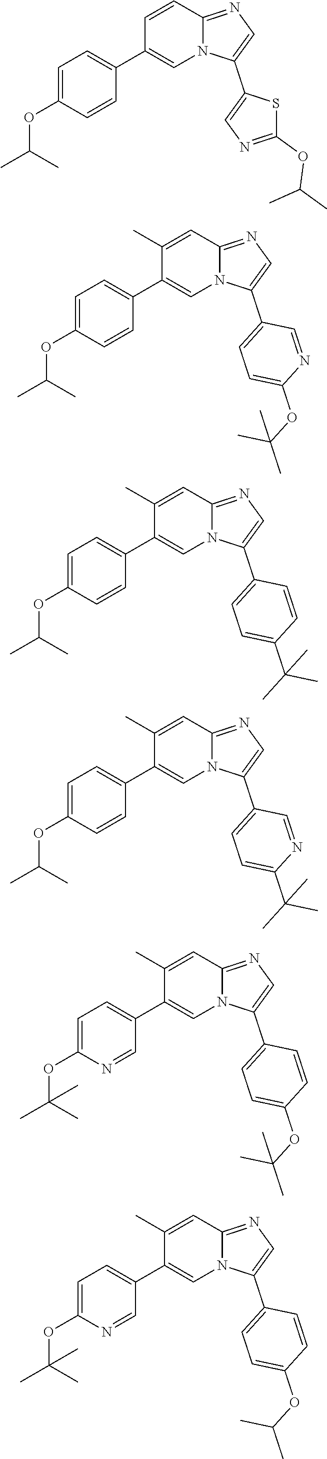

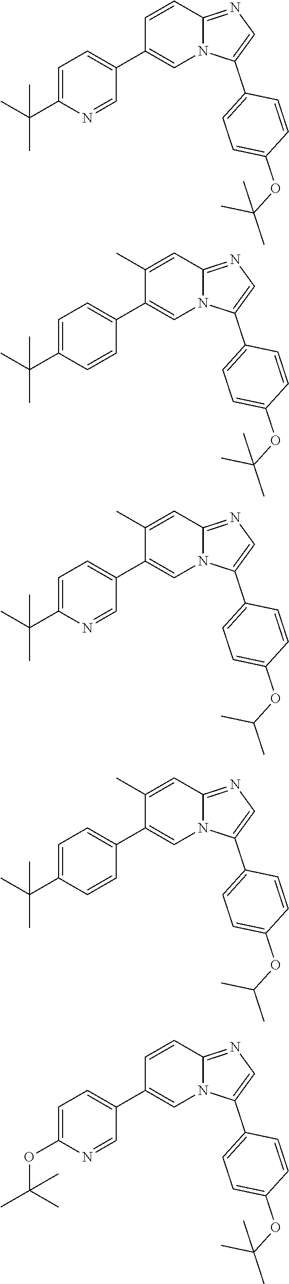

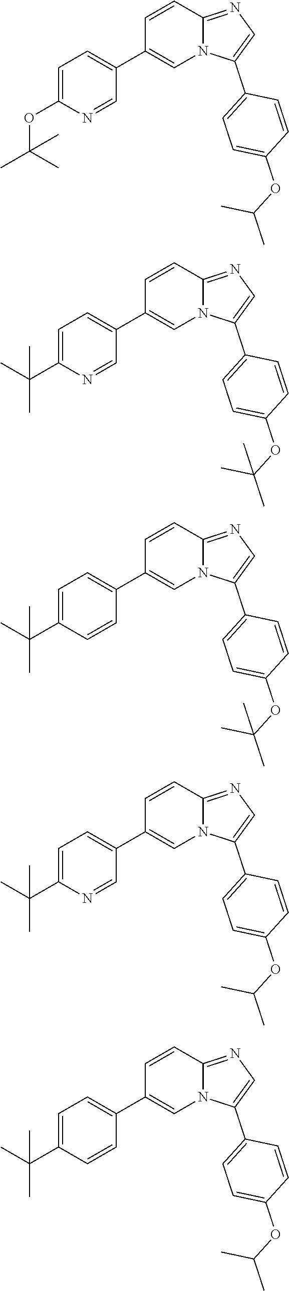

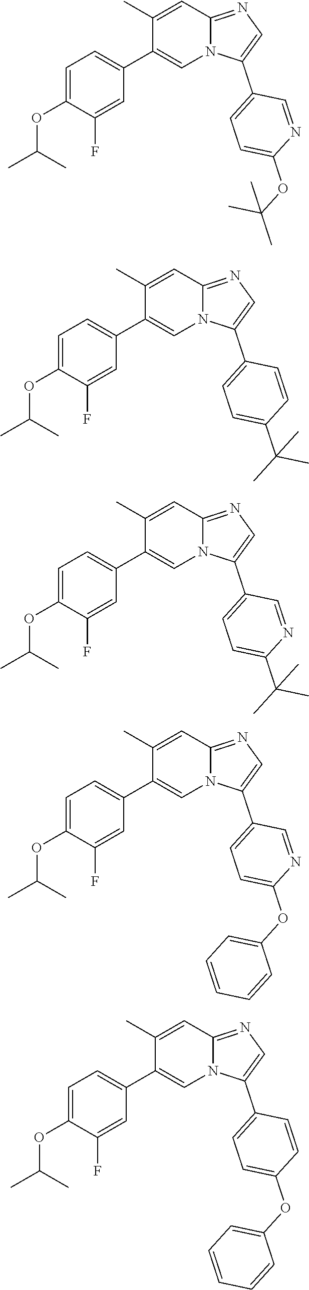

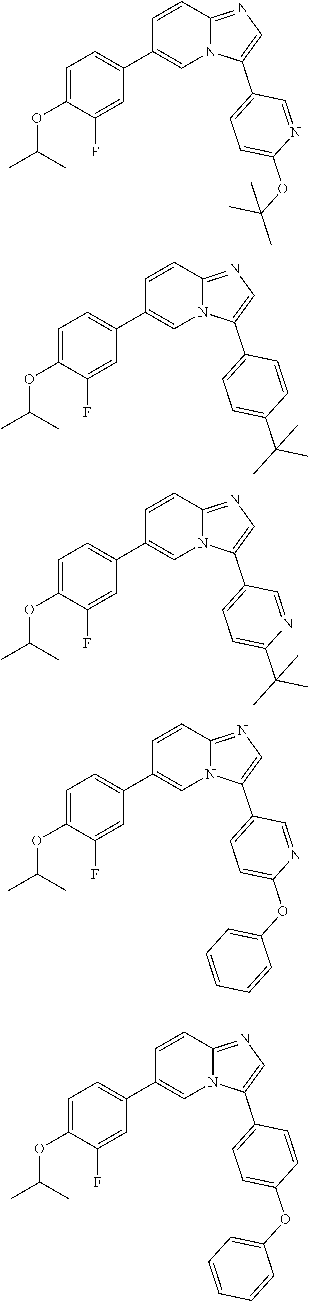

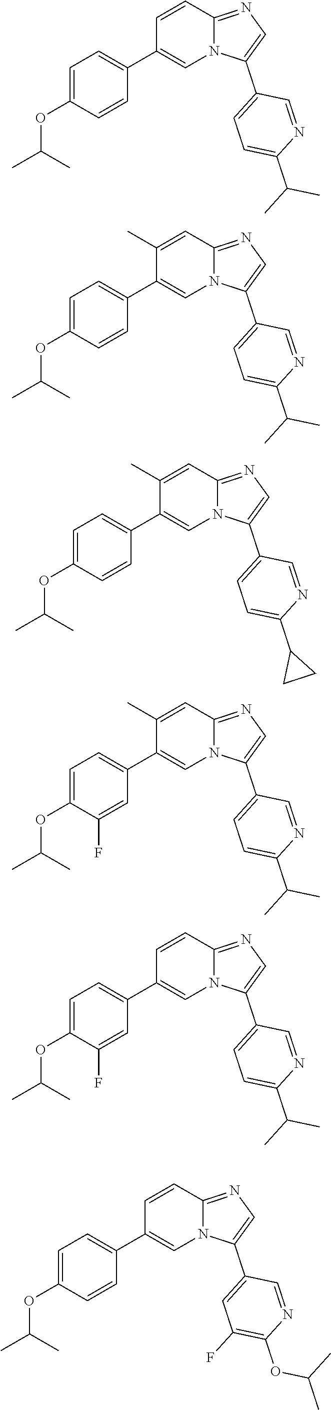

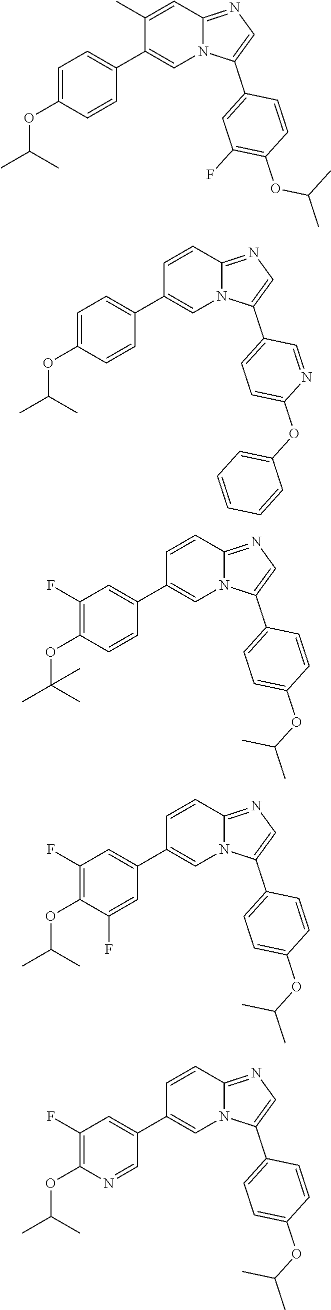

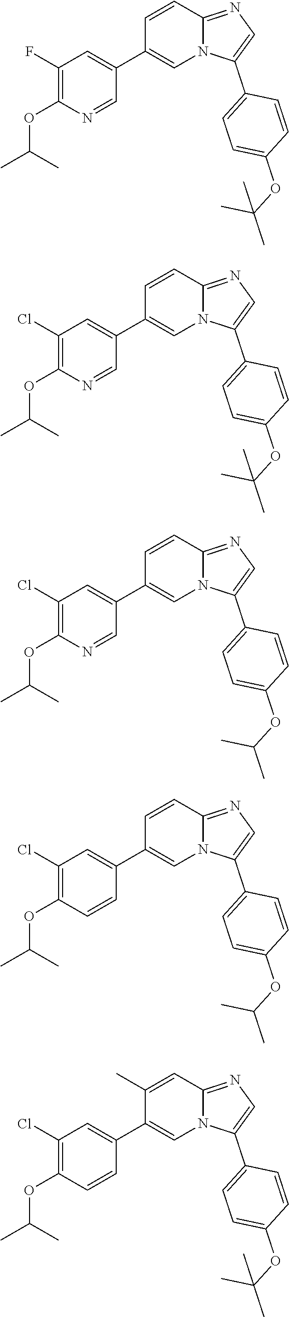

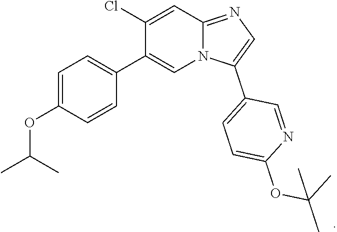 [SMILES: CC(C)Oc1ccc(-c2cn3c(-c4ccc(OC(C)(C)C)nc4)cnc3cc2Cl)cc1]

34. The compound of claim 33 wherein the compound is selected from the group consisting of

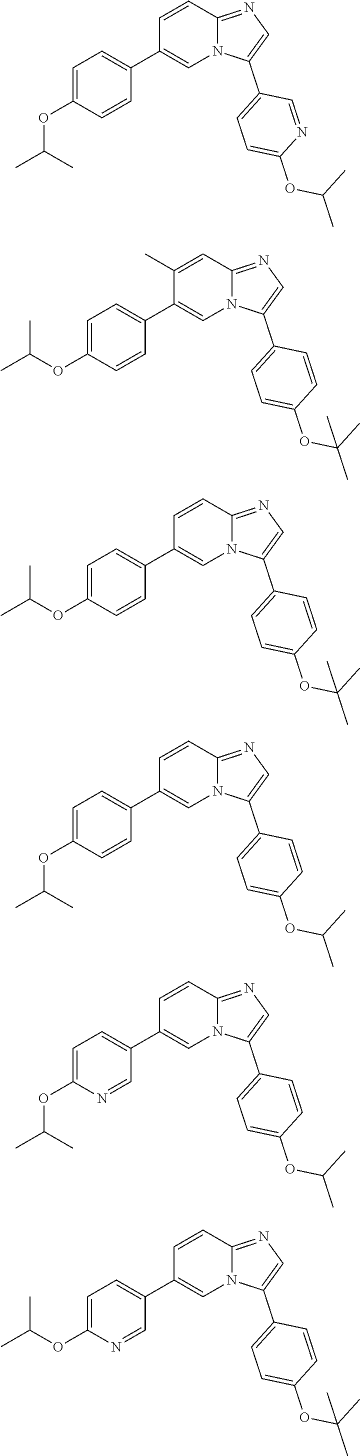

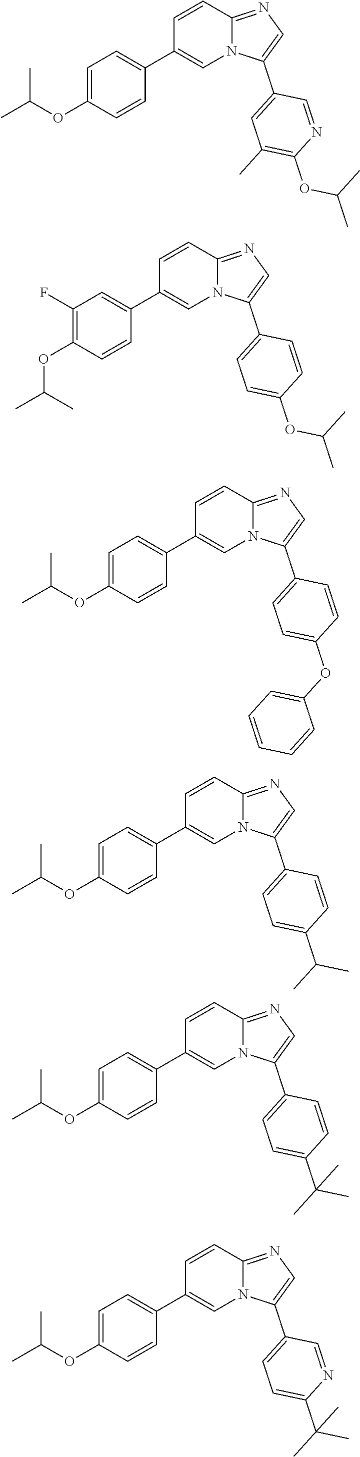

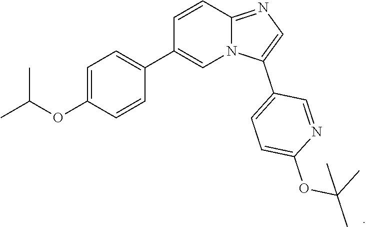 [SMILES: CC(C)Oc1ccc(-c2ccc3ncc(-c4ccc(OC(C)(C)C)nc4)n3c2)cc1]

35. A pharmaceutical composition comprising a therapeutically effective amount of a compound, selected from:

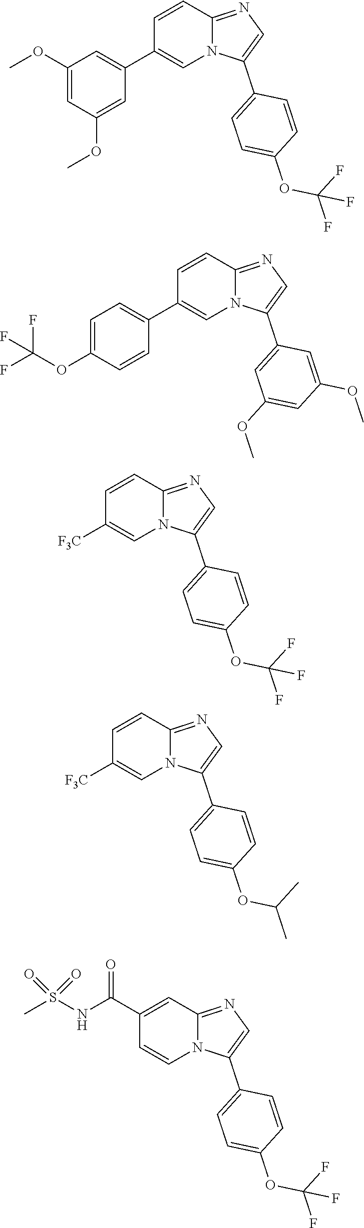

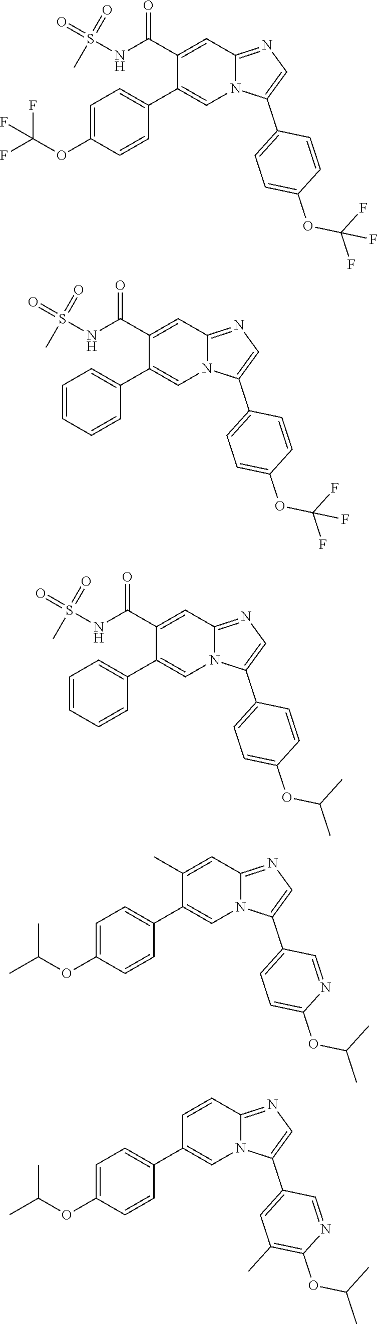

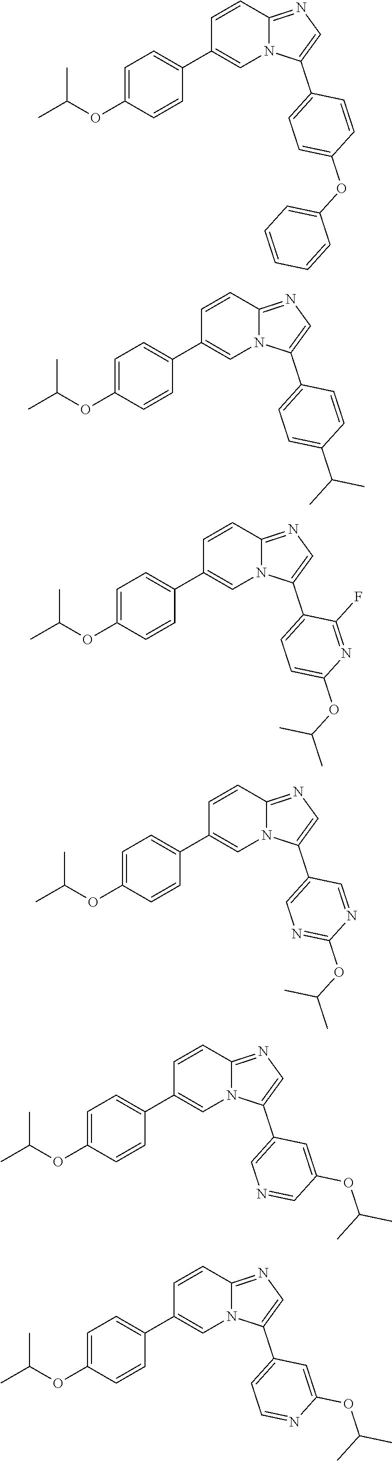

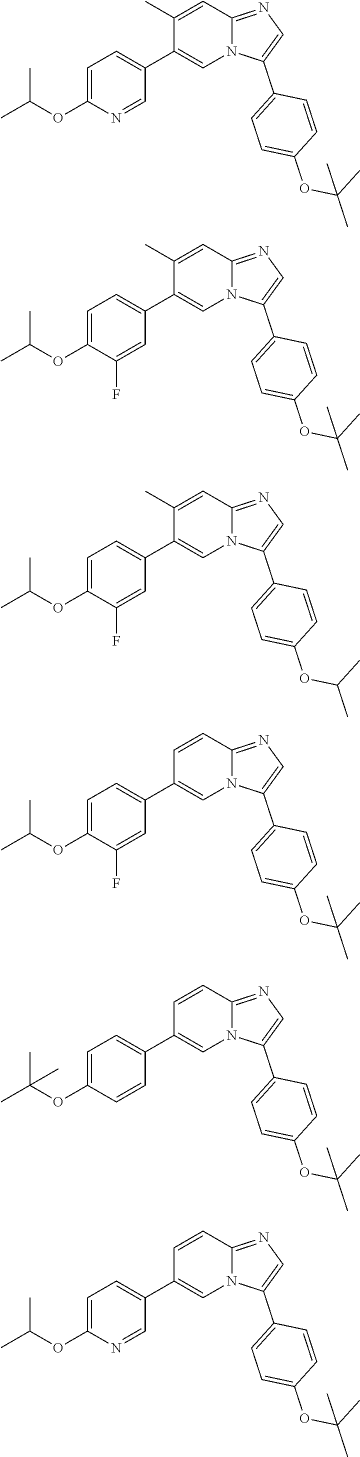

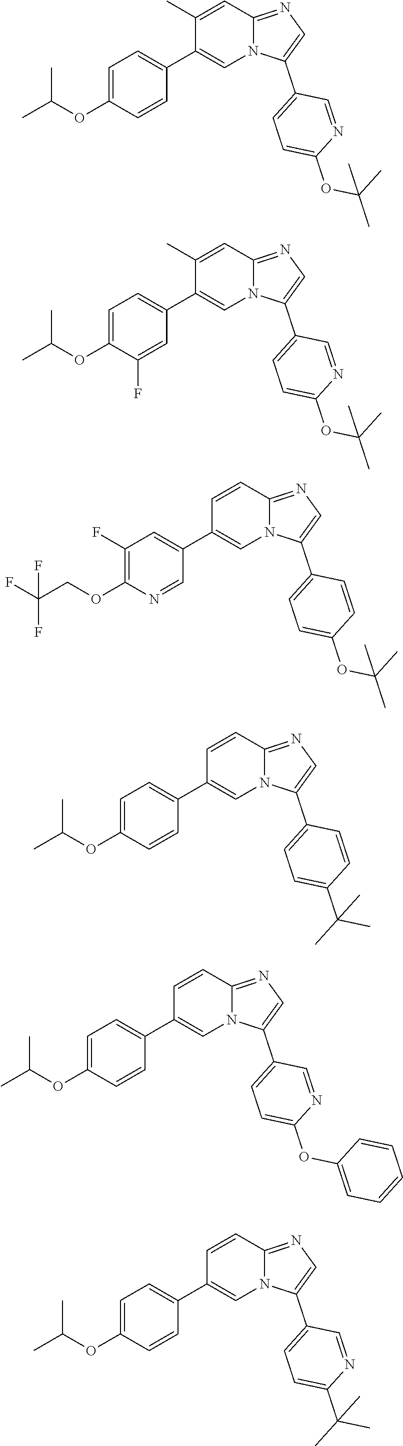

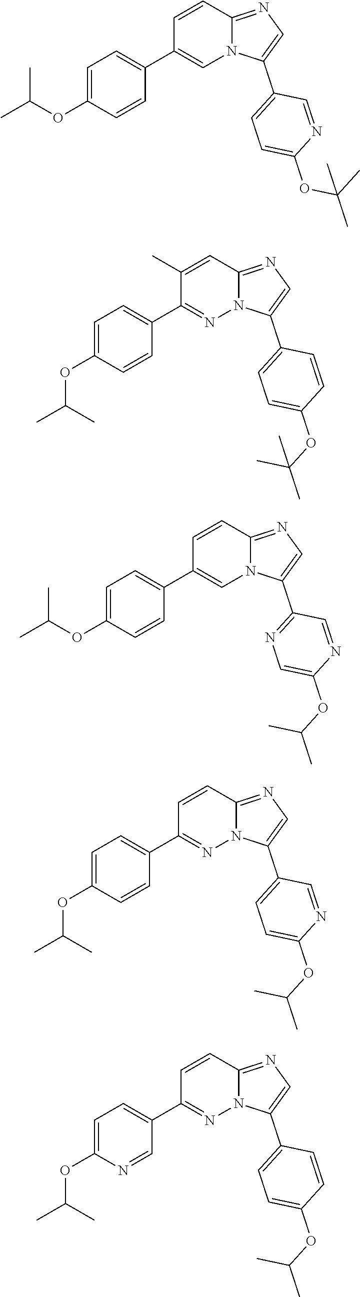

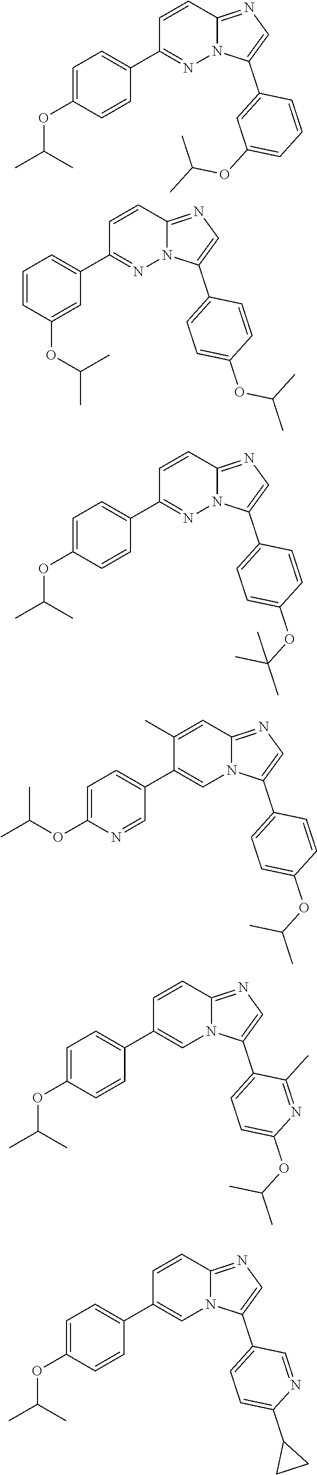

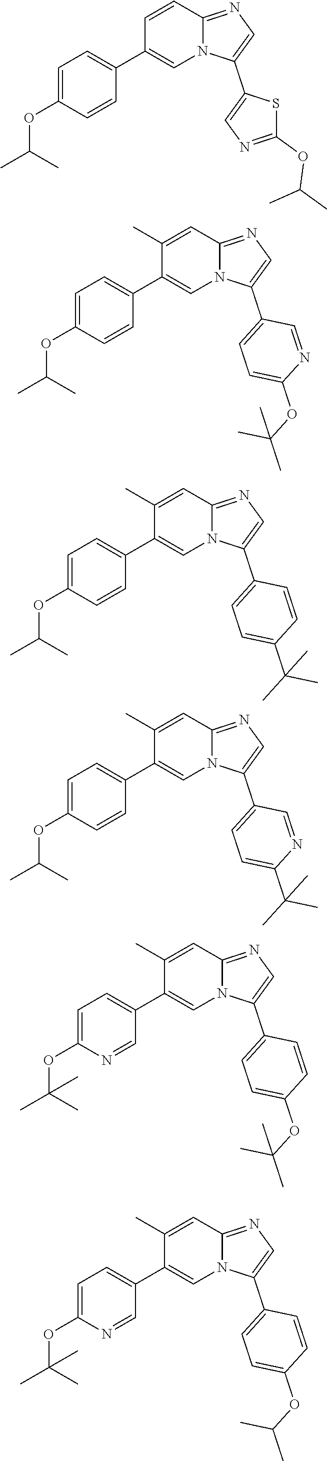

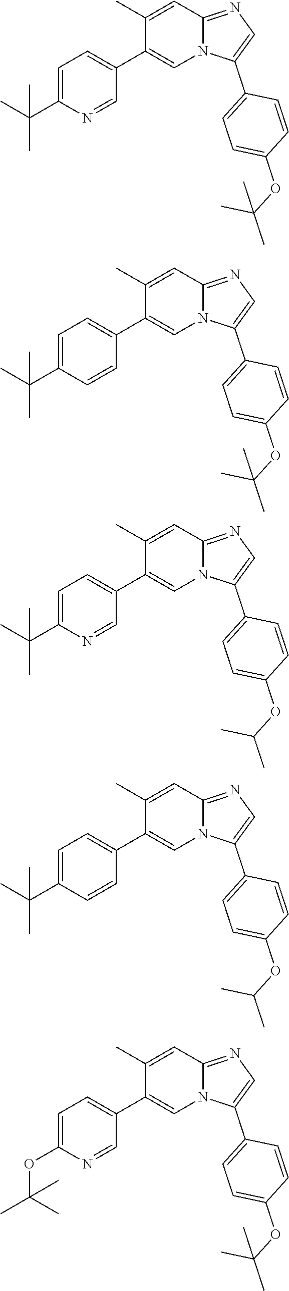

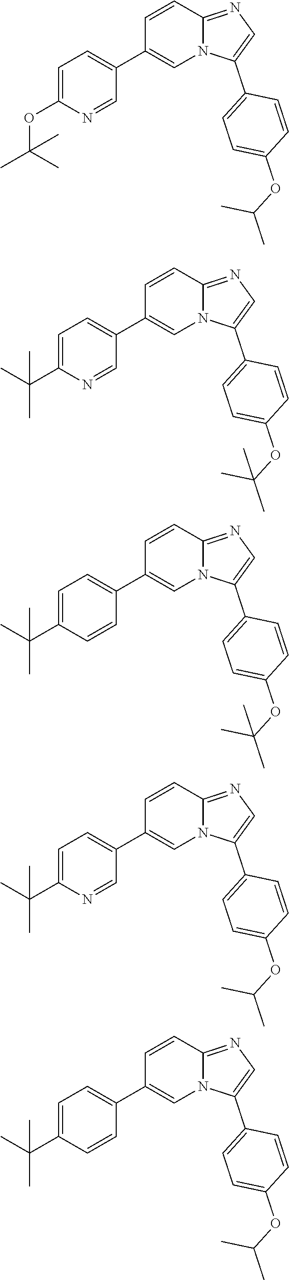

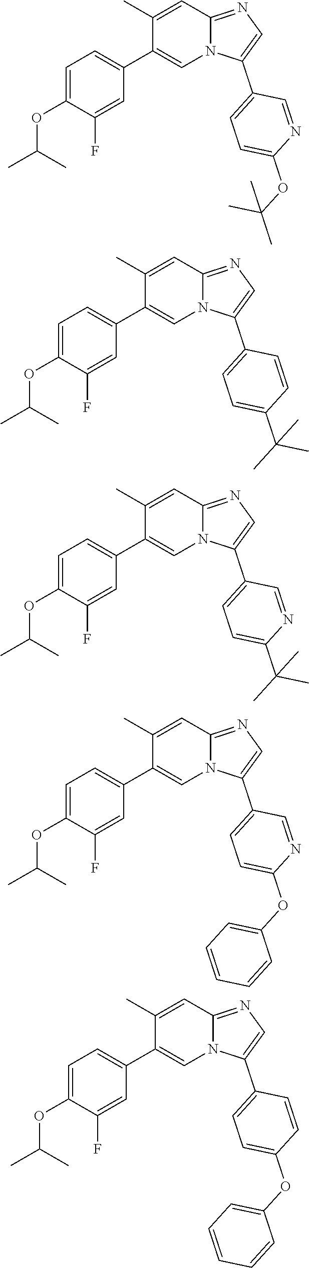

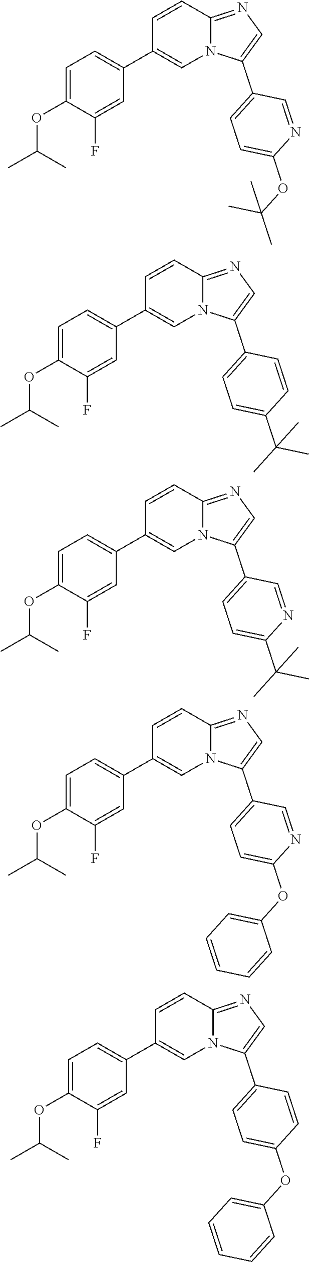

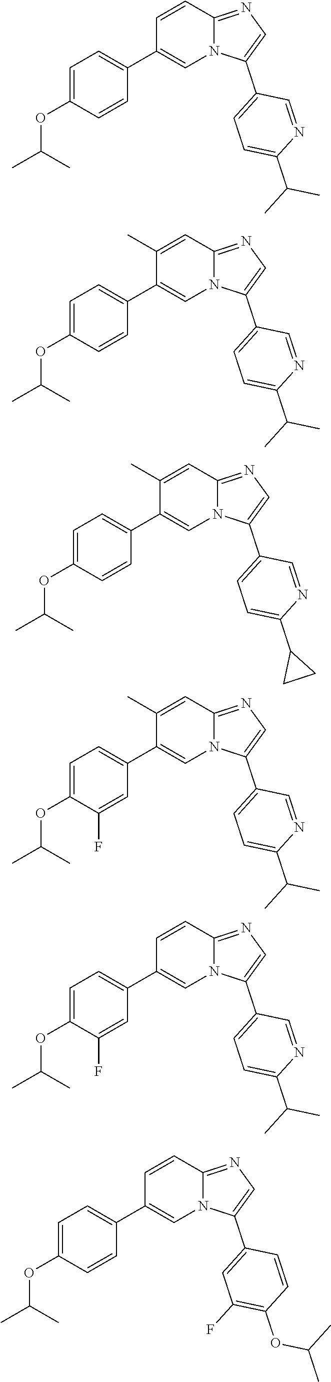

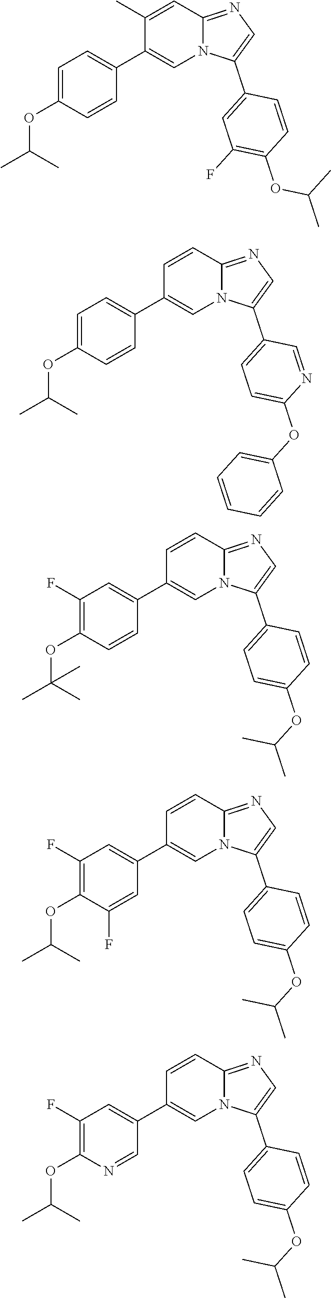

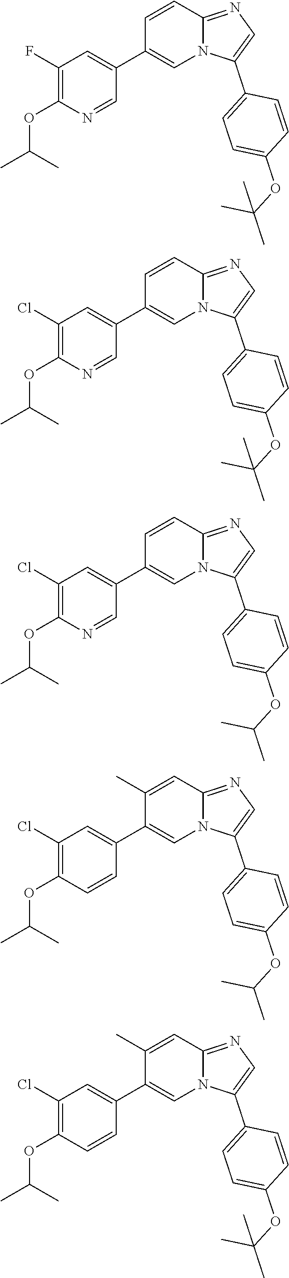

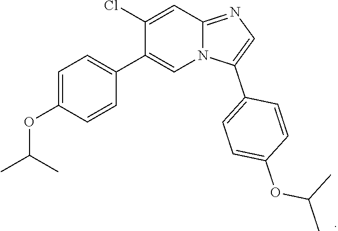 [SMILES: CC(C)Oc1ccc(-c2cn3c(-c4ccc(OC(C)C)cc4)cnc3cc2Cl)cc1]

36. The pharmaceutical composition of claim 35 wherein the compound is selected from the group consisting of 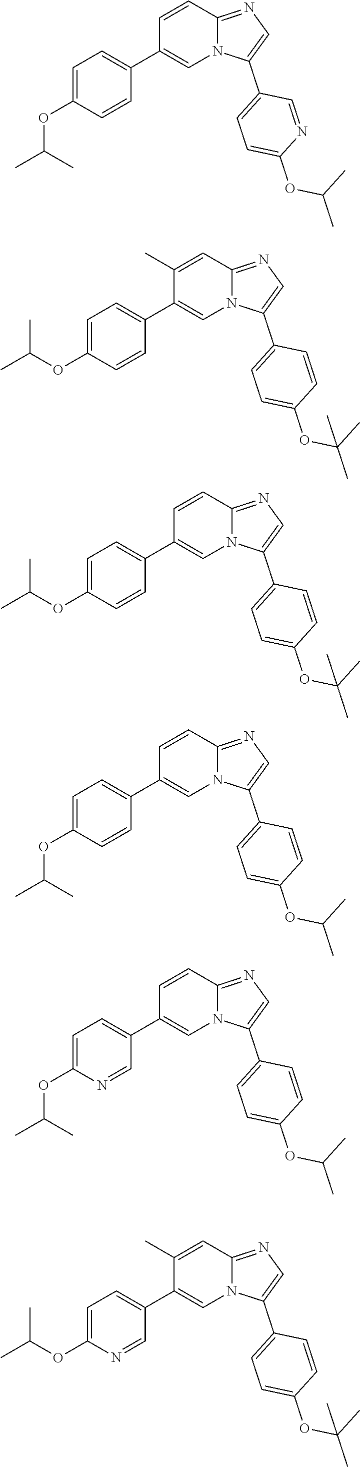 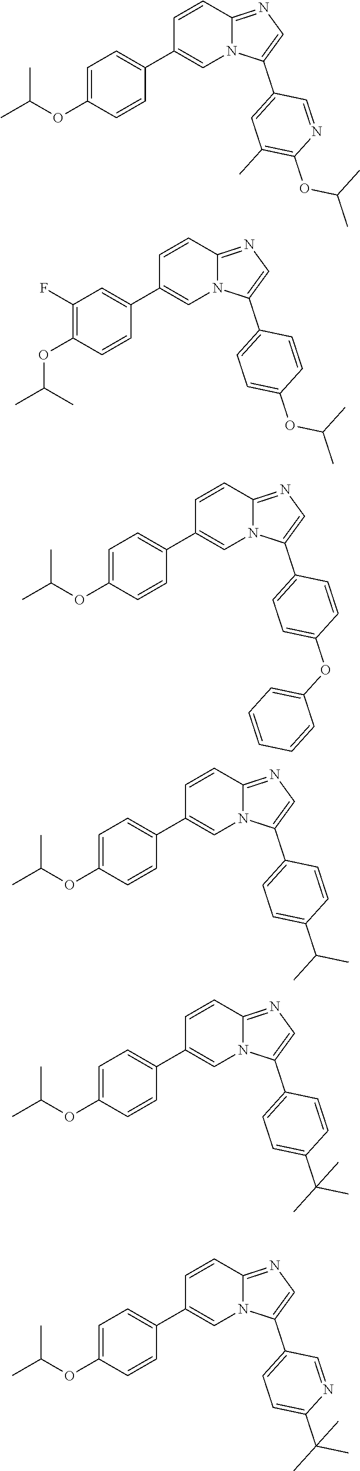 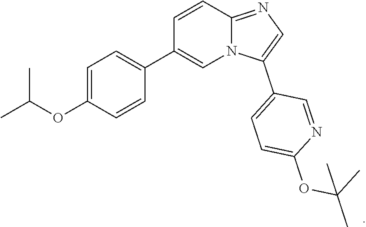 [SMILES: CC(C)Oc1ccc(-c2ccc3ncc(-c4ccc(OC(C)(C)C)nc4)n3c2)cc1]
